SEQUENCE LISTING (235 sequences)

SEQ ID NO: 1 - protein, 19 aa, synthetic construct
MGWSCIILFLVATATGVHS

SEQ ID NO: 2 - protein, 676 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 218 aa, synthetic construct
DIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - skipped

SEQ ID NO: 5 - DNA, 57 bp, synthetic construct
  source 1..57: mol_type = other DNA
atgggctggagctgcattatcctgttcctggtggccacagccaccggcgtgcacagc

SEQ ID NO: 6 - DNA, 2028 bp, synthetic construct
  source 1..2028: mol_type = other DNA
caggtgcagctggtgcagtccggcgtggaggtgaagaagccaggcgcctccgtgaaggtgtcttgcaaggccagcggctacacattcaccaactactatatgtattgggtgcggcaggcacctggacagggcctggagtggatgggaggcatcaacccatccaatggcggcacaaacttcaatgagaagtttaagaatcgggtgaccctgaccacagatagctccaccacaaccgcctacatggagctgaagtctctgcagtttgacgatacagccgtgtactattgcgcccggagagactacagattcgatatgggctttgactattggggccagggcacactggtgaccgtgtctagcgccagcaccaagggtcctagcgtgttccctctggccccatgctcaagaagcacttccgagtctaccgccgctctggggtgtctggtgaaggactacttccccgaacctgtcaccgtgagctggaactccggggccctgacttccggtgtccacacctttcccgctgtgctgcagtctagtggcctgtactctctgagcagcgtggtcactgtgccttcctctagtctgggaactaagacctatacatgcaacgtggaccataaaccatccaataccaaggtcgataaacgcgtggagtctaagtacggaccaccatgccctccatgtccagcaccagagttcgaaggcggaccttccgtgttcctgtttccccctaagccaaaagacaccctgatgatctcccgaacaccagaggtcacttgcgtggtcgtggacgtgtctcaggaggaccccgaagtccagttcaactggtatgtggatggcgtcgaagtgcacaatgctaagaccaaaccaagagaggaacagtttaatagtacttaccgcgtcgtgtcagtcctgaccgtgctgcatcaggattggctgaacggcaaggagtataagtgcaaagtgagcaataagggactgccctcaagcatcgagaaaacaattagcaaggctaaaggacagcccagggagcctcaggtgtacactctgccacccagtcaggaggaaatgaccaagaaccaggtcagcctgacctgtctggtgaaagggttctatccctctgacattgcagtggagtgggaaagtaatggtcagcctgagaacaattacaagactacccctccagtgctggactctgatggaagtttctttctgtatagcagactgacagtggataaatccaggtggcaggaggggaacgtctttagttgctcagtgatgcacgaagccctgcacaatcattatactcagaagagcctgtccctgtctctgggcagcaccggttctaagtacggaccaccatgccctccatgtccagcaccagagttcgaaggcggaccttccgtgttcctgtttccccctaagccaaaagacaccctgatgatctcccgaacaccagaggtcacttgcgtggtcgtggacgtgtctcaggaggaccccgaagtccagttcaactggtatgtggatggcgtcgaagtgcacaatgctaagaccaaaccaagagaggaacagtttaatagtacttaccgcgtcgtgtcagtcctgaccgtgctgcatcaggattggctgaacggcaaggagtataagtgcaaagtgagcaataagggactgccctcaagcatcgagaaaacaattagcaaggctaaaggacagcccagggagcctcaggtgtacactctgccacccagtcaggaggaaatgaccaagaaccaggtcagcctgacctgtctggtgaaagggttctatccctctgacattgcagtggagtgggaaagtaatggtcagcctgagaacaattacaagactacccctccagtgctggactctgatggaagtttctttctgtatagcagactgacagtggataaatccaggtggcaggaggggaacgtctttagttgctcagtgatgcacgaagccctgcacaatcattatactcagaagagcctgtccctgtctctgggc

SEQ ID NO: 7 - DNA, 654 bp, synthetic construct
  source 1..654: mol_type = other DNA
gacatcgtgctgacacagtctccagccaccctgagcctgtccccaggagagagggccacactgtcctgtcgcgcctctaagggcgtgtctaccagcggctacagctatctgcactggtaccagcagaagccaggacaggcacctaggctgctgatctacctggcaagctatctggagtccggagtgccagcaagattctccggatctggaagcggaaccgacttcaccctgaccatctcctctctggagcccgaggatttcgccgtgtactattgtcagcacagcagggacctgcctctgacatttggcggcggcaccaaggtggagatcaagcgcacagtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 8 - protein, 668 aa, synthetic construct
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 9 - protein, 182 aa, synthetic construct
RDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 10 - protein, 92 aa, synthetic construct
LRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 11 - protein, 628 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 12 - protein, 538 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 13 - protein, 15 aa, synthetic construct
GGGGSGGGGSGGGGS

SEQ ID NO: 14 - protein, 673 aa, synthetic construct
EVKLLESGGGLVQPGGSLKLSCAASGFDFSRYWMSWVRQAPGKGLEWIGEINPDSSTIKYTPSLKDKFIISRDNAKNTLCLQLSKVRSEDSALYYCARMGYRLFDSWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 15 - protein, 218 aa, synthetic construct
NIVMTQATPSVPVTPGESVSISCRSSKSLLHSDGDTYLYWFLQRPGQSPQLLIYRMSNLVSGVPDRFSGSGSGTAFTLRISRVEAEDVGVYYCMQHLEYPYTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 676 aa, synthetic construct
EVQLQQSGTVLARPGASVKMSCKASGNTFTSYWMHWVKQRPGQGLEWIGAIYPGYSDTRYNQKFKGKATLTAVTSTSTAYMELSSLTNEDSAVYYCTKFIATVGGYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 17 - protein, 216 aa, synthetic construct
QIVLTQSPAIMSSSLGERVTMTCTASSSVSSSYLHWYQQKPGSSPKLWIYSTSNLASGVPARFSGSGSGTSYSLTISSMEAEDAATYYCHQYHRSPPIFTFGSGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 677 aa, synthetic construct
QIQLVQSGPELKKPGETVKISCKASGYTFTDYSMHWVKQAPGKGLKWMGWIKIETGNPTYADDFKGRFAFSLETSASTAYLQINNLKNEDTATYFCARDYYGYYYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 214 aa, synthetic construct
QIVLTQSPAIMSASLGERVTMTCTVSSSISSSYLHWYQQKPGSSPKLWIYSTSNLASGVPARFSGSGSGTSYSLTISSMEAEDAATYYCHQYHRSPLTFGAGTKLELKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 678 aa, synthetic construct
EVQLVESGGGLVQPGGSLRLSCAASGFTFSHYGMSWVRQTPDKRLELVATIDNNGGNTYYPDSVKGRFTISRDNAKNTLYLQMSSLKSEDTAMYYCARDAYYTYDYWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 21 - protein, 213 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYHKLPWTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 677 aa, synthetic construct
EVKLVESGGGLVQPGGSLKLSCAASGFTFSSYTMSWVRQTPEKRLEWVAYISYGGGDTYYPDTVKGRFTISRDNAKNTLYLQMSSLKSEDTAMYYCARQKVDGYFVAMDYWSQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 23 - protein, 217 aa, synthetic construct
VIVLTQSPASLAVSLGQRATISCRASERVDDYGISFMNWFQQKPGQPPKLLIYAASNQGSGVPARFSGSGSGTDFSLNIHPMEEDDTAMYFCQQNKEVPWTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 676 aa, synthetic construct
EVKLVESGGGLVKPGGSLKLSCAASGFTFSSYGMSWVRQTPEKRLEWVATISGGGRYTYYPDSVKGRFTISRDNAKNILYLQMSSLRSEDTALYYCASPYDGYYGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 25 - protein, 217 aa, synthetic construct
DIVLTQSPASLAVSLGQRATISCRASESVDNSGISFMNWFQQKPGQPPKLLIYAASNQGSGVPARFSGSGSGTDFSLNIHPMEEDDTAMYFCQQSKEVPWTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 671 aa, synthetic construct
EVQLQQSGTVLARPGASVRMSCKASGYSFSTYWMHWVKQRPGQGLEWIGGIYPGNSDTNYNQKFKGKAKLTAVTSASTAYMDLSSLTNEDSAVYYCTGGYFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 27 - protein, 213 aa, synthetic construct
DIKMTQSPSSLYASLGERVTITCKASQDINRYLTWFQQKPGKSPKTLIFRANRLVTGVPSRFIGSGSGQEYSLTISSLEYEDMGIYFCLQYDESPYTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 673 aa, synthetic construct
QIQLQQSGPEQVKPGASVKISCKASGYMFIDYYMNWVKQKPGQGLEWIGWIYPGSGSTKDNENFKGKATLTVDTSSSTAYMQLSSLTSEDTAVYFCARYGPRFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 213 aa, synthetic construct
DIQMTQSPSSLSASLGERVNLTCRASQEISGYLSWLQQKPDGTIKRLIYVASTLDSGVPERFSGSRSGSDYSLTISSLESEDFADYYCLQYASYPYTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 673 aa, synthetic construct
EVKLLESGGGLVQPGGSLKLSCEVSGFDFSRDWMSWVRQAPGKGLEWIGQISPDSTSINYKPSLKDKFIISRDNAKNTLYLQLSGVRSEDTALYHCARDTSGYPDYWGQGTSLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 31 - protein, 213 aa, synthetic construct
DILLTQSPSSMSVSLGDTVSITCHASQDISRNIGWLRQKPGKSFKGLIYHGTNLEDGVPSRFSGSGSGADYSLTISSLESEDFADYYCVQYAQFPYTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 625 aa, synthetic construct
EVKLLESGGGLVQPGGSLKLSCAASGFDFSRYWMSWVRQAPGKGLEWIGEINPDSSTIKYTPSLKDKFIISRDNAKNTLCLQLSKVRSEDSALYYCARMGYRLFDSWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 33 - protein, 535 aa, synthetic construct
EVKLLESGGGLVQPGGSLKLSCAASGFDFSRYWMSWVRQAPGKGLEWIGEINPDSSTIKYTPSLKDKFIISRDNAKNTLCLQLSKVRSEDSALYYCARMGYRLFDSWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 34 - protein, 628 aa, synthetic construct
EVQLQQSGTVLARPGASVKMSCKASGNTFTSYWMHWVKQRPGQGLEWIGAIYPGYSDTRYNQKFKGKATLTAVTSTSTAYMELSSLTNEDSAVYYCTKFIATVGGYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 35 - protein, 538 aa, synthetic construct
EVQLQQSGTVLARPGASVKMSCKASGNTFTSYWMHWVKQRPGQGLEWIGAIYPGYSDTRYNQKFKGKATLTAVTSTSTAYMELSSLTNEDSAVYYCTKFIATVGGYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 36 - protein, 629 aa, synthetic construct
QIQLVQSGPELKKPGETVKISCKASGYTFTDYSMHWVKQAPGKGLKWMGWIKIETGNPTYADDFKGRFAFSLETSASTAYLQINNLKNEDTATYFCARDYYGYYYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 37 - protein, 539 aa, synthetic construct
QIQLVQSGPELKKPGETVKISCKASGYTFTDYSMHWVKQAPGKGLKWMGWIKIETGNPTYADDFKGRFAFSLETSASTAYLQINNLKNEDTATYFCARDYYGYYYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 38 - protein, 630 aa, synthetic construct
EVQLVESGGGLVQPGGSLRLSCAASGFTFSHYGMSWVRQTPDKRLELVATIDNNGGNTYYPDSVKGRFTISRDNAKNTLYLQMSSLKSEDTAMYYCARDAYYTYDYWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 39 - protein, 540 aa, synthetic construct
EVQLVESGGGLVQPGGSLRLSCAASGFTFSHYGMSWVRQTPDKRLELVATIDNNGGNTYYPDSVKGRFTISRDNAKNTLYLQMSSLKSEDTAMYYCARDAYYTYDYWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 40 - protein, 629 aa, synthetic construct
EVKLVESGGGLVQPGGSLKLSCAASGFTFSSYTMSWVRQTPEKRLEWVAYISYGGGDTYYPDTVKGRFTISRDNAKNTLYLQMSSLKSEDTAMYYCARQKVDGYFVAMDYWSQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 41 - protein, 539 aa, synthetic construct
EVKLVESGGGLVQPGGSLKLSCAASGFTFSSYTMSWVRQTPEKRLEWVAYISYGGGDTYYPDTVKGRFTISRDNAKNTLYLQMSSLKSEDTAMYYCARQKVDGYFVAMDYWSQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 42 - protein, 628 aa, synthetic construct
EVKLVESGGGLVKPGGSLKLSCAASGFTFSSYGMSWVRQTPEKRLEWVATISGGGRYTYYPDSVKGRFTISRDNAKNILYLQMSSLRSEDTALYYCASPYDGYYGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 43 - protein, 538 aa, synthetic construct
EVKLVESGGGLVKPGGSLKLSCAASGFTFSSYGMSWVRQTPEKRLEWVATISGGGRYTYYPDSVKGRFTISRDNAKNILYLQMSSLRSEDTALYYCASPYDGYYGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 44 - protein, 623 aa, synthetic construct
EVQLQQSGTVLARPGASVRMSCKASGYSFSTYWMHWVKQRPGQGLEWIGGIYPGNSDTNYNQKFKGKAKLTAVTSASTAYMDLSSLTNEDSAVYYCTGGYFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 45 - protein, 533 aa, synthetic construct
EVQLQQSGTVLARPGASVRMSCKASGYSFSTYWMHWVKQRPGQGLEWIGGIYPGNSDTNYNQKFKGKAKLTAVTSASTAYMDLSSLTNEDSAVYYCTGGYFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 46 - protein, 625 aa, synthetic construct
QIQLQQSGPEQVKPGASVKISCKASGYMFIDYYMNWVKQKPGQGLEWIGWIYPGSGSTKDNENFKGKATLTVDTSSSTAYMQLSSLTSEDTAVYFCARYGPRFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 47 - protein, 535 aa, synthetic construct
QIQLQQSGPEQVKPGASVKISCKASGYMFIDYYMNWVKQKPGQGLEWIGWIYPGSGSTKDNENFKGKATLTVDTSSSTAYMQLSSLTSEDTAVYFCARYGPRFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 48 - protein, 625 aa, synthetic construct
EVKLLESGGGLVQPGGSLKLSCEVSGFDFSRDWMSWVRQAPGKGLEWIGQISPDSTSINYKPSLKDKFIISRDNAKNTLYLQLSGVRSEDTALYHCARDTSGYPDYWGQGTSLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 49 - protein, 535 aa, synthetic construct
EVKLLESGGGLVQPGGSLKLSCEVSGFDFSRDWMSWVRQAPGKGLEWIGQISPDSTSINYKPSLKDKFIISRDNAKNTLYLQLSGVRSEDTALYHCARDTSGYPDYWGQGTSLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 50 - protein, 585 aa, synthetic construct [sequence omitted]

SEQ ID NO: 51 - protein, 1031 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 52 - protein, 1028 aa, synthetic construct
EVKLLESGGGLVQPGGSLKLSCAASGFDFSRYWMSWVRQAPGKGLEWIGEINPDSSTIKYTPSLKDKFIISRDNAKNTLCLQLSKVRSEDSALYYCARMGYRLFDSWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 53 - protein, 1030 aa, synthetic construct
VQLQQSGTVLARPGASVKMSCKASGNTFTSYWMHWVKQRPGQGLEWIGAIYPGYSDTRYNQKFKGKATLTAVTSTSTAYMELSSLTNEDSAVYYCTKFIATVGGYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 54 - protein, 1032 aa, synthetic construct
QIQLVQSGPELKKPGETVKISCKASGYTFTDYSMHWVKQAPGKGLKWMGWIKIETGNPTYADDFKGRFAFSLETSASTAYLQINNLKNEDTATYFCARDYYGYYYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 55 - protein, 1032 aa, synthetic construct
VQLVESGGGLVQPGGSLRLSCAASGFTFSHYGMSWVRQTPDKRLELVATIDNNGGNTYYPDSVKGRFTISRDNAKNTLYLQMSSLKSEDTAMYYCARDAYYTYDYWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 56 - protein, 1032 aa, synthetic construct
EVKLVESGGGLVQPGGSLKLSCAASGFTFSSYTMSWVRQTPEKRLEWVAYISYGGGDTYYPDTVKGRFTISRDNAKNTLYLQMSSLKSEDTAMYYCARQKVDGYFVAMDYWSQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 57 - protein, 1031 aa, synthetic construct
EVKLVESGGGLVKPGGSLKLSCAASGFTFSSYGMSWVRQTPEKRLEWVATISGGGRYTYYPDSVKGRFTISRDNAKNILYLQMSSLRSEDTALYYCASPYDGYYGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 58 - protein, 1026 aa, synthetic construct
EVQLQQSGTVLARPGASVRMSCKASGYSFSTYWMHWVKQRPGQGLEWIGGIYPGNSDTNYNQKFKGKAKLTAVTSASTAYMDLSSLTNEDSAVYYCTGGYFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 59 - protein, 1028 aa, synthetic construct
QIQLQQSGPEQVKPGASVKISCKASGYMFIDYYMNWVKQKPGQGLEWIGWIYPGSGSTKDNENFKGKATLTVDTSSSTAYMQLSSLTSEDTAVYFCARYGPRFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 60 - protein, 1028 aa, synthetic construct
EVKLLESGGGLVQPGGSLKLSCEVSGFDFSRDWMSWVRQAPGKGLEWIGQISPDSTSINYKPSLKDKFIISRDNAKNTLYLQLSGVRSEDTALYHCARDTSGYPDYWGQGTSLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 61 - protein, 47 aa, synthetic construct
LKEAKEKAIEELKKAGITSDYYFDLINKAKTVEGVNALKDEILKALP

SEQ ID NO: 62 - protein, 493 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLKEAKEKAIEELKKAGITSDYYFDLINKAKTVEGVNALKDEILKALP

SEQ ID NO: 63 - protein, 490 aa, synthetic construct
EVKLLESGGGLVQPGGSLKLSCAASGFDFSRYWMSWVRQAPGKGLEWIGEINPDSSTIKYTPSLKDKFIISRDNAKNTLCLQLSKVRSEDSALYYCARMGYRLFDSWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLKEAKEKAIEELKKAGITSDYYFDLINKAKTVEGVNALKDEILKALP

SEQ ID NO: 64 - protein, 492 aa, synthetic construct
VQLQQSGTVLARPGASVKMSCKASGNTFTSYWMHWVKQRPGQGLEWIGAIYPGYSDTRYNQKFKGKATLTAVTSTSTAYMELSSLTNEDSAVYYCTKFIATVGGYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLKEAKEKAIEELKKAGITSDYYFDLINKAKTVEGVNALKDEILKALP

SEQ ID NO: 65 - protein, 494 aa, synthetic construct
QIQLVQSGPELKKPGETVKISCKASGYTFTDYSMHWVKQAPGKGLKWMGWIKIETGNPTYADDFKGRFAFSLETSASTAYLQINNLKNEDTATYFCARDYYGYYYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLKEAKEKAIEELKKAGITSDYYFDLINKAKTVEGVNALKDEILKALP

SEQ ID NO: 66 - protein, 494 aa, synthetic construct
VQLVESGGGLVQPGGSLRLSCAASGFTFSHYGMSWVRQTPDKRLELVATIDNNGGNTYYPDSVKGRFTISRDNAKNTLYLQMSSLKSEDTAMYYCARDAYYTYDYWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLKEAKEKAIEELKKAGITSDYYFDLINKAKTVEGVNALKDEILKALP

SEQ ID NO: 67 - protein, 494 aa, synthetic construct
EVKLVESGGGLVQPGGSLKLSCAASGFTFSSYTMSWVRQTPEKRLEWVAYISYGGGDTYYPDTVKGRFTISRDNAKNTLYLQMSSLKSEDTAMYYCARQKVDGYFVAMDYWSQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLKEAKEKAIEELKKAGITSDYYFDLINKAKTVEGVNALKDEILKALP

SEQ ID NO: 68 - protein, 493 aa, synthetic construct
EVKLVESGGGLVKPGGSLKLSCAASGFTFSSYGMSWVRQTPEKRLEWVATISGGGRYTYYPDSVKGRFTISRDNAKNILYLQMSSLRSEDTALYYCASPYDGYYGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLKEAKEKAIEELKKAGITSDYYFDLINKAKTVEGVNALKDEILKALP

SEQ ID NO: 69 - protein, 488 aa, synthetic construct
EVQLQQSGTVLARPGASVRMSCKASGYSFSTYWMHWVKQRPGQGLEWIGGIYPGNSDTNYNQKFKGKAKLTAVTSASTAYMDLSSLTNEDSAVYYCTGGYFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLKEAKEKAIEELKKAGITSDYYFDLINKAKTVEGVNALKDEILKALP

SEQ ID NO: 70 - protein, 490 aa, synthetic construct
QIQLQQSGPEQVKPGASVKISCKASGYMFIDYYMNWVKQKPGQGLEWIGWIYPGSGSTKDNENFKGKATLTVDTSSSTAYMQLSSLTSEDTAVYFCARYGPRFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLKEAKEKAIEELKKAGITSDYYFDLINKAKTVEGVNALKDEILKALP

SEQ ID NO: 71 - protein, 490 aa, synthetic construct
EVKLLESGGGLVQPGGSLKLSCEVSGFDFSRDWMSWVRQAPGKGLEWIGQISPDSTSINYKPSLKDKFIISRDNAKNTLYLQLSGVRSEDTALYHCARDTSGYPDYWGQGTSLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGLKEAKEKAIEELKKAGITSDYYFDLINKAKTVEGVNALKDEILKALP

SEQ ID NO: 72 - protein, 117 aa, synthetic construct
EVKLLESGGGLVQPGGSLKLSCAASGFDFSRYWMSWVRQAPGKGLEWIGEINPDSSTIKYTPSLKDKFIISRDNAKNTLCLQLSKVRSEDSALYYCARMGYRLFDSWGQGTTLTVSS

SEQ ID NO: 73 - protein, 112 aa, synthetic construct
NIVMTQATPSVPVTPGESVSISCRSSKSLLHSDGDTYLYWFLQRPGQSPQLLIYRMSNLVSGVPDRFSGSGSGTAFTLRISRVEAEDVGVYYCMQHLEYPYTFGGGTKLEIK

SEQ ID NO: 74 - protein, 120 aa, synthetic construct
EVQLQQSGTVLARPGASVKMSCKASGNTFTSYWMHWVKQRPGQGLEWIGAIYPGYSDTRYNQKFKGKATLTAVTSTSTAYMELSSLTNEDSAVYYCTKFIATVGGYFDVWGAGTTVTVSS

SEQ ID NO: 75 - protein, 110 aa, synthetic construct
QIVLTQSPAIMSSSLGERVTMTCTASSSVSSSYLHWYQQKPGSSPKLWIYSTSNLASGVPARFSGSGSGTSYSLTISSMEAEDAATYYCHQYHRSPPIFTFGSGTKLEIK

SEQ ID NO: 76 - protein, 121 aa, synthetic construct
QIQLVQSGPELKKPGETVKISCKASGYTFTDYSMHWVKQAPGKGLKWMGWIKIETGNPTYADDFKGRFAFSLETSASTAYLQINNLKNEDTATYFCARDYYGYYYYAMDYWGQGTSVTVSS

SEQ ID NO: 77 - protein, 108 aa, synthetic construct
QIVLTQSPAIMSASLGERVTMTCTVSSSISSSYLHWYQQKPGSSPKLWIYSTSNLASGVPARFSGSGSGTSYSLTISSMEAEDAATYYCHQYHRSPLTFGAGTKLELK

SEQ ID NO: 78 - protein, 122 aa, synthetic construct
EVQLVESGGGLVQPGGSLRLSCAASGFTFSHYGMSWVRQTPDKRLELVATIDNNGGNTYYPDSVKGRFTISRDNAKNTLYLQMSSLKSEDTAMYYCARDAYYTYDYWYFDVWGAGTTVTVSS

SEQ ID NO: 79 - protein, 107 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYHKLPWTFGGGTKLEIK

SEQ ID NO: 80 - protein, 121 aa, synthetic construct
EVKLVESGGGLVQPGGSLKLSCAASGFTFSSYTMSWVRQTPEKRLEWVAYISYGGGDTYYPDTVKGRFTISRDNAKNTLYLQMSSLKSEDTAMYYCARQKVDGYFVAMDYWSQGTSVTVSS

SEQ ID NO: 81 - protein, 111 aa, synthetic construct
VIVLTQSPASLAVSLGQRATISCRASERVDDYGISFMNWFQQKPGQPPKLLIYAASNQGSGVPARFSGSGSGTDFSLNIHPMEEDDTAMYFCQQNKEVPWTFGGGTKLEIK

SEQ ID NO: 82 - protein, 120 aa, synthetic construct
EVKLVESGGGLVKPGGSLKLSCAASGFTFSSYGMSWVRQTPEKRLEWVATISGGGRYTYYPDSVKGRFTISRDNAKNILYLQMSSLRSEDTALYYCASPYDGYYGAMDYWGQGTSVTVSS

SEQ ID NO: 83 - protein, 111 aa, synthetic construct
DIVLTQSPASLAVSLGQRATISCRASESVDNSGISFMNWFQQKPGQPPKLLIYAASNQGSGVPARFSGSGSGTDFSLNIHPMEEDDTAMYFCQQSKEVPWTFGGGTKLEIK

SEQ ID NO: 84 - protein, 115 aa, synthetic construct
EVQLQQSGTVLARPGASVRMSCKASGYSFSTYWMHWVKQRPGQGLEWIGGIYPGNSDTNYNQKFKGKAKLTAVTSASTAYMDLSSLTNEDSAVYYCTGGYFFDVWGAGTTVTVSS

SEQ ID NO: 85 - protein, 107 aa, synthetic construct
DIKMTQSPSSLYASLGERVTITCKASQDINRYLTWFQQKPGKSPKTLIFRANRLVTGVPSRFIGSGSGQEYSLTISSLEYEDMGIYFCLQYDESPYTFGGGTKLEIK

SEQ ID NO: 86 - protein, 117 aa, synthetic construct
QIQLQQSGPEQVKPGASVKISCKASGYMFIDYYMNWVKQKPGQGLEWIGWIYPGSGSTKDNENFKGKATLTVDTSSSTAYMQLSSLTSEDTAVYFCARYGPRFFDVWGAGTTVTVSS

SEQ ID NO: 87 - protein, 107 aa, synthetic construct
DIQMTQSPSSLSASLGERVNLTCRASQEISGYLSWLQQKPDGTIKRLIYVASTLDSGVPERFSGSRSGSDYSLTISSLESEDFADYYCLQYASYPYTFGGGTKLEIK

SEQ ID NO: 88 - protein, 117 aa, synthetic construct
EVKLLESGGGLVQPGGSLKLSCEVSGFDFSRDWMSWVRQAPGKGLEWIGQISPDSTSINYKPSLKDKFIISRDNAKNTLYLQLSGVRSEDTALYHCARDTSGYPDYWGQGTSLTVSS

SEQ ID NO: 89 - protein, 107 aa, synthetic construct
DILLTQSPSSMSVSLGDTVSITCHASQDISRNIGWLRQKPGKSFKGLIYHGTNLEDGVPSRFSGSGSGADYSLTISSLESEDFADYYCVQYAQFPYTFGGGTKLEIK

SEQ ID NO: 90 - protein, 677 aa, synthetic construct
EVKLVESGGGLVQPGGSLKLSCAASGFTFSSYTMSWVRQTPEKRLEWVAYISYGGGDTYYSDTVKGRFTISRDNAKNTLYLQMSSLKSEDTAMYYCARQKVDGYYVGMDYWGQGSSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 91 - protein, 213 aa, synthetic construct
NIVMTQSPKSMSMSVGERVTLSCKASENVGTNVSWYQQKPEQSPRLLIYGASNRYTGVPDRFTGSGSVTDFTLTISSVQAEDLVDYHCGQSYSSPLTFGSGTKLELKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 676 aa, synthetic construct
EVQLVESGGGLVKPGGSLRLSCAASGFTFSSYGMSWVRQAPGKGLEWVATISGGGRYTYYPDSVKGRFTISRDNAKNSLYLQMNSLRAEDTAVYYCASPYDGYYGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGSTGSKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 93 - protein, 218 aa, synthetic construct
EIVLTQSPATLSLSPGERATLSCRASESVDNSGISFMNWYQQKPGQAPRLLIYAASNQGSGIPARFSGSGSGTDFTLTISSLEPEDFAVYYCQQSKEVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 5 aa, synthetic construct
RYWMS

SEQ ID NO: 95 - protein, 17 aa, synthetic construct
EINPDSSTIKYTPSLKD

SEQ ID NO: 96 - protein, 8 aa, synthetic construct
MGYRLFDS

SEQ ID NO: 97 - protein, 5 aa, synthetic construct
SYWMH

SEQ ID NO: 98 - protein, 17 aa, synthetic construct
AIYPGYSDTRYNQKFKG

SEQ ID NO: 99 - protein, 11 aa, synthetic construct
FIATVGGYFDV

SEQ ID NO: 100 - protein, 5 aa, synthetic construct
DYSMH

SEQ ID NO: 101 - protein, 17 aa, synthetic construct
WIKIETGNPTYADDFKG

SEQ ID NO: 102 - protein, 12 aa, synthetic construct
DYYGYYYYAMDY

SEQ ID NO: 103 - protein, 121 aa, synthetic construct
EVKLVESGGGLVQPGGSLKLSCAASGFTFSSYTMSWVRQTPEKRLEWVAYISYGGGDTYYSDTVKGRFTISRDNAKNTLYLQMSSLKSEDTAMYYCARQKVDGYYVGMDYWGQGSSVTVSS

SEQ ID NO: 104 - protein, 5 aa, synthetic construct
SYTMS

SEQ ID NO: 105 - protein, 17 aa, synthetic construct
YISYGGGDTYYSDTVKG

SEQ ID NO: 106 - protein, 12 aa, synthetic construct
QKVDGYYVGMDY

SEQ ID NO: 107 - protein, 5 aa, synthetic construct
HYGMS

SEQ ID NO: 108 - protein, 17 aa, synthetic construct
TIDNNGGNTYYPDSVKG

SEQ ID NO: 109 - protein, 13 aa, synthetic construct
DAYYTYDYWYFDV

SEQ ID NO: 110 - protein, 17 aa, synthetic construct
YISYGGGDTYYPDTVKG

SEQ ID NO: 111 - protein, 12 aa, synthetic construct
QKVDGYFVAMDY

SEQ ID NO: 112 - protein, 7 aa, synthetic construct
GFTFSSY

SEQ ID NO: 113 - protein, 6 aa, synthetic construct
SGGGRY

SEQ ID NO: 114 - protein, 15 aa, synthetic construct
CASPYDGYYGAMDYW

SEQ ID NO: 115 - protein, 5 aa, synthetic construct
TYWMH

SEQ ID NO: 116 - protein, 17 aa, synthetic construct
GIYPGNSDTNYNQKFKG

SEQ ID NO: 117 - protein, 6 aa, synthetic construct
GYFFDV

SEQ ID NO: 118 - protein, 5 aa, synthetic construct
DYYMN

SEQ ID NO: 119 - protein, 17 aa, synthetic construct
WIYPGSGSTKDNENFKG

SEQ ID NO: 120 - protein, 8 aa, synthetic construct
YGPRFFDV

SEQ ID NO: 121 - protein, 5 aa, synthetic construct
RDWMS

SEQ ID NO: 122 - protein, 17 aa, synthetic construct
QISPDSTSINYKPSLKD

SEQ ID NO: 123 - protein, 8 aa, synthetic construct
DTSGYPDY

SEQ ID NO: 124 - protein, 30 aa, synthetic construct
EVKLLESGGGLVQPGGSLKLSCAASGFDFS

SEQ ID NO: 125 - protein, 14 aa, synthetic construct
WVRQAPGKGLEWIG

SEQ ID NO: 126 - protein, 32 aa, synthetic construct
KFIISRDNAKNTLCLQLSKVRSEDSALYYCAR

SEQ ID NO: 127 - protein, 11 aa, synthetic construct
WGQGTTLTVSS

SEQ ID NO: 128 - protein, 30 aa, synthetic construct
EVQLQQSGTVLARPGASVKMSCKASGNTFT

SEQ ID NO: 129 - protein, 14 aa, synthetic construct
WVKQRPGQGLEWIG

SEQ ID NO: 130 - protein, 32 aa, synthetic construct
KATLTAVTSTSTAYMELSSLTNEDSAVYYCTK

SEQ ID NO: 131 - protein, 11 aa, synthetic construct
WGAGTTVTVSS

SEQ ID NO: 132 - protein, 30 aa, synthetic construct
QIQLVQSGPELKKPGETVKISCKASGYTFT

SEQ ID NO: 133 - protein, 14 aa, synthetic construct
WVKQAPGKGLKWMG

SEQ ID NO: 134 - protein, 32 aa, synthetic construct
RFAFSLETSASTAYLQINNLKNEDTATYFCAR

SEQ ID NO: 135 - protein, 11 aa, synthetic construct
WGQGTSVTVSS

SEQ ID NO: 136 - protein, 30 aa, synthetic construct
EVKLVESGGGLVQPGGSLKLSCAASGFTFS

SEQ ID NO: 137 - protein, 14 aa, synthetic construct
WVRQTPEKRLEWVA

SEQ ID NO: 138 - protein, 32 aa, synthetic construct
RFTISRDNAKNTLYLQMSSLKSEDTAMYYCAR

SEQ ID NO: 139 - protein, 11 aa, synthetic construct
WGQGSSVTVSS

SEQ ID NO: 140 - protein, 30 aa, synthetic construct
EVQLVESGGGLVQPGGSLRLSCAASGFTFS

SEQ ID NO: 141 - protein, 14 aa, synthetic construct
WVRQTPDKRLELVA

SEQ ID NO: 142 - protein, 11 aa, synthetic construct
WSQGTSVTVSS

SEQ ID NO: 143 - protein, 25 aa, synthetic construct
EVKLVESGGGLVKPGGSLKLSCAAS

SEQ ID NO: 144 - protein, 19 aa, synthetic construct
GMSWVRQTPEKRLEWVATI

SEQ ID NO: 145 - protein, 38 aa, synthetic construct
TYYPDSVKGRFTISRDNAKNILYLQMSSLRSEDTALYY

SEQ ID NO: 146 - protein, 10 aa, synthetic construct
GQGTSVTVSS

SEQ ID NO: 147 - protein, 30 aa, synthetic construct
EVQLQQSGTVLARPGASVRMSCKASGYSFS

SEQ ID NO: 148 - protein, 32 aa, synthetic construct
KAKLTAVTSASTAYMDLSSLTNEDSAVYYCTG

SEQ ID NO: 149 - protein, 30 aa, synthetic construct
QIQLQQSGPEQVKPGASVKISCKASGYMFI

SEQ ID NO: 150 - protein, 14 aa, synthetic construct
WVKQKPGQGLEWIG

SEQ ID NO: 151 - protein, 32 aa, synthetic construct
KATLTVDTSSSTAYMQLSSLTSEDTAVYFCAR

SEQ ID NO: 152 - protein, 30 aa, synthetic construct
EVKLLESGGGLVQPGGSLKLSCEVSGFDFS

SEQ ID NO: 153 - protein, 32 aa, synthetic construct
KFIISRDNAKNTLYLQLSGVRSEDTALYHCAR

SEQ ID NO: 154 - protein, 11 aa, synthetic construct
WGQGTSLTVSS

SEQ ID NO: 155 - protein, 16 aa, synthetic construct
RSSKSLLHSDGDTYLY

SEQ ID NO: 156 - protein, 7 aa, synthetic construct
RMSNLVS

SEQ ID NO: 157 - protein, 9 aa, synthetic construct
MQHLEYPYT

SEQ ID NO: 158 - protein, 12 aa, synthetic construct
TASSSVSSSYLH

SEQ ID NO: 159 - protein, 7 aa, synthetic construct
STSNLAS

SEQ ID NO: 160 - protein, 11 aa, synthetic construct
HQYHRSPPIFT

SEQ ID NO: 161 - protein, 12 aa, synthetic construct
TVSSSISSSYLH

SEQ ID NO: 162 - protein, 9 aa, synthetic construct
HQYHRSPLT

SEQ ID NO: 163 - protein, 107 aa, synthetic construct
NIVMTQSPKSMSMSVGERVTLSCKASENVGTNVSWYQQKPEQSPRLLIYGASNRYTGVPDRFTGSGSVTDFTLTISSVQAEDLVDYHCGQSYSSPLTFGSGTKLELK

SEQ ID NO: 164 - protein, 11 aa, synthetic construct
KASENVGTNVS

SEQ ID NO: 165 - protein, 7 aa, synthetic construct
GASNRYT

SEQ ID NO: 166 - protein, 9 aa, synthetic construct
GQSYSSPLT

SEQ ID NO: 167 - protein, 11 aa, synthetic construct
SASQGISNYLN

SEQ ID NO: 168 - protein, 7 aa, synthetic construct
YTSSLHS

SEQ ID NO: 169 - protein, 9 aa, synthetic construct
QQYHKLPWT

SEQ ID NO: 170 - protein, 15 aa, synthetic construct
RASERVDDYGISFMN

SEQ ID NO: 171 - protein, 7 aa, synthetic construct
AASNQGS

SEQ ID NO: 172 - protein, 9 aa, synthetic construct
QQNKEVPWT

SEQ ID NO: 173 - protein, 15 aa, synthetic construct
RASESVDNSGISFMN

SEQ ID NO: 174 - protein, 11 aa, synthetic construct
CQQSKEVPWTF

SEQ ID NO: 175 - protein, 11 aa, synthetic construct
KASQDINRYLT

SEQ ID NO: 176 - protein, 7 aa, synthetic construct
RANRLVT

SEQ ID NO: 177 - protein, 9 aa, synthetic construct
LQYDESPYT

SEQ ID NO: 178 - protein, 11 aa, synthetic construct
RASQEISGYLS

SEQ ID NO: 179 - protein, 7 aa, synthetic construct
VASTLDS

SEQ ID NO: 180 - protein, 9 aa, synthetic construct
LQYASYPYT

SEQ ID NO: 181 - protein, 11 aa, synthetic construct
HASQDISRNIG

SEQ ID NO: 182 - protein, 7 aa, synthetic construct
HGTNLED

SEQ ID NO: 183 - protein, 9 aa, synthetic construct
VQYAQFPYT

SEQ ID NO: 184 - protein, 23 aa, synthetic construct
NIVMTQATPSVPVTPGESVSISC

SEQ ID NO: 185 - protein, 15 aa, synthetic construct
WFLQRPGQSPQLLIY

SEQ ID NO: 186 - protein, 32 aa, synthetic construct
GVPDRFSGSGSGTAFTLRISRVEAEDVGVYYC

SEQ ID NO: 187 - protein, 10 aa, synthetic construct
FGGGTKLEIK

SEQ ID NO: 188 - protein, 23 aa, synthetic construct
QIVLTQSPAIMSSSLGERVTMTC

SEQ ID NO: 189 - protein, 15 aa, synthetic construct
WYQQKPGSSPKLWIY

SEQ ID NO: 190 - protein, 32 aa, synthetic construct
GVPARFSGSGSGTSYSLTISSMEAEDAATYYC

SEQ ID NO: 191 - protein, 10 aa, synthetic construct
FGSGTKLEIK

SEQ ID NO: 192 - protein, 23 aa, synthetic construct
QIVLTQSPAIMSASLGERVTMTC

SEQ ID NO: 193 - protein, 10 aa, synthetic construct
FGAGTKLELK

SEQ ID NO: 194 - protein, 23 aa, synthetic construct
NIVMTQSPKSMSMSVGERVTLSC

SEQ ID NO: 195 - protein, 15 aa, synthetic construct
WYQQKPEQSPRLLIY

SEQ ID NO: 196 - protein, 32 aa, synthetic construct
GVPDRFTGSGSVTDFTLTISSVQAEDLVDYHC

SEQ ID NO: 197 - protein, 10 aa, synthetic construct
FGSGTKLELK

SEQ ID NO: 198 - protein, 23 aa, synthetic construct
DIQMTQTTSSLSASLGDRVTISC

SEQ ID NO: 199 - protein, 15 aa, synthetic construct
WYQQKPDGTVKLLIY

SEQ ID NO: 200 - protein, 32 aa, synthetic construct
GVPSRFSGSGSGTDYSLTISNLEPEDIATYYC

SEQ ID NO: 201 - protein, 23 aa, synthetic construct
VIVLTQSPASLAVSLGQRATISC

SEQ ID NO: 202 - protein, 15 aa, synthetic construct
WFQQKPGQPPKLLIY

SEQ ID NO: 203 - protein, 32 aa, synthetic construct
GVPARFSGSGSGTDFSLNIHPMEEDDTAMYFC

SEQ ID NO: 204 - protein, 23 aa, synthetic construct
DIVLTQSPASLAVSLGQRATISC

SEQ ID NO: 205 - protein, 31 aa, synthetic construct
GVPARFSGSGSGTDFSLNIHPMEEDDTAMYF

SEQ ID NO: 206 - protein, 9 aa, synthetic construct
GGGTKLEIK

SEQ ID NO: 207 - protein, 23 aa, synthetic construct
DIKMTQSPSSLYASLGERVTITC

SEQ ID NO: 208 - protein, 15 aa, synthetic construct
WFQQKPGKSPKTLIF

SEQ ID NO: 209 - protein, 32 aa, synthetic construct
GVPSRFIGSGSGQEYSLTISSLEYEDMGIYFC

SEQ ID NO: 210 - protein, 23 aa, synthetic construct
DIQMTQSPSSLSASLGERVNLTC

SEQ ID NO: 211 - protein, 15 aa, synthetic construct
WLQQKPDGTIKRLIY

SEQ ID NO: 212 - protein, 32 aa, synthetic construct
GVPERFSGSRSGSDYSLTISSLESEDFADYYC

SEQ ID NO: 213 - protein, 23 aa, synthetic construct
DILLTQSPSSMSVSLGDTVSITC

SEQ ID NO: 214 - protein, 15 aa, synthetic construct
WLRQKPGKSFKGLIY

SEQ ID NO: 215 - protein, 32 aa, synthetic construct
GVPSRFSGSGSGADYSLTISSLESEDFADYYC

SEQ ID NO: 216 - DNA, 351 bp, synthetic construct
  source 1..351: mol_type = other DNA
gaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgataaaatacacgccatctctaaaggataaattcatcatctccagagacaacgccaaaaatacgctgtgcctgcaattgagcaaagtgagatctgaggactcagccctttattactgtgcaagaatgggctataggctctttgactcctggggccaaggcaccactctcacagtctcctca

SEQ ID NO: 217 - DNA, 336 bp, synthetic construct
  source 1..336: mol_type = other DNA
aatattgtgatgactcaggctacaccctctgtacctgtcactcctggagagtcagtatccatctcctgcaggtctagtaagagtctcctgcatagtgatggcgacacttacttgtattggttcctgcagaggccaggccagtctcctcagctcctgatatatcggatgtccaaccttgtctcaggagtcccagacaggttcagtggcagtgggtcaggaactgctttcacactgagaatcagtagagtggaggctgaggatgtgggtgtttattactgtatgcaacatctagaatatccgtacacgttcggaggggggaccaagctggaaataaaa

SEQ ID NO: 218 - DNA, 360 bp, synthetic construct
  source 1..360: mol_type = other DNA
gaggttcagctccagcagtctgggactgtgctggcaaggcctggggcttcagtgaagatgtcctgcaaggcttctggcaacacctttaccagctactggatgcactgggtaaaacagaggcctggacagggtctggaatggattggcgctatttatcctggatatagtgatactaggtacaatcagaagtttaagggcaaggccacactgactgcagtcacatccaccagcactgcctacatggagctcagcagcctgacaaatgaggactctgcggtctattactgtacaaaatttattgctacggtaggggggtacttcgatgtctggggcgcagggaccacggtcaccgtctcctca

SEQ ID NO: 219 - DNA, 330 bp, synthetic construct
  source 1..330: mol_type = other DNA
caaattgttctcacccagtctccagcaatcatgtcttcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacccatattcacgttcggctcggggacaaagttggaaataaaa

SEQ ID NO: 220 - DNA, 363 bp, synthetic construct
  source 1..363: mol_type = other DNA
cagatccagttggtgcagtctggacctgagctgaagaagcctggagagacagtcaagatctcctgcaaggcttctggttataccttcacagactattcaatgcactgggtgaagcaggctccaggaaagggtttaaagtggatgggctggataaaaattgagactggtaacccaacatatgcagatgacttcaagggacggtttgccttctctttggaaacctctgccagcactgcctatttgcagatcaacaacctcaaaaatgaggacacggctacatatttctgtgcgagagactactatggttattattactatgctatggactactggggtcaaggaacctcagtcaccgtctcctca

SEQ ID NO: 221 - DNA, 324 bp, synthetic construct
  source 1..324: mol_type = other DNA
caaattgttctcacccagtctccagcaatcatgtctgcatctctaggggaacgggtcaccatgacctgcactgttagctcaagtataagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccgctcacgttcggtgctgggaccaagctggagctgaaa

SEQ ID NO: 222 - DNA, 363 bp, synthetic construct
  source 1..363: mol_type = other DNA
gaagtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagttatggtggaggtgacacctactattcagacactgtaaagggccgattcaccatctccagagacaatgccaagaacaccctgtacctacaaatgagcagtctgaagtctgaggacacggccatgtattactgtgcaagacagaaggtcgatggttactacgttggtatggactactggggtcaaggatcctcagtcaccgtctcctca

SEQ ID NO: 223 - DNA, 321 bp, synthetic construct
  source 1..321: mol_type = other DNA
aacattgtaatgacccaatctcccaaatccatgtccatgtcagtaggagagagggtcaccttgagctgcaaggccagcgagaatgtgggtactaatgtatcctggtatcaacagaaaccagagcagtctcctagactgctgatatatggggcttccaaccggtacactggggtccccgatcgcttcacaggcagtggatctgtaacagatttcactctgaccatcagcagtgtgcaggctgaggaccttgtagattatcactgtggacagagttacagctctccgctcacgttcggttctgggaccaagctggagctgaaa

SEQ ID NO: 224 - DNA, 366 bp, synthetic construct
  source 1..366: mol_type = other DNA
gaggtgcagctggtggagtctgggggaggcttagtgcagcctggagggtccctgagactctcctgtgcagcctctggattcactttcagtcactatggcatgtcttgggttcgccagactccagacaagaggctggagttggtcgcaaccattgataataatggtggtaacacctattatccagacagtgtgaagggccgattcaccatctccagagacaatgccaagaacaccctgtacctgcaaatgagcagtctgaagtctgaggacacagccatgtattactgtgcaagagacgcctactatacttacgactactggtacttcgatgtctggggcgcagggaccacggtcaccgtctcctca

SEQ ID NO: 225 - DNA, 321 bp, synthetic construct
  source 1..321: mol_type = other DNA
gatatccagatgacacagactacatcctccctgtctgcctctctgggagacagagtcaccatcagttgcagtgcgagtcagggcattagtaattatttaaactggtatcagcagaaaccagatggaactgtgaaactcctgatctattacacatcaagtttacattcaggagtcccatcaaggttcagtggcagtgggtctgggacagattattctctcaccatcagcaacctagaacctgaagatattgccacttactattgtcaacagtatcataagcttccgtggacgttcggtggaggcaccaagctggaaatcaaa

SEQ ID NO: 226 - DNA, 363 bp, synthetic construct
  source 1..363: mol_type = other DNA
gaagtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagttatggtggaggtgacacgtactatccagacactgtaaagggccgattcaccatctccagagacaatgccaagaacaccctgtacctgcaaatgagcagtctgaagtctgaggacacggccatgtattactgtgccagacagaaggtcgatggttacttcgttgctatggactattggagtcaaggaacctcagtcaccgtctcctca

SEQ ID NO: 227 - DNA, 333 bp, synthetic construct
  source 1..333: mol_type = other DNA
gtcattgtgctgacccaatctccagcttctttggctgtgtctctagggcagagggccaccatctcctgcagagccagcgagcgtgttgatgattatggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaaataaggaggttccgtggacgttcggtggaggcaccaagctggaaatcaaa

SEQ ID NO: 228 - DNA, 360 bp, synthetic construct
  source 1..360: mol_type = other DNA
gaagtgaagctggtggagtctgggggaggcttagtgaagcctggagggtccctgaaactctcctgtgcagcctctggattcactttcagtagctatggcatgtcttgggttcgccagactccggagaagaggctggagtgggtcgcaaccatcagtggtggtggtagatacacctactatccagacagtgtgaaggggcgattcaccatctccagagacaatgccaagaacatcctgtacctgcaaatgagtagtctgaggtctgaggacacggccttgtattactgtgcaagcccctatgatggttactacggtgctatggactactggggtcaaggaacctcagtcaccgtctcctca

SEQ ID NO: 229 - DNA, 333 bp, synthetic construct
  source 1..333: mol_type = other DNA
gacattgtgctgacccaatctccagcttctttggctgtgtctctagggcagagggccaccatctcctgcagagccagcgaaagtgttgataattctggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccgtggacgttcggtggaggcaccaagctggaaatcaaa

SEQ ID NO: 230 - DNA, 345 bp, synthetic construct
  source 1..345: mol_type = other DNA
gaggttcagctccagcagtctgggactgtgctggcaaggcctggggcttccgtgaggatgtcctgcaaggcttctggctacagcttttccacctactggatgcactgggtaaaacagaggcctggacagggtctagaatggattggtggtatttatcctggaaatagtgacactaactacaaccagaagttcaagggcaaggccaaactgactgcagtcacttccgccagcactgcctacatggacctcagcagcctgacaaatgaggactctgcggtctattactgtacaggcggttacttcttcgatgtctggggcgcagggaccacggtcaccgtctcctca

SEQ ID NO: 231 - DNA, 321 bp, synthetic construct
  source 1..321: mol_type = other DNA
gacatcaagatgacccagtctccatcttccttgtatgcatctctaggagagagagtcactatcacttgcaaggcgagtcaggacattaatcgatatttaacctggttccagcagaaaccagggaaatctcctaagaccctgatctttcgtgcaaacagattggtaactggggtcccatcaaggttcattggcagtggatctgggcaagaatattctctcaccatcagcagcctggagtatgaagacatgggaatttatttttgtctacagtatgatgagtctccgtacacgttcggaggggggaccaagttggaaataaaa

SEQ ID NO: 232 - DNA, 351 bp, synthetic construct
  source 1..351: mol_type = other DNA
cagatccagctgcagcagtctggacctgagcaggtgaagcctggggcttcagtgaagatatcctgcaaggcttctggctacatgttcatcgactactatatgaactgggtgaagcagaagcctggacagggacttgagtggattggatggatttatcctggaagcggtagtactaaggacaatgagaatttcaagggcaaggccacattgactgtagacacatcctccagcacagcctacatgcagctcagtagcctgacatctgaggacactgctgtctatttctgtgcaagatatgggcccaggttcttcgatgtctggggcgcagggaccacggtcaccgtctcctca

SEQ ID NO: 233 - DNA, 321 bp, synthetic construct
  source 1..321: mol_type = other DNA
gacatccagatgacccagtctccatcctccttatctgcctctctgggagaaagagtcaatctcacttgtcgggcaagtcaggaaattagtggttacttaagctggcttcagcagaaaccagatggaactattaaacgcctgatctacgtcgcatccactttagattctggtgtcccagaaaggttcagtggcagtaggtctgggtcagattattctctcaccatcagcagccttgagtctgaggattttgcagactattactgtctacaatatgctagttatccgtacacgttcggaggggggaccaagctggaaataaaa

SEQ ID NO: 234 - DNA, 351 bp, synthetic construct
  source 1..351: mol_type = other DNA
gaggtgaagcttctcgagtctggaggtggcctggtgcagcctggaggatccctgaaactctcctgtgaagtctcaggattcgattttagcagagactggatgagttgggtccggcaggctccagggaaagggctagagtggattggacaaatttctccagatagcacttcgataaactataagccatctctaaaggataaattcatcatctccagagacaacgccaaaaatacgctgtacctgcaattgagcggagtgagatctgaggacacagccctttatcactgtgcaagagacacctcgggctaccccgactactggggccaaggcaccagtctcacagtctcctca

SEQ ID NO: 235 - DNA, 321 bp, synthetic construct
  source 1..321: mol_type = other DNA
gacatcctgttgacccaatctccatcctccatgtctgtttctctgggagacacagtcagcataacgtgtcatgcaagtcaggacattagcaggaatatagggtggttgcggcagaaaccagggaaatcatttaagggcctgatctatcatggaaccaatttggaagatggagttccatcaaggttcagcggcagtggatctggagcagattattctctcaccatcagcagcctggaatctgaagattttgcagactattactgtgtgcagtatgctcagtttccgtacacgttcggaggggggaccaaactggaaataaaa

(19) United States
(12) Patent Application Publication
(10) Pub. No.: US 2026/0217829 A1
(43) Pub. Date: Jul. 30, 2026

(54) MODIFICATION OF ANTI-PD-1 ANTIBODIES TO TREAT CANCER

(71) Applicant: The Trustees of Columbia University in the City of New York, New York, NY (US)

(72) Inventors: Adam MOR, Old Tappan, NJ (US); Ruijiang SONG, Forest Hills, NY (US)

(21) Appl. No.: 19/639,897

(22) Filed: Apr. 6, 2026

Related U.S. Application Data
(63) Continuation-in-part of application No. PCT/US24/50028, filed on Oct. 4, 2024.
(60) Provisional application No. 63/588,373, filed on Oct. 6, 2023.
(60) Provisional application No. 63/696,015, filed on Sep. 18, 2024.
(60) Provisional application No. 63/795,086, filed on Apr. 25, 2025.
(60) Provisional application No. 63/809,195, filed on May 20, 2025.

(51) Int. Cl. C07K 16/28; A61K 39/00; A61P 35/00; C07K 14/705; C07K 14/765; C07K 16/18; C12N 15/63

(52) U.S. Cl. CPC C07K 16/2818; A61P 35/00; C07K 14/70596; C07K 14/765; C07K 16/18; C12N 15/63; A61K 2039/505; C07K 2317/31; C07K 2317/522; C07K 2317/53; C07K 2317/624; C07K 2317/76

(57) ABSTRACT

The subject matter described here relates to anti-PD-1 monoclonal antibodies with increased size that are capable of modulating T-cell activity. In certain embodiments the monoclonal antibody comprises an additional Fc domain or CD22 domain.

This application is a continuation-in part of PCT/US2024/50028, filed Oct. 4, 2024, which claims the benefit of and priority to U.S. Provisional Ser. No. 63/588,373, filed Oct. 6, 2023, and U.S. Provisional Ser. No. 63/696,015 filed Sep. 18, 2024, the contents of each of which is hereby incorporated by reference in its entirety. This application also claims the benefit of and priority to U.S. Provisional Ser. No. 63/795,086, filed on Apr. 25, 2025, and US. Provisional Ser. No. 63/809,195, filed May 20, 2025, the contents of each of which are hereby incorporated by reference in their entirety.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### GOVERNMENT SUPPORT

This invention was made with government support under grant numbers AI125640 and AI150597 awarded by the National Institutes of Health (NIH). The government has certain rights in the invention.

### INCORPORATION BY REFERENCE

All documents cited herein are incorporated herein by reference in their entireties.

### SEQUENCE LISTING

The instant application contains a Sequence Listing which has been submitted electronically in XML format and is hereby incorporated by reference in its entirety. Said XML copy, created on Mar. 24, 2026, is named 0019240_01323US5_SL.xml and is 254,880 bytes in size.

### TECHNICAL FIELD

The present invention relates generally to antibodies and expression systems for producing antibodies. More particularly, the present invention relates to anti-PD-1 monoclonal antibodies with increased size.

### BACKGROUND

Targeting immune checkpoint receptors on T cells is a common cancer treatment strategy. Frequently, this is accomplished through monoclonal antibodies targeting the ligand binding sites of inhibitory co-receptors. Blocking the immune checkpoint PD-1 binding to its ligands PD-L1 and PD-L2 prevents downstream signaling and enhances specific T functions. Since 2013, the FDA has approved nine monoclonal antibodies to inhibit PD-1 and PD-L1 signaling. This therapeutic approach improved the care of patients with cancers. However, many patients are unresponsive, and some develop immune-related adverse events (irAEs). There is a need for prophylactic and/or therapeutic agents with improved specificity and efficacy, and reduced side effects.

### SUMMARY OF THE INVENTION

In certain aspects, the present application provides a monoclonal antibody or a fragment thereof, comprising: a first arm comprising a first variable heavy chain domain and a first variable light chain domain, wherein a portion of the first arm is capable of binding to a portion of a PD-1 protein; and a second arm comprising a second variable heavy chain domain and a second variable light chain domain, wherein a portion of the second arm is capable of binding to a portion of the PD-1 protein, wherein the first and second arms each further comprise a fragment crystallizable (Fc) domain wherein the first and second arms each further comprise at least one additional domain.

In some embodiments, the first and second arms each further comprise a CH1 domain, a hinge domain, and a CL domain.

In some embodiments, the at least one additional domain comprises a second Fc domain. In some embodiments, the first and second arms each further comprise one additional domain comprising a second Fc domain. In some embodiments, the at least one additional domain comprises a CD22 domain, a human serum albumin (HSA), or an albumin binding domain (ABD).

In some embodiments, the first and second arms each further comprise one additional domain comprising a CD22 domain. In some embodiments, the CD22 domain comprises the SEQ ID NO: 8, or a fragment thereof. In some embodiments, the CD22 domain comprises SEQ ID NO: 9. In some embodiments, the CD22 domain comprises SEQ ID NO: 10. In some embodiments, the monoclonal antibody comprises an ABD domain, wherein the ABD domain comprises any one of SEQ ID NOs: 62-71, or a fragment thereof. In some embodiments, the monoclonal antibody comprises a HSA domain, wherein the HSA domain comprises any one of SEQ ID NOs: 51-60, or a fragment thereof.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first PD-1 binding site, and the second variable heavy chain domain and second variable light chain domain form a second PD-1 binding site.

In some embodiments, the first and second PD-1 binding sites are the same.

In some embodiments, the first and third polypeptide chain each further encode a hinge domain, a CH1 domain, the Fc domain, and the at least one additional domain, and the second and fourth polypeptide chain each further encode a CL domain. In some embodiments, the first and third polypeptide chains comprise the same sequence, and the second and fourth polypeptide chains comprise the same sequence.

In some embodiments, the first variable heavy chain domain comprises an amino acid sequence of the variable heavy chain portion of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74 and the first variable light chain domain comprises an amino acid sequence of the variable light chain portion of SEQ ID NO: 3, 15, 17, 19, 21, 23, 25, 27, 29, 31, 73, or 75.

In some embodiments, the first arm and second arm each comprises an amino acid sequence comprising SEQ ID NO: 2 and SEQ ID NO: 3. In some embodiments, the first arm and second arm each comprises an amino acid sequence comprising SEQ ID NO: 11 and SEQ ID NO: 3. In some embodiments, the first arm and second arm each comprises an amino acid sequence comprising SEQ ID NO: 12 and SEQ ID NO: 3.

In some embodiments, the first and third polypeptide chain each comprises an amino acid sequence comprising SEQ ID NO: 2 and the second and fourth polypeptide chain each comprises an amino acid sequence comprising SEQ ID NO: 3. In some embodiments, the first and third polypeptide chain each comprises an amino acid sequence comprising SEQ ID NO: 11 and the second and fourth polypeptide chain each comprises an amino acid sequence comprising SEQ ID NO: 3. In some embodiments, the first and third polypeptide chain each comprises an amino acid sequence comprising SEQ ID NO: 12 and the second and fourth polypeptide chain each comprises an amino acid sequence comprising SEQ ID NO: 3.

In some embodiments, the first and second polypeptide chains are linked by one or more covalent disulfide bonds and the third and fourth polypeptide chains are linked by one or more covalent disulfide bonds.

In some embodiments, the first and third polypeptide chains are linked by one or more covalent disulfide bonds.

In some embodiments, the monoclonal antibody is capable of localizing a PD-1 protein away from an immune synapse.

In some embodiments, the monoclonal antibody is capable of disrupting downstream signaling of a PD-1 mediated response in a T cell.

In some embodiments, the monoclonal antibody is capable of enhancing T cell function.

In some embodiments, the PD-1 protein is located on a T cell, and the monoclonal antibody is capable of preventing the PD-1 protein from binding to a PDL-1 protein or a PDL-2 protein on a tumor cell.

In some embodiments, the monoclonal antibody is capable of inducing a cytokine secretion in a T cell. In some embodiments, the cytokine secretion is a secretion of IL-2.

In some embodiments, the monoclonal antibody is larger than 140 kD in size. In some embodiments, the monoclonal antibody is about 200 kD in size. In some embodiments, the monoclonal antibody is between about 160 kD and 230 kD in size.

In certain aspects, the present application provides a pharmaceutical composition comprising: a monoclonal antibody as described above and a pharmaceutically acceptable carrier.

In certain aspects, the present application provides a method of preventing or treating cancer in a subject comprising administering to the subject an effective amount of the composition of the composition described above.

In some embodiments, the cancer is selected from colorectal cancer, lung cancer, bladder cancer, breast cancer, cervical cancer, kidney cancer, leukemia, Hodgkin lymphoma, non-Hodgkin lymphoma, prostate cancer, skin cancer (e.g., melanoma), head and neck cancer, endometrial cancer, colon cancer, rectal cancer, liver cancer, thyroids cancer, esophageal cancer, renal cell cancer, and a combination thereof.

In certain aspects, the present application provides a method of preventing or treating a viral infection in a subject comprising administering to the subject an effective amount of the composition described above. In some embodiments, the viral infection is HIV.

In certain aspects, the present application provides a kit for generating a monoclonal antibody or fragment thereof, the kit comprising one or more vectors comprising a polynucleotide sequence encoding any of the monoclonal antibodies described above.

In certain aspects, the present application provides a kit for generating a monoclonal antibody or fragment thereof, the kit comprising: a first vector comprising a polynucleotide sequence encoding the first polypeptide chain of the monoclonal antibody with the first variable heavy chain domain of the first arm encoded by a first polypeptide chain; and a second vector comprising a polynucleotide sequence encoding the second polypeptide chain of the monoclonal antibody with the first variable light chain domain of the first arm encoded by a second polypeptide chain, the first variable heavy chain domain and first variable light chain domain form a first PD-1 binding site.

In some embodiments, the first and second PD-1 binding sites are the same. In some embodiments, the first and the third polypeptide chains each further encode a hinge domain, a CH1 domain, the Fc domain, and the at least one additional domain and the second and fourth polypeptide chain each further encode a CL domain. In some embodiments, the first and third polypeptide chains comprise the same sequence, and the second and fourth polypeptide chains comprise the same sequence. In some embodiments, the first variable heavy chain domain comprises an amino acid sequence of the variable heavy chain portion of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74 wherein the first variable light chain domain comprises an amino acid sequence of the variable light chain portion of SEQ ID NO: 3, 15, 17, 19, 21, 23, 25, 27, 29, 31, 73, or 75.

In some embodiments, the first arm and second arm each comprises an amino acid sequence comprising SEQ ID NO: 2 and SEQ ID NO: 3. In some embodiments, the first arm and second arm each comprises an amino acid sequence comprising SEQ ID NO: 11 and SEQ ID NO: 3. In some embodiments, the first arm and second arm each comprises an amino acid sequence comprising SEQ ID NO: 12 and SEQ ID NO: 3. In some embodiments, the first and third polypeptide chain each comprises an amino acid sequence comprising SEQ ID NO: 2 and the second and fourth polypeptide chain each comprises an amino acid sequence comprising SEQ ID NO: 3. In some embodiments, the first and third polypeptide chain each comprises an amino acid sequence comprising SEQ ID NO: 11 and the second and fourth polypeptide chain each comprises an amino acid sequence comprising SEQ ID NO: 3. In some embodiments, the first and third polypeptide chain each comprises an amino acid sequence comprising SEQ ID NO: 12 and the second and fourth polypeptide chain each comprises an amino acid sequence comprising SEQ ID NO: 3.

In some embodiments, the first vector and the second vector are the same vector. In some embodiments, the first vector and the second vector are two different vectors.

In certain aspects, the present application provides one or more host cells comprising: one or more vectors comprising a polynucleotide sequence encoding any of the monoclonal antibodies described above.

In certain aspects, the present application provides one or more host cells comprising: a first vector comprising a polynucleotide sequence encoding the first polypeptide chain of the monoclonal antibody with the first variable heavy chain domain of the first arm encoded by a first polypeptide chain; and a second vector comprising a polynucleotide sequence encoding the second polypeptide chain of the monoclonal antibody with the first variable light chain domain of the first arm encoded by a second polypeptide chain, the first variable heavy chain domain and first variable light chain domain form a first PD-1 binding site.

In some embodiments, the first and second PD-1 binding sites are the same. In some embodiments, the first and the third polypeptide chains each further encode a hinge domain, a CH1 domain, the Fc domain, and the at least one additional domain and the second and fourth polypeptide chain each further encode a CL domain. In some embodiments, the first and third polypeptide chains comprise the same sequence and the second and fourth polypeptide chains comprise the same sequence. In some embodiments, the first variable heavy chain domain comprises an amino acid sequence of the variable heavy chain portion of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74 wherein the first variable light chain domain comprises an amino acid sequence of the variable light chain portion of SEQ ID NO: 3, 15, 17, 19, 21, 23, 25, 27, 29, 31, 73, or 75.

In some embodiments, the first arm and second arm each comprises an amino acid sequence comprising SEQ ID NO: 2 and SEQ ID NO: 3. In some embodiments, the first arm and second arm each comprises an amino acid sequence comprising SEQ ID NO: 11 and SEQ ID NO: 3. In some embodiments, the first arm and second arm each comprises an amino acid sequence comprising SEQ ID NO: 12 and SEQ ID NO: 3. In some embodiments, the first and third polypeptide chain each comprises an amino acid sequence comprising SEQ ID NO: 2 and the second and fourth polypeptide chain each comprises an amino acid sequence comprising SEQ ID NO: 3. In some embodiments, the first and third polypeptide chain each comprises an amino acid sequence comprising SEQ ID NO: 11 and the second and fourth polypeptide chain each comprises an amino acid sequence comprising SEQ ID NO: 3. In some embodiments, the first and third polypeptide chain each comprises an amino acid sequence comprising SEQ ID NO: 12 and the second and fourth polypeptide chain each comprises an amino acid sequence comprising SEQ ID NO: 3.

In some embodiments, the first vector and the second vector are the same vector. In some embodiments, the first vector and the second vector are two different vectors.

In some embodiments, the one or more host cells are cultured under conditions suitable for an expression of the one or more vectors; and recovering the monoclonal antibody or fragment thereof. In some embodiments, the one or more host cells are cultured under conditions suitable for an expression of the first vector and the second vector; and recovering the monoclonal antibody or fragment thereof.

In certain aspects, the present application provides a composition comprising: one or more vectors comprising a polynucleotide sequence encoding any of the monoclonal antibodies described above.

In certain aspects, the present application provides a composition comprising: a first vector comprising a polynucleotide sequence encoding the first polypeptide chain of the monoclonal antibody with the first variable heavy chain domain of the first arm encoded by a first polypeptide chain; and a second vector comprising a polynucleotide sequence encoding the second polypeptide chain of the monoclonal antibody with the first variable light chain domain of the first arm encoded by a second polypeptide chain, the first variable heavy chain domain and first variable light chain domain form a first PD-1 binding site.

In some embodiments, the first and second PD-1 binding sites are the same. In some embodiments, the first and the third polypeptide chains each further encode a hinge domain, a CH1 domain, the Fc domain, and the at least one additional domain and the second and fourth polypeptide chain each further encode a CL domain. In some embodiments, the first and third polypeptide chains comprise the same sequence and the second and fourth polypeptide chains comprise the same sequence. In some embodiments, the first variable heavy chain domain comprises an amino acid sequence of the variable heavy chain portion of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74 wherein the first variable light chain domain comprises an amino acid sequence of the variable light chain portion of SEQ ID NO: 3, 15, 17, 19, 21, 23, 25, 27, 29, 31, 73, or 75.

In some embodiments, the first arm and second arm each comprises an amino acid sequence comprising SEQ ID NO: 2 and SEQ ID NO: 3. In some embodiments, the first arm and second arm each comprises an amino acid sequence comprising SEQ ID NO: 11 and SEQ ID NO: 3. In some embodiments, the first arm and second arm each comprises an amino acid sequence comprising SEQ ID NO: 12 and SEQ ID NO: 3. In some embodiments, the first and third polypeptide chain each comprises an amino acid sequence comprising SEQ ID NO: 2 and the second and fourth polypeptide chain each comprises an amino acid sequence comprising SEQ ID NO: 3. In some embodiments, the first and third polypeptide chain each comprises an amino acid sequence comprising SEQ ID NO: 11 and the second and fourth polypeptide chain each comprises an amino acid sequence comprising SEQ ID NO: 3. In some embodiments, the first and third polypeptide chain each comprises an amino acid sequence comprising SEQ ID NO: 12 and the second and fourth polypeptide chain each comprises an amino acid sequence comprising SEQ ID NO: 3.

In some embodiments, the first vector and the second vector are the same vector. In some embodiments, the first vector and the second vector are two different vectors.

In certain aspects, the present application provides a means for binding a portion of a PD-1 protein. In some embodiments, the means comprises a monoclonal antibody comprising a domain for increasing the size of the monoclonal antibody over 140 kD. In some embodiments, the monoclonal antibody or a fragment thereof comprises: a first arm comprising a first variable heavy chain domain and a first variable light chain domain, wherein a portion of the first arm is capable of binding to the portion of the PD-1 protein; and a second arm comprising a second variable heavy chain domain and a second variable light chain domain, wherein a portion of the second arm is capable of binding to the portion of the PD-1 protein wherein the first and second arms each further comprise a fragment, crystallizable (Fc) domain wherein the first and second arms each further comprise the domain for increasing the size of the monoclonal antibody over 140 kD.

In some embodiments, the first and second arms each further comprise a CH1 domain, a hinge domain, and a CL domain.

In some embodiments, the domain for increasing the size of the monoclonal antibody over 140 kD comprises a second Fc domain. In some embodiments, the domain for increasing the size of the monoclonal antibody over 140 kD comprises a CD22 domain.

In some embodiments, the first and second arms each further comprise one additional domain comprising a CD22 domain, a human serum albumin (HSA), or an albumin binding domain (ABD). In some embodiments, the CD22 domain comprises the SEQ ID NO: 8, or a fragment thereof. In some embodiments, the CD22 domain comprises SEQ ID NO: 9. In some embodiments, the CD22 domain comprises SEQ ID NO: 10. In some embodiments, the monoclonal antibody comprises an ABD domain, wherein the ABD domain comprises any one of SEQ ID NOs: 62-71, or a fragment thereof. In some embodiments, the monoclonal antibody comprises a HSA domain, wherein the HSA domain comprises any one of SEQ ID NOs: 51-60, or a fragment thereof.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first PD-1 binding site and wherein the second variable heavy chain domain and second variable light chain domain form a second PD-1 binding site. In some embodiments, the first and second PD-1 binding sites are the same.

In some embodiments, the first and third polypeptide chain each further encode a hinge domain, a CH1 domain, the Fc domain, and the at least one additional domain for increasing the size of the monoclonal antibody over 140 kD, and wherein the second and fourth polypeptide chain each further encode a CL domain. In some embodiments, the first and third polypeptide chains comprise the same sequence and the second and fourth polypeptide chains comprise the same sequence.

In some embodiments, the first variable heavy chain domain comprises an amino acid sequence of the variable heavy chain portion of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74, wherein the first variable light chain domain comprises an amino acid sequence of the variable light chain portion of SEQ ID NO: 3, 15, 17, 19, 21, 23, 25, 27, 29, 31, 73, or 75.

In some embodiments, the first arm and second arm each comprises an amino acid sequence comprising SEQ ID NO: 2 and SEQ ID NO: 3. In some embodiments, the first arm and second arm each comprises an amino acid sequence comprising SEQ ID NO: 11 and SEQ ID NO: 3. In some embodiments, the first arm and second arm each comprises an amino acid sequence comprising SEQ ID NO: 12 and SEQ ID NO: 3.

In some embodiments, the monoclonal antibody is capable of localizing a PD-1 protein away from an immune synapse.

In some embodiments, the monoclonal antibody is capable of disrupting downstream signaling of a PD-1 mediated response in a T cell. In some embodiments, the monoclonal antibody is capable of enhancing T cell function.

In some embodiments, the PD-1 protein is located on a T cell, and wherein the monoclonal antibody is capable of preventing the PD-1 protein from binding to a PDL-1 protein or a PDL-2 protein on a tumor cell.

In some embodiments, the monoclonal antibody is capable of inducing a cytokine secretion in a T cell. In some embodiments, the cytokine secretion is a secretion of IL-2.

In some embodiments, the monoclonal antibody is between about 160 kD and 200 kD in size.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following figures depict illustrative embodiments of the invention.

FIG. 1 shows that anti-PD-1 Ab and Anti-PD-1-Fc Ab bind to PD-1. ELISA wells were coated with the recombinant ectodomain of PD-1 (2 μg/ml) overnight before incubation with different concentrations of the anti-PD-1 or anti-PD-1-Fc antibodies at room temperate. After 45 min, wells were washed, and the level of bound antibodies was detected with tagged secondary antibodies using a plate reader at an OD of 450. A representative experiment is shown (n=2).

FIG. 2 shows that anti-PD-1-Fc Ab removes PD-1 away from the immune synapse. Jurkat T cells stably expressing GFP-PD-1 were cocultured with Raji B cells expressing mCherry-PD-L1. The Raji B cells were pretreated with SEE (50 μg/ml), and the Jurakt T cells were pretreated with specific antibodies (1 μg/ml or molar equivalent), as indicated, for 45 minutes. Live cocultured cells were imaged with confocal microscopy (Zeiss) for 30 minutes, and the number of the conjugates where PD-1 was enriched at the immune synapse was calculated using ImageJ. Representative images are shown on top, and the quantitation of four independent experiments is included (n=4, 50-70 cells per experiment, SEM, *p<0.05).

FIG. 3 shows that anti-PD-1-Fc Ab and anti-PD-1-CD22 increases IL-2 secretion of PBMC assay. PBMC were isolated from healthy volunteers before being pretreated with the indicated antibodies (1 ug/ml or molar equivalent) and SEE (50 ug/ml). Cells were cultured at 37 degrees C. overnight, and ELISA determined the levels of secreted IL-2 in the media (n=3, SEM, *p<0.001).

FIG. 4 shows alternative antibody designs for anti-PD-1 antibodies. The figure is adapted from FIG. 1 of Spiess C, Zhai Q, Carter P J, Alternative molecular formats and therapeutic applications for bispecific antibodies, Mol Immunol., 2015 October; 67 (2 Pt A): 95-106, the contents of which is hereby incorporated by reference in its entirety.

FIGS. 5A-B show binding curves to human-PD-1-His-coated plates quantified by ELISA. FIG. 5A shows binding curves for anti-PD-1 antibody clones 01, 02, 03, and 07, anti-human PD-1 antibody Penbio (pembrolizumab), anti-HEL-human IgG1 isotype control, and blank. FIG. 5B shows binding curves for anti-PD-1 antibody clones 09, 51, 55, 79, and 80, anti-human PD-1 antibody Penbio (pembrolizumab), anti-HEL-human IgG1 isotype control, and blank. EC50 values were calculated with GraphPad Prism (v10.2.1).

FIG. 6 shows binding of anti-PD-1 clones 01, 02, 03, 07, 09, 51, 55, 79, and 80 to cell-expressed PD-1 by flow cytometry.

FIG. 7 shows anti-PD-1 clones 01, 02, 03, 07, 09, 51, 55, 79, and 80 blocking the binding of rhPD-L2 to cell-expressed PD-1.

FIG. 8 shows IL-2 concentrations determined by ELISA following addition of anti-PD-1 clones 01, 02, 03, 07, 09, 51, 55, 79, or 80 and SEE (Staphylococcal Enterotoxin E) in Jurkat-Raji co-culture compared to no SEE and no antibody control and SEE and no antibody control.

FIG. 9 shows IL-2 concentrations determined by ELISA following addition of anti-PD-1 clones 01, 02, 03, 07, 09, 51, 55, 79, or 80 at different concentrations (10, 2, 0.5 or 0.1 ug/ml) and SEE (Staphylococcal Enterotoxin E) in Jurkat-Raji co-culture compared to no SEE and no antibody control and SEE and no antibody control.

FIG. 10 shows concentrations of IL-2, IFNγ, IL-6, IL-4 and IL-1β determined by ELISA in PBMCs in the presence of anti-PD-1 clones 01, 02, 03, 07, 09, 51, 55, 79, or 80 and SEE (Staphylococcal Enterotoxin E).

FIG. 11 shows the amino acid sequences for variable heavy chain and variable light chain portions of anti-PD-1 antibody clones 01, 02, 03, 07, 09, 51, 55, 79, and 80. Bolded and underlined amino acids indicates CDRs. Figure discloses SEQ ID NOS 72-89, respectively, in order of appearance.

FIG. 12 shows binding of Pembrolizumab (left) and HSA-Pembrolizumab (right) to cell-expressed PD-1 determined by flow cytometry.

FIG. 13 shows IFNg (top) and IL-2 (bottom) concentrations determined by ELISA following addition of SEE (Staphylococcal Enterotoxin E), Pembrolizumab with SEE, HSA-Pembrolizumab with SEE, or no antibody and no SEE.

FIG. 14 shows binding determined by ELISA of Pembrolizumab, ABD-Pembrolizumab, and Pembrolizumab-ABD to PD-1 (top), human serum albumin (HSA) (middle), and mouse serum albumin (MSA) (bottom).

FIG. 15 shows IL-2 concentrations determined by ELISA following addition of SEE (Staphylococcal Enterotoxin E), Pembrolizumab with SEE, ABD-Pembrolizumab with SEE and MSA, Pembrolizumab-ABD with SEE and MSA, or no antibody and no SEE.

FIG. 16 shows a schematic of SYB1.L mechanism of action and a Western Blot. SYB1.L in FIGS. 16-20 refers to anti-PD1-HSA as described herein.

FIGS. 17A-E show that SYB1.L is a novel antibody that targets the immune checkpoint PD-1. FIG. 17A shows Western blots of heavy and light chains of Pembroluzimab and SYB1.L. FIG. 17B shows immunofluorescence of T-cells treated with Pembroluzimab and SYB1.L. FIG. 17C shows PD-1 enrichment at T-cell immune synapse after treatment with Pembroluzimab and SYB1.L. FIG. 17D shows SYB1.L binds to PD-1 on the surface of cells better than Pembrolizumab. FIG. 17E shows SYB1.L is a stronger activator of T cells: PBMC cytokine secretion assay.

FIG. 18 shows treatment of H1299 human lung tumor cells with SYB1.L in vivo. Mice are MHCI/II double knockout mice which have been inoculated with H1299 tumor cells. Mice are treated with either Pembroluzimab or SYB1.L. Tumor volume is measured for 37 days after tumor inoculation.

FIG. 19 shows treatment of EO771 murine breast tumor cells with SYB1.L in vivo. Mice are MHCI/II double knockout mice which have been inoculated with EO771 tumor cells. This tumoe is known to be resistant to conventional PD-1 inhibition (Pembro). Mice are untreated or treated with either Pembroluzimab or SYB1.L. Tumor volume is measured for 23 days after tumor inoculation.

FIGS. 20A-C show SYB1.L increases the number of effector and decreases the number of naïve T cells in vivo better than Pembrolizumab. UMAP plots of CD8+ (FIG. 20A) and CD4+ (FIG. 20B) cells from human-PD-1 B6 mice carrying MC38 tumors which are further split up into untreated, Pembroluzimab-treated, and SYB1.L treated groups. FIG. 20C show % of naïve CD8+ T cells, effector CD8 T cells, naïve CD4+ T cells, and effector CD4+ T cells.

FIGS. 21A-D show in vivo efficacy of HSA-PD1 antibodies in hPD1 transgenic mice MC38 tumor model. FIG. 21A shows tumors were treated with clone 051-HSA and compared to clone 051 mAb as well as untreated mice. Each mice received 4 doses of treatment total. The dosage amounts of 051-HSA and 051 mAb are matched by molar concentration (051-HSA 385 μg/dose; 051 mAb 200 μg/dose). FIG. 21B shows average endpoint tumor mass of all conditions. FIG. 21C shows tumor volume of individual mice for untreated and clone 051 mAb treatments. FIG. 21D shows tumor volume of individual mice for untreated and clone 051-HSA mAb treatments.

### DETAILED DESCRIPTION

Disclosed herein are novel monoclonal antibodies that in some embodiments are useful for preventing or treating disease (e.g., cancer) or infection (e.g., human immunodeficiency virus (HIV)). One approach to cancer treatment consists of using monoclonal antibodies to target immune checkpoint receptors on T cells, inhibiting the interaction between the PD-1 immune checkpoint and its ligands. However, a significant number of patients do not respond to this treatment, and some may experience immune-related adverse events. A limitation of existing technologies that may lead to these outcomes is that they fail to adequately consider the localization of PD-1 on the surface of immune cells within the immune synapse. Described herein is the modification of an FDA-approved anti-PD-1 antibody, pembrolizumab, through the addition of another Fc domain, a CD22 domain, human serum albumin (HSA), or an albumin binding domain (ABD) to the Fc tail of pembrolizumab. The modified antibodies have an increased size of at least about 200 kD compared to the pembrolizumab (~140 kD). Unexpectedly, the modified antibodies removed the PD-1 protein from the immune synapse. In comparison to canonical pembrolizumab, this technology exhibits superior inhibition of PD-1 signaling, thereby improving cytokine secretion and primary T cell killing of tumor cells in vitro. As such, this approach offers a safer and more efficacious alternative to current immunotherapies for treating cancer patients. Also described herein is the modification of anti-PD-1 antibodies referred to as clones 01, 02. 03, 07, 09, 51, 55, 79, and 80, through the addition of another Fc domain, a CD22 domain, human serum albumin (HSA), or an albumin binding domain (ABD) to the Fc tail of pembrolizumab. The modified antibodies have an increased size of at least about 160 kD compared to a monoclonal antibody without the additional domain (~140 kD). In some embodiments, the modified antibodies have an increased size of at least about 200 kD compared to a monoclonal antibody without the additional domain (~140 kD). In some embodiments, the modified antibodies comprising an ABD can bind to albumin in vivo, for example, albumin present in the serum which contributes to an increased size of the monoclonal antibody.

### T Cells and The Immune Synapse

T cells have surface receptors that can act as immune checkpoint receptors, such as PD-1. See Speiser D E, Ho P C, Verdeil G. Regulatory Circuits of T Cell Function in Cancer. Nat Rev Immunol. 2016 October; 16 (10): pp. 599-611, incorporated by reference in its entirety herein. These receptors act as “checkpoints” to prevent excessive immune activation. However, cancer cells can exploit these checkpoint pathways to evade immune detection and attack. See Liu J, Chen Z, Li Y, Zhao W, Wu J and Zhang Z. PD-1/PD-L1 Checkpoint Inhibitors in Tumor Immunotherapy. Front. Pharmacol. 2021 September; pp. 12: p. 731798, incorporated by reference in its entirety herein. PD-1 is expressed on the surface of activated T cells while its ligands PD-L1 and PD-L2 are expressed on various cancer cells. See Liu, et al. (2021). The immune synapse is the interface between T cells and tumor cells. This interface, or microenvironment, includes the checkpoint receptors (e.g., PD-1, PDL-1, PDL-2) and other immune receptors and ligands needed for T cells to function. The immune synapse is organized into three compartments; every protein has a specific location. For example, with respect to proteins found on T cells, the T cell receptor (TCR) is localized to the center of the synapse, PD-1 and CD28 are located in the peripheral synapse, and LFA-1, CD43, and CD45 are found in the distal compartment of the synapse. See e.g., Delon J, Kaibuchi K, Germain R N. Exclusion of CD43 from the immunological synapse is mediated by phosphorylation-regulated relocation of the cytoskeletal adaptor moesin. Immunity. 2001 November; 15 (5): 691-701. doi: 10.1016/s1074-7613 (01) 00231-x. PMID: 11728332. This organization is critical for the function of the synapse, where specific clusters of proteins are formed.

Cancer immunotherapy drugs work by blocking these checkpoint receptors on T cells, thereby unleashing the immune system to recognize and destroy cancer cells more effectively. He X, Xu C. Immune Checkpoint Signaling and Cancer Immunotherapy. Cell Res. 2020 August; 30 (8): pp. 660-669, incorporated by reference in its entirety herein. For example, PD-1 inhibitors prevent PD-1 on T cells from binding to its ligands PDL-1 and PDL-2 on tumor cells, thereby preventing the tumor cells from evading the T cell immune response. Seven monoclonal antibodies targeting PD-1 have been approved by the FDA for cancer treatment. However, some individuals do not respond to this treatment, and some experience immune-related adverse events (irAEs).

Without intending to be bound by any particular theory, the design of antibodies for the prevention or treatment of cancer is improved by taking into account the localization of PD-1 on the surface of the immune cells in the context of the immune synapse. Without intending to be bound by any particular theory, in addition to blocking ligand binding, it is hypothesized that changing the location of PD-1 within the different compartments of the immune synapse could serve as an alternative, efficient, and safer approach to treating cancer patients.

Described herein are improved versions of anti-PD-1 antibodies. Novel anti-PD-1-Fc antibodies, anti-PD-1-CD22, anti-PD-1-HSA, and anti-PD-1-ABD antibodies were designed and are described herein. Without intending to be bound by any particular theory, the rationale behind the design of the antibodies disclosed herein was that disruption of selected protein interactions through altered localization would serve as a strategy to affect T cell function better. The PD-1 pathway provides a clear example to demonstrate how changing the localization of a protein on the cell surface can impact T cell function. In some embodiments, an advantage of removing PD-1 from the synapse with antibodies is its ability to prevent PD-1 ligand-independent downstream tonic signaling.

In some embodiments, described herein are anti-PD-1-Fc antibodies, anti-PD-1-CD22 antibodies, anti-PD-1-HSA antibodies, and anti-PD-1-ABD antibodies that are capable of removing PD-1 from the immune synapse. In some embodiments, the anti-PD-1-Fc antibodies are created by adding an additional Fc domain to the Fc tail of an anti-PD-1 antibody (e.g., but not limited to, pembrolizumab or the anti-PD-1 antibodies referred to as clones 01, 02, 03, 04, 07, 09, 51, humanized 51, 55, 79, and 80). In some embodiments, the anti-PD-1-CD22 antibodies are created by adding a CD22 domain to the Fc tail of an anti-PD-1 antibody (e.g., but not limited to, pembrolizumab or the anti-PD-1 antibodies referred to as clones 01, 02, 03, 04, 07, 09, 51, humanized 51,55, 79, and 80). The modified antibodies have an increased size of at least about 160 kD compared to the pembrolizumab (~140 kD). In some embodiments, the anti-PD-1-Fc antibodies are created by adding HSA to the Fc tail of an anti-PD-1 antibody (e.g., but not limited to, pembrolizumab or the anti-PD-1 antibodies referred to as clones 01, 02, 03, 04, 07, 09, 51, humanized 51, 55, 79, and 80). In some embodiments, the anti-PD-1-Fc antibodies are created by adding ABD to the Fc tail of an anti-PD-1 antibody, the ABD non-covalently binds to HSA (e.g., but not limited to, pembrolizumab or the anti-PD-1 antibodies referred to as clones 01, 02, 03, 04, 07, 09, 51, humanized 51, 55, 79, and 80). In some embodiments, the modified antibodies have an increased size of at least about 160 kD compared to an unmodified monoclonal antibody (~140 kD). In some embodiments, the modified antibodies have an increased size of at least about 200 kD compared to an unmodified monoclonal antibody (~140 kD). In some embodiments, one or more of the anti-PD-1-Fc antibodies or the anti-PD-1-CD22 antibodies described herein function by removing the PD-1 localization from the immune synapse, disrupting downstream signaling and effector pathways, and/or enhancing T-cell function. In some embodiments, the use of anti-PD-1-Fc antibodies, anti-PD-1-CD22, anti-PD-1-HSA antibodies, and anti-PD-1-ABD antibodies antibodies disclosed herein have one or more of the following advantages over conventional anti-PD-1 antibodies: 1) they prevent PD-1 interaction with its ligands and 2) they localize PD-1 away from the synapse and prevent it from interacting with its downstream effectors. In some embodiments, using one or more of the innovative monoclonal antibodies describes herein offer a more potent and safer technology to treat cancer patients resistant to current immunotherapies.

In some embodiments, an anti-PD-1-Fc, an anti-PD-1-CD22, an anti-PD-1-HSA, and/or an anti-PD-1-ABD antibody, as disclosed herein, is used to prevent or treat cancer in a subject. In some embodiments, the cancer is selected from colorectal cancer, lung cancer, bladder cancer, breast cancer, cervical cancer, kidney cancer, leukemia, Hodgkin lymphoma, non-Hodgkin lymphoma, prostate cancer, skin cancer (e.g., melanoma), head and neck cancer, endometrial cancer, colon cancer, rectal cancer, liver cancer, thyroids cancer, esophageal cancer, renal cell cancer, and a combination thereof. In some embodiments, an anti-PD-1-Fc, an anti-PD-1-CD22, an anti-PD-1-HSA, and/or an anti-PD-1-ABD antibody, as disclosed herein, is used to prevent or treat disease caused by any solid tumor that is not able to repair errors in its DNA that occur when the DNA is copied.

In some embodiments, an anti-PD-1-Fc, an anti-PD-1-CD22, an anti-PD-1-HSA, and/or an anti-PD-1-ABD antibody, as disclosed herein, is used to prevent or treat a viral infection. In some embodiments, the viral infection caused by HIV in a subject.

### Antibodies

There are five classes of human antibodies (i.e., IgA, IgD, IgE, IgG, and IgM) and each have various isotypes (e.g., IgG1, IgG2, IgG3, IgG4, IgA1, and IgA2). In some embodiments, the antibodies disclosed herein belong to the IgG class. IgG can be further divided into four subclasses: IgG1, IgG2, IgG3, and IgG4. Each subclass has a unique profile with respect to antigen binding, immune complex formation, complement activation, triggering of effector cells, half-life, and placental transport. E.g., see Gestur Vidarsson, et al., IgG Subclasses and Allotypes: From Structure to Effector Functions, 5 Frontiers in Immunology 520 (2014), incorporated by reference herein in its entirety. The term “immunoglobulin” (Ig) is used interchangeably with “antibody” herein.

The IgG immunoglobulin molecule consists of four polypeptide chains, two identical light (L) chains and two identical heavy (H) chains. The four chains are joined by disulfide bonds in a “Y” configuration wherein the light chains bracket the heavy chains starting at the mouth of the “Y” and continuing through the variable region to the dual ends of the “Y”. Each L chain is linked to an H chain by one covalent disulfide bond, while the two H chains are linked to each other by one or more disulfide bonds depending on the H chain isotype. Each H and L chain also has regularly spaced intrachain disulfide bridges. Each heavy chain consists of an N-terminal variable domain (VH) and three constant domains (CH1, CH2, CH3), with an additional “hinge region” between CH1 and CH2. Similarly, the light chains consist of an N-terminal variable domain (VL) and a constant domain (CL). The variable domains of the heavy chain and light chain may be referred to as “VH” and “VL”, respectively. These domains are generally the most variable parts of the antibody (relative to other antibodies of the same class) and contain the antigen binding sites. The VL is aligned with the VH and the CL is aligned with the first constant domain of the heavy chain (CH1). The pairing of a VH and VL together forms a single antigen-binding site. The part of the antibody formed by the lower hinge region and the CH2/CH3 domains of the heavy chain is called “Fc” (“fragment crystalline”). See e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6, incorporated by reference herein in its entirety.

The variability in an antibody sequence is concentrated in three segments called complementarity determining regions (CDRs) (also called hypervariable regions (HVRs)) both in the light-chain and the heavy chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. See Kabat et al, Sequences of Immunological Interest, Fifth Edition, National Institute of Health, Bethesda, MD (1991), incorporated by reference in its entirety herein. The constant domains are not involved directly in the binding of antibody to an antigen, but exhibit various effector functions, such as participation of the antibody in antibody-dependent cellular toxicity.

By way of example, CDRs may be defined using the nomenclature described by Kabat et al. (1991, NIH Publication 91-3242, National Technical Information Service, Springfield, Va.), incorporated by reference in its entirety herein. Specifically, residues 31-35 (CDR-H1), 50-65 (CDR-H2), and 95-102 (CDR-H3) in the heavy chain variable region and residues 24-34 (CDR-L1), 50-56 (CDR-L2), and 89-97 (CDR-L3) in the light chain variable region.

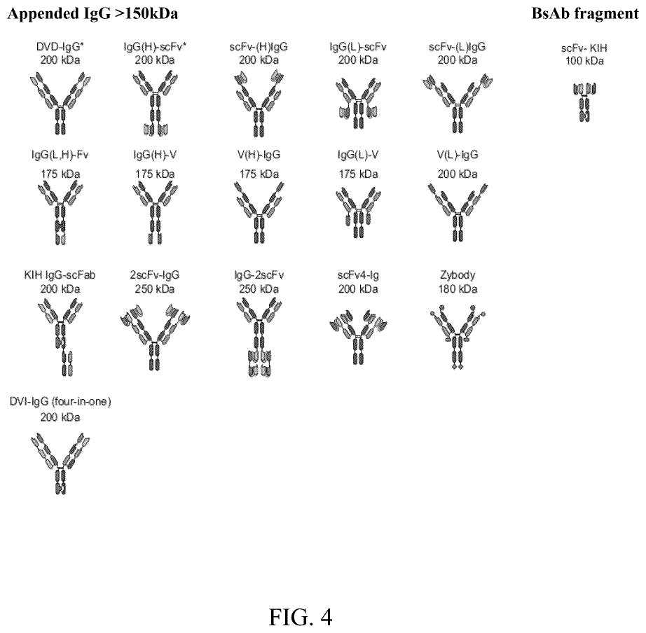

The antibodies disclosed herein (e.g., monoclonal antibodies) includes, but is not limited to, full-length antibodies that are larger in size than 140 kD and also includes other immunologically reactive/antigen-binding molecules that are larger in size than 140 kD. The antibodies of the various embodiments disclosed herein can include one or more of synthetic antibodies, monoclonal antibodies, oligoclonal or polyclonal antibodies, multiclonal antibodies, recombinantly produced antibodies, monospecific antibodies, monovalent antibodies, human antibodies, humanized antibodies, chimeric antibodies, CDR-grafted antibodies, primatized antibodies, single-chain Fv-Fcs (scFv-Fc)), bivalent with four scFv (scFv-Fc-scFv), IgG-scFv, IgM, IgA, trispecific, IgG-dAb, CrossMab 2:1 or 2:2, DVD-IgG, IgG (L)-scFv2, DVD-IgG, IgG (H)-scFv, scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG (four-in-one), scFv-KIH, and any other immunologically-reactive/antigen-binding molecules. In some embodiments, the antibody is a monoclonal antibody (see FIG. 4).

In some embodiments, the monoclonal antibody comprises a first, second, third and fourth chain. In some embodiments, the first and third chains each comprise a VH domain and the second and fourth chains each comprise a VL domain. In some embodiments, the first and third chains each further comprises a CH1 domain, a hinge domain, a Fc domain, and at least one additional domain attached to the Fc domain. In some embodiments, the additional domain is another Fc domain. In some embodiment, the additional domain is a CD22 domain. In some embodiments, the additional domain is HSA. In some embodiments, the additional domain is an ABD. In some embodiments, the second and fourth chains each further comprises a CL domain. The pairing of the VH and VL of the first and second chains together forms a single antigen-binding site specific for an epitope on PD-1 and the pairing of the VH and VL of the third and fourth chains together forms a single antigen-binding site specific for the same epitope. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size than 140 kD. In some embodiments, the monoclonal antibody is at least about 200 kD. In some embodiments, the monoclonal antibody is about 200 kD.

However, the antibodies disclosed herein are not limited to full-length IgG like antibodies. Other immunologically reactive/antigen-binding molecules that are larger in size than 140 kD (including but not limited to, single-chain Fv-Fcs (scFv-Fc), bivalent with four scFv (scFv-Fc-scFv), IgG-scFv, IgM, IgA, trispecific, IgG-dAb, CrossMab 2:1 or 2:2, DVD-IgG, IgG (L)-scFv2, DVD-IgG, IgG (H)-scFv, scFv-(H) IgG, IgG (L)-scFv, scFv-(L) IgG, IgG (L,H)-Fv, IgG (H)-V, V(H)-IgG, IgG (L)-V, V(L)-IgG, KIH IgG-scFab, 2scFv-IgG, IgG-2scFv, scFv4-Ig, Zybody, DVI-IgG (four-in-one), and scFv-KIH are also contemplated herein and a person of skill in the art can readily synthesize such molecules using the sequences and identified domains of the heavy and light chains of the anti-PD-1 antibodies disclosed here. For example, in some embodiments, the monoclonal antibody comprises a first and second chain that associate together. In some embodiments, the first chain and second chain each comprises an scFv with specificity for an epitope on PD-1 and the first and second chains each further comprise a Fc domain and at least one additional domain attached to the Fc domain. In some embodiments, the additional domain is another Fc domain. In some embodiment, the additional domain is a CD22 domain. An scFv comprises a variable heavy domain and variable light chain domain separated by a linker. In some embodiments, the linker is a glycine-serine linker. In some embodiments, the Fc domain of the first chain comprises knob mutations and the Fc domain of the second chain comprise hole mutations, or vice versa. In some embodiments the antibody is a scFv-Fc antibody comprising a first and second chain that associate together, each chain comprising a variable heavy chain (VH) domain, a linker, a variable light chain (VL) domain, an Fc domain, and at least one additional domain. In some embodiments, the additional domain is another Fc domain. In some embodiment, the additional domain is a CD22 domain. In some embodiments, the additional domain is HSA. In some embodiments, the additional domain is an ABD.

For example, in some embodiments, the monoclonal antibody comprises a first and second chain that associate together. In some embodiments, the first chain and second chain each comprise two scFvs with specificity for an epitope on PD-1 and the first and second chains each further comprise a Fc domain. An scFv comprises a variable heavy domain and variable light chain domain separated by a linker. In some embodiments, the linker is a glycine-serine linker. In some embodiments, the Fc domain of the first chain comprises knob mutations and the Fc domain of the second chain comprise hole mutations, or vice versa. In some embodiments the antibody is a scFv-Fc-scFv antibody comprising a first and second chain that associate together, each chain comprising a first variable heavy chain (VH) domain, a first linker, a first variable light chain (VL) domain, an Fc domain, a second variable heavy chain (VH) domain, a second linker, and a second variable light chain (VL) domain.

In some embodiments, the monoclonal antibodies disclosed herein contain various modifications, substitutions, additions, or deletions to the variable or binding regions of one or more arms of an anti-PD-1-Fc antibody or an anti-PD-1-CD22 antibody or an anti-PD-1-HSA antibody or an anti-PD-1-ABD antibody disclosed herein. In some embodiments, the monoclonal antibodies disclosed herein may contain substitutions or modifications of the constant region (i.e., the Fc domain). The antibodies disclosed herein may contain one or more additional amino acid residue substitutions, mutations and/or modifications, which result in a compound with preferred characteristics including, but not limited to: altered pharmacokinetics, increased serum half-life, increase binding affinity, reduced binding affinity, reduced immunogenicity, increased production, altered Fc ligand binding, enhanced or reduced ADCC or CDC activity, altered glycosylation and/or disulfide bonds and modified binding specificity.

### Amino Acid Sequences of Anti-PD-1-Fc Antibodies

In some embodiments, the anti-PD-1-Fc antibody comprises two polypeptide heavy chains each comprising a variable heavy chain (VH) domain, CH1 domain, a hinge domain, a first Fc domain (CH2-CH3), and a second Fc domain (CH2-CH3), and two polypeptide light chains comprising a variable light chain (VL) domain and a CL domain.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

In some embodiments, the amino acid sequence of the anti-PD-1-Fc antibody heavy chain comprises SEQ ID NO: 2. Underlined amino acids represent the VH domain. Underlined and bolded amino acids represent the respective complementarity determining regions (CDRs). Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 2)
QVQLVQSGVEVKKPGASVKVSCKASGYTFTNYYMYWVRQAPGQGLE
WMGGINPSNGGTNFNEKFKNRVTLTTDSSTTTAYMELKSLQFDDTA
VYYCARRDYRFDMGFDYWGQGTLVTVSSASTKGPSVFPLAPCSRST
SESTAALGCLVKDYFPEPVTVSWNSGALTSGVHTFPAVLQSSGLYS
LSSVVTVPSSSLGTKTYTCNVDHKPSNTKVDKRVESKYGPPCPPCP
APEFEGGPSVFLFPPKPKDTLMISRTPEVTCVVVDVSQEDPEVQFN
WYVDGVEVHNAKTKPREEQFNSTYRVVSVLTVLHQDWLNGKEYKCK
VSNKGLPSSIEKTISKAKGQPREPQVYTLPPSQEEMTKNQVSLTCL
VKGFYPSDIAVEWESNGQPENNYKTTPPVLDSDGSFFLYSRLTVDK
SRWQEGNVFSCSVMHEALHNHYTQKSLSLSLGSTGSKYGPPCPPCP
APEFEGGPSVFLFPPKPKDTLMISRTPEVTCVVVDVSQEDPEVQFN
WYVDGVEVHNAKTKPREEQFNSTYRVVSVLTVLHQDWLNGKEYKCK
VSNKGLPSSIEKTISKAKGQPREPQVYTLPPSQEEMTKNQVSLTCL
VKGFYPSDIAVEWESNGQPENNYKTTPPVLDSDGSFFLYSRLTVDK
SRWQEGNVFSCSVMHEALHNHYTQKSLSLSLG.

In some embodiments, the amino acid sequence of the anti-PD-1-Fc antibody heavy chain comprises SEQ ID NO: 1 immediately followed by SEQ ID NO: 2. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 3. Underlined amino acids represent the VL domain. Underlined and bolded amino acids represent the respective complementarity determining regions (CDRs). Italicized amino acids represent the CL domain.

(SEQ ID NO: 3)
DIVLTQSPATLSLSPGERATLSCRASKGVSTSGYSYLHWYQQKPGQ
APRLLIYLASYLESGVPARFSGSGSGTDFTLTISSLEPEDFAVYYC
QHSRDLPLTFGGGTKVEIKRTVAAPSVFIFPPSDEQLKSGTASVVC
LLNNFYPREAKVQWKVDNALQSGNSQESVTEQDSKDSTYSLSSTLT
LSKADYEKHKVYACEVTHQGLSSPVTKSFNRGEC.

In some embodiments, the amino acid sequence of the anti-PD-1-Fc antibody light chain comprises SEQ ID NO: 1 immediately followed by SEQ ID NO: 3. In some embodiments, the signal peptide is cleaved during post-translational modifications that occur in vitro or in vivo.

In some embodiments, the monoclonal antibody comprises a first and third chain each comprises SEQ ID NO: 2 and a second and fourth chains each comprises SEQ ID NO: 3. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 2. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated above (in bold and underline) in SEQ ID NO: 2 and SEQ ID NO: 3. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 2 and SEQ ID NO: 3.

In some embodiments, one or more amino acids of an amino acid sequence encoding one or more CDRs of SEQ ID NO: 2 or SEQ ID No: 3 is substituted. In some embodiments, one or more amino acids of an amino acid sequence encoding one or more variable heavy chain CDRs of SEQ ID NO: 2 is substituted. In some embodiments, one or more amino acids of an amino acid sequence encoding one or more variable light chain CDRs of SEQ ID NO: 3 is substituted.

In some embodiments the monoclonal antibody is a scFv-Fc antibody comprising a first and second chain that associate together, each chain comprising a variable heavy chain (VH) domain of SEQ ID NO: 2, a linker

(e.g., GGGGSGGGGSGGGGS (SEQ ID NO: 13)),

a variable light chain (VL) domain of SEQ ID NO: 3, an Fc domain of SEQ ID NO: 2, and at least one additional domain. In some embodiments, the additional domain is another Fc domain of SEQ ID NO: 2.

In some embodiments the monoclonal antibody is a scFv-Fc-scFv antibody comprising a first and second chain that associate together, each chain comprising a first variable heavy chain (VH) domain of SEQ ID NO:2, a first linker

(e.g., GGGGSGGGGSGGGGS (SEQ ID NO: 13)),

a first variable light chain (VL) domain of SEQ ID NO: 3, an Fc domain of SEQ ID NO:2, a second variable heavy chain (VH) domain of SEQ ID NO: 2, a second linker

(e.g., GGGGSGGGGSGGGGS (SEQ ID NO: 13)),

and a second variable light chain (VL) domain of SEQ ID NO:3.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

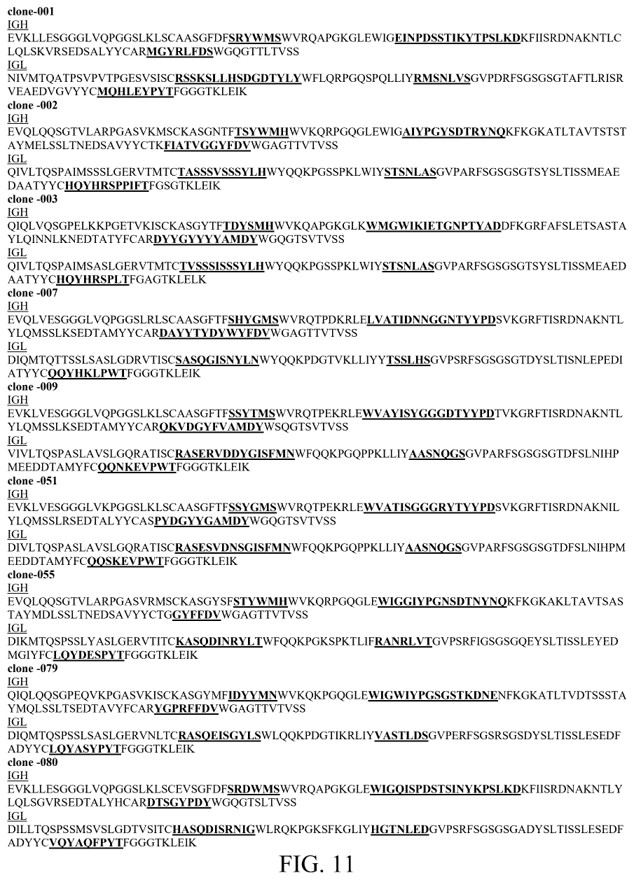

In some embodiments, the amino acid sequence of the anti-PD-1-Fc antibody heavy chain comprises SEQ ID NO: 14. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 14)
EVKLLESGGGLVQPGGSLKLSCAASGFDFSRYWMSWVRQAPGKGLEWIGEINPDSSTIK
YTPSLKDKFIISRDNAKNTLCLQLSKVRSEDSALYYCARMGYRLFDSWGQGTTLTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSRWQEGNVFSCSVM
HEALHNHYTQKSLSLSLGSTGSKYGPPCPPCPAPEFEGGPSVFLFPPKPKDTLMISRTPEVT
CVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHN
HYTQKSLSLSLG.

In some embodiments, the amino acid sequence of the anti-PD-1-Fc antibody heavy chain comprises SEQ ID NO: 1 immediately followed by SEQ ID NO: 14. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 15. Underlined amino acids represent the VL domain. In some embodiments, the underlined and bolded amino acids shown in FIG. 11 represent the respective complementarity determining regions (CDRs). In some embodiments, the CDRs are as provided in Table 3. Italicized amino acids represent the CL domain.

(SEQ ID NO: 15)
NIVMTQATPSVPVTPGESVSISCRSSKSLLHSDGDTYLYWFLQRPGQSPQLLIYRMSNLV
SGVPDRFSGSGSGTAFTLRISRVEAEDVGVYYCMQHLEYPYTFGGGTKLEIKTVAAPSVF
IFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGEC.

In some embodiments, the amino acid sequence of the anti-PD-1-Fc antibody light chain comprises SEQ ID NO: 1 immediately followed by SEQ ID NO: 15. In some embodiments, the signal peptide is cleaved during post-translational modifications that occur in vitro or in vivo.

In some embodiments, the monoclonal antibody comprises a first and third chain each comprises SEQ ID NO: 14 and a second and fourth chains each comprises SEQ ID NO: 15. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 14. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 15. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated in FIG. 11 or in Tables 1 and 3. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 14 and SEQ ID NO: 15 or the FRs in Tables 2 and 4.

In some embodiments, one or more amino acids of an amino acid sequence encoding one or more CDRs of SEQ ID NO: 14 or SEQ ID No: 15 is substituted. In some embodiments, one or more amino acids of an amino acid sequence encoding one or more variable heavy chain CDRs of SEQ ID NO: 14 is substituted. In some embodiments, one or more amino acids of an amino acid sequence encoding one or more variable light chain CDRs of SEQ ID NO: 15 is substituted.

In some embodiments the monoclonal antibody is a scFv-Fc antibody comprising a first and second chain that associate together, each chain comprising a variable heavy chain (VH) domain of SEQ ID NO: 14, a linker

(e.g., GGGGSGGGGSGGGGS), (SEQ ID NO: 13)),

a variable light chain (VL) domain of SEQ ID NO: 15, an Fc domain of SEQ ID NO: 14, and at least one additional domain. In some embodiments, the additional domain is another Fc domain of SEQ ID NO: 14.

In some embodiments the monoclonal antibody is a scFv-Fc-scFv antibody comprising a first and second chain that associate together, each chain comprising a first variable heavy chain (VH) domain of SEQ ID NO: 14, a first linker

(e.g., GGGGSGGGGSGGGGS (SEQ ID NO: 13)),

a first variable light chain (VL) domain of SEQ ID NO: 15, an Fc domain of SEQ ID NO: 14, a second variable heavy chain (VH) domain of SEQ ID NO: 14, a second linker

(e.g., GGGGSGGGGSGGGGS (SEQ ID NO: 13)),

and a second variable light chain (VL) domain of SEQ ID NO: 15.

An antibody comprising the VH and VL domains of SEQ ID NO: 14 and SEQ ID NO: 15 is represented by antibody “Clone 001” or “Clone 01” in embodiments described and depicted in this disclosure.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

In some embodiments, the amino acid sequence of the anti-PD-1-Fc antibody heavy chain comprises SEQ ID NO: 16. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 16)
EVQLQQSGTVLARPGASVKMSCKASGNTFTSYWMHWVKQRPGQGLEWIGAIYPGYSD
TRYNQKFKGKATLTAVTSTSTAYMELSSLTNEDSAVYYCTKFIATVGGYFDVWGAGTT
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEG
GPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGNVFS
CSVMHEALHNHYTQKSLSLSLGSTGSKYGPPCPPCPAPEFEGGPSVFFPPKPKDTLMISRT
PEVTCVVVDVSQEDPEVQFNWYVDGVEVHNAKTKPREEQFNSTYRVVSVLTVLHQDW
LNGKEYKCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVMHE
ALHNHYTQKSLSLSLG.

In some embodiments, the amino acid sequence of the anti-PD-1-Fc antibody heavy chain comprises SEQ ID NO: 1 immediately followed by SEQ ID NO: 16. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 17. Underlined amino acids represent the VL domain. In some embodiments, the underlined and bolded amino acids shown in FIG. 11 represent the respective complementarity determining regions (CDRs). In some embodiments, the CDRs are as provided in Table 3. Italicized amino acids represent the CL domain.

(SEQ ID NO: 17)
QIVLTQSPAIMSSSLGERVTMTCTASSSVSSSYLHWYQQKPGSSPKLWIYSTSNLASGVP
ARFSGSGSGTSYSLTISSMEAEDAATYYCHQYHRSPPIFTFGSGTKLEIKTVAAPSVFIFPP
SDEQLKSGTASVVCLLNNFYPREAKVQWKVDNALQSGNSQESVTEQDSKDSTYSLSSTLTLSK
ADYEKHKVYACEVTHQGLSSPVTKSFNRGEC.

In some embodiments, the amino acid sequence of the anti-PD-1-Fc antibody light chain comprises SEQ ID NO: 1 immediately followed by SEQ ID NO: 17. In some embodiments, the signal peptide is cleaved during post-translational modifications that occur in vitro or in vivo.

In some embodiments, the monoclonal antibody comprises a first and third chain each comprises SEQ ID NO: 16 and a second and fourth chains each comprises SEQ ID NO: 17. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 16. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 17. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated in FIG. 11 or in Tables 1 and 3. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 16 and SEQ ID NO: 17 or the FRs in Tables 2 and 4.

In some embodiments, one or more amino acids of an amino acid sequence encoding one or more CDRs of SEQ ID NO: 16 or SEQ ID No: 17 is substituted. In some embodiments, one or more amino acids of an amino acid sequence encoding one or more variable heavy chain CDRs of SEQ ID NO: 16 is substituted. In some embodiments, one or more amino acids of an amino acid sequence encoding one or more variable light chain CDRs of SEQ ID NO: 17 is substituted.

In some embodiments the monoclonal antibody is a scFv-Fc antibody comprising a first and second chain that associate together, each chain comprising a variable heavy chain (VH) domain of SEQ ID NO: 16, a linker

(e.g., GGGGSGGGGSGGGGS), (SEQ ID NO: 13)),

a variable light chain (VL) domain of SEQ ID NO: 17, an Fc domain of SEQ ID NO: 16, and at least one additional domain. In some embodiments, the additional domain is another Fc domain of SEQ ID NO: 16.

In some embodiments the monoclonal antibody is a scFv-Fc-scFv antibody comprising a first and second chain that associate together, each chain comprising a first variable heavy chain (VH) domain of SEQ ID NO:16, a first linker (e.g.,

GGGGSGGGGSGGGGS (SEQ ID NO: 13)),

a first variable light chain (VL) domain of SEQ ID NO: 17, an Fc domain of SEQ ID NO: 16, a second variable heavy chain (VH) domain of SEQ ID NO: 16, a second linker

(e.g., GGGGSGGGGSGGGGS (SEQ ID NO: 13)),

and a second variable light chain (VL) domain of SEQ ID NO: 17.

An antibody comprising the VH and VL of SEQ ID NO: 16 and SEQ ID NO: 17 is represented by antibody “Clone 002” or “Clone 02” in embodiments described and depicted in this disclosure.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

In some embodiments, the amino acid sequence of the anti-PD-1-Fc antibody heavy chain comprises SEQ ID NO: 18. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 18)
QIQLVQSGPELKKPGETVKISCKASGYTFTDYSMHWVKQAPGKGLKWMGWIKIETGNP
TYADDFKGRFAFSLETSASTAYLQINNLKNEDTATYFCARDYYGYYYYAMDYWGQGT
SVTVSSASTKGPSVFPLAPCSRSTSESTAALGCLVKDYFPEPVTVSWNSGALTSGVHTFP
AVLQSSGLYSLSSVVTVPSSSLGTKTYTCNVDHKPSNTKVDKRVESKYGPPCPPCPAPEFE
GGPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTVDKSRWQEGN
VFSCSVMHEALHNHYTQKSLSLSLGSTGSKYGPPCPPCPAPEFEGGPSVFLFPPKPKDTLMI
SRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSV
MHEALHNHYTQKSLSLSLG.

In some embodiments, the amino acid sequence of the anti-PD-1-Fc antibody heavy chain comprises SEQ ID NO: 1 immediately followed by SEQ ID NO: 18. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 19. Underlined amino acids represent the VL domain. In some embodiments, the underlined and bolded amino acids shown in FIG. 11 represent the respective complementarity determining regions (CDRs). In some embodiments, the CDRs are as provided in Table 3. Italicized amino acids represent the CL domain.

(SEQ ID NO: 19)
QIVLTQSPAIMSASLGERVTMTCTVSSSISSSYLHWYQQKPGSSPKLWIYSTSNLASGVP
ARFSGSGSGTSYSLTISSMEAEDAATYYCHQYHRSPLTFGAGTKLELKTVAAPSVFIFPPS
DEQLKSGTASVVCLLNNFYPREAKVQWKVDNALQSGNSQESVTEQDSKDSTYSLSSTLTLSKA
DYEKHKVYACEVTHQGLSSPVTKSFNRGEC.

In some embodiments, the amino acid sequence of the anti-PD-1-Fc antibody light chain comprises SEQ ID NO: 1 immediately followed by SEQ ID NO: 19. In some embodiments, the signal peptide is cleaved during post-translational modifications that occur in vitro or in vivo.

In some embodiments, the monoclonal antibody comprises a first and third chain each comprises SEQ ID NO: 18 and a second and fourth chains each comprises SEQ ID NO: 19. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 18. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 19. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated in FIG. 11 or in Tables 1 and 3. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 18 and SEQ ID NO: 19 or the FRs in Tables 2 and 4.

In some embodiments, one or more amino acids of an amino acid sequence encoding one or more CDRs of SEQ ID NO: 18 or SEQ ID No: 19 is substituted. In some embodiments, one or more amino acids of an amino acid sequence encoding one or more variable heavy chain CDRs of SEQ ID NO: 18 is substituted. In some embodiments, one or more amino acids of an amino acid sequence encoding one or more variable light chain CDRs of SEQ ID NO: 19 is substituted.

In some embodiments the monoclonal antibody is a scFv-Fc antibody comprising a first and second chain that associate together, each chain comprising a variable heavy chain (VH) domain of SEQ ID NO: 18, a linker

(e.g., GGGGSGGGGSGGGGS), (SEQ ID NO: 13)),

a variable light chain (VL) domain of SEQ ID NO: 19, an Fc domain of SEQ ID NO: 18, and at least one additional domain. In some embodiments, the additional domain is another Fc domain of SEQ ID NO: 18.

In some embodiments the monoclonal antibody is a scFv-Fc-scFv antibody comprising a first and second chain that associate together, each chain comprising a first variable heavy chain (VH) domain of SEQ ID NO:18, a first linker

(e.g., GGGGSGGGGSGGGGS (SEQ ID NO: 13)),

a first variable light chain (VL) domain of SEQ ID NO: 19, an Fc domain of SEQ ID NO: 18, a second variable heavy chain (VH) domain of SEQ ID NO: 18, a second linker

(e.g., GGGGSGGGGSGGGGS (SEQ ID NO: 13)),

and a second variable light chain (VL) domain of SEQ ID NO: 19.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

An antibody comprising the VH and VL of SEQ ID NO: 18 and SEQ ID NO: 19 is represented by antibody “Clone 003” or “Clone 03” in embodiments described and depicted in this disclosure.

In some embodiments, the amino acid sequence of the anti-PD-1-Fc antibody heavy chain comprises SEQ ID NO: 20. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 20)
EVQLVESGGGLVQPGGSLRLSCAASGFTFSHYGMSWVRQTPDKRLELVATIDNNGGNT
YYPDSVKGRFTISRDNAKNTLYLQMSSLKSEDTAMYYCARDAYYTYDYWYFDVWGA
GTTVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCPAPE
FEGGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSRWQE
GNVFSCSVMHEALHNHYTQKSLSLSLGSTGSKYGPPCPPCPAPEFEGGPSVFLFPPKPKDTL
MISRTPEVTCVVVDVSQEDPEVQFNWYVDGVEVHNAKTKPREEQFNSTYRVVSVLTVL
HQDWLNGKEYKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQEGNVFSCSV
MHEALHNHYTQKSLSLSLG.

In some embodiments, the amino acid sequence of the anti-PD-1-Fc antibody heavy chain comprises SEQ ID NO: 1 immediately followed by SEQ ID NO: 20. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 21. Underlined amino acids represent the VL domain. In some embodiments, the underlined and bolded amino acids shown in FIG. 11 represent the respective complementarity determining regions (CDRs). In some embodiments, the CDRs are as provided in Table 1. Italicized amino acids represent the CL domain.

(SEQ ID NO: 21)
DIQMTQTTSSLSASLGDRVTISCSASQGISNYLNWYQQKPDGTVKLLIYYTSSLHSGVPS
RFSGSGSGTDYSLTISNLEPEDIATYYCQQYHKLPWTFGGGTKLEIKTVAAPSVFIFPPSDE
QLKSGTASVVCLLNNFYPREAKVQWKVDNALQSGNSQESVTEQDSKDSTYSLSSTLTLSKADY
EKHKVYACEVTHQGLSSPVTKSFNRGEC.

In some embodiments, the amino acid sequence of the anti-PD-1-Fc antibody light chain comprises SEQ ID NO: 1 immediately followed by SEQ ID NO: 21. In some embodiments, the signal peptide is cleaved during post-translational modifications that occur in vitro or in vivo.

In some embodiments, the monoclonal antibody comprises a first and third chain each comprises SEQ ID NO: 20 and a second and fourth chains each comprises SEQ ID NO: 21. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 20. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 21. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated in FIG. 11 or in Tables 1 and 3. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 20 and SEQ ID NO: 21 or the FRs in Tables 2 and 4.

In some embodiments, one or more amino acids of an amino acid sequence encoding one or more CDRs of SEQ ID NO: 20 or SEQ ID No: 21 is substituted. In some embodiments, one or more amino acids of an amino acid sequence encoding one or more variable heavy chain CDRs of SEQ ID NO: 20 is substituted. In some embodiments, one or more amino acids of an amino acid sequence encoding one or more variable light chain CDRs of SEQ ID NO: 21 is substituted.

In some embodiments the monoclonal antibody is a scFv-Fc antibody comprising a first and second chain that associate together, each chain comprising a variable heavy chain (VH) domain of SEQ ID NO: 20, a linker

(e.g., GGGGSGGGGSGGGGS), (SEQ ID NO: 13)),

a variable light chain (VL) domain of SEQ ID NO: 21, an Fc domain of SEQ ID NO: 20, and at least one additional domain. In some embodiments, the additional domain is another Fc domain of SEQ ID NO: 20.

In some embodiments the monoclonal antibody is a scFv-Fc-scFv antibody comprising a first and second chain that associate together, each chain comprising a first variable heavy chain (VH) domain of SEQ ID NO: 20, a first linker

(e.g., GGGGSGGGGSGGGGS (SEQ ID NO: 13)),

a first variable light chain (VL) domain of SEQ ID NO: 21, an Fc domain of SEQ ID NO: 20, a second variable heavy chain (VH) domain of SEQ ID NO: 20, a second linker

(e.g., GGGGSGGGGSGGGGS (SEQ ID NO: 13)),

and a second variable light chain (VL) domain of SEQ ID NO: 21.

An antibody comprising the VH and VL SEQ ID NO: 20 and SEQ ID NO: 21 is represented by antibody “Clone 007” or “Clone 07” in embodiments described and depicted in this disclosure.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

In some embodiments, the amino acid sequence of the anti-PD-1-Fc antibody heavy chain comprises SEQ ID NO: 22. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 22)
EVKLVESGGGLVQPGGSLKLSCAASGFTFSSYTMSWVRQTPEKRLEWVAYISYGGGDT
YYPDTVKGRFTISRDNAKNTLYLQMSSLKSEDTAMYYCARQKVDGYFVAMDYWSQG
TSVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRWQEG
NVFSCSVMHEALHNHYTQKSLSLSLGSTGSKYGPPCPPCPAPEFEGGPSVFLFPPKPKDTLM
ISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSV
MHEALHNHYTQKSLSLSLG.

In some embodiments, the amino acid sequence of the anti-PD-1-Fc antibody heavy chain comprises SEQ ID NO: 1 immediately followed by SEQ ID NO: 22. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 23. Underlined amino acids represent the VL domain. In some embodiments, the underlined and bolded amino acids shown in FIG. 11 represent the respective complementarity determining regions (CDRs). In some embodiments, the CDRs are as provided in Table 1. Italicized amino acids represent the CL domain.

(SEQ ID NO: 23)
VIVLTQSPASLAVSLGQRATISCRASERVDDYGISFMNWFQQKPGQPPKLLIYAASNQGS
GVPARFSGSGSGTDFSLNIHPMEEDDTAMYFCQQNKEVPWTFGGGTKLEIKTVAAPSVFI
FPPSDEQLKSGTASVVCLLNNFYPREAKVQWKVDNALQSGNSQESVTEQDSKDSTYSLSSTLT
LSKADYEKHKVYACEVTHQGLSSPVTKSFNRGEC.

In some embodiments, the amino acid sequence of the anti-PD-1-Fc antibody light chain comprises SEQ ID NO: 1 immediately followed by SEQ ID NO: 23. In some embodiments, the signal peptide is cleaved during post-translational modifications that occur in vitro or in vivo.

In some embodiments, the monoclonal antibody comprises a first and third chain each comprises SEQ ID NO: 22 and a second and fourth chains each comprises SEQ ID NO: 23. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 22. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 23. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated in FIG. 11 or in Tables 1 and 3. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 22 and SEQ ID NO: 23 or the FRs in Tables 2 and 4.

In some embodiments, one or more amino acids of an amino acid sequence encoding one or more CDRs of SEQ ID NO: 22 or SEQ ID No: 23 is substituted. In some embodiments, one or more amino acids of an amino acid sequence encoding one or more variable heavy chain CDRs of SEQ ID NO: 22 is substituted. In some embodiments, one or more amino acids of an amino acid sequence encoding one or more variable light chain CDRs of SEQ ID NO: 23 is substituted.

In some embodiments the monoclonal antibody is a scFv-Fc antibody comprising a first and second chain that associate together, each chain comprising a variable heavy chain (VH) domain of SEQ ID NO: 22, a linker

(e.g., GGGGSGGGGSGGGGS), (SEQ ID NO: 13)),

a variable light chain (VL) domain of SEQ ID NO: 23, an Fc domain of SEQ ID NO: 22, and at least one additional domain. In some embodiments, the additional domain is another Fc domain of SEQ ID NO: 22.

In some embodiments the monoclonal antibody is a scFv-Fc-scFv antibody comprising a first and second chain that associate together, each chain comprising a first variable heavy chain (VH) domain of SEQ ID NO: 22, a first linker

(e.g., GGGGSGGGGSGGGGS (SEQ ID NO: 13)),

a first variable light chain (VL) domain of SEQ ID NO: 23, an Fc domain of SEQ ID NO: 22, a second variable heavy chain (VH) domain of SEQ ID NO: 22, a second linker

(e.g., GGGGSGGGGSGGGGS (SEQ ID NO: 13)),

and a second variable light chain (VL) domain of SEQ ID NO: 23.

An antibody comprising the VH and VL of SEQ ID NO: 22 and SEQ ID NO: 23 is represented by antibody “Clone 009” or “Clone 09” in embodiments described and depicted in this disclosure.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

In some embodiments, the amino acid sequence of the anti-PD-1-Fc antibody heavy chain comprises SEQ ID NO: 24. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 24)
EVKLVESGGGLVKPGGSLKLSCAASGFTFSSYGMSWVRQTPEKRLEWVATISGGGRYT
YYPDSVKGRFTISRDNAKNILYLQMSSLRSEDTALYYCASPYDGYYGAMDYWGQGTS
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEG
GPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGNVFS
CSVMHEALHNHYTQKSLSLSLGSTGSKYGPPCPPCPAPEFEGGPSVFLFPPKPKDTLMISRT
PEVTCVVVDVSQEDPEVQFNWYVDGVEVHNAKTKPREEQFNSTYRVVSVLTVLHQDW
LNGKEYKCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVMHE
ALHNHYTQKSLSLSLG.

In some embodiments, the amino acid sequence of the anti-PD-1-Fc antibody heavy chain comprises SEQ ID NO: 1 immediately followed by SEQ ID NO: 24. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 25. Underlined amino acids represent the VL domain. In some embodiments, the underlined and bolded amino acids shown in FIG. 11 represent the respective complementarity determining regions (CDRs). In some embodiments, the CDRs are as provided in Table 1. Italicized amino acids represent the CL domain.

(SEQ ID NO: 25)
DIVLTQSPASLAVSLGQRATISCRASESVDNSGISFMNWFQQKPGQPPKLLIYAASNQGS
GVPARFSGSGSGTDFSLNIHPMEEDDTAMYFCQQSKEVPWTFGGGTKLEIKTVAAPSVFI
FPPSDEQLKSGTASVVCLLNNFYPREAKVQWKVDNALQSGNSQESVTEQDSKDSTYSLSSTLT
LSKADYEKHKVYACEVTHQGLSSPVTKSFNRGEC.

In some embodiments, the amino acid sequence of the anti-PD-1-Fc antibody light chain comprises SEQ ID NO: 1 immediately followed by SEQ ID NO: 25. In some embodiments, the signal peptide is cleaved during post-translational modifications that occur in vitro or in vivo.

In some embodiments, the monoclonal antibody comprises a first and third chain each comprises SEQ ID NO: 24 and a second and fourth chains each comprises SEQ ID NO: 25. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 24. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 25. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated in FIG. 11 or in Tables 1 and 3. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 24 and SEQ ID NO: 25 or the FRs in Tables 2 and 4.

In some embodiments, one or more amino acids of an amino acid sequence encoding one or more CDRs of SEQ ID NO: 24 or SEQ ID No: 25 is substituted. In some embodiments, one or more amino acids of an amino acid sequence encoding one or more variable heavy chain CDRs of SEQ ID NO: 24 is substituted. In some embodiments, one or more amino acids of an amino acid sequence encoding one or more variable light chain CDRs of SEQ ID NO: 25 is substituted.

In some embodiments the monoclonal antibody is a scFv-Fc antibody comprising a first and second chain that associate together, each chain comprising a variable heavy chain (VH) domain of SEQ ID NO: 24, a linker

(e.g., GGGGSGGGGSGGGGS), (SEQ ID NO: 13)),

a variable light chain (VL) domain of SEQ ID NO: 25, an Fc domain of SEQ ID NO: 24, and at least one additional domain. In some embodiments, the additional domain is another Fc domain of SEQ ID NO: 24.

In some embodiments the monoclonal antibody is a scFv-Fc-scFv antibody comprising a first and second chain that associate together, each chain comprising a first variable heavy chain (VH) domain of SEQ ID NO: 24, a first linker

(e.g., GGGGSGGGGSGGGGS (SEQ ID NO: 13)),

a first variable light chain (VL) domain of SEQ ID NO: 25, an Fc domain of SEQ ID NO: 24, a second variable heavy chain (VH) domain of SEQ ID NO: 24, a second linker

(e.g., GGGGSGGGGSGGGGS (SEQ ID NO: 13)),

and a second variable light chain (VL) domain of SEQ ID NO: 25.

An antibody comprising the VH and VL of SEQ ID NO: 24 and SEQ ID NO: 25 is represented by antibody “Clone 051” or “Clone 51” in embodiments described and depicted in this disclosure.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

In some embodiments, the amino acid sequence of the anti-PD-1-Fc antibody heavy chain comprises SEQ ID NO: 92. Underlined amino acids represent the VH domain. Underlined and bolded amino acids represent the respective complementarity determining regions (CDRs). Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 92)
EVQLVESGGGLVKPGGSLRLSCAASGFTFSSYGMSWVRQAPGKGLEWVATISGGGRY
TYYPDSVKGRFTISRDNAKNSLYLQMNSLRAEDTAVYYCASPYDGYYGAMDYWGQG
TSVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRWEG
NVFSCSVMHEALHNHYTQKSLSLSLGSTGSKYGPPCPPCPAPEFEGGPSVFLFPPKPKDTLM
ISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSV
MHEALHNHYTQKSLSLSLGMHEALHNHYTQKSLSLSLG.

In some embodiments, the amino acid sequence of the anti-PD-1-Fc antibody heavy chain comprises SEQ ID NO: 1 immediately followed by SEQ ID NO: 92. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 93. Underlined amino acids represent the VL domain. Underlined and bolded amino acids represent the respective complementarity determining regions (CDRs). Italicized amino acids represent the CL domain.

(SEQ ID NO: 93)
EIVLTQSPATLSLSPGERATLSCRASESVDNSGISFMNWYQQKPGQAPRLLIYAASNQG
SGIPARFSGSGSGTDFTLTISSLEPEDFAVYYCQQSKEVPWTFGGGTKLEIKRTVAAPSVF
IFPPSDEQLKSGTASVVCLLNNFYPREAKVQWKVDNALQSGNSQESVTEQDSKDSTYSLSSTL
TLSKADYEKHKVYACEVTHQGLSSPVTKSFNRGEC.

In some embodiments, the amino acid sequence of the anti-PD-1-Fc antibody light chain comprises SEQ ID NO: 1 immediately followed by SEQ ID NO: 93. In some embodiments, the signal peptide is cleaved during post-translational modifications that occur in vitro or in vivo.

In some embodiments, the monoclonal antibody comprises a first and third chain each comprises SEQ ID NO: 74 and a second and fourth chains each comprises SEQ ID NO: 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 74. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 75. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated above. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs in Tables 2 and 4.

In some embodiments, one or more amino acids of an amino acid sequence encoding one or more CDRs of SEQ ID NO: 74 or SEQ ID No: 75 is substituted. In some embodiments, one or more amino acids of an amino acid sequence encoding one or more variable heavy chain CDRs of SEQ ID NO: 74 is substituted. In some embodiments, one or more amino acids of an amino acid sequence encoding one or more variable light chain CDRs of SEQ ID NO: 75 is substituted.

In some embodiments the monoclonal antibody is a scFv-Fc antibody comprising a first and second chain that associate together, each chain comprising a variable heavy chain (VH) domain of SEQ ID NO: 74, a linker

(e.g., GGGGSGGGGSGGGGS (SEQ ID NO: 13)),

a variable light chain (VL) domain of SEQ ID NO: 75, an Fc domain of SEQ ID NO: 74, and at least one additional domain. In some embodiments, the additional domain is another Fc domain of SEQ ID NO: 74.

In some embodiments the monoclonal antibody is a scFv-Fc-scFv antibody comprising a first and second chain that associate together, each chain comprising a first variable heavy chain (VH) domain of SEQ ID NO: 74, a first linker

(e.g., GGGGSGGGGSGGGGS (SEQ ID NO: 13)),

a first variable light chain (VL) domain of SEQ ID NO: 75, an Fc domain of SEQ ID NO: 74, a second variable heavy chain (VH) domain of SEQ ID NO: 74, a second linker

(e.g., GGGGSGGGGSGGGGS (SEQ ID NO: 13)),

and a second variable light chain (VL) domain of SEQ ID NO: 75.

An antibody comprising the VH and VL of SEQ ID NO: 74 and SEQ ID NO: 75 is a humanized version of the VH and VL portions of antibody “Clone 051” or “Clone 51” in embodiments described and depicted in this disclosure.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

In some embodiments, the amino acid sequence of the anti-PD-1-Fc antibody heavy chain comprises SEQ ID NO: 26. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 26)
EVQLQQSGTVLARPGASVRMSCKASGYSFSTYWMHWVKQRPGQGLEWIGGIYPGNSD
TNYNQKFKGKAKLTAVTSASTAYMDLSSLTNEDSAVYYCTGGYFFDVWGAGTTVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPSV
FLFPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSV
MHEALHNHYTQKSLSLSLGSTGSKYGPPCPPCPAPEFEGGPSVFLFPPKPKDTLMISRTPEV
TCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCSVMHEALH
NHYTQKSLSLSLG.

In some embodiments, the amino acid sequence of the anti-PD-1-Fc antibody heavy chain comprises SEQ ID NO: 1 immediately followed by SEQ ID NO: 26. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 27. Underlined amino acids represent the VL domain. In some embodiments, the underlined and bolded amino acids shown in FIG. 11 represent the respective complementarity determining regions (CDRs). In some embodiments, the CDRs are as provided in Table 1. Italicized amino acids represent the CL domain.

(SEQ ID NO: 27)
DIKMTQSPSSLYASLGERVTITCKASQDINRYLTWFQQKPGKSPKTLIFRANRLVTGVPS
RFIGSGSGQEYSLTISSLEYEDMGIYFCLQYDESPYTFGGGTKLEIKTVAAPSVFIFPPSDE
QLKSGTASVVCLLNNFYPREAKVQWKVDNALQSGNSQESVTEQDSKDSTYSLSSTLTLSKADY
EKHKVYACEVTHQGLSSPVTKSFNRGEC.

In some embodiments, the amino acid sequence of the anti-PD-1-Fc antibody light chain comprises SEQ ID NO: 1 immediately followed by SEQ ID NO: 27. In some embodiments, the signal peptide is cleaved during post-translational modifications that occur in vitro or in vivo.

In some embodiments, the monoclonal antibody comprises a first and third chain each comprises SEQ ID NO: 26 and a second and fourth chains each comprises SEQ ID NO: 27. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 26. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 27. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated in FIG. 11 or in Tables 1 and 3. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 26 and SEQ ID NO: 27 or the FRs in Tables 2 and 4.

In some embodiments, one or more amino acids of an amino acid sequence encoding one or more CDRs of SEQ ID NO: 26 or SEQ ID No: 27 is substituted. In some embodiments, one or more amino acids of an amino acid sequence encoding one or more variable heavy chain CDRs of SEQ ID NO: 26 is substituted. In some embodiments, one or more amino acids of an amino acid sequence encoding one or more variable light chain CDRs of SEQ ID NO: 27 is substituted.

In some embodiments the monoclonal antibody is a scFv-Fc antibody comprising a first and second chain that associate together, each chain comprising a variable heavy chain (VH) domain of SEQ ID NO: 26, a linker

(e.g., GGGGSGGGGSGGGGS (SEQ ID NO: 13)),

a variable light chain (VL) domain of SEQ ID NO: 27, an Fc domain of SEQ ID NO: 26, and at least one additional domain. In some embodiments, the additional domain is another Fc domain of SEQ ID NO: 26.

In some embodiments the monoclonal antibody is a scFv-Fc-scFv antibody comprising a first and second chain that associate together, each chain comprising a first variable heavy chain (VH) domain of SEQ ID NO: 26, a first linker

(e.g., GGGGSGGGGSGGGGS (SEQ ID NO: 13)),

a first variable light chain (VL) domain of SEQ ID NO: 27, an Fc domain of SEQ ID NO: 26, a second variable heavy chain (VH) domain of SEQ ID NO: 26, a second linker

(e.g., GGGGSGGGGSGGGGS (SEQ ID NO: 13)),

and a second variable light chain (VL) domain of SEQ ID NO: 27.

An antibody comprising the VH and VL of SEQ ID NO: 26 and SEQ ID NO: 27 is represented by antibody “Clone 055” or “Clone 55” in embodiments described and depicted in this disclosure.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

In some embodiments, the amino acid sequence of the anti-PD-1-Fc antibody heavy chain comprises SEQ ID NO: 28. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 28)
QIQLQQSGPEQVKPGASVKISCKASGYMFIDYYMNWVKQKPGQGLEWIGWIYPGSGST
KDNENFKGKATLTVDTSSSTAYMQLSSLTSEDTAVYFCARYGPRFFDVWGAGTTVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPSV
FLFPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSV
MHEALHNHYTQKSLSLSLGSTGSKYGPPCPPCPAPEFEGGPSVFLFPPKPKDTLMISRTPEV
TCVVVDVSQEDPEVQFNWYVDGVEVHNAKTKPREEQFNSTYRVVSVLTVLHQDWLN
GKEYKCKVSNKGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCSVMHEALH
NHYTQKSLSLSLG.

In some embodiments, the amino acid sequence of the anti-PD-1-Fc antibody heavy chain comprises SEQ ID NO: 1 immediately followed by SEQ ID NO: 28. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 29. Underlined amino acids represent the VL domain. In some embodiments, the underlined and bolded amino acids shown in FIG. 11 represent the respective complementarity determining regions (CDRs). In some embodiments, the CDRs are as provided in Table 1. Italicized amino acids represent the CL domain.

(SEQ ID NO: 29)
DIQMTQSPSSLSASLGERVNLTCRASQEISGYLSWLQQKPDGTIKRLIYVASTLDSGVPE
RFSGSRSGSDYSLTISSLESEDFADYYCLQYASYPYTFGGGTKLEIKTVAAPSVFIFPPSDE
QLKSGTASVVCLLNNFYPREAKVQWKVDNALQSGNSQESVTEQDSKDSTYSLSSTLTLSKADY
EKHKVYACEVTHQGLSSPVTKSFNRGEC.

In some embodiments, the amino acid sequence of the anti-PD-1-Fc antibody light chain comprises SEQ ID NO: 1 immediately followed by SEQ ID NO: 29. In some embodiments, the signal peptide is cleaved during post-translational modifications that occur in vitro or in vivo.

In some embodiments, the monoclonal antibody comprises a first and third chain each comprises SEQ ID NO: 28 and a second and fourth chains each comprises SEQ ID NO: 29. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 28. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 29. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated in FIG. 11 or in Tables 1 and 3. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 28 and SEQ ID NO: 29 or the FRs in Tables 2 and 4.

In some embodiments, one or more amino acids of an amino acid sequence encoding one or more CDRs of SEQ ID NO: 28 or SEQ ID No: 29 is substituted. In some embodiments, one or more amino acids of an amino acid sequence encoding one or more variable heavy chain CDRs of SEQ ID NO: 28 is substituted. In some embodiments, one or more amino acids of an amino acid sequence encoding one or more variable light chain CDRs of SEQ ID NO: 29 is substituted.

In some embodiments the monoclonal antibody is a scFv-Fc antibody comprising a first and second chain that associate together, each chain comprising a variable heavy chain (VH) domain of SEQ ID NO: 28, a linker.

(e.g., GGGGSGGGGSGGGGS (SEQ ID NO: 13)),

a variable light chain (VL) domain of SEQ ID NO: 29, an Fc domain of SEQ ID NO: 28, and at least one additional domain. In some embodiments, the additional domain is another Fc domain of SEQ ID NO: 28.

In some embodiments the monoclonal antibody is a scFv-Fc-scFv antibody comprising a first and second chain that associate together, each chain comprising a first variable heavy chain (VH) domain of SEQ ID NO: 28, a first linker

(e.g., GGGGSGGGGSGGGGS (SEQ ID NO: 13)),

a first variable light chain (VL) domain of SEQ ID NO: 29, an Fc domain of SEQ ID NO: 28, a second variable heavy chain (VH) domain of SEQ ID NO: 28, a second linker

(e.g., GGGGSGGGGSGGGGS (SEQ ID NO: 13)),

and a second variable light chain (VL) domain of SEQ ID NO: 29.

An antibody comprising the VH and VL of SEQ ID NO: 28 and SEQ ID NO: 29 is represented by antibody “Clone 079” or “Clone 79” in embodiments described and depicted in this disclosure.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

In some embodiments, the amino acid sequence of the anti-PD-1-Fc antibody heavy chain comprises SEQ ID NO: 30. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 30)
EVKLLESGGGLVQPGGSLKLSCEVSGFDFSRDWMSWVRQAPGKGLEWIGQISPDSTSIN
YKPSLKDKFIISRDNAKNTLYLQLSGVRSEDTALYHCARDTSGYPDYWGQGTSLTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSRWQEGNVFSCSVM
HEALHNHYTQKSLSLSLGSTGSKYGPPCPPCPAPEFEGGPSVFLFPPKPKDTLMISRTPEVT
CVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHN
HYTQKSLSLSLG.

In some embodiments, the amino acid sequence of the anti-PD-1-Fc antibody heavy chain comprises SEQ ID NO: 1 immediately followed by SEQ ID NO: 30. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 31. Underlined amino acids represent the VL domain. In some embodiments, the underlined and bolded amino acids shown in FIG. 11 represent the respective complementarity determining regions (CDRs). In some embodiments, the CDRs are as provided in Table 1. Italicized amino acids represent the CL domain.

(SEQ ID NO: 31)
DILLTQSPSSMSVSLGDTVSITCHASQDISRNIGWLRQKPGKSFKGLIYHGTNLEDGVPSR
FSGSGSGADYSLTISSLESEDFADYYCVQYAQFPYTFGGGTKLEIKTVAAPSVFIFPPSDE
QLKSGTASVVCLLNNFYPREAKVQWKVDNALQSGNSQESVTEQDSKDSTYSLSSTLTLSKADY
EKHKVYACEVTHQGLSSPVTKSFNRGEC.

In some embodiments, the amino acid sequence of the anti-PD-1-Fc antibody light chain comprises SEQ ID NO: 1 immediately followed by SEQ ID NO: 31. In some embodiments, the signal peptide is cleaved during post-translational modifications that occur in vitro or in vivo.

In some embodiments, the monoclonal antibody comprises a first and third chain each comprises SEQ ID NO: 30 and a second and fourth chains each comprises SEQ ID NO: 31. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 30. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 31. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated in FIG. 11 or in Tables 1 and 3. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 30 and SEQ ID NO: 31 or the FRs in Tables 2 and 4.

In some embodiments, one or more amino acids of an amino acid sequence encoding one or more CDRs of SEQ ID NO: 30 or SEQ ID No: 31 is substituted. In some embodiments, one or more amino acids of an amino acid sequence encoding one or more variable heavy chain CDRs of SEQ ID NO: 30 is substituted. In some embodiments, one or more amino acids of an amino acid sequence encoding one or more variable light chain CDRs of SEQ ID NO: 31 is substituted.

In some embodiments the monoclonal antibody is a scFv-Fc antibody comprising a first and second chain that associate together, each chain comprising a variable heavy chain (VH) domain of SEQ ID NO: 30, a linker

(e.g.,
(SEQ ID NO: 13)
GGGGSGGGGSGGGGS),

a variable light chain (VL) domain of SEQ ID NO: 31, an Fc domain of SEQ ID NO: 30, and at least one additional domain. In some embodiments, the additional domain is another Fc domain of SEQ ID NO: 30.

In some embodiments the monoclonal antibody is a scFv-Fc-scFv antibody comprising a first and second chain that associate together, each chain comprising a first variable heavy chain (VH) domain of SEQ ID NO: 30, a first linker

(e.g.,
(SEQ ID NO: 13)
GGGGSGGGGSGGGGS),

a first variable light chain (VL) domain of SEQ ID NO: 31, an Fc domain of SEQ ID NO: 30, a second variable heavy chain (VH) domain of SEQ ID NO: 30, a second linker

(e.g.,
(SEQ ID NO: 13)
GGGGSGGGGSGGGGS),

and a second variable light chain (VL) domain of SEQ ID NO: 31.

An antibody comprising the VH and VL of SEQ ID NO: 30 and SEQ ID NO: 31 is represented by antibody “Clone 080” or “Clone 80” in embodiments described and depicted in this disclosure.

In some embodiments, the amino acid sequence of the anti-PD-1-Fc antibody heavy chain comprises a signal peptide comprising SEQ ID NO: 1. MGWSCIILFLVATATGVHS (SEQ ID NO: 1).

In some embodiments, the amino acid sequence of the anti-PD-1-Fc antibody heavy chain comprises SEQ ID NO: 92. Underlined amino acids represent the VH domain. Amino acids for the respective complementarity determining regions (CDRs) are provided in Table 1. Italicized and bold amino acids represent the CH1 domain. Italicized and underlined amino acids represent the hinge domain. Italicized amino acids represent the Fc domain. Double underlined amino acids represent the additional Fc domain.

(SEQ ID NO: 90)
EVKLVESGGGLVQPGGSLKLSCAASGFTFSSYTMSWVRQTPEKRLE
WVAYISYGGGDTYYSDTVKGRFTISRDNAKNTLYLQMSSLKSEDT
AMYYCARQKVDGYYVGMDYWGQGSSVTVSSASTKGPSVFPLAPCS
RSTSESTAALGCLVKDYFPEPVTVSWNSGALTSGVHTFPAVLQSS
GLYSLSSVVTVPSSSLGTKTYTCNVDHKPSNTKVDKRVESKYGPP
CPPCPAPEFEGGPSVFLFPPKPKDTLMISRTPEVTCVVVDVSQED
PEVQFNWYVDGVEVHNAKTKPREEQFNSTYRVVSVLTVLHQDWLN
GKEYKCKVSNKGLPSSIEKTISKAKGQPREPQVYTLPPSQEEMTK
NQVSLTCLVKGFYPSDIAVEWESNGQPENNYKTTPPVLDSDGSFF
LYSRLTVDKSRWQEGNVFSCSVMHEALHNHYTQKSLSLSLGSTGS
KYGPPCPPCPAPEFEGGPSVFLFPPKPKDTLMISRTPEVTCVVVD
VSQEDPEVQFNWYVDGVEVHNAKTKPREEQENSTYRVVSVLTVLH
QDWLNGKEYKCKVSNKGLPSSIEKTISKAKGQPREPQVYTLPPSQ
EEMTKNQVSLTCLVKGFYPSDIAVEWESNGQPENNYKTTPPVLDS
DGSFFLYSRLTVDKSRWQEGNVESCSVMHEALHNHYTQKSLSLSL
G.

In some embodiments, the amino acid sequence of the anti-PD-1-Fc antibody heavy chain comprises SEQ ID NO: 1 immediately followed by SEQ ID NO: 90. In some embodiments, the signal peptide is cleaved during post-translational modifications that occur in vitro or in vivo.

In some embodiments, the amino acid sequence of the anti-PD-1-Fc antibody light chain (without the signal peptide) comprises SEQ ID NO: 91. Underlined amino acids represent the VL domain. Amino acids for the respective complementarity determining regions (CDRs) are provided in Table 3. Italicized amino acids represent the CL domain.

(SEQ ID NO: 91)
NIVMTQSPKSMSMSVGERVTLSCKASENVGTNVSWYQQKPEQSPRL
LIYGASNRYTGVPDRFTGSGSVTDFTLTISSVQAEDLVDYHCGQS
YSSPLTFGSGTKLELKTVAAPSVFIFPPSDEQLKSGTASVVCLLN
NFYPREAKVQWKVDNALQSGNSQESVTEQDSKDSTYSLSSTLTLS
KADYEKHKVYACEVTHOGLSSPVTKSENRGEC.

In some embodiments, the amino acid sequence of the anti-PD-1-Fc antibody light chain comprises SEQ ID NO: 1 immediately followed by SEQ ID NO: 91. In some embodiments, the signal peptide is cleaved during post-translational modifications that occur in vitro or in vivo.

In some embodiments, the monoclonal antibody comprises a first and third chain each comprises SEQ ID NO: 72 and a second and fourth chains each comprises SEQ ID NO: 73. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-Fc antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-Fc antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 72. In some embodiments, the anti-PD-1-Fc antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 73. In some embodiments, the anti-PD-1-Fc antibody comprises an amino acid sequence comprising the CDRs of the variable heavy chain domain and the CDRs of the variable light chain domain of the anti-PD-1-Fc antibody, wherein the CDR sequences are indicated in Tables 1 and 3. In some embodiments, the framework regions (FRs) of the variable heavy chain domain and the FRs of the variable light chain domain of the anti-PD-1-Fc antibody comprise an amino acid sequence 80, 81, 82, 83, 84, 85, 86, 87, 88, 89, 90, 91, 92, 93, 94, 95, 96, 97, 98 or 99% identical to the FRs in Tables 2 and 4.

In some embodiments, one or more amino acids of an amino acid sequence encoding one or more CDRs of SEQ ID NO: 72 or SEQ ID No: 73 is substituted. In some embodiments, one or more amino acids of an amino acid sequence encoding one or more variable heavy chain CDRs of SEQ ID NO: 72 is substituted. In some embodiments, one or more amino acids of an amino acid sequence encoding one or more variable light chain CDRs of SEQ ID NO: 73 is substituted.

In some embodiments the monoclonal antibody is a scFv-Fc antibody comprising a first and second chain that associate together, each chain comprising a variable heavy chain (VH) domain of SEQ ID NO: 72, a linker

(e.g.,
(SEQ ID NO: 13)
GGGGSGGGGSGGGGS),

a variable light chain (VL) domain of SEQ ID NO: 73, an Fc domain of SEQ ID NO: 72, and at least one additional domain. In some embodiments, the additional domain is another Fc domain of SEQ ID NO: 72.

In some embodiments the monoclonal antibody is a scFv-Fc-scFv antibody comprising a first and second chain that associate together, each chain comprising a first variable heavy chain (VH) domain of SEQ ID NO: 72, a first linker

(e.g.,
(SEQ ID NO: 13)
GGGGSGGGGSGGGGS),

a first variable light chain (VL) domain of SEQ ID NO: 73, an Fc domain of SEQ ID NO: 72, a second variable heavy chain (VH) domain of SEQ ID NO: 72, a second linker

(e.g.,
(SEQ ID NO: 13)
GGGGSGGGGSGGGGS),

and a second variable light chain (VL) domain of SEQ ID NO: 73.

An antibody comprising the VH and VL of SEQ ID NO: 72 and SEQ ID NO: 73 is represented by antibody “Clone 004” or “Clone 4” in embodiments described and depicted in this disclosure.

### Nucleic Acid Sequences of Anti-PD-1-Fc Antibodies

A person skilled in the art can identify the nucleic acid sequences encoding the features identified in the corresponding amino acid sequences (e.g., CDRs, variable heavy domain, variable light domain, CH1 domain, hinge domain, Fc domain, and any additional FC domain) by translating the nucleic acid sequences into amino acid sequences. In some embodiments, the nucleic acid sequences may be codon optimized. . . . In some embodiments, nucleic acids encoding VH and VL sequences disclosed herein are provided in Table 5.

In some embodiments, the nucleic acid sequence of the anti-PD-1-Fc antibody comprises a nucleic acid sequence encoding a signal peptide comprising SEQ ID NO: 5. ATGGGCTGGAGCTGCATTATCCTGTTCCTGGTGGCCACAGCCACCGGCGTGCACAG C (SEQ ID NO: 5).

In some embodiments, the amino acid sequence of the anti-PD-1-Fc antibody heavy chain comprises SEQ ID NO: 2, 14, 16, 18, 20, 22, 24, 26, 28, 30, 72, or 74.

In some embodiments, the nucleic acid sequence encoding the anti-PD-1-Fc antibody heavy chain comprises SEQ ID NO: 6. CAGGTGCAGCTGGTGCAGTCCGGCGTGGAGGTGAAGAAGCCAGGCGCCTCCGTGAA GGTGTCTTGCAAGGCCAGCGGCTACACATTCACCAACTACTATATGTATTGGGTGCG GCAGGCACCTGGACAGGGCCTGGAGTGGATGGGAGGCATCAACCCATCCAATGGC GGCACAAACTTCAATGAGAAGTTTAAGAATCGGGTGACCCTGACCACAGATAGCTC CACCACAACCGCCTACATGGAGCTGAAGTCTCTGCAGTTTGACGATACAGCCGTGT ACTATTGCGCCCGGAGAGACTACAGATTCGATATGGGCTTTGACTATTGGGGCCAG GGCACACTGGTGACCGTGTCTAGCGCCAGCACCAAGGGTCCTAGCGTGTTCCCTCTG GCCCCATGCTCAAGAAGCACTTCCGAGTCTACCGCCGCTCTGGGGTGTCTGGTGAA GGACTACTTCCCCGAACCTGTCACCGTGAGCTGGAACTCCGGGGCCCTGACTTCCGG TGTCCACACCTTTCCCGCTGTGCTGCAGTCTAGTGGCCTGTACTCTCTGAGCAGCGT GGTCACTGTGCCTTCCTCTAGTCTGGGAACTAAGACCTATACATGCAACGTGGACCA TAAACCATCCAATACCAAGGTCGATAAACGCGTGGAGTCTAAGTACGGACCACCAT GCCCTCCATGTCCAGCACCAGAGTTCGAAGGCGGACCTTCCGTGTTCCTGTTTCCCC CTAAGCCAAAAGACACCCTGATGATCTCCCGAACACCAGAGGTCACTTGCGTGGTC GTGGACGTGTCTCAGGAGGACCCCGAAGTCCAGTTCAACTGGTATGTGGATGGCGT CGAAGTGCACAATGCTAAGACCAAACCAAGAGAGGAACAGTTTAATAGTACTTACC GCGTCGTGTCAGTCCTGACCGTGCTGCATCAGGATTGGCTGAACGGCAAGGAGTAT AAGTGCAAAGTGAGCAATAAGGGACTGCCCTCAAGCATCGAGAAAACAATTAGCA AGGCTAAAGGACAGCCCAGGGAGCCTCAGGTGTACACTCTGCCACCCAGTCAGGAG GAAATGACCAAGAACCAGGTCAGCCTGACCTGTCTGGTGAAAGGGTTCTATCCCTC TGACATTGCAGTGGAGTGGGAAAGTAATGGTCAGCCTGAGAACAATTACAAGACTA CCCCTCCAGTGCTGGACTCTGATGGAAGTTTCTTTCTGTATAGCAGACTGACAGTGG ATAAATCCAGGTGGCAGGAGGGGAACGTCTTTAGTTGCTCAGTGATGCACGAAGCC CTGCACAATCATTATACTCAGAAGAGCCTGTCCCTGTCTCTGGGCAGCACCGGTTCT AAGTACGGACCACCATGCCCTCCATGTCCAGCACCAGAGTTCGAAGGCGGACCTTC CGTGTTCCTGTTTCCCCCTAAGCCAAAAGACACCCTGATGATCTCCCGAACACCAGA GGTCACTTGCGTGGTCGTGGACGTGTCTCAGGAGGACCCCGAAGTCCAGTTCAACT GGTATGTGGATGGCGTCGAAGTGCACAATGCTAAGACCAAACCAAGAGAGGAACA GTTTAATAGTACTTACCGCGTCGTGTCAGTCCTGACCGTGCTGCATCAGGATTGGCT GAACGGCAAGGAGTATAAGTGCAAAGTGAGCAATAAGGGACTGCCCTCAAGCATC GAGAAAACAATTAGCAAGGCTAAAGGACAGCCCAGGGAGCCTCAGGTGTACACTC TGCCACCCAGTCAGGAGGAAATGACCAAGAACCAGGTCAGCCTGACCTGTCTGGTG AAAGGGTTCTATCCCTCTGACATTGCAGTGGAGTGGGAAAGTAATGGTCAGCCTGA GAACAATTACAAGACTACCCCTCCAGTGCTGGACTCTGATGGAAGTTTCTTTCTGTA TAGCAGACTGACAGTGGATAAATCCAGGTGGCAGGAGGGGAACGTCTTTAGTTGCT CAGTGATGCACGAAGCCCTGCACAATCATTATACTCAGAAGAGCCTGTCCCTGTCTC TGGGC (SEQ ID NO: 6). In some embodiments, the nucleic acid sequence encoding the anti-PD-1-Fc antibody heavy chain comprises SEQ ID NO: 5 immediately followed by SEQ ID NO: 6. In some embodiments, the signal peptide is cleaved during post-translational modifications that occur in vitro or in vivo. Nucleic acid sequences encoding the respective CDRs, VH domain, CH1 domain, hinge domain, Fc domain and additional Fc domain in SEQ ID NO:6 can be determined from the amino acid sequence of SEQ ID NO: 2 as identified herein by a person of skill in the art.

In some embodiments, the nucleic acid sequence encoding the anti-PD-1-Fc antibody heavy chain encodes an amino acid sequence comprising SEQ ID NO: 2, 14, 16, 18, 20, 22, 24, 26, 28, 30, 72, or 74.

In some embodiments, the amino acid sequence of the anti-PD-1-Fc antibody light chain comprises SEQ ID NO: 3, 15, 17, 19, 21, 23, 25, 27, 29, 31, 73, or 75.

In some embodiments, the nucleic acid sequence encoding the anti-PD-1-Fc antibody light chain comprises SEQ ID NO: 7. GACATCGTGCTGACACAGTCTCCAGCCACCCTGAGCCTGTCCCCAGGAGAGAGGGC CACACTGTCCTGTCGCGCCTCTAAGGGCGTGTCTACCAGCGGCTACAGCTATCTGCA CTGGTACCAGCAGAAGCCAGGACAGGCACCTAGGCTGCTGATCTACCTGGCAAGCT ATCTGGAGTCCGGAGTGCCAGCAAGATTCTCCGGATCTGGAAGCGGAACCGACTTC ACCCTGACCATCTCCTCTCTGGAGCCCGAGGATTTCGCCGTGTACTATTGTCAGCAC AGCAGGGACCTGCCTCTGACATTTGGCGGCGGCACCAAGGTGGAGATCAAGCGCAC AGTGGCCGCTCCCAGCGTGTTCATCTTCCCACCTAGCGACGAGCAGCTGAAGTCCG GCACAGCCTCTGTCGTGTGCCTGCTGAACAACTTCTACCCCCGCGAGGCCAAGGTGC AGTGGAAGGTGGACAATGCCCTGCAGAGCGGCAACAGCCAGGAAAGCGTGACCGA GCAGGACAGCAAGGACTCCACCTACAGCCTGAGCAGCACCCTGACCCTGAGCAAGG CCGACTACGAGAAGCACAAGGTGTACGCCTGCGAAGTGACCCACCAGGGCCTGTCT AGCCCCGTGACCAAGAGCTTCAACCGGGGCGAGTGT (SEQ ID NO: 7). In some embodiments, the nucleic acid sequence encoding the anti-PD-1-Fc antibody light chain comprises SEQ ID NO: 5 immediately followed by SEQ ID NO: 7. In some embodiments, the signal peptide is cleaved during post-translational modifications that occur in vitro or in vivo. Nucleic acid sequences encoding the respective CDRs, VL domain and CL domain in SEQ ID NO: 7 can be determined from the amino acid sequence of SEQ ID NO: 3 as identified herein by a person of skill in the art.

In some embodiments, the nucleic acid sequence encoding the anti-PD-1-Fc antibody light chain encodes an amino acid sequence comprising SEQ ID NO: 15, 17, 19, 21, 23, 25, 27, 29, 31, 73, or 75.

In some embodiments, the nucleic acid sequence encoding a signal peptide of the anti-PD-1-Fc antibody heavy or light chain comprise a nucleic acid sequence 80, 81, 82, 83, 84, 85, 86, 87, 88, 89, 90, 91, 92, 93, 94, 95, 96, 97, 98 or 99% identical to SEQ ID NO: 5. In some embodiments, the nucleic acid sequence encoding an anti-PD-1-Fc heavy chain (without the signal peptide) comprise a nucleic acid sequence 80, 81, 82, 83, 84, 85, 86, 87, 88, 89, 90, 91, 92, 93, 94, 95, 96, 97, 98 or 99% identical to SEQ ID NO: 6. In some embodiments, the nucleic acid sequence encoding an anti-PD-1-Fc light chain (without the signal peptide) comprise a nucleic acid sequence 80, 81, 82, 83, 84, 85, 86, 87, 88, 89, 90, 91, 92, 93, 94, 95, 96, 97, 98 or 99% identical to SEQ ID NO: 7. In some embodiments, the nucleic acid sequence encodes CDRs of the variable heavy chain domain and the CDRs of the variable light chain domain of the anti-PD-1-Fc antibody, wherein the CDR sequences are indicated above (in bold and underline) in SEQ ID NO: 2 and SEQ ID NO:3. In some embodiments, the nucleic acid sequence encoding framework regions (FRs) of the variable heavy chain domain and the FRs of the variable light chain domain of the anti-PD-1-Fc antibody comprise a nucleic acid sequence 80, 81, 82, 83, 84, 85, 86, 87, 88, 89, 90, 91, 92, 93, 94, 95, 96, 97, 98 or 99% identical to the FRs of SEQ ID NO: 6 and SEQ ID NO: 7.

In some embodiments, one or more nucleic acids of a nucleic acid sequence encoding one or more CDRs of SEQ ID NO: 6 or SEQ ID NO: 7 is substituted. In some embodiments, one or more nucleic acids of a nucleic acid sequence encoding one or more variable heavy chain CDRs of SEQ ID NO: 6 is substituted. In some embodiments, one or more nucleic acids of a nucleic acid sequence encoding one or more variable light chain CDRs of SEQ ID NO: 7 is substituted.

In some embodiments, the nucleic acid sequence encoding an anti-PD-1-Fc heavy chain (without the signal peptide) comprise a nucleic acid sequence encoding an amino acid sequence 80, 81, 82, 83, 84, 85, 86, 87, 88, 89, 90, 91, 92, 93, 94, 95, 96, 97, 98 or 99% identical to SEQ ID NO: 2, 14, 16, 18, 20, 22, 24, 26, 28, 30, 72, or 74. In some embodiments, the nucleic acid sequence encoding an anti-PD-1-Fc light chain (without the signal peptide) comprise a nucleic acid sequence encoding an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the nucleic acid sequence encodes CDRs of the variable heavy chain domain and the CDRs of the variable light chain domain of the anti-PD-1-Fc antibody, wherein the CDR sequences are indicated above (in bold and underline), in FIG. 11, or in Tables 1 and 3 for SEQ ID NO: 2, 3, 14, 15, 16, 17, 18, 19, 20, 21, 22, 23, 24, 25, 26, 27, 28, 29, 30, 31, 72, 73, 74, and 75. In some embodiments, the nucleic acid sequence encoding framework regions (FRs) of the variable heavy chain domain and the FRs of the variable light chain domain of the anti-PD-1-Fc antibody comprise a nucleic acid sequence encoding an amino acid sequence 80, 81, 82, 83, 84, 85, 86, 87, 88, 89, 90, 91, 92, 93, 94, 95, 96, 97, 98 or 99% identical to the FRs of SEQ ID NO: 2, 3, 14, 15, 16, 17, 18, 19, 20, 21, 22, 23, 24, 25, 26, 27, 28, 29, 30, 31, 72, 73, 74, or 75, or the FRs in Tables 2 and 4.

In some embodiments, one or more nucleic acids of a nucleic acid sequence encoding one or more CDRs of SEQ ID NO: 2, 3, 14, 15, 16, 17, 18, 19, 20, 21, 22, 23, 24, 25, 26, 27, 28, 29, 30, 31, 72, 73, 74, or 75 is substituted. In some embodiments, one or more nucleic acids of a nucleic acid sequence encoding one or more variable heavy chain CDRs of SEQ ID NO: 2, 14, 16, 18, 20, 22, 24, 26, 28, 30, 72, or 74 is substituted. In some embodiments, one or more nucleic acids of a nucleic acid sequence encoding one or more variable light chain CDRs of SEQ ID NO: 3, 15, 17, 19, 21, 23, 25, 27, 29, 31, 73, or 75 is substituted.

In some embodiments, the nucleic acid sequence comprises a nucleic acid sequence encoding the scFv-Fc and scFv-Fc-scFv described herein.

### Amino Acid Sequences of Anti-PD-1-CD22 Antibodies

In some embodiments, the anti-PD-1-CD22 antibody comprises two polypeptide heavy chains each comprising a variable heavy chain (VH) domain, CH1 domain, a hinge domain, a first Fc domain (CH2-CH3), and a CD22 domain and two polypeptide light chains comprising a variable light chain (VL) domain and a CL domain.

In some embodiments, the amino acid sequence of the anti-PD-1-CD22 antibody heavy chain comprises a signal peptide comprising SEQ ID NO: 1.

In some embodiments, the amino acid sequence of the CD22 domain comprises the extracellular domain of CD22 (SEQ ID NO: 8):

(SEQ ID NO: 8)
DSSKWVFEHPETLYAWEGACVWIPCTYRALDGDLESFILFHNPEY
NKNTSKFDGTRLYESTKDGKVPSEQKRVQFLGDKNKNCTLSIHPV
HLNDSGQLGLRMESKTEKWMERIHLNVSERPFPPHIQLPPEIQES
QEVTLTCLLNFSCYGYPIQLQWLLEGVPMRQAAVTSTSLTIKSVF
TRSELKFSPQWSHHGKIVTCQLQDADGKFLSNDTVQLNVKHTPKL
EIKVTPSDAIVREGDSVTMTCEVSSSNPEYTTVSWLKDGTSLKKQ
NTFTLNLREVTKDQSGKYCCQVSNDVGPGRSEEVFLQVQYAPEPS
TVQILHSPAVEGSQVEFLCMSLANPLPTNYTWYHNGKEMQGRTEE
KVHIPKILPWHAGTYSCVAENILGTGQRGPGAELDVQYPPKKVTT
VIQNPMPIREGDTVTLSCNYNSSNPSVTRYEWKPHGAWEEPSLGV
LKIQNVGWDNTTIACAACNSWCSWASPVALNVQYAPRDVRVRKIK
PLSEIHSGNSVSLQCDFSSSHPKEVQFFWEKNGRLLGKESQLNFD
SISPEDAGSYSCWVNNSIGQTASKAWTLEVLYAPRRLRVSMSPGD
QVMEGKSATLTCESDANPPVSHYTWFDWNNQSLPYHSQKLRLEPV
KVQHSGAYWCQGTNSVGKGRSPLSTLTVYYSPETIGRR.

In some embodiments, the amino acid sequence of the CD22 domain comprises amino acids 505-687 of CD22 (SEQ ID NO: 9):

(SEQ ID NO: 9)
RDVRVRKIKPLSEIHSGNSVSLQCDFSSSHPKEVQFFWEKNGRLL
GKESQLNFDSISPEDAGSYSCWVNNSIGQTASKAWTLEVLYAPRR
LRVSMSPGDQVMEGKSATLTCESDANPPVSHYTWFDWNNQSLPYH
SQKLRLEPVKVQHSGAYWCQGTNSVGKGRSPLSTLTVYYSPETIG
RR.

In some embodiments, the amino acid sequence of the CD22 domain comprises amino acids 593-687 of CD22 (SEQ ID NO: 10):

(SEQ ID NO: 10)
LRVSMSPGDQVMEGKSATLTCESDANPPVSHYTWFDWNNQSL
PYHSQKLRLEPVKVQHSGAYWCQGTNSVGKGRSPLSTLTVYY
SPETIGRR.

In some embodiments, the amino acid sequence of the CD22 domain comprises any fragment of SEQ ID NO: 8 as long as the CD22 domain increases the monoclonal antibody size to at least 150 kD, at least 160 kD, at least 170 kD, at least 180 kD, at least 190 kD, or at least 200 kD. In some embodiments, the amino acid sequence of the CD22 domain comprises any fragment of SEQ ID NO: 8 as long as the CD22 domain increases the monoclonal antibody size to at least about 200 kD. In some embodiments, the amino acid sequence of the CD22 domain comprises any fragment of SEQ ID NO: 8 as long as the CD22 domain increases the monoclonal antibody size to about 200 kD.

In some embodiments, the amino acid sequence of the anti-PD-1-CD22 antibody heavy chain comprises SEQ ID NO: 11. Underlined amino acids represent the VH domain. Underlined and bolded amino acids represent the respective complementarity determining regions (CDRs). Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 11)
QVQLVQSGVEVKKPGASVKVSCKASGYTFTNYYMYWVRQAPGQGLE
WMGGINPSNGGTNFNEKFKNRVTLTTDSSTTTAYMELKSLQFDDT
AVYYCARRDYRFDMGFDYWGQGTLVTVSSASTKGPSVFPLAPCSR
STSESTAALGCLVKDYFPEPVTVSWNSGALTSGVHTFPAVLQSSG
LYSLSSVVTVPSSSLGTKTYTCNVDHKPSNTKVDKRVESKYGPPC
PPCPAPEFEGGPSVFLFPPKPKDTLMISRTPEVTCVVVDVSQEDP
EVQFNWYVDGVEVHNAKTKPREEQFNSTYRVVSVLTVLHQDWLNG
KEYKCKVSNKGLPSSIEKTISKAKGQPREPQVYTLPPSQEEMTKN
QVSLTCLVKGFYPSDIAVEWESNGQPENNYKTTPPVLDSDGSFFL
YSRLTVDKSRWQEGNVFSCSVMHEALHNHYTQKSLSLSLGRDVRV
RKIKPLSEIHSGNSVSLQCDESSSHPKEVQFFWEKNGRLLGKESQ
LNEDSISPEDAGSYSCWVNNSIGQTASKAWTLEVLYAPRRLRVSM
SPGDQVMEGKSATLTCESDANPPVSHYTWFDWNNQSLPYHSQKLR
LEPVKVQHSGAYWCQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 11. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 12. Underlined amino acids represent the VH domain. Underlined and bolded amino acids represent the respective complementarity determining regions (CDRs). Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 12)
QVQLVQSGVEVKKPGASVKVSCKASGYTFTNYYMYWVRQAPGQGLE
WMGGINPSNGGTNFNEKFKNRVTLTTDSSTTTAYMELKSLQFDDTA
VYYCARRDYRFDMGFDYWGQGTLVTVSSASTKGPSVFPLAPCSRST
SESTAALGCLVKDYFPEPVTVSWNSGALTSGVHTFPAVLQSSGLYS
LSSVVTVPSSSLGTKTYTCNVDHKPSNTKVDKRVESKYGPPCPPCP
APEFEGGPSVFLFPPKPKDTLMISRTPEVTCVVVDVSQEDPEVQFN
WYVDGVEVHNAKTKPREEQFNSTYRVVSVLTVLHQDWLNGKEYKCK
VSNKGLPSSIEKTISKAKGQPREPQVYTLPPSQEEMTKNQVSLTCL
VKGFYPSDIAVEWESNGQPENNYKTTPPVLDSDGSFFLYSRLTVDK
SRWQEGNVFSCSVMHEALHNHYTQKSLSLSLGLRVSMSPGDQVMEG
KSATLTCESDANPPVSHYTWFDWNNQSLPYHSQKLRLEPVKVQHSG
AYWCQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 12. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 1 immediately followed by SEQ ID NO: 11 except the double underlined portion is replaced with SEQ ID NO: 8. In some embodiments, the amino acid sequence of the anti-PD-1-CD22 antibody heavy chain comprises SEQ ID NO: 1 immediately followed by SEQ ID NO: 12 except the double underlined portion is replaced with SEQ ID NO: 8. In some embodiments, the signal peptide is cleaved during post-translational modifications that occur in vitro or in vivo.

In some embodiments, the amino acid sequence of the anti-PD-1-CD22 antibody light chain (without the signal peptide) comprises SEQ ID NO: 3, 15, 17, 19, 21, 23, 25, 27, 29, 31, 73, or 75 as provided above for the anti-PD1-Fc antibody. In some embodiments, the amino acid sequence of the anti-PD-1-CD22 antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 11 and a second and fourth chains each comprises SEQ ID NO: 3, 15, 17, 19, 21, 23, 25, 27, 29, 31, 73 or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 12 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 11 except the double underlined portion is replaced with SEQ ID NO: 8 and a second and fourth chains each comprises SEQ ID NO: 3, 15, 17, 19, 21, 23, 25, 27, 29, 31, 73 or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 12 except the double underlined portion is replaced with SEQ ID NO: 8 and a second and fourth chains each comprises SEQ ID NO: 3, 15, 17, 19, 21, 23, 25, 27, 29, 31, 73 or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-CD22 antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 11. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 12. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 11 with the double underlined portion replaced with SEQ ID NO: 8. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 12 with the double underlined portion replaced with SEQ ID NO: 8. In some embodiments, the anti-PD-1-CD22 antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, or 31. In some embodiments, the anti-PD-1-CD22 antibody comprises an amino acid sequence comprising the CDRs of the variable heavy chain domain and the CDRs of the variable light chain domain of the anti-PD-1-CD22 antibody, wherein the CDR sequences are indicated above (in bold and underline), in FIG. 11, or in Tables 1 and 3 for SEQ ID NO: 3, 11, 12, 14, 15, 16, 17, 18, 19, 20, 21, 22, 23, 24, 25, 26, 27, 28, 29, 30, 31, 72, 73, 74, and 75. In some embodiments, the framework regions (FRs) of the variable heavy chain domain and the FRs of the variable light chain domain of the anti-PD-1-CD22 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 11, 12, 14, 15, 16, 17, 18, 19, 20, 21, 22, 23, 24, 25, 26, 27, 28, 29, 30, 31, 72, 73, 74, or 75, or the FRs in Tables 2 and 4.

In some embodiments, one or more amino acids of an amino acid sequence encoding one or more CDRs of SEQ ID NO: 3, 11, 12, 14, 15, 16, 17, 18, 19, 20, 21, 22, 23, 24, 25, 26, 27, 28, 29, 30, 31, 72, 73, 74, or 75 is substituted. In some embodiments, one or more amino acids of an amino acid sequence encoding one or more variable heavy chain CDRs of SEQ ID NO: 11, 12, 14, 16, 18, 20, 22, 24, 26, 28, 30, 72, or 74 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments the monoclonal antibody is a scFv-Fc antibody comprising a first and second chain that associate together, each chain comprising a variable heavy chain (VH) domain of SEQ ID NO: 2, 14, 16, 18, 20, 22, 24, 26, 28, 30, 72, or 74, a linker

(e.g., GGGGSGGGGSGGGGS (SEQ ID NO: 13)),

a variable light chain (VL) domain of SEQ ID NO: 3, 15, 17, 19, 21, 23, 25, 27, 29, 31 or 73 or 75, an Fc domain of SEQ ID NO: 2, and at least one additional domain. In some embodiments, the additional domain is a CD22 domain of SEQ ID NOs: 8, 9, or 10. In some embodiments, the amino acid sequence of the CD22 domain comprises any fragment of SEQ ID NO: 8 as long as the CD22 domain increases the antibody size to at least 150 kD, at least 160 kD, at least 170 kD, at least 180 kD, at least 190 kD, or at least 200 kD. In some embodiments, the amino acid sequence of the CD22 domain comprises any fragment of SEQ ID NO: 8 as long as the CD22 domain increases the antibody size to at least about 200 kD. In some embodiments, the amino acid sequence of the CD22 domain comprises any fragment of SEQ ID NO: 8 as long as the CD22 domain increases the antibody size to about 200 kD.

In some embodiments, the amino acid sequence of the anti-PD-1-CD22 antibody heavy chain comprises SEQ ID NO: 32. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 32)
EVKLLESGGGLVQPGGSLKLSCAASGFDFSRYWMSWVRQAPGKGLE
WIGEINPDSSTIKYTPSLKDKFIISRDNAKNTLCLQLSKVRSEDSA
LYYCARMGYRLFDSWGQGTTLTVSSASTKGPSVFPLAPCSRSTSES
TAALGCLVKDYFPEPVTVSWNSGALTSGVHTFPAVLQSSGLYSLSS
VVTVPSSSLGTKTYTCNVDHKPSNTKVDKRVESKYGPPCPPCPAPE
FEGGPSVFLFPPKPKDTLMISRTPEVTCVVVDVSQEDPEVQFNWYV
DGVEVHNAKTKPREEQFNSTYRVVSVLTVLHQDWLNGKEYKCKVSN
KGLPSSIEKTISKAKGQPREPQVYTLPPSQEEMTKNQVSLTCLVKG
FYPSDIAVEWESNGQPENNYKTTPPVLDSDGSFFLYSRLTVDKSRW
QEGNVFSCSVMHEALHNHYTQKSLSLSLGRDVRVRKIKPLSEIHSG
NSVSLQCDFSSSHPKEVQFFWEKNGRLLGKESQLNFDSISPEDAGS
YSCWVNNSIGQTASKAWTLEVLYAPRRLRVSMSPGDQVMEGKSATL
TCESDANPPVSHYTWFDWNNQSLPYHSQKLRLEPVKVQHSGAYWCQ
GTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 32. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 33. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 33)
EVKLLESGGGLVQPGGSLKLSCAASGFDFSRYWMSWVRQAPGKGLE
WIGEINPDSSTIKYTPSLKDKFIISRDNAKNTLCLQLSKVRSEDSA
LYYCARMGYRLFDSWGQGTTLTVSSASTKGPSVFPLAPCSRSTSES
TAALGCLVKDYFPEPVTVSWNSGALTSGVHTFPAVLQSSGLYSLSS
VVTVPSSSLGTKTYTCNVDHKPSNTKVDKRVESKYGPPCPPCPAPE
FEGGPSVFLFPPKPKDTLMISRTPEVTCVVVDVSQEDPEVQFNWYV
DGVEVHNAKTKPREEQFNSTYRVVSVLTVLHQDWLNGKEYKCKVSN
KGLPSSIEKTISKAKGQPREPQVYTLPPSQEEMTKNQVSLTCLVKG
FYPSDIAVEWESNGQPENNYKTTPPVLDSDGSFFLYSRLTVDKSRW
QEGNVFSCSVMHEALHNHYTQKSLSLSLGLRVSMSPGDQVMEGKSA
TLTCESDANPPVSHYTWFDWNNQSLPYHSQKLRLEPVKVQHSGAYW
CQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 33. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 1 immediately followed by SEQ ID NO: 32 except the double underlined portion is replaced with SEQ ID NO: 8. In some embodiments, the amino acid sequence of the anti-PD-1-CD22 antibody heavy chain comprises SEQ ID NO: 1 immediately followed by SEQ ID NO: 33 except the double underlined portion is replaced with SEQ ID NO: 8. In some embodiments, the signal peptide is cleaved during post-translational modifications that occur in vitro or in vivo.

In some embodiments, the amino acid sequence of the anti-PD-1-CD22 antibody light chain (without the signal peptide) comprises SEQ ID NO: 3, 15, 17, 19, 21, 23, 25, 27, 29, 31, 73 or 75 as provided above for the anti-PD1-Fc antibody. In some embodiments, the amino acid sequence of the anti-PD-1-CD22 antibody light chain comprises SEQ ID NO: 1 immediately followed by SEQ ID NO: 3, 15, 17, 19, 21, 23, 25, 27, 29, 31, 73 or 75. In some embodiments, the signal peptide is cleaved during post-translational modifications that occur in vitro or in vivo.

In some embodiments, the monoclonal antibody comprises a first and third chain each comprises SEQ ID NO: 32 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 33 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 32 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 33 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-CD22 antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 32. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 33. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 32 with the double underlined portion replaced with SEQ ID NO: 8. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 33 with the double underlined portion replaced with SEQ ID NO: 8. In some embodiments, the anti-PD-1-CD22 antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-CD22 antibody comprises an amino acid sequence comprising the CDRs of the variable heavy chain domain and the CDRs of the variable light chain domain of the anti-PD-1-CD22 antibody, wherein the CDR sequences are indicated above (in bold and underline), in FIG. 11, or in Tables 1 and 3 for SEQ ID NO: 32 and 33 and SEQ ID NO: 3, 15, 17, 19, 21, 23, 25, 27, 29, 31, 73, or 75. In some embodiments, the framework regions (FRs) of the variable heavy chain domain and the FRs of the variable light chain domain of the anti-PD-1-CD22 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 32 and 33 and SEQ ID NO: 3, 15, 17, 19, 21, 23, 25, 27, 29, 31, 73, or 75, or the FRs in Tables 2 and 4.

In some embodiments, one or more amino acids of an amino acid sequence encoding one or more CDRs of SEQ ID NO: 32, 33, 3, 15, 17, 19, 21, 23, 25, 27, 29, 31, 73, or 75 is substituted. In some embodiments, one or more amino acids of an amino acid sequence encoding one or more variable heavy chain CDRs of SEQ ID NO: 32 or SEQ ID NO: 33 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-CD22 antibody heavy chain comprises SEQ ID NO: 34. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 34)
EVQLQQSGTVLARPGASVKMSCKASGNTFTSYWMHWVKQRPGQGLE
WIGAIYPGYSDTRYNQKFKGKATLTAVTSTSTAYMELSSLTNEDSA
VYYCTKFIATVGGYFDVWGAGTTVTVSSASTKGPSVFPLAPCSRSTS
ESTAALGCLVKDYFPEPVTVSWNSGALTSGVHTFPAVLQSSGLYSLS
SVVTVPSSSLGTKTYTCNVDHKPSNTKVDKRVESKYGPPCPPCPAPE
FEGGPSVFLFPPKPKDTLMISRTPEVTCVVVDVSQEDPEVQFNWYV
DGVEVHNAKTKPREEQFNSTYRVVSVLTVLHQDWLNGKEYKCKVSN
KGLPSSIEKTISKAKGQPREPQVYTLPPSQEEMTKNQVSLTCLVKG
FYPSDIAVEWESNGQPENNYKTTPPVLDSDGSFFLYSRLTVDKSRW
QEGNVFSCSVMHEALHNHYTQKSLSLSLGRDVRVRKIKPLSEIHSG
NSVSLQCDFSSSHPKEVQFFWEKNGRLLGKESQLNFDSISPEDAGS
YSCWVNNSIGQTASKAWTLEVLYAPRRLRVSMSPGDQVMEGKSATL
TCESDANPPVSHYTWFDWNNQSLPYHSQKLRLEPVKVQHSGAYWCQ
GTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 34. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 35. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 35)
EVQLQQSGTVLARPGASVKMSCKASGNTFTSYWMHWVKQRPGQGLE
WIGAIYPGYSDTRYNQKFKGKATLTAVTSTSTAYMELSSLTNEDSA
VYYCTKFIATVGGYFDVWGAGTTVTVSSASTKGPSVFPLAPCSRST
SESTAALGCLVKDYFPEPVTVSWNSGALTSGVHTFPAVLQSSGLYS
LSSVVTVPSSSLGTKTYTCNVDHKPSNTKVDKRVESKYGPPCPPCP
APEFEGGPSVFLFPPKPKDTLMISRTPEVTCVVVDVSQEDPEVQFN
WYVDGVEVHNAKTKPREEQFNSTYRVVSVLTVLHQDWLNGKEYKCK
VSNKGLPSSIEKTISKAKGQPREPQVYTLPPSQEEMTKNQVSLTCL
VKGFYPSDIAVEWESNGQPENNYKTTPPVLDSDGSFFLYSRLTVDK
SRWQEGNVFSCSVMHEALHNHYTQKSLSLSLGLRVSMSPGDQVMEG
KSATLTCESDANPPVSHYTWFDWNNQSLPYHSQKLRLEPVKVQHSG
AYWCQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 35. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 1 immediately followed by SEQ ID NO: 34 except the double underlined portion is replaced with SEQ ID NO: 8. In some embodiments, the amino acid sequence of the anti-PD-1-CD22 antibody heavy chain comprises SEQ ID NO: 1 immediately followed by SEQ ID NO: 35 except the double underlined portion is replaced with SEQ ID NO: 8. In some embodiments, the signal peptide is cleaved during post-translational modifications that occur in vitro or in vivo.

In some embodiments, the amino acid sequence of the anti-PD-1-CD22 antibody light chain (without the signal peptide) comprises SEQ ID NO: 3, 15, 17, 19, 21, 23, 25, 27, 29, 31, 73, or 75 as provided above for the anti-PD1-Fc antibody. In some embodiments, the amino acid sequence of the anti-PD-1-CD22 antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 34 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 35 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 34 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 35 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-CD22 antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 34. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 35. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 34 with the double underlined portion replaced with SEQ ID NO: 8. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 35 with the double underlined portion replaced with SEQ ID NO: 8. In some embodiments, the anti-PD-1-CD22 antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-CD22 antibody comprises an amino acid sequence comprising the CDRs of the variable heavy chain domain and the CDRs of the variable light chain domain of the anti-PD-1-CD22 antibody, wherein the CDR sequences are indicated above (in bold and underline), in FIG. 11, or in Tables 1 and 3 for SEQ ID NO: 34 and 35, and SEQ ID NO: 3, 15, 17, 19, 21, 23, 25, 27, 29, 31, 73, or 75. In some embodiments, the framework regions (FRs) of the variable heavy chain domain and the FRs of the variable light chain domain of the anti-PD-1-CD22 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 34 and 35, and SEQ ID NO: 3, 15, 17, 19, 21, 23, 25, 27, 29, 31, 73, or 75, or the FRs in Tables 2 and 4.

In some embodiments, one or more amino acids of an amino acid sequence encoding one or more CDRs of SEQ ID NO: 34, 35, 3, 15, 17, 19, 21, 23, 25, 27, 29, 31, 73, or 75 is substituted. In some embodiments, one or more amino acids of an amino acid sequence encoding one or more variable heavy chain CDRs of SEQ ID NO: 34 or SEQ ID NO: 35 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-CD22 antibody heavy chain comprises SEQ ID NO: 36. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 36)
QIQLVQSGPELKKPGETVKISCKASGYTFTDYSMHWVKQAPGKGLK
WMGWIKIETGNPTYADDFKGRFAFSLETSASTAYLQINNLKNEDTA
TYFCARDYYGYYYYAMDYWGQGTSVTVSSASTKGPSVFPLAPCSRS
TSESTAALGCLVKDYFPEPVTVSWNSGALTSGVHTFPAVLQSSGLY
SLSSVVTVPSSSLGTKTYTCNVDHKPSNTKVDKRVESKYGPPCPPC
PAPEFEGGPSVFLFPPKPKDTLMISRTPEVTCVVVDVSQEDPEVQF
NWYVDGVEVHNAKTKPREEQFNSTYRVVSVLTVLHQDWLNGKEYKC
KVSNKGLPSSIEKTISKAKGQPREPQVYTLPPSQEEMTKNQVSLTC
LVKGFYPSDIAVEWESNGQPENNYKTTPPVLDSDGSFFLYSRLTVD
KSRWQEGNVFSCSVMHEALHNHYTQKSLSLSLGRDVRVRKIKPLSE
IHSGNSVSLQCDFSSSHPKEVQFFWEKNGRLLGKESQLNFDSISPE
DAGSYSCWVNNSIGQTASKAWTLEVLYAPRRLRVSMSPGGQVMEGK
SATLTCESDANPPVSHYTWFDWNNQSLPYHSQKLRLEPVKVQHSGA
YWCQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 36. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 37. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 37)
QIQLVQSGPELKKPGETVKISCKASGYTFTDYSMHWVKQAPGKGLKWMGWIKIETGNP
TYADDFKGRFAFSLETSASTAYLQINNLKNEDTATYFCARDYYGYYYYAMDYWGQGT
SVTVSSASTKGPSVFPLAPCSRSTSESTAALGCLVKDYFPEPVTVSWNSGALTSGVHITFP
AVLQSSGLYSLSSVVTVPSSSLGTKTYTCNVDHKPSNTKVDKRVESKYGPPCPPCPAPEFE
GGPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTVDKSRWQEGN
VFSCSVMHEALHNHYTQKSLSLSLGLRVSMSPGDQVMEGKSATLTCESDANPPVSHYTWF
DWNNQSLPYHSQKLRLEPVKVQHSGAYWCQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 37. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 1 immediately followed by SEQ ID NO: 36 except the double underlined portion is replaced with SEQ ID NO: 8. In some embodiments, the amino acid sequence of the anti-PD-1-CD22 antibody heavy chain comprises SEQ ID NO: 1 immediately followed by SEQ ID NO: 37 except the double underlined portion is replaced with SEQ ID NO: 8. In some embodiments, the signal peptide is cleaved during post-translational modifications that occur in vitro or in vivo.

In some embodiments, the amino acid sequence of the anti-PD-1-CD22 antibody light chain (without the signal peptide) comprises SEQ ID NO: 3, 15, 17, 19, 21, 23, 25, 27, 29, 31, 73, or 75 as provided above for the anti-PD1-Fc antibody. In some embodiments, the amino acid sequence of the anti-PD-1-CD22 antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 36 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 37 and a second and fourth chains each comprises SEQ ID NO: 3, 15, 17, 19, 21, 23, 25, 27, 29, 31, 73, or 75 In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 36 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 37 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-CD22 antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 36. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 37. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 36 with the double underlined portion replaced with SEQ ID NO: 8. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 37 with the double underlined portion replaced with SEQ ID NO: 8. In some embodiments, the anti-PD-1-CD22 antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-CD22 antibody comprises an amino acid sequence comprising the CDRs of the variable heavy chain domain and the CDRs of the variable light chain domain of the anti-PD-1-CD22 antibody, wherein the CDR sequences are indicated above (in bold and underline), in FIG. 11, or in Tables 1 and 3 for SEQ ID NO: 36 and 37, and SEQ ID NO: 3, 15, 17, 19, 21, 23, 25, 27, 29, 31, 73, or 75. In some embodiments, the framework regions (FRs) of the variable heavy chain domain and the FRs of the variable light chain domain of the anti-PD-1-CD22 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 36 and 37, and SEQ ID NO: 3, 15, 17, 19, 21, 23, 25, 27, 29, 31, 73, or 75, or the FRs in Tables 2 and 4.

In some embodiments, one or more amino acids of an amino acid sequence encoding one or more CDRs of SEQ ID NO: 36, 37, 3, 15, 17, 19, 21, 23, 25, 27, 29, 31, 73, or 75 is substituted. In some embodiments, one or more amino acids of an amino acid sequence encoding one or more variable heavy chain CDRs of SEQ ID NO: 36 or SEQ ID NO: 37 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-CD22 antibody heavy chain comprises SEQ ID NO: 38. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 38)
EVQLVESGGGLVQPGGSLRLSCAASGFTFSHYGMSWVRQTPDKRLELVATIDNNGGNT
YYPDSVKGRFTISRDNAKNTLYLQMSSLKSEDTAMYYCARDAYYTYDYWYFDVWGA
GTTVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCPAPE
FEGGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSRWQE
GNVFSCSVMHEALHNHYTQKSLSLSLGRDVRVRKIKPLSEIHSGNSVSLQCDFSSSHPKEV
QFFWEKNGRLLGKESQLNFDSISPEDAGSYSCWVNNSIGQTASKAWTLEVLYAPRRLR
VSMSPGDQVMEGKSATLTCESDANPPVSHYTWFDWNNQSLPYHSQKLRLEPVKVQHS
GAYWCQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 38. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 39. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 39)
EVQLVESGGGLVQPGGSLRLSCAASGFTFSHYGMSWVRQTPDKRLELVATIDNNGGNT
YYPDSVKGRFTISRDNAKNTLYLQMSSLKSEDTAMYYCARDAYYTYDYWYFDVWGA
GTTVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCPAPE
FEGGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSRWQE
GNVFSCSVMHEALHNHYTQKSLSLSLGLRVSMSPGDQVMEGKSATLTCESDANPPVSHYT
WFDWNNQSLPYHSQKLRLEPVKVQHSGAYWCQGTNSVGKGRSPLSTLTVYYSPETIGR
R.

In some embodiments, the amino acid sequence of the anti-PD-1-CD22 antibody heavy chain comprises SEQ ID NO: 1 immediately followed by SEQ ID NO: 39. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 1 immediately followed by SEQ ID NO: 38 except the double underlined portion is replaced with SEQ ID NO: 8. In some embodiments, the amino acid sequence of the anti-PD-1-CD22 antibody heavy chain comprises SEQ ID NO: 1 immediately followed by SEQ ID NO: 39 except the double underlined portion is replaced with SEQ ID NO: 8. In some embodiments, the signal peptide is cleaved during post-translational modifications that occur in vitro or in vivo.

In some embodiments, the amino acid sequence of the anti-PD-1-CD22 antibody light chain (without the signal peptide) comprises SEQ ID NO: 3, 15, 17, 19, 21, 23, 25, 27, 29, 31, 73, or 75 as provided above for the anti-PD1-Fc antibody. In some embodiments, the amino acid sequence of the anti-PD-1-CD22 antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 38 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 39 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 38 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 39 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-CD22 antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 38. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 39. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 38 with the double underlined portion replaced with SEQ ID NO: 8. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 39 with the double underlined portion replaced with SEQ ID NO: 8. In some embodiments, the anti-PD-1-CD22 antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-CD22 antibody comprises an amino acid sequence comprising the CDRs of the variable heavy chain domain and the CDRs of the variable light chain domain of the anti-PD-1-CD22 antibody, wherein the CDR sequences are indicated above (in bold and underline), in FIG. 11, or in Tables 1 and 3 for SEQ ID NO: 38 and 39, and SEQ ID NO: 3, 15, 17, 19, 21, 23, 25, 27, 29, 31, 73, or 75. In some embodiments, the framework regions (FRs) of the variable heavy chain domain and the FRs of the variable light chain domain of the anti-PD-1-CD22 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 38 and 39, and SEQ ID NO: 3, 15, 17, 19, 21, 23, 25, 27, 29, 31, 73, or 75, or the FRs in Tables 2 and 4.

In some embodiments, one or more amino acids of an amino acid sequence encoding one or more CDRs of SEQ ID NO: 38, 39, 3, 15, 17, 19, 21, 23, 25, 27, 29, 31, 73, or 75 is substituted. In some embodiments, one or more amino acids of an amino acid sequence encoding one or more variable heavy chain CDRs of SEQ ID NO: 38 or SEQ ID NO: 39 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-CD22 antibody heavy chain comprises SEQ ID NO: 40. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 40)
EVKLVESGGGLVQPGGSLKLSCAASGFTFSSYTMSWVRQTPEKRLEWVAYISYGGGDT
YYPDTVKGRFTISRDNAKNTLYLQMSSLKSEDTAMYYCARQKVDGYFVAMDYWSQG
TSVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRWQEG
NVFSCSVMHEALHNHYTQKSLSLSLGRDVRVRKIKPLSEIHSGNSVSLQCDFSSSHPKEVQF
FWEKNGRLLGKESQLNFDSISPEDAGSYSCWVNNSIGQTASKAWTLEVLYAPRRLRVS
MSPGDQVMEGKSATLTCESDANPPVSHYTWFDWNNQSLPYHSQKLRLEPVKVQHSGA
YWCQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 40. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 41. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 41)
EVKLVESGGGLVQPGGSLKLSCAASGFTFSSYTMSWVRQTPEKRLEWVAYISYGGGDT
YYPDTVKGRFTISRDNAKNTLYLQMSSLKSEDTAMYYCARQKVDGYFVAMDYWSQG
TSVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDGVEVHNAKTKPREEQ
FNSTYRVVSVLTVLHODWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRWQEG
NVFSCSVMHEALHNHYTQKSLSLSLGLRVSMSPGDQVMEGKSATLTCESDANPPVSHYT
WFDWNNQSLPYHSQKLRLEPVKVQHSGAYWCQGTNSVGKGRSPLSTLTVYYSPETIGR
R.

In some embodiments, the amino acid sequence of the anti-PD-1-CD22 antibody heavy chain comprises SEQ ID NO: 1 immediately followed by SEQ ID NO: 41. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 1 immediately followed by SEQ ID NO: 40 except the double underlined portion is replaced with SEQ ID NO: 8. In some embodiments, the amino acid sequence of the anti-PD-1-CD22 antibody heavy chain comprises SEQ ID NO: 1 immediately followed by SEQ ID NO: 41 except the double underlined portion is replaced with SEQ ID NO: 8. In some embodiments, the signal peptide is cleaved during post-translational modifications that occur in vitro or in vivo.

In some embodiments, the amino acid sequence of the anti-PD-1-CD22 antibody light chain (without the signal peptide) comprises SEQ ID NO: 3, 15, 17, 19, 21, 23, 25, 27, 29, 31, 73, or 75 as provided above for the anti-PD1-Fc antibody. In some embodiments, the amino acid sequence of the anti-PD-1-CD22 antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 40 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 41 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 40 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 41 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-CD22 antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 40. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 41. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 40 with the double underlined portion replaced with SEQ ID NO: 8. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 41 with the double underlined portion replaced with SEQ ID NO: 8. In some embodiments, the anti-PD-1-CD22 antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-CD22 antibody comprises an amino acid sequence comprising the CDRs of the variable heavy chain domain and the CDRs of the variable light chain domain of the anti-PD-1-CD22 antibody, wherein the CDR sequences are indicated above (in bold and underline), in FIG. 11, or in Tables 1 and 3 for SEQ ID NO: 40, 41, 3, 15, 17, 19, 21, 23, 25, 27, 29, 31, 73 or 75. In some embodiments, the framework regions (FRs) of the variable heavy chain domain and the FRs of the variable light chain domain of the anti-PD-1-CD22 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 40, 41, 3, 15, 17, 19, 21, 23, 25, 27, 29, 31, 73, or 75, or the FRs in Tables 2 and 4.

In some embodiments, one or more amino acids of an amino acid sequence encoding one or more CDRs of SEQ ID NO: 40, 41, 3, 15, 17, 19, 21, 23, 25, 27, 29, 31, 73, or 75 is substituted. In some embodiments, one or more amino acids of an amino acid sequence encoding one or more variable heavy chain CDRs of SEQ ID NO: 40 or SEQ ID NO: 41 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-CD22 antibody heavy chain comprises SEQ ID NO: 42. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 42)
EVKLVESGGGLVKPGGSLKLSCAASGFTFSSYGMSWVRQTPEKRLEWVATISGGGRYT
YYPDSVKGRFTISRDNAKNILYLQMSSLRSEDTALYYCASPYDGYYGAMDYWGQGTS
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEG
GPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGNVFS
CSVMHEALHNHYTQKSLSLSLGRDVRVRKIKPLSEIHSGNSVSLQCDFSSSHPKEVQFFWE
KNGRLLGKESQLNFDSISPEDAGSYSCWVNNSIGQTASKAWTLEVLYAPRRLRVSMSPG
DQVMEGKSATLTCESDANPPVSHYTWFDWNNQSLPYHSQKLRLEPVKVQHSGAYWC
QGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 42. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 43. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 43)
EVKLVESGGGLVKPGGSLKLSCAASGFTFSSYGMSWVRQTPEKRLEWVATISGGGRYT
YYPDSVKGRFTISRDNAKNILYLQMSSLRSEDTALYYCASPYDGYYGAMDYWGQGTS
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEG
GPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGNVFS
CSVMHEALHNHYTQKSLSLSLGLRVSMSPGDQVMEGKSATLTCESDANPPVSHYTWFDW
NNQSLPYHSQKLRLEPVKVQHSGAYWCQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 43. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 1 immediately followed by SEQ ID NO: 42 except the double underlined portion is replaced with SEQ ID NO: 8. In some embodiments, the amino acid sequence of the anti-PD-1-CD22 antibody heavy chain comprises SEQ ID NO: 1 immediately followed by SEQ ID NO: 43 except the double underlined portion is replaced with SEQ ID NO: 8. In some embodiments, the signal peptide is cleaved during post-translational modifications that occur in vitro or in vivo.

In some embodiments, the amino acid sequence of the anti-PD-1-CD22 antibody light chain (without the signal peptide) comprises SEQ ID NO: 3, 15, 17, 19, 21, 23, 25, 27, 29, 31, 73, or 75 as provided above for the anti-PD1-Fc antibody. In some embodiments, the amino acid sequence of the anti-PD-1-CD22 antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 42 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 43 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 42 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 43 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-CD22 antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 42. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 43. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 42 with the double underlined portion replaced with SEQ ID NO: 8. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 43 with the double underlined portion replaced with SEQ ID NO: 8. In some embodiments, the anti-PD-1-CD22 antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-CD22 antibody comprises an amino acid sequence comprising the CDRs of the variable heavy chain domain and the CDRs of the variable light chain domain of the anti-PD-1-CD22 antibody, wherein the CDR sequences are indicated above (in bold and underline), in FIG. 11, or in Tables 1 and 3 for in SEQ ID NO: 42, 43, 3, 15, 17, 19, 21, 23, 25, 27, 29, 31, 73 or 75. In some embodiments, the framework regions (FRs) of the variable heavy chain domain and the FRs of the variable light chain domain of the anti-PD-1-CD22 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 42, 43, 3, 15, 17, 19, 21, 23, 25, 27, 29, 31, 73, or 75, or the FRs in Tables 2 and 4.

In some embodiments, one or more amino acids of an amino acid sequence encoding one or more CDRs of SEQ ID NO: 42, 43, 3, 15, 17, 19, 21, 23, 25, 27, 29, 31, 73, or 75 is substituted. In some embodiments, one or more amino acids of an amino acid sequence encoding one or more variable heavy chain CDRs of SEQ ID NO: 42 or SEQ ID NO: 43 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-CD22 antibody heavy chain comprises SEQ ID NO: 44. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 44)
EVQLQQSGTVLARPGASVRMSCKASGYSFSTYWMHWVKQRPGQGLEWIGGIYPGNSD
TNYNQKFKGKAKLTAVTSASTAYMDLSSLTNEDSAVYYCTGGYFFDVWGAGTTVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPSV
FLFPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSV
MHEALHNHYTQKSLSLSLGRDVRVRKIKPLSEIHSGNSVSLQCDFSSSHPKEVQFFWEKN
GRLLGKESQLNFDSISPEDAGSYSCWVNNSIGQTASKAWTLEVLYAPRRLRVSMSPGDQ
VMEGKSATLTCESDANPPVSHYTWFDWNNQSLPYHSQKLRLEPVKVQHSGAYWCQGT
NSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 44. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 45. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 45)
EVQLQQSGTVLARPGASVRMSCKASGYSFSTYWMHWVKQRPGQGLEWIGGIYPGNSD
TNYNQKFKGKAKLTAVTSASTAYMDLSSLTNEDSAVYYCTGGYFFDVWGAGTTVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPSV
FLFPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSV
MHEALHNHYTQKSLSLSLGLRVSMSPGDQVMEGKSATLTCESDANPPVSHYTWFDWNN
QSLPYHSQKLRLEPVKVQHSGAYWCQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 45. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 1 immediately followed by SEQ ID NO: 44 except the double underlined portion is replaced with SEQ ID NO: 8. In some embodiments, the amino acid sequence of the anti-PD-1-CD22 antibody heavy chain comprises SEQ ID NO: 1 immediately followed by SEQ ID NO: 45 except the double underlined portion is replaced with SEQ ID NO: 8. In some embodiments, the signal peptide is cleaved during post-translational modifications that occur in vitro or in vivo.

In some embodiments, the amino acid sequence of the anti-PD-1-CD22 antibody light chain (without the signal peptide) comprises SEQ ID NO: 3, 15, 17, 19, 21, 23, 25, 27, 29, 31, 73, or 75 as provided above for the anti-PD1-Fc antibody. In some embodiments, the amino acid sequence of the anti-PD-1-CD22 antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 44 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 45 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 44 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 45 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-CD22 antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 44. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 45. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 44 with the double underlined portion replaced with SEQ ID NO: 8. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 45 with the double underlined portion replaced with SEQ ID NO: 8. In some embodiments, the anti-PD-1-CD22 antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-CD22 antibody comprises an amino acid sequence comprising the CDRs of the variable heavy chain domain and the CDRs of the variable light chain domain of the anti-PD-1-CD22 antibody, wherein the CDR sequences are indicated above (in bold and underline), in FIG. 11, or in Tables 1 and 3 for SEQ ID NO: 44, 45, 3, 15, 17, 19, 21, 23, 25, 27, 29, 31, 73 or 75. In some embodiments, the framework regions (FRs) of the variable heavy chain domain and the FRs of the variable light chain domain of the anti-PD-1-CD22 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 44, 45, 3, 15, 17, 19, 21, 23, 25, 27, 29, 31, 73, or 75, or the FRs in Tables 2 and 4.

In some embodiments, one or more amino acids of an amino acid sequence encoding one or more CDRs of SEQ ID NO: 44, 45, 3, 15, 17, 19, 21, 23, 25, 27, 29, 31, 73, or 75 is substituted. In some embodiments, one or more amino acids of an amino acid sequence encoding one or more variable heavy chain CDRs of SEQ ID NO: 44 or SEQ ID NO: 45 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-CD22 antibody heavy chain comprises SEQ ID NO: 46. Underlined amino acids represent the VH domain In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 46)
QIQLQQSGPEQVKPGASVKISCKASGYMFIDYYMNWVKQKPGQGLEWIGWIYPGSGST
KDNENFKGKATLTVDTSSSTAYMQLSSLTSEDTAVYFCARYGPRFFDVWGAGTTVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPSV
FLFPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSV
MHEALHNHYTQKSLSLSLGRDVRVRKIKPLSEIHSGNSVSLQCDFSSSHPKEVQFFWEKN
GRLLGKESQLNFDSISPEDAGSYSCWVNNSIGQTASKAWTLEVLYAPRRLRVSMSPGDQ
VMEGKSATLTCESDANPPVSHYTWFDWNNQSLPYHSQKLRLEPVKVQHSGAYWCQGT
NSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 46. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 47. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 47)
QIQLQQSGPEQVKPGASVKISCKASGYMFIDYYMNWVKQKPGQGLEWIGWIYPGSGST
KDNENFKGKATLTVDTSSSTAYMQLSSLTSEDTAVYFCARYGPRFFDVWGAGTTVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPSV
FLFPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSV
MHEALHNHYTQKSLSLSLGLRVSMSPGDQVMEGKSATLTCESDANPPVSHYTWFDWNN
QSLPYHSQKLRLEPVKVQHSGAYWCQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 47. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 1 immediately followed by SEQ ID NO: 46 except the double underlined portion is replaced with SEQ ID NO: 8. In some embodiments, the amino acid sequence of the anti-PD-1-CD22 antibody heavy chain comprises SEQ ID NO: 1 immediately followed by SEQ ID NO: 47 except the double underlined portion is replaced with SEQ ID NO: 8. In some embodiments, the signal peptide is cleaved during post-translational modifications that occur in vitro or in vivo.

In some embodiments, the amino acid sequence of the anti-PD-1-CD22 antibody light chain (without the signal peptide) comprises SEQ ID NO: 3, 15, 17, 19, 21, 23, 25, 27, 29, 31, 73, or 75 as provided above for the anti-PD1-Fc antibody. In some embodiments, the amino acid sequence of the anti-PD-1-CD22 antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 46 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 47 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 46 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 47 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-CD22 antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 46. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 47. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 46 with the double underlined portion replaced with SEQ ID NO: 8. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 47 with the double underlined portion replaced with SEQ ID NO: 8. In some embodiments, the anti-PD-1-CD22 antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-CD22 antibody comprises an amino acid sequence comprising the CDRs of the variable heavy chain domain and the CDRs of the variable light chain domain of the anti-PD-1-CD22 antibody, wherein the CDR sequences are indicated above (in bold and underline), in FIG. 11, or in Tables 1 and 3 for SEQ ID NO: 46, 47, 3, 15, 17, 19, 21, 23, 25, 27, 29, 31, 73 or 75. In some embodiments, the framework regions (FRs) of the variable heavy chain domain and the FRs of the variable light chain domain of the anti-PD-1-CD22 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 46, 47, 3, 15, 17, 19, 21, 23, 25, 27, 29, 31, 73, or 75, or the FRs in Tables 2 and 4.

In some embodiments, one or more amino acids of an amino acid sequence encoding one or more CDRs of SEQ ID NO: 46, 47, 3, 15, 17, 19, 21, 23, 25, 27, 29, 31, 73, or 75 is substituted. In some embodiments, one or more amino acids of an amino acid sequence encoding one or more variable heavy chain CDRs of SEQ ID NO: 46 or SEQ ID NO: 47 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-CD22 antibody heavy chain comprises SEQ ID NO: 48. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 48)
EVKLLESGGGLVQPGGSLKLSCEVSGFDFSRDWMSWVRQAPGKGLEWIGQISPDSTSIN
YKPSLKDKFIISRDNAKNTLYLQLSGVRSEDTALYHCARDTSGYPDYWGQGTSLTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSRWQEGNVFSCSVM
HEALHNHYTQKSLSLSLGRDVRVRKIKPLSEIHSGNSVSLQCDFSSSHPKEVQFFWEKNGR
LLGKESQLNFDSISPEDAGSYSCWVNNSIGQTASKAWTLEVLYAPRRLRVSMSPGDQV
MEGKSATLTCESDANPPVSHYTWFDWNNQSLPYHSQKLRLEPVKVQHSGAYWCQGTN
SVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 48. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 49. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the CD22 domain.

(SEQ ID NO: 49)
EVKLLESGGGLVQPGGSLKLSCEVSGFDFSRDWMSWVRQAPGKGLEWIGQISPDSTSIN
YKPSLKDKFIISRDNAKNTLYLQLSGVRSEDTALYHCARDTSGYPDYWGQGTSLTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSRWQEGNVFSCSVM
HEALHNHYTQKSLSLSLGLRVSMSPGDQVMEGKSATLTCESDANPPVSHYTWFDWNNQ
SLPYHSQKLRLEPVKVQHSGAYWCQGTNSVGKGRSPLSTLTVYYSPETIGRR.

In some embodiments, the amino acid sequence of the anti-PD-1-CD22 antibody heavy chain comprises SEQ ID NO: 1 immediately followed by SEQ ID NO: 49. In some embodiments, the signal peptide is cleaved during post-translational modifications that occur in vitro or in vivo.

In some embodiments, the amino acid sequence of the anti-PD-1-CD22 antibody heavy chain comprises SEQ ID NO: 1 immediately followed by SEQ ID NO: 48 except the double underlined portion is replaced with SEQ ID NO: 8. In some embodiments, the amino acid sequence of the anti-PD-1-CD22 antibody heavy chain comprises SEQ ID NO: 1 immediately followed by SEQ ID NO: 49 except the double underlined portion is replaced with SEQ ID NO: 8. In some embodiments, the signal peptide is cleaved during post-translational modifications that occur in vitro or in vivo.

In some embodiments, the amino acid sequence of the anti-PD-1-CD22 antibody light chain (without the signal peptide) comprises SEQ ID NO: 3, 15, 17, 19, 21, 23, 25, 27, 29, 31, 73, or 75 as provided above for the anti-PD1-Fc antibody. In some embodiments, the amino acid sequence of the anti-PD-1-CD22 antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 48 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 49 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 48 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the monoclonal antibody comprises a first and third chain each comprises SEQ ID NO: 49 except the double underlined portion is replaced with SEQ ID NO: 8 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-CD22 antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 48. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 49. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 48 with the double underlined portion replaced with SEQ ID NO: 8. In some embodiments, the anti-PD-1-CD22 antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 49 with the double underlined portion replaced with SEQ ID NO: 8. In some embodiments, the anti-PD-1-CD22 antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-CD22 antibody comprises an amino acid sequence comprising the CDRs of the variable heavy chain domain and the CDRs of the variable light chain domain of the anti-PD-1-CD22 antibody, wherein the CDR sequences are indicated above (in bold and underline), in FIG. 11, or in Tables 1 and 3 for SEQ ID NO: 48, 49, 3, 15, 17, 19, 21, 23, 25, 27, 29, 31, 73 or 75. In some embodiments, the framework regions (FRs) of the variable heavy chain domain and the FRs of the variable light chain domain of the anti-PD-1-CD22 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 48, 49, 3, 15, 17, 19, 21, 23, 25, 27, 29, 31, 73, or 75, or the FRs in Tables 2 and 4.

In some embodiments, one or more amino acids of an amino acid sequence encoding one or more CDRs of SEQ ID NO: 48, 49, 3, 15, 17, 19, 21, 23, 25, 27, 29, 31, 73, or 75 is substituted. In some embodiments, one or more amino acids of an amino acid sequence encoding one or more variable heavy chain CDRs of SEQ ID NO: 48 or SEQ ID NO: 49 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-CD22 antibody described herein can comprise a VH and VL of humanized clone 051 as provided in SEQ ID NO: 74 and 75 or a VH and VL of clone 004 as provided in SEQ ID NO: 72 and 73. In some embodiments, the amino acid sequence of the anti-PD-1-CD22 antibody described herein can comprise CDRs of any of the VH and VL sequences provided herein (see e.g., Tables 1 and 3).

### Nucleic Acid Sequences of Anti-PD-1-CD22 Antibodies

A person skilled in the art can identify the nucleic acid sequences encoding the features identified in the corresponding amino acid sequences (e.g., CDRs, variable heavy domain, variable light domain, CH1 domain, hinge domain, Fc domain, and CD22 domain) by translating the nucleic acid sequences into amino acid sequences. In some embodiments, the nucleic acid sequences may be codon optimized. In some embodiments, nucleic acids encoding VH and VL sequences disclosed herein are provided in Table 5.

In some embodiments, the nucleic acid sequence of the anti-PD-1-CD22 antibody comprises a nucleic acid sequence encoding a signal peptide comprising SEQ ID NO: 5.

In some embodiments, the nucleic acid sequence encoding the anti-PD-1-CD22 antibody heavy chain comprises one of SEQ ID NO: 11, 12, or 32-49. In some embodiments, the nucleic acid sequence encoding the anti-PD-1-CD22 antibody light chain comprises SEQ ID NO: 5 immediately followed by one of SEQ ID NO: 11, 12, or 32-49. In some embodiments, the signal peptide is cleaved during post-translational modifications that occur in vitro or in vivo. Nucleic acid sequences encoding the respective CDRs, VH domain and CH domain in SEQ ID NO: 11, 12, or 32-49, 72, or 74 can be determined from the amino acid sequence of SEQ ID NO: 11, 12, or 32-49, 72 or 74 as identified herein by a person of skill in the art.

In some embodiments, the amino acid sequence of the anti-PD-1-CD22 antibody heavy chain comprises SEQ ID NO: 11, 32, 34, 36, 38, 40, 42, 44, 46, or 48.

In some embodiments, the nucleic acid sequence encoding the anti-PD-1-CD22 antibody heavy chain comprises a nucleic acid sequence encoding SEQ ID NO: 11, 32, 34, 36, 38, 40, 42, 44, 46, or 48. In some embodiments, the nucleic acid sequence encoding the anti-PD-1-CD22 antibody heavy chain comprises SEQ ID NO: 5 immediately followed by a nucleic acid sequence encoding SEQ ID NO: 11, 32, 34, 36, 38, 40, 42, 44, 46, or 48. In some embodiments, the signal peptide is cleaved during post-translational modifications that occur in vitro or in vivo. Nucleic acid sequences encoding the anti-PD-1-CD22 antibody heavy chain and respective CDRs, VH domain, CH1 domain, hinge domain, Fc domain and CD22 domain can be determined from the amino acid sequence of SEQ ID NO: 11, 32, 34, 36, 38, 40, 42, 44, 46, or 48 by a person of skill in the art.

In some embodiments, the amino acid sequence of the anti-PD-1-CD22 antibody heavy chain comprises SEQ ID NO: 12, 33, 35, 37, 39, 41, 43, 45, 47, or 49.

In some embodiments, the nucleic acid sequence encoding the anti-PD-1-CD22 antibody heavy chain comprises a nucleic acid sequence encoding SEQ ID NO: 12, 33, 35, 37, 39, 41, 43, 45, 47, or 49. In some embodiments, the nucleic acid sequence encoding the anti-PD-1-CD22 antibody heavy chain comprises SEQ ID NO: 5 immediately followed by a nucleic acid sequence encoding SEQ ID NO: 12, 33, 35, 37, 39, 41, 43, 45, 47, or 49. In some embodiments, the signal peptide is cleaved during post-translational modifications that occur in vitro or in vivo. Nucleic acid sequences encoding the anti-PD-1-CD22 antibody heavy chain and respective CDRs, VH domain, CH1 domain, hinge domain, Fc domain and CD22 domain can be determined from the amino acid sequence of SEQ ID NO: 12, 33, 35, 37, 39, 41, 43, 45, 47, or 49 by a person of skill in the art.

In some embodiments, the nucleic acid sequence encoding the anti-PD-1-CD22 antibody light chain comprises SEQ ID NO: 7 provided above for anti-PD-1-Fc. In some embodiments, the nucleic acid sequence encoding the anti-PD-1-Fc antibody light chain comprises SEQ ID NO: 5 immediately followed by SEQ ID NO: 7. In some embodiments, the signal peptide is cleaved during post-translational modifications that occur in vitro or in vivo. Nucleic acid sequences encoding the respective CDRs, VL domain and CL domain in SEQ ID NO: 7 can be determined from the amino acid sequence of SEQ ID NO: 3 as identified herein by a person of skill in the art.

In some embodiments, the amino acid sequence of the anti-PD-1-CD22 antibody light chain comprises SEQ ID NO: 3, 15, 17, 19, 21, 23, 25, 27, 29, 31, 73, or 75.

In some embodiments, the nucleic acid sequence encoding the anti-PD-1-CD22 antibody light chain comprises a nucleic acid sequence encoding SEQ ID NO: 3, 15, 17, 19, 21, 23, 25, 27, 29, 31, 73, or 75. In some embodiments, the nucleic acid sequence encoding the anti-PD-1-CD22 antibody light chain comprises SEQ ID NO: 5 immediately followed by a nucleic acid sequence encoding SEQ ID NO: 3, 15, 17, 19, 21, 23, 25, 27, 29, 31, 73, or 75. In some embodiments, the signal peptide is cleaved during post-translational modifications that occur in vitro or in vivo. Nucleic acid sequences encoding the anti-PD-1-CD22 antibody light chain and respective CDRs, VL domain, and CLI domain can be determined from the amino acid sequence of SEQ ID NO: 3, 15, 17, 19, 21, 23, 25, 27, 29, 31, 73, or 75 by a person of skill in the art.

In some embodiments, the nucleic acid sequence encoding a signal peptide of the anti-PD-1-CD22 antibody heavy or light chain comprise a nucleic acid sequence 80, 81, 82, 83, 84, 85, 86, 87, 88, 89, 90, 91, 92, 93, 94, 95, 96, 97, 98 or 99% identical to SEQ ID NO: 5. In some embodiments, the nucleic acid sequence encoding an anti-PD-1-CD22 heavy chain (without the signal peptide) comprise a nucleic acid sequence 80, 81, 82, 83, 84, 85, 86, 87, 88, 89, 90, 91, 92, 93, 94, 95, 96, 97, 98 or 99% identical to SEQ ID NO: 11, 12, or 32-49. In some embodiments, the nucleic acid sequence encoding an anti-PD-1-CD22 light chain (without the signal peptide) comprise a nucleic acid sequence 80, 81, 82, 83, 84, 85, 86, 87, 88, 89, 90, 91, 92, 93, 94, 95, 96, 97, 98 or 99% identical to SEQ ID NO: 7.

In some embodiments, the nucleic acid sequence encoding a signal peptide of the anti-PD-1-CD22 antibody heavy or light chain comprise a nucleic acid sequence encoding an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the nucleic acid sequence encoding an anti-PD-1-CD22 heavy chain (without the signal peptide) comprise a nucleic acid sequence encoding an amino acid sequence 80, 81, 82, 83, 84, 85, 86, 87, 88, 89, 90, 91, 92, 93, 94, 95, 96, 97, 98 or 99% identical to SEQ ID NO: 11, 12, or 32-49. In some embodiments, the nucleic acid sequence encoding an anti-PD-1-CD22 light chain (without the signal peptide) comprise a nucleic acid sequence encoding an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75.

In some embodiments, the nucleic acid sequence encodes CDRs of the variable heavy chain domain and the CDRs of the variable light chain domain of the anti-PD-1-CD22 antibody, wherein the CDR sequences are indicated above (in bold and underline) in SEQ ID NO: 2, 3, 11, 12, 14-49. In some embodiments, the nucleic acid sequence encoding framework regions (FRs) of the variable heavy chain domain and the FRs of the variable light chain domain of the anti-PD-1-CD22 antibody comprise a nucleic acid sequence 80, 81, 82, 83, 84, 85, 86, 87, 88, 89, 90, 91, 92, 93, 94, 95, 96, 97, 98 or 99% identical to the FRs of SEQ ID NO: 2, 3, 11, 12, 14-49. In some embodiments, one or more nucleic acids of a nucleic acid sequence encoding one or more CDRs of SEQ ID NO: 2, 3, 11, 12, 14-49 is substituted. In some embodiments, one or more nucleic acids of a nucleic acid sequence encoding one or more variable heavy chain CDRs of SEQ ID NO: 2, 11, 12, 14, 16, 18, 20, 22, 24, 26, 28, 30, 32-49 is substituted. In some embodiments, one or more nucleic acids of a nucleic acid sequence encoding one or more variable light chain CDRs of SEQ ID NO: 3, 15, 17, 19, 21, 23, 25, 27, 29, 31, 73, or 75 is substituted.

In some embodiments, the nucleic acid sequence comprises a nucleic acid sequence encoding the scFv-Fc described herein.

### Amino Acid Sequences of Anti-PD-1-HSA Antibodies

In some embodiments, the anti-PD-1-HSA antibody comprises two polypeptide heavy chains each comprising a variable heavy chain (VH) domain, CH1 domain, a hinge domain, a first Fc domain (CH2-CH3), and a HSA domain and two polypeptide light chains comprising a variable light chain (VL) domain and a CL domain.

In some embodiments, the amino acid sequence of the anti-PD-1-HSA antibody heavy chain comprises a signal peptide comprising SEQ ID NO: 1.

In some embodiments, the amino acid sequence of the HSA domain comprises Human Serum Albumin (SEQ ID NO: 50):

(SEQ ID NO: 50)
DAHKSEVAHRFKDLGEENFKALVLIAFAQYLQQCPFEDHVKLVNEVTEFAKTCVADES
AENCDKSLHTLFGDKLCTVATLRETYGEMADCCAKQEPERNECFLQHKDDNPNLPRLV
RPEVDVMCTAFHDNEETFLKKYLYEIARRHPYFYAPELLFFAKRYKAAFTECCQAADK
AACLLPKLDELRDEGKASSAKQRLKCASLQKFGERAFKAWAVARLSQRFPKAEFAEVS
KLVTDLTKVHTECCHGDLLECADDRADLAKYICENQDSISSKLKECCEKPLLEKSHCIA
EVENDEMPADLPSLAADFVESKDVCKNYAEAKDVFLGMFLYEYARRHPDYSVVLLLR
LAKTYETTLEKCCAAADPHECYAKVFDEFKPLVEEPQNLIKQNCELFEQLGEYKFQNAL
LVRYTKKVPQVSTPTLVEVSRNLGKVGSKCCKHPEAKRMPCAEDYLSVVLNQLCVLHE
KTPVSDRVTKCCTESLVNRRPCFSALEVDETYVPKEFNAETFTFHADICTLSEKERQIKK
QTALVELVKHKPKATKEQLKAVMDDFAAFVEKCCKADDKETCFAEEGKKLVAASQA
ALGL.

In some embodiments, the amino acid sequence of the HSA domain comprises any fragment of SEQ ID NO: 50 as long as the HSA domain increases the monoclonal antibody size to at least 150 kD, at least 160 kD, at least 170 kD, at least 180 kD, at least 190 kD, or at least 200 kD. In some embodiments, the amino acid sequence of the HSA domain comprises any fragment of SEQ ID NO: 50 as long as the HSA domain increases the monoclonal antibody size to at least about 200 kD. In some embodiments, the amino acid sequence of the HSA domain comprises any fragment of SEQ ID NO: 50 as long as the HSA domain increases the monoclonal antibody size to about 200 kD.

In some embodiments, the amino acid sequence of the anti-PD-1-HSA antibody heavy chain comprises SEQ ID NO: 51. Underlined amino acids represent the VH domain. Underlined and bolded amino acids shown in FIG. 11 represent the respective complementarity determining regions (CDRs). Italicized and bold amino acids represent the CH1 domain. Italicized and underlined amino acids represent the hinge domain. Italicized amino acids represent the Fc domain. Double underlined amino acids represent the HSA domain.

(SEQ ID NO: 51)
QVQLVQSGVEVKKPGASVKVSCKASGYTFTNYYMYWVRQAPGQGLEWMGGINPSN
GGTNFNEKFKNRVTLTTDSSTTTAYMELKSLQFDDTAVYYCARRDYRFDMGFDYWG
QGTLVTVSSASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVESKYGPPCPPCPAP
EFEGGPSVFLFPPKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFLYSRLTVDKSRWQ
EGNVFSCSVMHEALHNHYTQKSLSLSLGDAHKSEVAHRFKDLGEENFKALVLIAFAQYLQ
QCPFEDHVKLVNEVTEFAKTCVADESAENCDKSLHTLFGDKLCTVATLRETYGEMADC
CAKQEPERNECFLQHKDDNPNLPRLVRPEVDVMCTAFHDNEETELKKYLYEIARRHPY
FYAPELLFFAKRYKAAFTECCQAADKAACLLPKLDELRDEGKASSAKQRLKCASLQKF
GERAFKAWAVARLSQRFPKAEFAEVSKLVTDLTKVHTECCHGDLLECADDRADLAKYI
CENQDSISSKLKECCEKPLLEKSHCIAEVENDEMPADLPSLAADFVESKDVCKNYAEAK
DVFLGMFLYEYARRHPDYSVVLLLRLAKTYETTLEKCCAAADPHECYAKVFDEFKPLV
EEPQNLIKQNCELFEQLGEYKFQNALLVRYTKKVPQVSTPTLVEVSRNLGKVGSKCCK
HPEAKRMPCAEDYLSVVLNQLCVLHEKTPVSDRVTKCCTESLVNRRPCFSALEVDETY
VPKEFNAETFTFHADICTLSEKERQIKKQTALVELVKHKPKATKEQLKAVMDDFAAFVE
KCCKADDKETCFAEEGKKLVAASQAALGL.

In some embodiments, the amino acid sequence of the anti-PD-1-HSA antibody heavy chain comprises SEQ ID NO: 1 immediately followed by SEQ ID NO: 51. In some embodiments, the signal peptide is cleaved during post-translational modifications that occur in vitro or in vivo.

In some embodiments, the amino acid sequence of the anti-PD-1-HSA antibody light chain (without the signal peptide) comprises SEQ ID NO: 3, 15, 17, 19, 21, 23, 25, 27, 29, 31, 73, or 75 as provided above for the anti-PD1-HSA antibody. In some embodiments, the amino acid sequence of the anti-PD-1-HSA antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 51 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-HSA antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-HSA antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 51. In some embodiments, the anti-PD-1-HSA antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-HSA antibody comprises an amino acid sequence comprising the CDRs of the variable heavy chain domain and the CDRs of the variable light chain domain of the anti-PD-1-HSA antibody, wherein the CDR sequences are indicated above (in bold and underline), in FIG. 11, or in Tables 1 and 3 for SEQ ID NO: 3, 51, 15, 17, 19, 21, 23, 25, 27, 29, 31, 72, 73, 74, and 75. In some embodiments, the framework regions (FRs) of the variable heavy chain domain and the FRs of the variable light chain domain of the anti-PD-1-HSA antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 51, 15, 17, 19, 21, 23, 25, 27, 29, 31, 72, 73, 74, or 75, or the FRs in Tables 2 and 4.

In some embodiments, one or more amino acids of an amino acid sequence encoding one or more CDRs of SEQ ID NO: 3, 51, 15, 17, 19, 21, 23, 25, 27, 29, 31, 73, or 75 is substituted. In some embodiments, one or more amino acids of an amino acid sequence encoding one or more variable heavy chain CDRs of SEQ ID NO: 51 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-HSA antibody heavy chain comprises SEQ ID NO: 52. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the HSA domain.

(SEQ ID NO: 52)
EVKLLESGGGLVQPGGSLKLSCAASGFDFSRYWMSWVRQAPGKGLEWIGEINPDSSTIK
YTPSLKDKFIISRDNAKNTLCLQLSKVRSEDSALYYCARMGYRLFDSWGQGTTLTVSSA
STKGPSVFPLAPCSRSTSESTAALGCLVKDYFPEPVTVSWNSGALTSGVHTFPAVLQSSG
LYSLSSVVTVPSSSLGTKTYTCNVDHKPSNTKVDKRVESKYGPPCPPCPAPEFEGGPSVFL
FPPKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKSRWQEGNVFSCSVM
HEALHNHYTQKSLSLSLGDAHKSEVAHRFKDLGEENFKALVLIAFAQYLQQCPFEDHVK
LVNEVTEFAKTCVADESAENCDKSLHTLFGDKLCTVATLRETYGEMADCCAKQEPERN
ECFLQHKDDNPNLPRLVRPEVDVMCTAFHDNEETFLKKYLYEIARRHPYFYAPELLFFA
KRYKAAFTECCQAADKAACLLPKLDELRDEGKASSAKQRLKCASLQKFGERAFKAWA
VARLSQRFPKAEFAEVSKLVTDLTKVHTECCHGDLLECADDRADLAKYICENQDSISSK
LKECCEKPLLEKSHCIAEVENDEMPADLPSLAADFVESKDVCKNYAEAKDVELGMFLY
EYARRHPDYSVVLLLRLAKTYETTLEKCCAAADPHECYAKVFDEFKPLVEEPQNLIKQ
NCELFEQLGEYKFQNALLVRYTKKVPQVSTPTLVEVSRNLGKVGSKCCKHPEAKRMPC
AEDYLSVVLNQLCVLHEKTPVSDRVTKCCTESLVNRRPCFSALEVDETYVPKEFNAETF
TFHADICTLSEKERQIKKQTALVELVKHKPKATKEQLKAVMDDFAAFVEKCCKADDKE
TCFAEEGKKLVAASQAALGL.

In some embodiments, the amino acid sequence of the anti-PD-1-HSA antibody heavy chain comprises SEQ ID NO: 1 immediately followed by SEQ ID NO: 52. In some embodiments, the signal peptide is cleaved during post-translational modifications that occur in vitro or in vivo.

In some embodiments, the amino acid sequence of the anti-PD-1-HSA antibody light chain (without the signal peptide) comprises SEQ ID NO: 3, 15, 17, 19, 21, 23, 25, 27, 29, 31, 73, or 75 as provided above for the anti-PD1-HSA antibody. In some embodiments, the amino acid sequence of the anti-PD-1-HSA antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 52 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-HSA antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-HSA antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 52. In some embodiments, the anti-PD-1-HSA antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-HSA antibody comprises an amino acid sequence comprising the CDRs of the variable heavy chain domain and the CDRs of the variable light chain domain of the anti-PD-1-HSA antibody, wherein the CDR sequences are indicated above (in bold and underline), in FIG. 11, or in Tables 1 and 3 for SEQ ID NO: 3, 15, 17, 19, 21, 23, 25, 27, 29, 31, 52, 73 and 75. In some embodiments, the framework regions (FRs) of the variable heavy chain domain and the FRs of the variable light chain domain of the anti-PD-1-HSA antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 52, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 52, 73, or 75 is substituted. In some embodiments, one or more amino acids of an amino acid sequence encoding one or more variable heavy chain CDRs of SEQ ID NO: 52 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-HSA antibody heavy chain comprises SEQ ID NO: 53. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the HSA domain.

(SEQ ID NO: 53)
VQLQQSGTVLARPGASVKMSCKASGNTFTSYWMHWVKQRPGQGLEWIGAIYPGYSDT
RYNQKFKGKATLTAVTSTSTAYMELSSLTNEDSAVYYCTKFIATVGGYFDVWGAGTTV
TVSSASTKGPSVFPLAPCSRSTSESTAALGCLVKDYFPEPVTVSWNSGALTSGVHTFPAV
LQSSGLYSLSSVVTVPSSSLGTKTYTCNVDHKPSNTKVDKRVESKYGPPCPPCPAPEFEGG
PSVFLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGNVFS
CSVMHEALHNHYTQKSLSLSLGDAHKSEVAHRFKDLGEENFKALVLIAFAQYLQQCPFED
HVKLVNEVTEFAKTCVADESAENCDKSLHTLFGDKLCTVATLRETYGEMADCCAKQE
PERNECFLQHKDDNPNLPRLVRPEVDVMCTAFHDNEETELKKYLYEIARRHPYFYAPEL
LFFAKRYKAAFTECCQAADKAACLLPKLDELRDEGKASSAKQRLKCASLQKFGERAFK
AWAVARLSQREPKAEFAEVSKLVTDLTKVHTECCHGDLLECADDRADLAKYICENQDS
ISSKLKECCEKPLLEKSHCIAEVENDEMPADLPSLAADEVESKDVCKNYAEAKDVFLGM
FLYEYARRHPDYSVVLLLRLAKTYETTLEKCCAAADPHECYAKVFDEEKPLVEEPQNLI
KQNCELFEQLGEYKFQNALLVRYTKKVPQVSTPTLVEVSRNLGKVGSKCCKHPEAKR
MPCAEDYLSVVLNQLCVLHEKTPVSDRVTKCCTESLVNRRPCFSALEVDETYVPKEFN
AETFTFHADICTLSEKERQIKKQTALVELVKHKPKATKEQLKAVMDDFAAFVEKCCKA
DDKETCFAEEGKKLVAASQAALGL.

In some embodiments, the amino acid sequence of the anti-PD-1-HSA antibody heavy chain comprises SEQ ID NO: 1 immediately followed by SEQ ID NO: 53. In some embodiments, the signal peptide is cleaved during post-translational modifications that occur in vitro or in vivo.

In some embodiments, the amino acid sequence of the anti-PD-1-HSA antibody light chain (without the signal peptide) comprises SEQ ID NO: 3, 15, 17, 19, 21, 23, 25, 27, 29, 31, 73, or 75 as provided above for the anti-PD1-HSA antibody. In some embodiments, the amino acid sequence of the anti-PD-1-HSA antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 53 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-HSA antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-HSA antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 53. In some embodiments, the anti-PD-1-HSA antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-HSA antibody comprises an amino acid sequence comprising the CDRs of the variable heavy chain domain and the CDRs of the variable light chain domain of the anti-PD-1-HSA antibody, wherein the CDR sequences are indicated above (in bold and underline), in FIG. 11, or in Tables 1 and 3 for SEQ ID NO: 3, 15, 17, 19, 21, 23, 25, 27, 29, 31, 53, 73, and 75. In some embodiments, the framework regions (FRs) of the variable heavy chain domain and the FRs of the variable light chain domain of the anti-PD-1-HSA antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 53, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 53, 73 or 75 is substituted. In some embodiments, one or more amino acids of an amino acid sequence encoding one or more variable heavy chain CDRs of SEQ ID NO: 53 is substituted. In some embodiments, one or more amino acids of an amino acid sequence encoding one or more variable light chain CDRs of SEQ ID NO: 3, 15, 17, 19, 21, 23, 25, 27, 29, 31, 73 or 75 is substituted.

In some embodiments, the amino acid sequence of the anti-PD-1-HSA antibody heavy chain comprises SEQ ID NO: 54. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the HSA domain.

(SEQ ID NO: 54)
QIQLVQSGPELKKPGETVKISCKASGYTFTDYSMHWVKQAPGKGLKWMGWIKIETGNP
TYADDFKGRFAFSLETSASTAYLQINNLKNEDTATYFCARDYYGYYYYAMDYWGQGT
SVTVSSASTKGPSVFPLAPCSRSTSESTAALGCLVKDYFPEPVTVSWNSGALTSGVHTFP
AVLQSSGLYSLSSVVTVPSSSLGTKTYTCNVDHKPSNTKVDKRVESKYGPPCPPCPAPEFE
GGPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTVDKSRWQEGN
VFSCSVMHEALHNHYTQKSLSLSLGDAHKSEVAHRFKDLGEENFKALVLIAFAQYLQQCP
FEDHVKLVNEVTEFAKTCVADESAENCDKSLHTLFGDKLCTVATLRETYGEMADCCA
KQEPERNECFLQHKDDNPNLPRLVRPEVDVMCTAFHDNEETELKKYLYEIARRHPYFY
APELLEFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICE
NQDSISSKLKECCEKPLLEKSHCIAEVENDEMPADLPSLAADEVESKDVCKNYAEAKDV
FLGMFLYEYARRHPDYSVVLLLRLAKTYETTLEKCCAAADPHECYAKVFDEFKPLVEE
PQNLIKQNCELFEQLGEYKFQNALLVRYTKKVPQVSTPTLVEVSRNLGKVGSKCCKHPE
AKRMPCAEDYLSVVLNQLCVLHEKTPVSDRVTKCCTESLVNRRPCFSALEVDETYVPK
EFNAETFTFHADICTLSEKERQIKKQTALVELVKHKPKATKEQLKAVMDDEAAFVEKC
CKADDKETCFAEEGKKLVAASQAALGL.

In some embodiments, the amino acid sequence of the anti-PD-1-HSA antibody heavy chain comprises SEQ ID NO: 1 immediately followed by SEQ ID NO: 54. In some embodiments, the signal peptide is cleaved during post-translational modifications that occur in vitro or in vivo.

In some embodiments, the amino acid sequence of the anti-PD-1-HSA antibody light chain (without the signal peptide) comprises SEQ ID NO: 3, 15, 17, 19, 21, 23, 25, 27, 29, 31, 73, or 75 as provided above for the anti-PD1-HSA antibody. In some embodiments, the amino acid sequence of the anti-PD-1-HSA antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 54 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-HSA antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-HSA antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 54. In some embodiments, the anti-PD-1-HSA antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-HSA antibody comprises an amino acid sequence comprising the CDRs of the variable heavy chain domain and the CDRs of the variable light chain domain of the anti-PD-1-HSA antibody, wherein the CDR sequences are indicated above (in bold and underline), in FIG. 11, or in Tables 1 and 3 for SEQ ID NO: 3, 15, 17, 19, 21, 23, 25, 27, 29, 31, 54, 73, and 75. In some embodiments, the framework regions (FRs) of the variable heavy chain domain and the FRs of the variable light chain domain of the anti-PD-1-HSA antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 54, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 54, 73, or 75 is substituted. In some embodiments, one or more amino acids of an amino acid sequence encoding one or more variable heavy chain CDRs of SEQ ID NO: 54 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-HSA antibody heavy chain comprises SEQ ID NO: 55. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the HSA domain.

(SEQ ID NO: 55)
VQLVESGGGLVQPGGSLRLSCAASGFTFSHYGMSWVRQTPDKRLELVATIDNNGGNTY
YPDSVKGRFTISRDNAKNTLYLQMSSLKSEDTAMYYCARDAYYTYDYWYFDVWGAG
TTVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRWQEG
NVFSCSVMHEALHNHYTQKSLSLSLGDAHKSEVAHRFKDLGEENFKALVLIAFAQYLQQC
PFEDHVKLVNEVTEFAKTCVADESAENCDKSLHTLFGDKLCTVATLRETYGEMADCCA
KQEPERNECFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICE
NQDSISSKLKECCEKPLLEKSHCIAEVENDEMPADLPSLAADFVESKDVCKNYAEAKDV
ELGMFLYEYARRHPDYSVVLLLRLAKTYETTLEKCCAAADPHECYAKVFDEFKPLVEE
PQNLIKQNCELFEQLGEYKFQNALLVRYTKKVPQVSTPTLVEVSRNLGKVGSKCCKHPE
AKRMPCAEDYLSVVLNQLCVLHEKTPVSDRVTKCCTESLVNRRPCESALEVDETYVPK
EFNAETFTFHADICTLSEKERQIKKQTALVELVKHKPKATKEQLKAVMDDFAAFVEKC
CKADDKETCFAEEGKKLVAASQAALGL.

In some embodiments, the amino acid sequence of the anti-PD-1-HSA antibody heavy chain comprises SEQ ID NO: 1 immediately followed by SEQ ID NO: 55. In some embodiments, the signal peptide is cleaved during post-translational modifications that occur in vitro or in vivo.

In some embodiments, the amino acid sequence of the anti-PD-1-HSA antibody light chain (without the signal peptide) comprises SEQ ID NO: 3, 15, 17, 19, 21, 23, 25, 27, 29, 31, 73, or 75 as provided above for the anti-PD1-HSA antibody. In some embodiments, the amino acid sequence of the anti-PD-1-HSA antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 55 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-HSA antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-HSA antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 55. In some embodiments, the anti-PD-1-HSA antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-HSA antibody comprises an amino acid sequence comprising the CDRs of the variable heavy chain domain and the CDRs of the variable light chain domain of the anti-PD-1-HSA antibody, wherein the CDR sequences are indicated above (in bold and underline), in FIG. 11, or in Tables 1 and 3 for SEQ ID NO: 3, 15, 17, 19, 21, 23, 25, 27, 29, 31, 55, 73, and 75. In some embodiments, the framework regions (FRs) of the variable heavy chain domain and the FRs of the variable light chain domain of the anti-PD-1-HSA antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 55, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 55, 73 or 75 is substituted. In some embodiments, one or more amino acids of an amino acid sequence encoding one or more variable heavy chain CDRs of SEQ ID NO: 55 is substituted. In some embodiments, one or more amino acids of an amino acid sequence encoding one or more variable light chain CDRs of SEQ ID NO: 3, 15, 17, 19, 21, 23, 25, 27, 29, 31, 73 or 75 is substituted.

In some embodiments, the amino acid sequence of the anti-PD-1-HSA antibody heavy chain comprises SEQ ID NO: 56. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the HSA domain.

(SEQ ID NO: 56)
EVKLVESGGGLVQPGGSLKLSCAASGFTFSSYTMSWVRQTPEKRLEWVAYISYGGGDT
YYPDTVKGRFTISRDNAKNTLYLQMSSLKSEDTAMYYCARQKVDGYFVAMDYWSQG
TSVTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRWQEG
NVFSCSVMHEALHNHYTQKSLSLSLGDAHKSEVAHRFKDLGEENFKALVLIAFAQYLQQC
PFEDHVKLVNEVTEFAKTCVADESAENCDKSLHTLFGDKLCTVATLRETYGEMADCCA
KQEPERNECELQHKDDNPNLPRLVRPEVDVMCTAFHDNEETFLKKYLYEIARRHPYFY
APELLEFAKRYKAAFTECCQAADKAACLLPKLDELRDEGKASSAKQRLKCASLQKFGE
RAFKAWAVARLSQRFPKAEFAEVSKLVTDLTKVHTECCHGDLLECADDRADLAKYICE
NQDSISSKLKECCEKPLLEKSHCIAEVENDEMPADLPSLAADFVESKDVCKNYAEAKDV
FLGMFLYEYARRHPDYSVVLLLRLAKTYETTLEKCCAAADPHECYAKVFDEFKPLVEE
PQNLIKQNCELFEQLGEYKFQNALLVRYTKKVPQVSTPTLVEVSRNLGKVGSKCCKHPE
AKRMPCAEDYLSVVLNQLCVLHEKTPVSDRVTKCCTESLVNRRPCFSALEVDETYVPK
EFNAETFTEHADICTLSEKERQIKKQTALVELVKHKPKATKEQLKAVMDDEAAFVEKC
CKADDKETCEAEEGKKLVAASQAALGL.

In some embodiments, the amino acid sequence of the anti-PD-1-HSA antibody heavy chain comprises SEQ ID NO: 1 immediately followed by SEQ ID NO: 56. In some embodiments, the signal peptide is cleaved during post-translational modifications that occur in vitro or in vivo.

In some embodiments, the amino acid sequence of the anti-PD-1-HSA antibody light chain (without the signal peptide) comprises SEQ ID NO: 3, 15, 17, 19, 21, 23, 25, 27, 29, 31, 73 or 75 as provided above for the anti-PD1-HSA antibody. In some embodiments, the amino acid sequence of the anti-PD-1-HSA antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 56 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-HSA antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-HSA antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 56. In some embodiments, the anti-PD-1-HSA antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-HSA antibody comprises an amino acid sequence comprising the CDRs of the variable heavy chain domain and the CDRs of the variable light chain domain of the anti-PD-1-HSA antibody, wherein the CDR sequences are indicated above (in bold and underline), in FIG. 11, or in Tables 1 and 3 for SEQ ID NO: 3, 15, 17, 19, 21, 23, 25, 27, 29, 31, 56, 73, and 75. In some embodiments, the framework regions (FRs) of the variable heavy chain domain and the FRs of the variable light chain domain of the anti-PD-1-HSA antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 56, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 56, 73, or 75 is substituted. In some embodiments, one or more amino acids of an amino acid sequence encoding one or more variable heavy chain CDRs of SEQ ID NO: 56 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-HSA antibody heavy chain comprises SEQ ID NO: 57. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the HSA domain.

(SEQ ID NO: 57)
EVKLVESGGGLVKPGGSLKLSCAASGFTFSSYGMSWVRQTPEKRLEWVATISGGGRYT
YYPDSVKGRFTISRDNAKNILYLQMSSLRSEDTALYYCASPYDGYYGAMDYWGQGTS
VTVSSASTKGPSVFPLAPCSRSTSESTAALGCLVKDYFPEPVTVSWNSGALTSGVHTFPA
VLQSSGLYSLSSVVTVPSSSLGTKTYTCNVDHKPSNTKVDKRVESKYGPPCPPCPAPEFEG
GPSVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMTKN
QVSLTCLVKGFYPSDIAVEWESNGQPENNYKTTPPVLDSDGSFFLYSRLTVDKSRWQEGNVFS
CSVMHEALHNHYTQKSLSLSLGDAHKSEVAHRFKDLGEENFKALVLIAFAQYLQQCPFED
HVKLVNEVTEFAKTCVADESAENCDKSLHTLFGDKLCTVATLRETYGEMADCCAKQE
PERNECFLQHKDDNPNLPRLVRPEVDVMCTAFHDNEETFLKKYLYEIARRHPYFYAPEL
LFFAKRYKAAFTECCQAADKAACLLPKLDELRDEGKASSAKQRLKCASLQKFGERAFK
AWAVARLSQRFPKAEFAEVSKLVTDLTKVHTECCHGDLLECADDRADLAKYICENQDS
ISSKLKECCEKPLLEKSHCIAEVENDEMPADLPSLAADFVESKDVCKNYAEAKDVFLGM
FLYEYARRHPDYSVVLLLRLAKTYETTLEKCCAAADPHECYAKVFDEFKPLVEEPQNLI
KQNCELFEQLGEYKFQNALLVRYTKKVPQVSTPTLVEVSRNLGKVGSKCCKHPEAKR
MPCAEDYLSVVLNQLCVLHEKTPVSDRVTKCCTESLVNRRPCFSALEVDETYVPKEFN
AETFTFHADICTLSEKERQIKKQTALVELVKHKPKATKEQLKAVMDDFAAFVEKCCKA
DDKETCFAEEGKKLVAASQAALGL.

In some embodiments, the amino acid sequence of the anti-PD-1-HSA antibody heavy chain comprises SEQ ID NO: 1 immediately followed by SEQ ID NO: 57. In some embodiments, the signal peptide is cleaved during post-translational modifications that occur in vitro or in vivo.

In some embodiments, the amino acid sequence of the anti-PD-1-HSA antibody light chain (without the signal peptide) comprises SEQ ID NO: 3, 15, 17, 19, 21, 23, 25, 27, 29, 31, 73, or 75 as provided above for the anti-PD1-HSA antibody. In some embodiments, the amino acid sequence of the anti-PD-1-HSA antibody light chain comprises SEQ ID NO: 1 immediately followed by SEQ ID NO: 3, 15, 17, 19, 21, 23, 25, 27, 29, 31, 73 or 75. In some embodiments, the signal peptide is cleaved during post-translational modifications that occur in vitro or in vivo.

In some embodiments, the monoclonal antibody comprises a first and third chain each comprises SEQ ID NO: 57 and a second and fourth chains each comprises SEQ ID NO: 3, 15, 17, 19, 21, 23, 25, 27, 29, 31, 73 or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-HSA antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-HSA antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 57. In some embodiments, the anti-PD-1-HSA antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73 or 75. In some embodiments, the anti-PD-1-HSA antibody comprises an amino acid sequence comprising the CDRs of the variable heavy chain domain and the CDRs of the variable light chain domain of the anti-PD-1-HSA antibody, wherein the CDR sequences are indicated above (in bold and underline), in FIG. 11, or in Tables 1 and 3 for SEQ ID NO: 3, 15, 17, 19, 21, 23, 25, 27, 29, 31, 57, 73, and 75. In some embodiments, the framework regions (FRs) of the variable heavy chain domain and the FRs of the variable light chain domain of the anti-PD-1-HSA antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 57, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 57, 73, or 75 is substituted. In some embodiments, one or more amino acids of an amino acid sequence encoding one or more variable heavy chain CDRs of SEQ ID NO: 57 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-HSA antibody heavy chain comprises SEQ ID NO: 58. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the HSA domain.

(SEQ ID NO: 58)
EVQLQQSGTVLARPGASVRMSCKASGYSFSTYWMHWVKQRPGQGLEWIGGIYPGNSD
TNYNQKFKGKAKLTAVTSASTAYMDLSSLTNEDSAVYYCTGGYFFDVWGAGTTVTVS
SASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFEGGPSV
FLFPPKPKDTLMISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDKSRWQEGNVFSCSV
MHEALHNHYTQKSLSLSLGDAHKSEVAHRFKDLGEENFKALVLIAFAQYLQQCPFEDHV
KLVNEVTEFAKTCVADESAENCDKSLHTLFGDKLCTVATLRETYGEMADCCAKQEPER
NECFLQHKDDNPNLPRLVRPEVDVMCTAFHDNEETFLKKYLYEIARRHPYFYAPELLFF
AKRYKAAFTECCQAADKAACLLPKLDELRDEGKASSAKQRLKCASLQKFGERAFKAW
AVARLSQRFPKAEFAEVSKLVTDLTKVHTECCHGDLLECADDRADLAKYICENQDSISS
KLKECCEKPLLEKSHCIAEVENDEMPADLPSLAADFVESKDVCKNYAEAKDVFLGMGL
YEYARRHPDYSVVLLLRLAKTYETTLEKCCAAADPHECYAKVFDEFKPLVEEPQNLIK
QNCELFEQLGEYKFQNALLVRYTKKVPQVSTPTLVEVSRNLKVGSKCKHPEAKRMP
CAEDYLSVVLNQLCVLHEKTPVSDRVTKCCTESLVNRRPCFSALEVDETYVPKEFNAET
FTFHADICTLSEKERQIKKQTALVELVKHKPKATKEQLKAVMDDFAAFVEKCCKADDK
ETCFAEEGKKLVAASQAALGL.

In some embodiments, the amino acid sequence of the anti-PD-1-HSA antibody heavy chain comprises SEQ ID NO: 1 immediately followed by SEQ ID NO: 58. In some embodiments, the signal peptide is cleaved during post-translational modifications that occur in vitro or in vivo.

In some embodiments, the amino acid sequence of the anti-PD-1-HSA antibody light chain (without the signal peptide) comprises SEQ ID NO: 3, 15, 17, 19, 21, 23, 25, 27, 29, 31, 73, or 75 as provided above for the anti-PD1-HSA antibody. In some embodiments, the amino acid sequence of the anti-PD-1-HSA antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 58 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-HSA antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-HSA antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 58. In some embodiments, the anti-PD-1-HSA antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-HSA antibody comprises an amino acid sequence comprising the CDRs of the variable heavy chain domain and the CDRs of the variable light chain domain of the anti-PD-1-HSA antibody, wherein the CDR sequences are indicated above (in bold and underline), in FIG. 11, or in Tables 1 and 3 for SEQ ID NO: 3, 15, 17, 19, 21, 23, 25, 27, 29, 31, 58, 73, and 75. In some embodiments, the framework regions (FRs) of the variable heavy chain domain and the FRs of the variable light chain domain of the anti-PD-1-HSA antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 58, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 58, 73, or 75 is substituted. In some embodiments, one or more amino acids of an amino acid sequence encoding one or more variable heavy chain CDRs of SEQ ID NO: 58 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-HSA antibody heavy chain comprises SEQ ID NO: 59. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the HSA domain.

(SEQ ID NO: 59)
QIQLQQSGPEQVKPGASVKISCKASGYMFIDYYMNWVKQKPGQGLE
WIGWIYPGSGSTKDNENFKGKATLTVDTSSSTAYMQLSSLTSEDT
AVYFCARYGPRFFDVWGAGTTVTVSSASTKGPSVFPLAPCSRSTS
ESTAALGCLVKDYFPEPVTVSWNSGALTSGVHTFPAVLQSSGLYS
LSSVVTVPSSSLGTKTYTCNVDHKPSNTKVDKRVESKYGPPCPPC
PAPEFEGGPSVFLFPPKPKDTLMISRTPEVTCVVVDVSQEDPEVQ
FNWYVDGVEVHNAKTKPREEQFNSTYRTVSVLTVLHQDWLNGKEY
KCKVSNKGLPSSIEKTISKAKGQPREPQVYTLPPSQEEMTKNQVS
ITCLVKGFYPSDIAVEWESNGQPENNYKTTPPVLDSDGSFFLYSR
LTVDKSRWQEGNVFSCSVMHEALHNHYTQKSLSLSLGDAHKSEVA
HREKDLGEENEKALVLIAFAQYLQQCPEEDHVKLVNEVTEFAKTC
VADESAENCDKSLHTLFGDKLCTVATLRETYGEMADCCAKQEPER
NECFLQHKDDNPNLPRLVRPEVDVMCTAFHDNEETELKKYLYEIA
RRHPYFYAPELLFEAKRYKAAFTECCQAADKAACLLPKLDELRDE
GKASSAKQRLKCASLQKFGERAFKAWAVARLSQRFPKAEFAEVSK
LVTDLTKVHTECCHGDLLECADDRADLAKYICENQDSISSKLKEC
CEKPLLEKSHCIAEVENDEMPADLPSLAADEVESKDVCKNYAEAK
DVELGMELYEYARRHPDYSVVLLLRLAKTYETTLEKCCAAADPHE
CYAKVEDEFKPLVEEPQNLIKQNCELFEQLGEYKFQNALLVRYTK
KVPQVSTPTLVEVSRNLGKVGSKCCKHPEAKRMPCAEDYLSVVLN
QLCVLHEKTPVSDRVTKCCTESLVNRRPCESALEVDETYVPKEEN
AETFTFHADICTLSEKERQIKKQTALVELVKHKPKATKEQLKAVM
DDEAAFVEKCCKADDKETCFAEEGKKLVAASQAALGL.

In some embodiments, the amino acid sequence of the anti-PD-1-HSA antibody heavy chain comprises SEQ ID NO: 1 immediately followed by SEQ ID NO: 59. In some embodiments, the signal peptide is cleaved during post-translational modifications that occur in vitro or in vivo.

In some embodiments, the amino acid sequence of the anti-PD-1-HSA antibody light chain (without the signal peptide) comprises SEQ ID NO: 3, 15, 17, 19, 21, 23, 25, 27, 29, 31, 73, or 75 as provided above for the anti-PD1-HSA antibody. In some embodiments, the amino acid sequence of the anti-PD-1-HSA antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 59 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-HSA antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-HSA antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 59. In some embodiments, the anti-PD-1-HSA antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-HSA antibody comprises an amino acid sequence comprising the CDRs of the variable heavy chain domain and the CDRs of the variable light chain domain of the anti-PD-1-HSA antibody, wherein the CDR sequences are indicated above (in bold and underline), in FIG. 11, or in Tables 1 and 3 for SEQ ID NO: 3, 15, 17, 19, 21, 23, 25, 27, 29, 31, 59, 73, and 75. In some embodiments, the framework regions (FRs) of the variable heavy chain domain and the FRs of the variable light chain domain of the anti-PD-1-HSA antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 59, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 59, 73, or 75 is substituted. In some embodiments, one or more amino acids of an amino acid sequence encoding one or more variable heavy chain CDRs of SEQ ID NO: 59 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-HSA antibody heavy chain comprises SEQ ID NO: 60. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the HSA domain.

(SEQ ID NO: 60)
EVKLLESGGGLVQPGGSLKLSCEVSGFDFSRDWMSWVRQAPGKGL
EWIGQISPDSTSINYKPSLKDKFIISRDNAKNTLYLQLSGVRSED
TALYHCARDTSGYPDYWGQGTSLTVSSASTKGPSVFPLAPCSRST
SESTAALGCLVKDYFPEPVTVSWNSGALTSGVHTFPAVLQSSGLY
SLSSVVTVPSSSLGTKTYTCNVDHKPSNTKVDKRVESKYGPPCPP
CPAPEFEGGPSVFLFPPKPKDTLMISRTPEVTCVVVDVSQEDPEV
QFNWYVDGVEVHNAKTKPREEQFNSTYRVVSVLTVLHQDWLNGKE
YKCKVSNKGLPSSIEKTISKAKGQPREPQVYTLPPSQEEMTKNQV
SLTCLVKGFYPSDIAVEWESNGQPENNYKTTPPVLDSDGSFFLYS
RLTVDKSRWQEGNVFSCSVMHEALHNHYTQKSLSLSLGDAHKSEV
AHREKDLGEENEKALVLIAFAQYLQQCPEEDHVKLVNEVTEFAKT
CVADESAENCDKSLHTLFGDKLCTVATLRETYGEMADCCAKQEPE
RNECELQHKDDNPNLPRLVRPEVDVMCTAFHDNEETELKKYLYEI
ARRHPYFYAPELLEEAKRYKAAFTECCQAADKAACLLPKLDELRD
EGKASSAKQRLKCASLQKFGERAFKAWAVARLSQREPKAEFAEVS
KLVTDLTKVHTECCHGDLLECADDRADLAKYICENQDSISSKLKE
CCEKPLLEKSHCIAEVENDEMPADLPSLAADEVESKDVCKNYAEA
KDVELGMELYEYARRHPDYSVVLLLRLAKTYETTLEKCCAAADPH
ECYAKVEDEFKPLVEEPQNLIKQNCELFEQLGEYKFQNALLVRYT
KKVPQVSTPTLVEVSRNLGKVGSKCCKHPEAKRMPCAEDYLSVVL
NQLCVLHEKTPVSDRVTKCCTESLVNRRPCESALEVDETYVPKEF
NAETETFHADICTLSEKERQIKKQTALVELVKHKPKATKEQLKAV
MDDFAAFVEKCCKADDKETCEAEEGKKLVAASQAALGL.

In some embodiments, the amino acid sequence of the anti-PD-1-HSA antibody heavy chain comprises SEQ ID NO: 1 immediately followed by SEQ ID NO: 60. In some embodiments, the signal peptide is cleaved during post-translational modifications that occur in vitro or in vivo.

In some embodiments, the amino acid sequence of the anti-PD-1-HSA antibody light chain (without the signal peptide) comprises SEQ ID NO: 3, 15, 17, 19, 21, 23, 25, 27, 29, 31, 73, or 75 as provided above for the anti-PD1-HSA antibody. In some embodiments, the amino acid sequence of the anti-PD-1-HSA antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 60 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 200 kD. In some embodiments, the monoclonal antibody is about 200 kD.

In some embodiments, the signal peptide of the anti-PD-1-HSA antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-HSA antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 60. In some embodiments, the anti-PD-1-HSA antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-HSA antibody comprises an amino acid sequence comprising the CDRs of the variable heavy chain domain and the CDRs of the variable light chain domain of the anti-PD-1-HSA antibody, wherein the CDR sequences are indicated above (in bold and underline), in FIG. 11, or in Tables 1 and 3 for SEQ ID NO: 3, 15, 17, 19, 21, 23, 25, 27, 29, 31, 60, 73, and 75. In some embodiments, the framework regions (FRs) of the variable heavy chain domain and the FRs of the variable light chain domain of the anti-PD-1-HSA antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 60, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 60, 73, or 75 is substituted. In some embodiments, one or more amino acids of an amino acid sequence encoding one or more variable heavy chain CDRs of SEQ ID NO: 60 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments the monoclonal antibody is a scFv-Fc antibody comprising a first and second chain that associate together, each chain comprising a variable heavy chain (VH) domain of SEQ ID NO: 2, 14, 16, 18, 20, 22, 24, 26, 28, 30, 72, or 74 a linker (e.g., GGGGSGGGGSGGGGS (SEQ ID NO: 13)), a variable light chain (VL) domain of SEQ ID NO: 3, 15, 17, 19, 21, 23, 25, 27, 29, 31, 73, or 75, an Fc domain of SEQ ID NO: 2, and at least one additional domain. In some embodiments, the additional domain is an HSA domain of SEQ ID NO: 50. In some embodiments, the amino acid sequence of the HSA domain comprises any fragment of SEQ ID NO: 50 as long as the HSA domain increases the antibody size to at least 150 kD, at least 160 kD, at least 170 kD, at least 180 kD, at least 190 kD, or at least 200 kD. In some embodiments, the amino acid sequence of the HSA domain comprises any fragment of SEQ ID NO: 50 as long as the HSA domain increases the antibody size to at least about 200 kD.

In some embodiments, the amino acid sequence of the HSA domain comprises any fragment of SEQ ID NO: 50 as long as the HSA domain increases the antibody size to about 200 kD.

In some embodiments, the amino acid sequence of the anti-PD-1-HSA antibody described herein can comprise a VH and VL of humanized clone 051 as provided in SEQ ID NO: 74 and 75 or a VH and VL of clone 004 as provided in SEQ ID NO: 72 and 73. In some embodiments, the amino acid sequence of the anti-PD-1-CD22 antibody described herein can comprise CDRs of any of the VH and VL sequences provided herein (see e.g., Tables 1 and 3).

### Nucleic Acid Sequences of Anti-PD-1-HSA Antibodies

A person skilled in the art can identify the nucleic acid sequences encoding the features identified in the corresponding amino acid sequences (e.g., CDRs, variable heavy domain, variable light domain, CH1 domain, hinge domain, Fc domain, and HSA domain) by translating the nucleic acid sequences into amino acid sequences. In some embodiments, the nucleic acid sequences may be codon optimized. In some embodiments, nucleic acids encoding VH and VL sequences disclosed herein are provided in Table 5.

In some embodiments, the nucleic acid sequence of the anti-PD-1-HSA antibody comprises a nucleic acid sequence encoding a signal peptide comprising SEQ ID NO: 5.

In some embodiments, the nucleic acid sequence encoding the anti-PD-1-HSA antibody heavy chain comprises any of SEQ ID NOs: 51-60 provided above for anti-PD-1-HSA. In some embodiments, the nucleic acid sequence encoding the anti-PD-1-HSA antibody light chain comprises SEQ ID NO: 5 immediately followed by the nucleic acid that encodes for any of SEQ ID NO: 51-60. In some embodiments, the signal peptide is cleaved during post-translational modifications that occur in vitro or in vivo. Nucleic acid sequences encoding the respective CDRs, VH domain and CH domain in SEQ ID NOs: 51-60 can be determined from the amino acid sequence of SEQ ID NOs: 51-60 as identified herein by a person of skill in the art.

In some embodiments, the amino acid sequence of the anti-PD-1-HSA antibody heavy chain comprises one of SEQ ID NOs: 51-60.

In some embodiments, the nucleic acid sequence encoding the anti-PD-1-HSA antibody heavy chain comprises a nucleic acid sequence encoding one of SEQ ID NOs: 51-60.

In some embodiments, the nucleic acid sequence encoding the anti-PD-1-HSA antibody heavy chain comprises SEQ ID NO: 5 immediately followed by a nucleic acid sequence encoding any of SEQ ID NOs: 51-60. In some embodiments, the signal peptide is cleaved during post-translational modifications that occur in vitro or in vivo. Nucleic acid sequences encoding the anti-PD-1-HSA antibody heavy chain and respective CDRs, VH domain, CH1 domain, hinge domain, Fc domain and HSA domain can be determined from the amino acid sequence of SEQ ID NOS: 51-60 by a person of skill in the art.

In some embodiments, the amino acid sequence of the anti-PD-1-HSA antibody heavy chain comprises any of SEQ ID NOs: 51-60.

In some embodiments, the nucleic acid sequence encoding the anti-PD-1-HSA antibody heavy chain comprises a nucleic acid sequence encoding any of SEQ ID NOs: 51-60. In some embodiments, the nucleic acid sequence encoding the anti-PD-1-HSA antibody heavy chain comprises SEQ ID NO: 5 immediately followed by a nucleic acid sequence encoding any of SEQ ID NOs: 51-60. In some embodiments, the signal peptide is cleaved during post-translational modifications that occur in vitro or in vivo. Nucleic acid sequences encoding the anti-PD-1-HSA antibody heavy chain and respective CDRs, VH domain, CH1 domain, hinge domain, Fc domain and HSA domain can be determined from the amino acid sequence of any of SEQ ID NOs: 51-60 by a person of skill in the art.

In some embodiments, the nucleic acid sequence encoding the anti-PD-1-HSA antibody light chain comprises SEQ ID NO: 3, 15, 17, 19, 21, 23, 25, 27, 29, 31, 73, or 75. In some embodiments, the nucleic acid sequence encoding the anti-PD-1-HSA antibody light chain comprises SEQ ID NO: 5 immediately followed by SEQ ID NO: 3, 15, 17, 19, 21, 23, 25, 27, 29, 31, 73 or 75. In some embodiments, the signal peptide is cleaved during post-translational modifications that occur in vitro or in vivo. Nucleic acid sequences encoding the respective CDRs, VL domain and CL domain in SEQ ID NO: 3, 15, 17, 19, 21, 23, 25, 27, 29, 31, 73, or 75 can be determined from the amino acid sequence of SEQ ID NO: 3, 15, 17, 19, 21, 23, 25, 27, 29, 31, 73 or 75 as identified herein by a person of skill in the art.

In some embodiments, the amino acid sequence of the anti-PD-1-HSA antibody light chain comprises SEQ ID NO: 3, 15, 17, 19, 21, 23, 25, 27, 29, 31, 73, or 75.

In some embodiments, the nucleic acid sequence encoding the anti-PD-1-HSA antibody light chain comprises a nucleic acid sequence encoding SEQ ID NO: 3, 15, 17, 19, 21, 23, 25, 27, 29, 31, 73, or 75. In some embodiments, the nucleic acid sequence encoding the anti-PD-1-HSA antibody light chain comprises SEQ ID NO: 5 immediately followed by a nucleic acid sequence encoding SEQ ID NO: 3, 15, 17, 19, 21, 23, 25, 27, 29, 31, 73, or 75. In some embodiments, the signal peptide is cleaved during post-translational modifications that occur in vitro or in vivo. Nucleic acid sequences encoding the anti-PD-1-HSA antibody light chain and respective CDRs, VL domain, and CLI domain can be determined from the amino acid sequence of SEQ ID NO: 3, 15, 17, 19, 21, 23, 25, 27, 29, 31, 73, or 75 by a person of skill in the art.

In some embodiments, the nucleic acid sequence encoding a signal peptide of the anti-PD-1-HSA antibody heavy or light chain comprise a nucleic acid sequence 80, 81, 82, 83, 84, 85, 86, 87, 88, 89, 90, 91, 92, 93, 94, 95, 96, 97, 98 or 99% identical to SEQ ID NO: 5. In some embodiments, the nucleic acid sequence encoding an anti-PD-1-HSA heavy chain (without the signal peptide) comprise a nucleic acid sequence 80, 81, 82, 83, 84, 85, 86, 87, 88, 89, 90, 91, 92, 93, 94, 95, 96, 97, 98 or 99% identical to one of SEQ ID NOs: 51-60. In some embodiments, the nucleic acid sequence encoding an anti-PD-1-HSA light chain (without the signal peptide) comprise a nucleic acid sequence 80, 81, 82, 83, 84, 85, 86, 87, 88, 89, 90, 91, 92, 93, 94, 95, 96, 97, 98 or 99% identical to one of SEQ ID NOs: 51-60.

In some embodiments, the nucleic acid sequence encoding a signal peptide of the anti-PD-1-HSA antibody heavy or light chain comprise a nucleic acid sequence encoding an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the nucleic acid sequence encoding an anti-PD-1-HSA heavy chain (without the signal peptide) comprise a nucleic acid sequence encoding an amino acid sequence 80, 81, 82, 83, 84, 85, 86, 87, 88, 89, 90, 91, 92, 93, 94, 95, 96, 97, 98 or 99% identical to one of SEQ ID NOs: 51-60. In some embodiments, the nucleic acid sequence encoding an anti-PD-1-HSA light chain (without the signal peptide) comprise a nucleic acid sequence encoding an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75.

In some embodiments, the nucleic acid sequence encodes CDRs of the variable heavy chain domain and the CDRs of the variable light chain domain of the anti-PD-1-HSA antibody, wherein the CDR sequences are indicated above (in bold and underline) in SEQ ID NOS: 51-60. In some embodiments, the nucleic acid sequence encoding framework regions

(FRs) of the variable heavy chain domain and the FRs of the variable light chain domain of the anti-PD-1-HSA antibody comprise a nucleic acid sequence 80, 81, 82, 83, 84, 85, 86, 87, 88, 89, 90, 91, 92, 93, 94, 95, 96, 97, 98 or 99% identical to the FRs of one of SEQ ID NOs: 51-60. In some embodiments, one or more nucleic acids of a nucleic acid sequence encoding one or more CDRs of SEQ ID NOs: 51-60 is substituted. In some embodiments, one or more nucleic acids of a nucleic acid sequence encoding one or more variable heavy chain CDRs of any of SEQ ID NOs: 51-60 is substituted. In some embodiments, one or more nucleic acids of a nucleic acid sequence encoding one or more variable light chain CDRs of SEQ ID NO: 3, 15, 17, 19, 21, 23, 25, 27, 29, 31, 73, or 75 is substituted.

In some embodiments, the nucleic acid sequence comprises a nucleic acid sequence encoding the scFv-Fc described herein.

### Amino Acid Sequences of Anti-PD-1-ABD Antibodies

In some embodiments, the anti-PD-1-ABD antibody comprises two polypeptide heavy chains each comprising a variable heavy chain (VH) domain, CH1 domain, a hinge domain, a first Fc domain (CH2-CH3), and a ABD domain and two polypeptide light chains comprising a variable light chain (VL) domain and a CL domain.

In some embodiments, the amino acid sequence of the anti-PD-1-ABD antibody heavy chain comprises a signal peptide comprising SEQ ID NO: 1.

In some embodiments, the amino acid sequence of the ABD domain comprises albumin binding domain (SEQ ID NO: 61):

(SEQ ID NO: 61)
LKEAKEKAIEELKKAGITSDYYFDLINKAKTVEGVNALKDEILKALP.

In some embodiments, the amino acid sequence of the ABD domain comprises any fragment of SEQ ID NO: 61 as long as the ABD domain increases the monoclonal antibody size to at least 150 kD, at least 160 kD, at least 170 kD, at least 180 kD, at least 190 kD, or at least 160 kD. In some embodiments, the amino acid sequence of the ABD domain comprises any fragment of SEQ ID NO: 61 as long as the ABD domain increases the monoclonal antibody size to at least about 160 kD. In some embodiments, the amino acid sequence of the ABD domain comprises any fragment of SEQ ID NO: 61 as long as the ABD domain increases the monoclonal antibody size to about 160 kD.

In some embodiments, the amino acid sequence of the anti-PD-1-ABD antibody heavy chain comprises SEQ ID NO: 62. Underlined amino acids represent the VH domain. Underlined and bolded amino acids shown in FIG. 11 represent the respective complementarity determining regions (CDRs). Italicized and bold amino acids represent the CH1 domain. Italicized and underlined amino acids represent the hinge domain. Italicized amino acids represent the Fc domain. Double underlined amino acids represent the ABD domain.

(SEQ ID NO: 62)
QVQLVQSGVEVKKPGASVKVSCKASGYTFTNYYMYWVRQAPGQGL
EWMGGINPSNGGTNFNEKFKNRVTLTTDSSTTTAYMELKSLQFDD
TAVYYCARRDYRFDMGFDYWGQGTLVTVSSASTKGPSVFPLAPCS
RSTSESTAALGCLVKDYFPEPVTVSWNSGALTSGVHTFPAVLQSS
GLYSLSSVVTVPSSSLGTKTYTCNVDHKPSNTKVDKRVESKYGPP
CPPCPAPEFEGGPSVFLFPPKPKDTLMISRTPEVTCVVVDVSQED
PEVQFNWYVDGVEVHNAKTKPREEQFNSTYRVVSVLTVLHQDWLN
GKEYKCKVSNKGLPSSIEKTISKAKGQPREPQVYTLPPSQEEMTK
NQVSLTCLVKGFYPSDIAVEWESNGQPENNYKTTPPVLDSDGSFF
LYSRLTVDKSRWQEGNVESCSVMHEALHNHYTQKSLSLSLGLKEA
KEKAIEELKKAGITSDYYEDLINKAKTVEGVNALKDEILKALP.

In some embodiments, the amino acid sequence of the anti-PD-1-ABD antibody heavy chain comprises SEQ ID NO: 1 immediately followed by SEQ ID NO: 62. In some embodiments, the signal peptide is cleaved during post-translational modifications that occur in vitro or in vivo.

In some embodiments, the amino acid sequence of the anti-PD-1-ABD antibody light chain (without the signal peptide) comprises SEQ ID NO: 3, 15, 17, 19, 21, 23, 25, 27, 29, 31, 73, or 75 as provided above for the anti-PD1-ABD antibody. In some embodiments, the amino acid sequence of the anti-PD-1-ABD antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 62 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 160 kD. In some embodiments, the monoclonal antibody is about 160 kD.

In some embodiments, the signal peptide of the anti-PD-1-ABD antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-ABD antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 62. In some embodiments, the anti-PD-1-ABD antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-ABD antibody comprises an amino acid sequence comprising the CDRs of the variable heavy chain domain and the CDRs of the variable light chain domain of the anti-PD-1-ABD antibody, wherein the CDR sequences are indicated above (in bold and underline), in FIG. 11, or in Tables 1 and 3 for SEQ ID NO: 3, 15, 17, 19, 21, 23, 25, 27, 29, 31, 62, 72, 73, 74, and 75. In some embodiments, the framework regions (FRs) of the variable heavy chain domain and the FRs of the variable light chain domain of the anti-PD-1-ABD antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 62, 72, 73, 74, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 62, 72, or 75 is substituted. In some embodiments, one or more amino acids of an amino acid sequence encoding one or more variable heavy chain CDRs of SEQ ID NO: 62 is substituted. In some embodiments, one or more amino acids of an amino acid sequence encoding one or more variable light chain CDRs of SEQ ID NO: 3, 15, 17, 19, 21, 23, 25, 27, 29, 31, 72, or 75 is substituted.

In some embodiments, the amino acid sequence of the anti-PD-1-ABD antibody heavy chain comprises SEQ ID NO: 63. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the ABD domain.

(SEQ ID NO: 63)
EVKLLESGGGLVQPGGSLKLSCAASGFDFSRYWMSWVRQAPGKGL
EWIGEINPDSSTIKYTPSLKDKFIISRDNAKNTLCLQLSKVRSED
SALYYCARMGYRLFDSWGQGTTLTVSSASTKGPSVFPLAPCSRST
SESTAALGCLVKDYFPEPVTVSWNSGALTSGVHTFPAVLQSSGLY
SLSSVVTVPSSSLGTKTYTCNVDHKPSNTKVDKRVESKYGPPCPP
CPAPEFEGGPSVFLFPPKPKDTLMISRTPEVTCVVVDVSQEDPEV
QFNWYVDGVEVHNAKTKPREEQFNSTYRVVSVLTVLHQDWLNGKE
YKCKVSNKGLPSSIEKTISKAKGQPREPQVYTLPPSQEEMTKNQV
SLTCLVKGFYPSDIAVEWESNGQPENNYKTTPPVLDSDGSFFLYS
RLTVDKSRWQEGNVFSCSVMHEALHNHYTQKSLSLSLGLKEAKEK
AIEELKKAGITSDYYEDLINKAKTVEGVNALKDEILKALP.

In some embodiments, the amino acid sequence of the anti-PD-1-ABD antibody heavy chain comprises SEQ ID NO: 1 immediately followed by SEQ ID NO: 63. In some embodiments, the signal peptide is cleaved during post-translational modifications that occur in vitro or in vivo.

In some embodiments, the amino acid sequence of the anti-PD-1-ABD antibody light chain (without the signal peptide) comprises SEQ ID NO: 3, 15, 17, 19, 21, 23, 25, 27, 29, 31, 73 or 75 as provided above for the anti-PD1-ABD antibody. In some embodiments, the amino acid sequence of the anti-PD-1-ABD antibody light chain comprises SEQ ID NO: 1 immediately followed by SEQ ID NO: 3, 15, 17, 19, 21, 23, 25, 27, 29, 31, 73 or 75. In some embodiments, the signal peptide is cleaved during post-translational modifications that occur in vitro or in vivo.

In some embodiments, the monoclonal antibody comprises a first and third chain each comprises SEQ ID NO: 63 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 160 kD. In some embodiments, the monoclonal antibody is about 160 kD.

In some embodiments, the signal peptide of the anti-PD-1-ABD antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-ABD antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 63. In some embodiments, the anti-PD-1-ABD antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-ABD antibody comprises an amino acid sequence comprising the CDRs of the variable heavy chain domain and the CDRs of the variable light chain domain of the anti-PD-1-ABD antibody, wherein the CDR sequences are indicated above (in bold and underline), in FIG. 11, or in Tables 1 and 3 for SEQ ID NO: 3, 15, 17, 19, 21, 23, 25, 27, 29, 31, 63, 73, and 75. In some embodiments, the framework regions (FRs) of the variable heavy chain domain and the FRs of the variable light chain domain of the anti-PD-1-ABD antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 63, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 63, 73, or 75 is substituted. In some embodiments, one or more amino acids of an amino acid sequence encoding one or more variable heavy chain CDRs of SEQ ID NO: 63 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-ABD antibody heavy chain comprises SEQ ID NO: 64. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the ABD domain.

(SEQ ID NO: 64)
VQLQQSGTVLARPGASVKMSCKASGNTFTSYWMHWVKQRPGQGLE
WIGAIYPGYSDTRYNQKFKGKATLTAVTSTSTAYMELSSLTNEDS
AVYYCTKFIATVGGYFDVWGAGTTVTVSSASTKGPSVFPLAPCSR
STSESTAALGCLVKDYFPEPVTVSWNSGALTSGVHTFPAVLQSSG
LYSLSSVVTVPSSSLGTKTYTCNVDHKPSNTKVDKRVESKYGPPC
PPCPAPEFEGGPSVFLFPPKPKDTLMISRTPEVTCVVVDVSQEDP
EVQFNWYVDGVEVHNAKTKPREEQFNSTYRVVSVLTVLHQDWLNG
KEYKCKVSNKGLPSSIEKTISKAKGQPREPQVYTLPPSQEEMTKN
QVSLTCLVKGFYPSDIAVEWESNGQPENNYKTTPPVLDSDGSFFL
YSRLTVDKSRWQEGNVFSCSVMHEALHNHYTQKSLSLSLGLKEAK
EKAIEELKKAGITSDYYEDLINKAKTVEGVNALKDEILKALP.

In some embodiments, the amino acid sequence of the anti-PD-1-ABD antibody heavy chain comprises SEQ ID NO: 1 immediately followed by SEQ ID NO: 64. In some embodiments, the signal peptide is cleaved during post-translational modifications that occur in vitro or in vivo.

In some embodiments, the amino acid sequence of the anti-PD-1-ABD antibody light chain (without the signal peptide) comprises SEQ ID NO: 3, 15, 17, 19, 21, 23, 25, 27, 29, 31, 73, or 75 as provided above for the anti-PD1-ABD antibody. In some embodiments, the amino acid sequence of the anti-PD-1-ABD antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 64 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 160 kD. In some embodiments, the monoclonal antibody is about 160 kD.

In some embodiments, the signal peptide of the anti-PD-1-ABD antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-ABD antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 64. In some embodiments, the anti-PD-1-ABD antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-ABD antibody comprises an amino acid sequence comprising the CDRs of the variable heavy chain domain and the CDRs of the variable light chain domain of the anti-PD-1-ABD antibody, wherein the CDR sequences are indicated above (in bold and underline), in FIG. 11, or in Tables 1 and 3 for SEQ ID NO: 3, 15, 17, 19, 21, 23, 25, 27, 29, 31, 64, 73, and 75. In some embodiments, the framework regions (FRs) of the variable heavy chain domain and the FRs of the variable light chain domain of the anti-PD-1-ABD antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 64, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 64, 73, or 75 is substituted. In some embodiments, one or more amino acids of an amino acid sequence encoding one or more variable heavy chain CDRs of SEQ ID NO: 64 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-ABD antibody heavy chain comprises SEQ ID NO: 65. Underlined amino acids represent the VH domain In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the ABD domain.

(SEQ ID NO: 65)
QIQLVQSGPELKKPGETVKISCKASGYTFTDYSMHWVKQAPGKGL
KWMGWIKIETGNPTYADDFKGRFAFSLETSASTAYLQINNLKNED
TATYFCARDYYGYYYYAMDYWGQGTSVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFEGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLTCLVKGFYPSDIAVEWESNGQPENNYKTTPPVLDSDGSF
FLYSRLTVDKSRWQEGNVFSCSVMHEALHNHYTQKSLSLSLGLKE
AKEKAIEELKKAGITSDYYFDLINKAKTVEGVNALKDEILKALP.

In some embodiments, the amino acid sequence of the anti-PD-1-ABD antibody heavy chain comprises SEQ ID NO: 1 immediately followed by SEQ ID NO: 65. In some embodiments, the signal peptide is cleaved during post-translational modifications that occur in vitro or in vivo.

In some embodiments, the amino acid sequence of the anti-PD-1-ABD antibody light chain (without the signal peptide) comprises SEQ ID NO: 3, 15, 17, 19, 21, 23, 25, 27, 29, 31, 73, or 75 as provided above for the anti-PD1-ABD antibody. In some embodiments, the amino acid sequence of the anti-PD-1-ABD antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 65 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 160 kD. In some embodiments, the monoclonal antibody is about 160 kD.

In some embodiments, the signal peptide of the anti-PD-1-ABD antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-ABD antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 65. In some embodiments, the anti-PD-1-ABD antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-ABD antibody comprises an amino acid sequence comprising the CDRs of the variable heavy chain domain and the CDRs of the variable light chain domain of the anti-PD-1-ABD antibody, wherein the CDR sequences are indicated above (in bold and underline), in FIG. 11, or in Tables 1 and 3 for SEQ ID NO: 3, 15, 17, 19, 21, 23, 25, 27, 29, 31, 65, 73, and 75. In some embodiments, the framework regions (FRs) of the variable heavy chain domain and the FRs of the variable light chain domain of the anti-PD-1-ABD antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 65, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 65, 73, or 75 is substituted. In some embodiments, one or more amino acids of an amino acid sequence encoding one or more variable heavy chain CDRs of SEQ ID NO: 65 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-ABD antibody heavy chain comprises SEQ ID NO: 66. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the ABD domain.

(SEQ ID NO: 66)
VQLVESGGGLVQPGGSLRLSCAASGFTFSHYGMSWVRQTPDKRL
ELVATIDNNGGNTYYPDSVKGRFTISRDNAKNTLYLQMSSLKSE
DTAMYYCARDAYYTYDYWYFDVWGAGTTVTVSSASTKGPSVFPL
APCSRSTSESTAALGCLVKDYFPEPVTVSWNSGALTSGVHTFPA
VLQSSGLYSLSSVVTVPSSSLGTKTYTCNVDHKPSNTKVDKRVE
SKYGPPCPPCPAPEFEGGPSVFLFPPKPKDTLMISRTPEVTCVV
VDVSQEDPEVQFNWYVDGVEVHNAKTKPREEQFNSTYRVVSVLT
VLHQDWLNGKEYKCKVSNKGLPSSIEKTISKAKGQPREPQVYTL
PPSQEEMTKNQVSLTCLVKGFYPSDIAVEWESNGQPENNYKTTP
PVLDSDGSFFLYSRLTVDKSRWQEGNVFSCSVMHEALHNHYTQK
SLSLSLGLKEAKEKAIEELKKAGITSDYYFDLINKAKTVEGVNA
LKDEILKALP.

In some embodiments, the amino acid sequence of the anti-PD-1-ABD antibody heavy chain comprises SEQ ID NO: 1 immediately followed by SEQ ID NO: 66. In some embodiments, the signal peptide is cleaved during post-translational modifications that occur in vitro or in vivo.

In some embodiments, the amino acid sequence of the anti-PD-1-ABD antibody light chain (without the signal peptide) comprises SEQ ID NO: 3, 15, 17, 19, 21, 23, 25, 27, 29, 31, 73, or 75 as provided above for the anti-PD1-ABD antibody. In some embodiments, the amino acid sequence of the anti-PD-1-ABD antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 66 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 160 kD. In some embodiments, the monoclonal antibody is about 160 kD.

In some embodiments, the signal peptide of the anti-PD-1-ABD antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-ABD antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 66. In some embodiments, the anti-PD-1-ABD antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-ABD antibody comprises an amino acid sequence comprising the CDRs of the variable heavy chain domain and the CDRs of the variable light chain domain of the anti-PD-1-ABD antibody, wherein the CDR sequences are indicated above (in bold and underline), in FIG. 11, or in Tables 1 and 3 for SEQ ID NO: 3, 15, 17, 19, 21, 23, 25, 27, 29, 31, 66, 73, and 75. In some embodiments, the framework regions (FRs) of the variable heavy chain domain and the FRs of the variable light chain domain of the anti-PD-1-ABD antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 66, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 66, 73, or 75 is substituted. In some embodiments, one or more amino acids of an amino acid sequence encoding one or more variable heavy chain CDRs of SEQ ID NO: 66 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-ABD antibody heavy chain comprises SEQ ID NO: 67. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the ABD domain.

(SEQ ID NO: 67)
EVKLVESGGGLVQPGGSLKLSCAASGFTFSSYTMSWVRQTPEKR
LEWVAYISYGGGDTYYPDTVKGRFTISRDNAKNTLYLQMSSLKS
EDTAMYYCARQKVDGYFVAMDYWSQGTSVTVSSASTKGPSVFPL
APCSRSTSESTAALGCLVKDYFPEPVTVSWNSGALTSGVHTFPA
VLQSSGLYSLSSVVTVPSSSLGTKTYTCNVDHKPSNTKVDKRVE
SKYGPPCPPCPAPEFEGGPSVFLFPPKPKDTLMISRTPEVTCVV
VDVSQEDPEVQFNWYVDGVEVHNAKTKPREEQFNSTYRVVSVLT
VLHQDWLNGKEYKCKVSNKGLPSSIEKTISKAKGQPREPQVYTL
PPSQEEMTKNQVSLTCLVKGFYPSDIAVEWESNGQPENNYKTTP
PVLDSDGSFFLYSRLTVDKSRWQEGNVFSCSVMHEALHNHYTQK
SLSLSLGLKEAKEKAIEELKKAGITSDYYFDLINKAKTVEGVNA
LKDEILKALP.

In some embodiments, the amino acid sequence of the anti-PD-1-ABD antibody heavy chain comprises SEQ ID NO: 1 immediately followed by SEQ ID NO: 67. In some embodiments, the signal peptide is cleaved during post-translational modifications that occur in vitro or in vivo.

In some embodiments, the amino acid sequence of the anti-PD-1-ABD antibody light chain (without the signal peptide) comprises SEQ ID NO: 3, 15, 17, 19, 21, 23, 25, 27, 29, 31, 73, or 75 as provided above for the anti-PD1-ABD antibody. In some embodiments, the amino acid sequence of the anti-PD-1-ABD antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 67 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 160 kD. In some embodiments, the monoclonal antibody is about 160 kD.

In some embodiments, the signal peptide of the anti-PD-1-ABD antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-ABD antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 67. In some embodiments, the anti-PD-1-ABD antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-ABD antibody comprises an amino acid sequence comprising the CDRs of the variable heavy chain domain and the CDRs of the variable light chain domain of the anti-PD-1-ABD antibody, wherein the CDR sequences are indicated above (in bold and underline), in FIG. 11, or in Tables 1 and 3 for SEQ ID NO: 3, 15, 17, 19, 21, 23, 25, 27, 29, 31, 67, 73, and 75. In some embodiments, the framework regions (FRs) of the variable heavy chain domain and the FRs of the variable light chain domain of the anti-PD-1-ABD antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 67, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 67, 73, or 75 is substituted. In some embodiments, one or more amino acids of an amino acid sequence encoding one or more variable heavy chain CDRs of SEQ ID NO: 67 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-ABD antibody heavy chain comprises SEQ ID NO: 68. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the ABD domain.

(SEQ ID NO: 68)
EVKLVESGGGLVKPGGSLKLSCAASGFTFSSYGMSWVRQTPEKR
LEWVATISGGGRYTYYPDSVKGRFTISRDNAKNILYLQMSSLRS
EDTALYYCASPYDGYYGAMDYWGQGTSVTVSSASTKGPSVFPLA
PCSRSTSESTAALGCLVKDYFPEPVTVSWNSGALTSGVHTFPAV
LQSSGLYSLSSVVTVPSSSLGTKTYTCNVDHKPSNTKVDKRVES
KYGPPCPPCPAPEFEGGPSVFLFPPKPKDTLMISRTPEVTCVVV
DVSQEDPEVQFNWYVDGVEVHNAKTKPREEQFNSTYRVVSVLTV
LHQDWLNGKEYKCKVSNKGLPSSIEKTISKAKGQPREPQVYTLP
PSQEEMTKNQVSLTCLVKGFYPSDIAVEWESNGQPENNYKTTPP
VLDSDGSFFLYSRLTVDKSRWQEGNVFSCSVMHEALHNHYTQKS
LSLSLGLKEAKEKAIEELKKAGITSDYYFDLINKAKTVEGVNAL
KDEILKALP.

In some embodiments, the amino acid sequence of the anti-PD-1-ABD antibody heavy chain comprises SEQ ID NO: 1 immediately followed by SEQ ID NO: 68. In some embodiments, the signal peptide is cleaved during post-translational modifications that occur in vitro or in vivo.

In some embodiments, the amino acid sequence of the anti-PD-1-ABD antibody light chain (without the signal peptide) comprises SEQ ID NO: 3, 15, 17, 19, 21, 23, 25, 27, 29, 31, 73, or 75 as provided above for the anti-PD1-ABD antibody. In some embodiments, the amino acid sequence of the anti-PD-1-ABD antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 68 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 160 kD. In some embodiments, the monoclonal antibody is about 160 kD.

In some embodiments, the signal peptide of the anti-PD-1-ABD antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-ABD antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 68. In some embodiments, the anti-PD-1-ABD antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-ABD antibody comprises an amino acid sequence comprising the CDRs of the variable heavy chain domain and the CDRs of the variable light chain domain of the anti-PD-1-ABD antibody, wherein the CDR sequences are indicated above (in bold and underline), in FIG. 11, or in Tables 1 and 3 for SEQ ID NO: 3, 15, 17, 19, 21, 23, 25, 27, 29, 31, 68, 73, and 75. In some embodiments, the framework regions (FRs) of the variable heavy chain domain and the FRs of the variable light chain domain of the anti-PD-1-ABD antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 68, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 68, 73, or 75 is substituted. In some embodiments, one or more amino acids of an amino acid sequence encoding one or more variable heavy chain CDRs of SEQ ID NO: 68 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-ABD antibody heavy chain comprises SEQ ID NO: 69. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the ABD domain.

(SEQ ID NO: 69)
EVQLQQSGTVLARPGASVRMSCKASGYSFSTYWMHWVKQRP
GQGLEWIGGIYPGNSDTNYNQKFKGKAKLTAVTSASTAYMD
LSSLTNEDSAVYYCTGGYFFDVWGAGTTVTVSSASTKGPSV
FPLAPCSRSTSESTAALGCLVKDYFPEPVTVSWNSGALTSG
VHTFPAVLQSSGLYSLSSVVTVPSSSLGTKTYTCNVDHKPS
NTKVDKRVESKYGPPCPPCPAPEFEGGPSVFLFPPKPKDTL
MISRTPEVTCVVVDVSQEDPEVQFNWYVDGVEVHNAKTKPR
EEQFNSTYRVVSVLTVLHQDWLNGKEYKCKVSNKGLPSSIE
KTISKAKGQPREPQVYTLPPSQEEMTKNQVSLTCLVKGFYP
SDIAVEWESNGQPENNYKTTPPVLDSDGSFFLYSRLTVDKS
RWQEGNVFSCSVMHEALHNHYTQKSLSLSLGLKEAKEKAIE
ELKKAGITSDYYFDLINKAKTVEGVNALKDEILKALP.

In some embodiments, the amino acid sequence of the anti-PD-1-ABD antibody heavy chain comprises SEQ ID NO: 1 immediately followed by SEQ ID NO: 69. In some embodiments, the signal peptide is cleaved during post-translational modifications that occur in vitro or in vivo.

In some embodiments, the amino acid sequence of the anti-PD-1-ABD antibody light chain (without the signal peptide) comprises SEQ ID NO: 3, 15, 17, 19, 21, 23, 25, 27, 29, 31, 73, or 75 as provided above for the anti-PD1-ABD antibody. In some embodiments, the amino acid sequence of the anti-PD-1-ABD antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 69 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 160 kD. In some embodiments, the monoclonal antibody is about 160 kD.

In some embodiments, the signal peptide of the anti-PD-1-ABD antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-ABD antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 69. In some embodiments, the anti-PD-1-ABD antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-ABD antibody comprises an amino acid sequence comprising the CDRs of the variable heavy chain domain and the CDRs of the variable light chain domain of the anti-PD-1-ABD antibody, wherein the CDR sequences are indicated above (in bold and underline), in FIG. 11, or in Tables 1 and 3 for SEQ ID NO: 3, 15, 17, 19, 21, 23, 25, 27, 29, 31, 69, 73 and 75. In some embodiments, the framework regions (FRs) of the variable heavy chain domain and the FRs of the variable light chain domain of the anti-PD-1-ABD antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 69, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 69, 73, or 75 is substituted. In some embodiments, one or more amino acids of an amino acid sequence encoding one or more variable heavy chain CDRs of SEQ ID NO: 69 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-ABD antibody heavy chain comprises SEQ ID NO: 70. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the ABD domain.

(SEQ ID NO: 70)
QIQLQQSGPEQVKPGASVKISCKASGYMFIDYYMNWVKQKPGQGLEWIGW
IYPGSGSTKDNENFKGKATLTVDTSSSTAYMQLSSLTSEDTAVYFCARYG
PRFFDVWGAGTTVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFEGGPSVFLFPPKPKDTLMI
SRTPEVTCVVVDVSQEDPEVWQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGLKEAKE
KAIEELKKAGITSDYYFDLINKAKTVEGVNALKDEILKALP.

In some embodiments, the amino acid sequence of the anti-PD-1-ABD antibody heavy chain comprises SEQ ID NO: 1 immediately followed by SEQ ID NO: 70. In some embodiments, the signal peptide is cleaved during post-translational modifications that occur in vitro or in vivo.

In some embodiments, the amino acid sequence of the anti-PD-1-ABD antibody light chain (without the signal peptide) comprises SEQ ID NO: 3, 15, 17, 19, 21, 23, 25, 27, 29, 31, 73, or 75 as provided above for the anti-PD1-ABD antibody. In some embodiments, the amino acid sequence of the anti-PD-1-ABD antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 70 and a second and fourth chains each comprises SEQ ID NO: 3, 15, 17, 19, 21, 23, 25, 27, 29, 31, 73 or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 160 kD. In some embodiments, the monoclonal antibody is about 160 kD.

In some embodiments, the signal peptide of the anti-PD-1-ABD antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-ABD antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 70. In some embodiments, the anti-PD-1-ABD antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-ABD antibody comprises an amino acid sequence comprising the CDRs of the variable heavy chain domain and the CDRs of the variable light chain domain of the anti-PD-1-ABD antibody, wherein the CDR sequences are indicated above (in bold and underline) in FIG. 11, or in Tables 1 and 3 for in SEQ ID NO: 3, 15, 17, 19, 21, 23, 25, 27, 29, 31, 70, 73, or 75. In some embodiments, the framework regions (FRs) of the variable heavy chain domain and the FRs of the variable light chain domain of the anti-PD-1-ABD antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 70, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 70, 73, or 75 is substituted. In some embodiments, one or more amino acids of an amino acid sequence encoding one or more variable heavy chain CDRs of SEQ ID NO: 70 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments, the amino acid sequence of the anti-PD-1-ABD antibody heavy chain comprises SEQ ID NO: 71. Underlined amino acids represent the VH domain. In some embodiments, the underlined and bolded amino acids shown in FIG. 11 represent the respective complementarity determining regions (CDRs). In some embodiments, the CDRs are as provided in Table 1. Italicized and bold amino acids represent the CH1 domain. Italicized and underlined amino acids represent the hinge domain. Italicized amino acids represent the Fc domain. Double underlined amino acids represent the ABD domain.

(SEQ ID NO: 71)
EVKLLESGGGLVQPGGSLKLSCEVSGFDFSRDWMSWVRQAPGKGLEWIGQ
ISPDSTSINYKPSLKDKFIISRDNAKNTLYLQLSGVRSEDTALYHCARDT
SGYPDYWGQGTSLTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFEGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGLKEAKE
KAIEELKKAGITSDYYFDLINKAKTVEGVNALKDEILKALP.

In some embodiments, the amino acid sequence of the anti-PD-1-ABD antibody heavy chain comprises SEQ ID NO: 1 immediately followed by SEQ ID NO: 71. In some embodiments, the signal peptide is cleaved during post-translational modifications that occur in vitro or in vivo.

In some embodiments, the amino acid sequence of the anti-PD-1-ABD antibody light chain (without the signal peptide) comprises SEQ ID NO: 3, 15, 17, 19, 21, 23, 25, 27, 29, 31, 73, or 75 as provided above for the anti-PD1-ABD antibody. In some embodiments, the amino acid sequence of the anti-PD-1-ABD antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises SEQ ID NO: 71 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is larger in size that 140 kD. In some embodiments, the monoclonal antibody is at least about 160 kD. In some embodiments, the monoclonal antibody is about 160 kD.

In some embodiments, the signal peptide of the anti-PD-1-ABD antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1-ABD antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 71. In some embodiments, the anti-PD-1-ABD antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1-ABD antibody comprises an amino acid sequence comprising the CDRs of the variable heavy chain domain and the CDRs of the variable light chain domain of the anti-PD-1-ABD antibody, wherein the CDR sequences are indicated above (in bold and underline), in FIG. 11, or in Tables 1 and 3 for SEQ ID NO: 3, 15, 17, 19, 21, 23, 25, 27, 29, 31, 71, 73, and 75. In some embodiments, the framework regions (FRs) of the variable heavy chain domain and the FRs of the variable light chain domain of the anti-PD-1-ABD antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 3, 15, 17, 19, 21, 23, 25, 27, 29, 31, 71, 73, or 75, or the FRs in Tables 2 and 4.

In some embodiments, one or more amino acids of an amino acid sequence encoding one or more CDRs of SEQ ID NO: 3, 15, 17, 19, 21, 23, 25, 27, 29, 31, 71, 7, or 75 is substituted. In some embodiments, one or more amino acids of an amino acid sequence encoding one or more variable heavy chain CDRs of SEQ ID NO: 71 is substituted. In some embodiments, one or more amino acids of an amino acid sequence encoding one or more variable light chain CDRs of SEQ ID NO: 3, 15, 17, 19, 21, 23, 25, 27, 29, 31, 73, or 75 is substituted.

In some embodiments the monoclonal antibody is a scFv-Fc antibody comprising a first and second chain that associate together, each chain comprising a variable heavy chain (VH) domain of SEQ ID NO: 2, 14, 16, 18, 20, 22, 24, 26, 28, 30, 72, or 74 a linker

(SEQ ID NO: 13)
(e.g., GGGGSGGGGSGGGGS),

a variable light chain (VL) domain of SEQ ID NO: 3, 15, 17, 19, 21, 23, 25, 27, 29, 31, 73, or 75 an Fc domain of SEQ ID NO: 2, and at least one additional domain. In some embodiments, the additional domain is an ABD domain of SEQ ID NO: 61. In some embodiments, the amino acid sequence of the ABD domain comprises any fragment of SEQ ID NO: 61 as long as the ABD domain increases the antibody size to at least 150 kD, at least 160 kD, at least 170 kD, at least 180 kD, at least 190 kD, or at least 160 kD. In some embodiments, the amino acid sequence of the ABD domain comprises any fragment of SEQ ID NO: 61 as long as the ABD domain increases the antibody size to at least about 160 kD. In some embodiments, the amino acid sequence of the ABD domain comprises any fragment of SEQ ID NO: 61 as long as the ABD domain increases the antibody size to about 160 kD.

In some embodiments, the ABD peptide is at the N-terminus of the heavy chain of the anti-PD-1-ABD antibody (after the signal peptide is removed) instead of at the C-terminus of the heavy chain of the anti-PD-1-ABD antibody. In some embodiments the heavy chain of the anti-PD-1-ABD antibody comprises any of the heavy chain anti-PD-1-ABD antibody sequences described herein (any of SEQ ID NOs: 62-71) except that the location of the ABD is at the N-terminus of any of SEQ ID NOs: 62-71 instead of the C-terminus. In some embodiments, the heavy chain of the anti-PD-1-ABD antibody comprises a signal peptide (SEQ ID NO: 1), followed by the ABD peptide (SEQ ID NO: 61), followed by the anti-PD-1 portion (VH, CH1, hinge domain, and Fc) of any of SEQ ID NOs: 62-71 (without the ABD). In some embodiments, the signal peptide is cleaved.

In some embodiments, the amino acid sequence of the anti-PD-1-ABD antibody described herein can comprise a VH and VL of humanized clone 051 as provided in SEQ ID NO: 74 and 75 or a VH and VL of clone 004 as provided in SEQ ID NO: 72 and 73. In some embodiments, the amino acid sequence of the anti-PD-1-ABD antibody described herein can comprise CDRs of any of the VH and VL sequences provided herein (see e.g., Tables 1 and 3). Nucleic Acid Sequences of Anti-PD-1-ABD Antibodies

A person skilled in the art can identify the nucleic acid sequences encoding the features identified in the corresponding amino acid sequences (e.g., CDRs, variable heavy domain, variable light domain, CH1 domain, hinge domain, Fc domain, and ABD domain) by translating the nucleic acid sequences into amino acid sequences. In some embodiments, the nucleic acid sequences may be codon optimized. In some embodiments, nucleic acids encoding VH and VL sequences disclosed herein are provided in Table 5.

In some embodiments, the nucleic acid sequence of the anti-PD-1-ABD antibody comprises a nucleic acid sequence encoding a signal peptide comprising SEQ ID NO: 5.

In some embodiments, the nucleic acid sequence encoding the anti-PD-1-ABD antibody heavy chain comprises any of SEQ ID NOs: 62-71 provided above for anti-PD-1-ABD. In some embodiments, the nucleic acid sequence encoding the anti-PD-1-ABD antibody light chain comprises SEQ ID NO: 5 immediately followed by the nucleic acid that encodes for any of SEQ ID NO: 62-71. In some embodiments, the signal peptide is cleaved during post-translational modifications that occur in vitro or in vivo. Nucleic acid sequences encoding the respective CDRs, VH domain and CH domain in SEQ ID NOs: 62-71 can be determined from the amino acid sequence of SEQ ID NOs: 62-71 as identified herein by a person of skill in the art.

In some embodiments, the amino acid sequence of the anti-PD-1-ABD antibody heavy chain comprises one of SEQ ID NOs: 62-71.

In some embodiments, the nucleic acid sequence encoding the anti-PD-1-ABD antibody heavy chain comprises a nucleic acid sequence encoding one of SEQ ID NOs: 62-71.

In some embodiments, the nucleic acid sequence encoding the anti-PD-1-ABD antibody heavy chain comprises SEQ ID NO: 5 immediately followed by a nucleic acid sequence encoding any of SEQ ID NOs: 62-71. In some embodiments, the signal peptide is cleaved during post-translational modifications that occur in vitro or in vivo. Nucleic acid sequences encoding the anti-PD-1-ABD antibody heavy chain and respective CDRs, VH domain, CH1 domain, hinge domain, Fc domain and ABD domain can be determined from the amino acid sequence of SEQ ID NOS: 62-71 by a person of skill in the art.

In some embodiments, the amino acid sequence of the anti-PD-1-ABD antibody heavy chain comprises any of SEQ ID NOs: 62-71.

In some embodiments, the nucleic acid sequence encoding the anti-PD-1-ABD antibody heavy chain comprises a nucleic acid sequence encoding any of SEQ ID NOs: 62-71. In some embodiments, the nucleic acid sequence encoding the anti-PD-1-ABD antibody heavy chain comprises SEQ ID NO: 5 immediately followed by a nucleic acid sequence encoding any of SEQ ID NOs: 62-71. In some embodiments, the signal peptide is cleaved during post-translational modifications that occur in vitro or in vivo. Nucleic acid sequences encoding the anti-PD-1-ABD antibody heavy chain and respective CDRs, VH domain, CH1 domain, hinge domain, Fc domain and ABD domain can be determined from the amino acid sequence of any of SEQ ID NOs: 62-71 by a person of skill in the art.

In some embodiments, the nucleic acid sequence encoding the anti-PD-1-ABD antibody light chain comprises SEQ ID NO: 3, 15, 17, 19, 21, 23, 25, 27, 29, 31, 73, or 75. In some embodiments, the nucleic acid sequence encoding the anti-PD-1-ABD antibody light chain comprises SEQ ID NO: 5 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo. Nucleic acid sequences encoding the respective CDRs, VL domain and CL domain in SEQ ID NO: 3, 15, 17, 19, 21, 23, 25, 27, 29, 31, 73, or 75 can be determined from the amino acid sequence of SEQ ID NO: 3, 15, 17, 19, 21, 23, 25, 27, 29, 31, 73, or 75 as identified herein by a person of skill in the art.

In some embodiments, the amino acid sequence of the anti-PD-1-ABD antibody light chain comprises SEQ ID NO: 3, 15, 17, 19, 21, 23, 25, 27, 29, 31, 73, or 75.

In some embodiments, the nucleic acid sequence encoding the anti-PD-1-ABD antibody light chain comprises a nucleic acid sequence encoding SEQ ID NO: 3, 15, 17, 19, 21, 23, 25, 27, 29, 31, 73, or 75. In some embodiments, the nucleic acid sequence encoding the anti-PD-1-ABD antibody light chain comprises SEQ ID NO: 5 immediately followed by a nucleic acid sequence encoding SEQ ID NO: 3, 15, 17, 19, 21, 23, 25, 27, 29, 31, 73, or 75. In some embodiments, the signal peptide is cleaved during post-translational modifications that occur in vitro or in vivo. Nucleic acid sequences encoding the anti-PD-1-ABD antibody light chain and respective CDRs, VL domain, and CLI domain can be determined from the amino acid sequence of SEQ ID NO: 3, 15, 17, 19, 21, 23, 25, 27, 29, 31, 73, or 75 by a person of skill in the art.

In some embodiments, the nucleic acid sequence encoding a signal peptide of the anti-PD-1-ABD antibody heavy or light chain comprise a nucleic acid sequence 80, 81, 82, 83, 84, 85, 86, 87, 88, 89, 90, 91, 92, 93, 94, 95, 96, 97, 98 or 99% identical to SEQ ID NO: 5. In some embodiments, the nucleic acid sequence encoding an anti-PD-1-ABD heavy chain (without the signal peptide) comprise a nucleic acid sequence 80, 81, 82, 83, 84, 85, 86, 87, 88, 89, 90, 91, 92, 93, 94, 95, 96, 97, 98 or 99% identical to one of SEQ ID NOs: 62-71. In some embodiments, the nucleic acid sequence encoding an anti-PD-1-ABD light chain (without the signal peptide) comprise a nucleic acid sequence 80, 81, 82, 83, 84, 85, 86, 87, 88, 89, 90, 91, 92, 93, 94, 95, 96, 97, 98 or 99% identical to one of SEQ ID NOs: 62-71.

In some embodiments, the nucleic acid sequence encoding a signal peptide of the anti-PD-1-ABD antibody heavy or light chain comprise a nucleic acid sequence encoding an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the nucleic acid sequence encoding an anti-PD-1-ABD heavy chain (without the signal peptide) comprise a nucleic acid sequence encoding an amino acid sequence 80, 81, 82, 83, 84, 85, 86, 87, 88, 89, 90, 91, 92, 93, 94, 95, 96, 97, 98 or 99% identical to one of SEQ ID NOs: 62-71. In some embodiments, the nucleic acid sequence encoding an anti-PD-1-ABD light chain (without the signal peptide) comprise a nucleic acid sequence encoding an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75.

In some embodiments, the nucleic acid sequence encodes CDRs of the variable heavy chain domain and the CDRs of the variable light chain domain of the anti-PD-1-ABD antibody, wherein the CDR sequences are indicated above (in bold and underline) in SEQ ID NOS: 62-71. In some embodiments, the nucleic acid sequence encoding framework regions

(FRs) of the variable heavy chain domain and the FRs of the variable light chain domain of the anti-PD-1-ABD antibody comprise a nucleic acid sequence 80, 81, 82, 83, 84, 85, 86, 87, 88, 89, 90, 91, 92, 93, 94, 95, 96, 97, 98 or 99% identical to the FRs of one of SEQ ID NOs: 62-71. In some embodiments, one or more nucleic acids of a nucleic acid sequence encoding one or more CDRs of SEQ ID NOs: 62-71 is substituted. In some embodiments, one or more nucleic acids of a nucleic acid sequence encoding one or more variable heavy chain CDRs of any of SEQ ID NOs: 62-71 is substituted. In some embodiments, one or more nucleic acids of a nucleic acid sequence encoding one or more variable light chain CDRs of SEQ ID NO: 3, 15, 17, 19, 21, 23, 25, 27, 29, 31, 73, or 75 is substituted.

In some embodiments, the nucleic acid sequence comprises a nucleic acid sequence encoding the scFv-Fc described herein.

### Amino Acid and Nucleic Acid Sequences of Anti-PD-1 Antibodies

In some embodiments, the anti-PD-1 portion of the anti-PD-1-Fc, anti-PD-1-CD22, anti-PD-1-HSA, and anti-PD1-ABD antibody comprises at least a portion of the amino acid sequence encoding the anti-PD-1 targeting portion of Nivolumab, Pembrolizumab, Cemiplimab, Retifanlimab, Dostarlimab, Zimberelimab, Tiselelizumab, Camrelizumab, Sintilimab, Penpulimab, or any other anti-PD-1 antibody known in the art. In some embodiments, the amino acid sequence of the anti-PD-1 portion of the anti-PD-1-Fc or anti-PD-1-CD22 antibody comprises at least a variable heavy and variable light chain portions of the amino acid sequence of Nivolumab, Pembrolizumab, Cemiplimab, Retifanlimab, Dostarlimab, Zimberelimab, Tiselelizumab, Camrelizumab, Sintilimab, Penpulimab, or any other anti-PD-1 antibody known in the art. In some embodiments, the amino acid sequence of the anti-PD-1-Fc or anti-PD-1-CD22 antibody comprises at least the CDRs of the variable heavy chain and the CDRs of the variable light chain portions of the amino acid sequence of Nivolumab, Pembrolizumab, Cemiplimab, Retifanlimab, Dostarlimab, Zimberelimab, Tiselelizumab, Camrelizumab, Sintilimab, Penpulimab, or any other anti-PD-1 antibody known in the art. In some embodiments, the nucleic acid sequence the anti-PD-1-Fc or anti-PD-1-CD22 antibody codes for an amino acid sequence that comprises at least a portion of the amino acid sequence of Nivolumab, Pembrolizumab, Cemiplimab, Retifanlimab, Dostarlimab, Zimberelimab, Tiselelizumab, Camrelizumab, Sintilimab, Penpulimab, or any other anti-PD-1 antibody known in the art. In some embodiments, the nucleic acid sequence of the anti-PD-1-Fc or anti-PD-1-CD22 antibody codes for an amino acid sequence that comprises at least a variable heavy and variable light chain portions of the amino acid sequence of Nivolumab, Pembrolizumab, Cemiplimab, Retifanlimab, Dostarlimab, Zimberelimab, Tiselelizumab, Camrelizumab, Sintilimab, Penpulimab, or any other anti-PD-1 antibody known in the art. In some embodiments, the nucleic acid sequence encoding the anti-PD-1-Fc or anti-PD-1-CD22 antibody codes for an amino acid sequence that comprises at least the CDRs of the variable heavy chain and the CDRs of the variable light chain portions of the amino acid sequence of Nivolumab, Pembrolizumab, Cemiplimab, Retifanlimab, Dostarlimab, Zimberelimab, Tiselelizumab, Camrelizumab, Sintilimab, Penpulimab, or any other anti-PD-1 antibody known in the art. The sequences of anti-PD-1 antibodies are described in the art and incorporated herein by reference as follows: Pembrolizumab (see U.S. Pat. Nos. 8,168,757, 8,354,509, 8,900,587, the contents of each of which is hereby incorporated by reference in its entirety), Cemiplimab (see U.S. Pat. No. 9,987,500 the contents of which is hereby incorporated by reference in its entirety), Retifanlimab (see US2019/0127467 the contents of which is hereby incorporated by reference in its entirety), Dostarlimab (see WO/2021/058711 the contents of which is hereby incorporated by reference in its entirety), Zimberelimab (see CN106432494 the contents of which is hereby incorporated by reference in its entirety), Tiselelizumab (see U.S. Pat. No. 8,735,553 the contents of which is hereby incorporated by reference in its entirety), Camrelizumab (see US2019/0309069 the contents of which is hereby incorporated by reference in its entirety), Sintilimab (see U.S. Pat. No. 10,316,089 the contents of which is hereby incorporated by reference in its entirety), Penpulimab (see US2019/0321466 the contents of which is hereby incorporated by reference in its entirety). The CDRs of certain anti-PD-1 antibodies are described in Jeong T J, Lee H T, Gu N, Jang Y J, Choi S B, Park U B, Lee S H, Heo Y S, The High-Resolution Structure Reveals Remarkable Similarity in PD-1 Binding of Cemiplimab and ostarlimab, the FDA-Approved Antibodies for Cancer Immunotherapy. Biomedicines, 2022 Dec. 6; 10 (12): 3154, the contents of which is hereby incorporated by reference in its entirety.

In some embodiments, the anti-PD-1 portion of the antibody comprises a VH domain with hypervariable regions HCDR1, HCDR2 and HCDR3 sequences disclosed as in either FIG. 11 or in Table 1, including combinations thereof (e.g. any HCDR1, HCDR2, HCDR3 of FIG. 11 or Table 1 in combination with any HCDR1, HCDR2, HCDR3 of FIG. 11 or Table 1). In some embodiments, the anti-PD-1 portion of the antibody comprises a FR region which comprises HFR regions HFR1, HFR2, HFR3, and HFR4 sequences disclosed in FIG. 11 or Table 2. In some embodiments, the anti-PD-1 portion of the antibody comprises a VL domain which comprises hypervariable regions LCDR1, LCDR2, and LCDR3 comprising the amino acid sequences disclosed in FIG. 11 or Table 3, including combinations thereof (e.g. any LCDR1, LCDR2, LCDR3 of FIG. 11 or Table 1 in combination with any LCDR1, LCDR2, LCDR3 of FIG. 11 or Table 1). In some embodiments, the anti-PD-1 portion of the antibody comprises a FR region which comprises LFR regions LFR1, LFR2, LFR3, and LFR4 sequences disclosed in FIG. 11 or Table 4.

TABLE 1
Alternative HCDR Sequences for anti-PD1 portion

| Clone | Heavy Chain Sequence | HCDR1 | HCDR2 | HCDR3 |
|---|---|---|---|---|
| 001 | EVKLLESGGGLVQPGGSLKLSCAASGFDFSRYWM | RYWMS | EINPDSSTIKYT | MGYRLFDS |
| | SWVRQAPGKGLEWIGEINPDSSTIKYTPSLKDKFII | (SEQ ID | PSLKD (SEQ ID | (SEQ ID NO: 96) |
| | SRDNAKNTLCLQLSKVRSEDSALYYCARMGYRLF | NO: 94) | NO: 95) | |
| | DSWGQGTTLTVSS (SEQ ID NO: 72) | | | |
| 002 | EVQLQQSGTVLARPGASVKMSCKASGNTFTSYW | SYWMH | AIYPGYSDTRY | FIATVGGYFDV |
| | MHWVKQRPGQGLEWIGAIYPGYSDTRYNQKFK | (SEQ ID | NQKFKG (SEQ | (SEQ ID NO: 99) |
| | GKATLTAVTSTSTAYMELSSLTNEDSAVYYCTKFIA | NO: 97) | ID NO: 98) | |
| | TVGGYFDVWGAGTTVTVSS (SEQ ID NO: 74) | | | |
| 003 | QIQLVQSGPELKKPGETVKISCKASGYTFTDYSMH | DYSMH | WIKIETGNPTY | DYYGYYYYAMDY |
| | WVKQAPGKGLKWMGWIKIETGNPTYADDFKGR | (SEQ ID | ADDFKG (SEQ | (SEQ ID NO: 102) |
| | FAFSLETSASTAYLQINNLKNEDTATYFCARDYYG | NO: 100) | ID NO: 101) | |
| | YYYYAMDYWGQGTSVTVSS (SEQ ID NO: 76) | | | |
| 004 | EVKLVESGGGLVQPGGSLKLSCAASGFTFSSYTMS | SYTMS | YISYGGGDTYY | QKVDGYYVGMDY |
| | WVRQTPEKRLEWVAYISYGGGDTYYSDTVKGRF | (SEQ ID | SDTVKG (SEQ | (SEQ ID NO: 106) |
| | TISRDNAKNTLYLQMSSLKSEDTAMYYCARQKVD | NO: 104) | ID NO: 105) | |
| | GYYVGMDYWGQGSSVTVSS (SEQ ID NO: 103) | | | |
| 007 | EVQLVESGGGLVQPGGSLRLSCAASGFTFSHYGM | HYGMS | TIDNNGGNTY | DAYYTYDYWYFDV |
| | SWVRQTPDKRLELVATIDNNGGNTYYPDSVKGR | (SEQ ID | YPDSVKG | (SEQ ID NO: 109) |
| | FTISRDNAKNTLYLQMSSLKSEDTAMYYCARDAY | NO: 107) | (SEQ ID NO: | |
| | YTYDYWYFDVWGAGTTVTVSS (SEQ ID NO: | | 108) | |
| | 78) | | | |
| 009 | EVKLVESGGGLVQPGGSLKLSCAASGFTFSSYTMS | SYTMS | YISYGGGDTYY | QKVDGYFVAMDY |
| | WVRQTPEKRLEWVAYISYGGGDTYYPDTVKGRF | (SEQ ID | PDTVKG (SEQ | (SEQ ID NO: 111) |
| | TISRDNAKNTLYLQMSSLKSEDTAMYYCARQKVD | NO: 104) | ID NO: 110) | |
| | GYFVAMDYWSQGTSVTVSS (SEQ ID NO: 80) | | | |
| 051 | EVKLVESGGGLVKPGGSLKLSCAASGFTFSSYGMS | GFTFSSY | SGGGRY (SEQ | CASPYDGYYGA |
| | WVRQTPEKRLEWVATISGGGRYTYYPDSVKGRFT | (SEQ ID | ID NO: 113) | MDYW (SEQ ID |
| | ISRDNAKNILYLQMSSLRSEDTALYYCASPYDGYY | NO: 112) | | NO: 114) |
| | GAMDYWGQGTSVTVSS (SEQ ID NO: 82) | | | |
| 055 | EVQLQQSGTVLARPGASVRMSCKASGYSFSTYW | TYWMH | GIYPGNSDTNY | GYFFDV (SEQ |
| | MHWVKQRPGQGLEWIGGIYPGNSDTNYNQKFK | (SEQ ID | NQKFKG (SEQ | ID NO: 117) |
| | GKAKLTAVTSASTAYMDLSSLTNEDSAVYYCTGG | NO: 115) | ID NO: 116) | |
| | YFFDVWGAGTTVTVSS (SEQ ID NO: 84) | | | |
| 079 | QIQLQQSGPEQVKPGASVKISCKASGYMFIDYYM | DYYMN | WIYPGSGSTK | YGPRFFDV |
| | NWVKQKPGQGLEWIGWIYPGSGSTKDNENFKG | (SEQ ID | DNENFKG | (SEQ ID NO: |
| | KATLTVDTSSSTAYMQLSSLTSEDTAVYFCARYGP | NO: 118) | (SEQ ID NO: | 120) |
| | RFFDVWGAGTTVTVSS (SEQ ID NO: 86) | | 119) | |
| 080 | EVKLLESGGGLVQPGGSLKLSCEVSGFDFSRDWM | RDWMS | QISPDSTSINYK | DTSGYPDY |
| | SWVRQAPGKGLEWIGQISPDSTSINYKPSLKDKFII | (SEQ ID | PSLKD (SEQ ID | (SEQ ID NO: |
| | SRDNAKNTLYLQLSGVRSEDTALYHCARDTSGYP | NO: 121) | NO: 122) | 123 |
| | DYWGQGTSLTVSS (SEQ ID NO: 88) | | | |

TABLE 2
Alternative HFR Sequences for anti-PD1 portion

| Clone | Heavy Chain Sequence | HFR1 | HFR2 | HFR3 | HFR4 |
|---|---|---|---|---|---|
| 001 | EVKLLESGGGLVQPGGSLKLSCAA | EVKLLESGG | WVRQAPG | KFIISRDNAKNTLCL | WGQGTTLTVSS |
| | SGFDFSRYWMSWVRQAPGKGLE | GLVQPGGSL | KGLEWIG | QLSKVRSEDSALYY | (SEQ ID NO: 127) |
| | WIGEINPDSSTIKYTPSLKDKFIISR | KLSCAASGF | (SEQ ID | CAR (SEQ ID NO: | |
| | DNAKNTLCLQLSKVRSEDSALYYC | DFS (SEQ ID | NO: 125) | 126) | |
| | ARMGYRLFDSWGQGTTLTVSS | NO: 124) | | | |
| | (SEQ ID NO: 72) | | | | |
| 002 | EVQLQQSGTVLARPGASVKMSCK | EVQLQQSGT | WVKQRPG | KATLTAVTSTSTAY | WGAGTTVTVSS |
| | ASGNTFTSYWMHWVKQRPGQG | VLARPGASV | QGLEWIG | MELSSLTNEDSAV | (SEQ ID NO: 131) |
| | LEWIGAIYPGYSDTRYNQKFKGKA | KMSCKASG | (SEQ ID | YYCTK (SEQ ID | |
| | TLTAVTSTSTAYMELSSLTNEDSA | NTFT (SEQ | NO: 129) | NO: 130) | |
| | VYYCTKFIATVGGYFDVWGAGTT | ID NO: 128) | | | |
| | VTVSS (SEQ ID NO: 74) | | | | |
| 003 | QIQLVQSGPELKKPGETVKISCKAS | QIQLVQSGP | WVKQAPG | RFAFSLETSASTAY | WGQGTSVTVSS |
| | GYTFTDYSMHWVKQAPGKGLK | ELKKPGETV | KGLKWMG | LQINNLKNEDTAT | (SEQ ID NO: 135) |
| | WMGWIKIETGNPTYADDFKGRF | KISCKASGYT | (SEQ ID | YFCAR (SEQ ID | |
| | AFSLETSASTAYLQINNLKNEDTAT | FT (SEQ ID | NO: 133) | NO: 134) | |
| | YFCARDYYGYYYYAMDYWGQGT | NO: 132) | | | |
| | SVTVSS (SEQ ID NO: 76) | | | | |
| 004 | EVKLVESGGGLVQPGGSLKLSCAA | EVKLVESGG | WVRQTPEK | RFTISRDNAKNTLY | WGQGSSVTVSS |
| | SGFTFSSYTMSWVRQTPEKRLEW | GLVQPGGSL | RLEWVA | LQMSSLKSEDTAM | (SEQ ID NO: 139) |
| | VAYISYGGGDTYYSDTVKGRFTISR | KLSCAASGFT | (SEQ ID | YYCAR (SEQ ID | |
| | DNAKNTLYLQMSSLKSEDTAMYY | FS (SEQ ID | NO: 137) | NO: 138) | |
| | CARQKVDGYYVGMDYWGQGSS | NO: 136) | | | |
| | VTVSS (SEQ ID NO: 103) | | | | |
| 007 | EVQLVESGGGLVQPGGSLRLSCA | EVQLVESGG | WVRQTPD | RFTISRDNAKNTLY | WGAGTTVTVSS |
| | ASGFTFSHYGMSWVRQTPDKRL | GLVQPGGSL | KRLELVA | LQMSSLKSEDTAM | (SEQ ID NO: 131) |
| | ELVATIDNNGGNTYYPDSVKGRF | RLSCAASGF | (SEQ ID | YYCAR (SEQ ID | |
| | TISRDNAKNTLYLQMSSLKSEDTA | TFS (SEQ ID | NO: 141) | NO: 138) | |
| | MYYCARDAYYTYDYWYFDVWG | NO: 140) | | | |
| | AGTTVTVSS (SEQ ID NO: 78) | | | | |
| 009 | EVKLVESGGGLVQPGGSLKLSCAA | EVKLVESGG | WVRQTPEK | RFTISRDNAKNTLY | WSQGTSVTVSS |
| | SGFTFSSYTMSWVRQTPEKRLEW | GLVQPGGSL | RLEWVA | LQMSSLKSEDTAM | (SEQ ID NO: 142) |
| | VAYISYGGGDTYYPDTVKGRFTIS | KLSCAASGFT | (SEQ ID | YYCAR (SEQ ID | |
| | RDNAKNTLYLQMSSLKSEDTAMY | FS (SEQ ID | NO: 137) | NO: 138) | |
| | YCARQKVDGYFVAMDYWSQGTS | NO: 136) | | | |
| | VTVSS (SEQ ID NO: 80) | | | | |
| 051 | EVKLVESGGGLVKPGGSLKLSCAA | EVKLVESGG | GMSWVRQ | TYYPDSVKGRFTIS | GQGTSVTVSS |
| | SGFTFSSYGMSWVRQTPEKRLE | GLVKPGGSL | TPEKRLEW | RDNAKNILYLQMS | (SEQ ID NO: 146) |
| | WVATISGGGRYTYYPDSVKGRFTI | KLSCAAS | VATI (SEQ | SLRSEDTALYY | |
| | SRDNAKNILYLQMSSLRSEDTALY | (SEQ ID NO: | ID NO: | (SEQ ID NO: 145) | |
| | YCASPYDGYYGAMDYWGQGTSV | 143) | 144) | | |
| | TVSS (SEQ ID NO: 82) | | | | |
| 055 | EVQLQQSGTVLARPGASVRMSC | EVQLQQSGT | WVKQRPG | KAKLTAVTSASTAY | WGAGTTVTVSS |
| | KASGYSFSTYWMHWVKQRPGQ | VLARPGASV | QGLEWIG | MDLSSLTNEDSAV | (SEQ ID NO: 131) |
| | GLEWIGGIYPGNSDTNYNQKFKG | RMSCKASGY | (SEQ ID | YYCTG (SEQ ID | |
| | KAKLTAVTSASTAYMDLSSLTNED | SFS (SEQ ID | NO: 129) | NO: 148) | |
| | SAVYYCTGGYFFDVWGAGTTVTV | NO: 147) | | | |
| | SS (SEQ ID NO: 84) | | | | |
| 079 | QIQLQQSGPEQVKPGASVKISCKA | QIQLQQSGP | WVKQKPG | KATLTVDTSSSTAY | WGAGTTVTVSS |
| | SGYMFIDYYMNWVKQKPGQGLE | EQVKPGASV | QGLEWIG | MQLSSLTSEDTAV | (SEQ ID NO: 131) |
| | WIGWIYPGSGSTKDNENFKGKAT | KISCKASGY | (SEQ ID | YFCAR (SEQ ID | |
| | LTVDTSSSTAYMQLSSLTSEDTAV | MFI (SEQ ID | NO: 150) | NO: 151) | |
| | YFCARYGPRFFDVWGAGTTVTVSS | NO: 149) | | | |
| | (SEQ ID NO: 86) | | | | |
| 080 | EVKLLESGGGLVQPGGSLKLSCEV | EVKLLESGG | WVRQAPG | KFIISRDNAKNTLYL | WGQGTSLTVSS |
| | SGFDFSRDWMSWVRQAPGKGL | GLVQPGGSL | KGLEWIG | QLSGVRSEDTALY | (SEQ ID NO: 154) |
| | EWIGQISPDSTSINYKPSLKDKFIIS | KLSCEVSGF | (SEQ ID | HCAR (SEQ ID NO: | |
| | RDNAKNTLYLQLSGVRSEDTALYH | DFS (SEQ ID | NO: 125) | 153) | |
| | CARDTSGYPDYWGQGTSLTVSS | NO: 152) | | | |
| | (SEQ ID NO: 88) | | | | |

TABLE 3
Alternative LCDR Sequences for anti-PD1 portion

| Clone | Light Chain Sequence | LCDR1 | LCDR2 | LCDR3 |
|---|---|---|---|---|
| 001 | NIVMTQATPSVPVTPGESVSISCRSSKSLL | RSSKSLL | RMSNLVS (SEQ ID | MQHLEYPYT (SEQ ID |
| | HSDGDTYLYWFLQRPGQSPQLLIYRMSN | HSDGDT | NO: 156) | NO: 157) |
| | LVSGVPDRFSGSGSGTAFTLRISRVEAED | YLY (SEQ | | |
| | VGVYYCMQHLEYPYTFGGGTKLEIK | ID NO: | | |
| | (SEQ ID NO: 73) | 155) | | |
| 002 | QIVLTQSPAIMSSSLGERVTMTCTASSSV | TASSSVS | STSNLAS (SEQ ID | HQYHRSPPIFT |
| | SSSYLHWYQQKPGSSPKLWIYSTSNLASG | SSYLH | NO: 159) | (SEQ ID NO: 160) |
| | VPARFSGSGSGTSYSLTISSMEAEDAATY | (SEQ ID | | |
| | YCHQYHRSPPIFTFGSGTKLEIK | NO: 158) | | |
| | (SEQ ID NO: 75) | | | |
| 003 | QIVLTQSPAIMSASLGERVTMTCTVSSSIS | TVSSSISS | STSNLAS (SEQ ID | HQYHRSPLT (SEQ ID |
| | SSYLHWYQQKPGSSPKLWIYSTSNLASG | SYLH | NO: 159) | NO: 162) |
| | VPARFSGSGSGTSYSLTISSMEAEDAATY | (SEQ ID | | |
| | YCHQYHRSPLTFGAGTKLELK (SEQ ID | NO: 161) | | |
| | NO: 77) | | | |
| 004 | NIVMTQSPKSMSMSVGERVTLSCKASEN | KASENV | GASNRYT (SEQ ID | GQSYSSPLT (SEQ ID |
| | VGTNVSWYQQKPEQSPRLLIYGASNRYT | GTNVS | NO: 165) | NO: 166) |
| | GVPDRFTGSGSVTDFTLTISSVQAEDLVD | (SEQ ID | | |
| | YHCGQSYSSPLTFGSGTKLELK (SEQ ID | NO: 164) | | |
| | NO: 163) | | | |
| 007 | DIQMTQTTSSLSASLGDRVTISCSASQGIS | SASQGIS | YTSSLHS (SEQ ID | QQYHKLPWT (SEQ ID |
| | NYLNWYQQKPDGTVKLLIYYTSSLHSGVP | NYLN | NO: 168) | NO: 169) |
| | SRFSGSGSGTDYSLTISNLEPEDIATYYCQ | (SEQ ID | | |
| | QYHKLPWTFGGGTKLEIK (SEQ ID NO: | NO: 167) | | |
| | 79) | | | |
| 009 | VIVLTQSPASLAVSLGQRATISCRASERVD | RASERVD | AASNQGS (SEQ ID | QQNKEVPWT (SEQ ID |
| | DYGISFMNWFQQKPGQPPKLLIYAASNQ | DYGISFM | NO: 171) | NO: 172) |
| | GSGVPARFSGSGSGTDFSLNIHPMEEDD | N (SEQ | | |
| | TAMYFCQQNKEVPWTFGGGTKLEIK | ID NO: | | |
| | (SEQ ID NO: 81) | 170) | | |
| 051 | DIVLTQSPASLAVSLGQRATISCRASESVD | RASESVD | AASNQGS (SEQ ID | CQQSKEVPWTF (SEQ |
| | NSGISFMNWFQQKPGQPPKLLIYAASNQ | NSGISFM | NO: 171) | ID NO: 174) |
| | GSGVPARFSGSGSGTDFSLNIHPMEEDD | N (SEQ | | |
| | TAMYFCQQSKEVPWTFGGGTKLEIK | ID NO: | | |
| | (SEQ ID NO: 83) | 173) | | |
| 055 | DIKMTQSPSSLYASLGERVTITCKASQDIN | KASQDIN | RANRLVT (SEQ ID | LQYDESPYT (SEQ ID |
| | RYLTWFQQKPGKSPKTLIFRANRLVTGVP | RYLT | NO: 176) | NO: 177) |
| | SRFIGSGSGQEYSLTISSLEYEDMGIYFCLQ | (SEQ ID | | |
| | YDESPYTFGGGTKLEIK | NO: 175) | | |
| | (SEQ ID NO: 85) | | | |
| 079 | DIQMTQSPSSLSASLGERVNLTCRASQEI | RASQEIS | VASTLDS (SEQ ID | LQYASYPYT (SEQ ID |
| | SGYLSWLQQKPDGTIKRLIYVASTLDSGV | GYLS | NO: 179) | NO: 180) |
| | PERFSGSRSGSDYSLTISSLESEDFADYYCL | (SEQ ID | | |
| | QYASYPYTFGGGTKLEIK | NO: 178) | | |
| | (SEQ ID NO: 87) | | | |
| 080 | DILLTQSPSSMSVSLGDTVSITCHASQDIS | HASQDIS | HGTNLED (SEQ ID | VQYAQFPYT (SEQ ID |
| | PSRFSGSGSGADYSLTISSLESEDFADYYC | RNIG | NO: 182) | NO: 183) |
| | RNIGWLRQKPGKSFKGLIYHGTNLEDGV | (SEQ ID | | |
| | VQYAQFPYTFGGGTKLEIK | NO: 181) | | |
| | (SEQ ID NO: 89) | | | |

TABLE 4
Alternative LFR Sequences for the anti-PD1 portion

| Clone | Light Chain Sequence | LFR1 | LFR2 | LFR3 | LFR4 |
|---|---|---|---|---|---|
| 001 | NIVMTQATPSVPVTPGESVSISCR | NIVMTQATP | WFLQRPG | GVPDRFSGSGSGT | FGGGTKLEIK (SEQ |
| | SSKSLLHSDGDTYLYWFLQRPGQS | SVPVTPGES | QSPQLLIY | AFTLRISRVEAEDV | ID NO: 187) |
| | PQLLIYRMSNLVSGVPDRFSGSGS | VSISC (SEQ | (SEQ ID | GVYYC (SEQ ID | |
| | GTAFTLRISRVEAEDVGVYYCMQ | ID NO: 184) | NO: 185) | NO: 186) | |
| | HLEYPYTFGGGTKLEIK (SEQ ID | | | | |
| | NO: 73) | | | | |
| 002 | QIVLTQSPAIMSSSLGERVTMTCT | QIVLTQSPAI | WYQQKPG | GVPARFSGSGSGT | FGSGTKLEIK (SEQ |
| | ASSSVSSSYLHWYQQKPGSSPKL | MSSSLGERV | SSPKLWIY | SYSLTISSMEAEDA | ID NO: 191) |
| | WIYSTSNLASGVPARFSGSGSGTS | TMTC (SEQ | (SEQ ID | ATYYC (SEQ ID | |
| | YSLTISSMEAEDAATYYCHQYHRS | ID NO: 188) | NO: 189) | NO: 190) | |
| | PPIFTFGSGTKLEIK (SEQ ID NO: | | | | |
| | 75) | | | | |
| 003 | QIVLTQSPAIMSASLGERVTMTCT | QIVLTQSPAI | WYQQKPG | GVPARFSGSGSGT | FGAGTKLELK (SEQ |
| | VSSSISSSYLHWYQQKPGSSPKLW | MSASLGERV | SSPKLWIY | SYSLTISSMEAEDA | ID NO: 193) |
| | IYSTSNLASGVPARFSGSGSGTSYS | TMTC (SEQ | (SEQ ID | ATYYC (SEQ ID | |
| | LTISSMEAEDAATYYCHQYHRSPL | ID NO: 192) | NO: 189) | NO: 190) | |
| | TFGAGTKLELK (SEQ ID NO: 77) | | | | |
| 004 | NIVMTQSPKSMSMSVGERVTLSC | NIVMTQSPK | WYQQKPE | GVPDRFTGSGSVT | FGSGTKLELK (SEQ |
| | KASENVGTNVSWYQQKPEQSPR | SMSMSVGE | QSPRLLIY | DFTLTISSVQAEDL | ID NO: 197) |
| | LLIYGASNRYTGVPDRFTGSGSVT | RVTLSC | (SEQ ID | VDYHC (SEQ ID | |
| | DFTLTISSVQAEDLVDYHCGQSYS | (SEQ ID NO: | NO: 195) | NO: 196) | |
| | SPLTFGSGTKLELK (SEQ ID NO: | 194) | | | |
| | 163) | | | | |
| 007 | DIQMTQTTSSLSASLGDRVTISCS | DIQMTQTTS | WYQQKPD | GVPSRFSGSGSGT | FGGGTKLEIK (SEQ |
| | ASQGISNYLNWYQQKPDGTVKLL | SLSASLGDR | GTVKLLIY | DYSLTISNLEPEDIA | ID NO: 187) |
| | IYYTSSLHSGVPSRFSGSGSGTDYS | VTISC (SEQ | (SEQ ID | TYYC | |
| | LTISNLEPEDIATYYCQQYHKLPW | ID NO: 198) | NO: 199) | (SEQ ID NO: 200) | |
| | TFGGGTKLEIK (SEQ ID NO: 79) | | | | |
| 009 | VIVLTQSPASLAVSLGQRATISCRA | VIVLTQSPAS | WFQQKPG | GVPARFSGSGSGT | FGGGTKLEIK (SEQ |
| | SERVDDYGISFMNWFQQKPGQP | LAVSLGQRA | QPPKLLIY | DFSLNIHPMEEDD | ID NO: 187) |
| | PKLLIYAASNQGSGVPARFSGSGS | TISC (SEQ ID | (SEQ ID | TAMYFC (SEQ ID | |
| | GTDFSLNIHPMEEDDTAMYFCQ | NO: 201) | NO: 202) | NO: 203) | |
| | QNKEVPWTFGGGTKLEIK (SEQ | | | | |
| | ID NO: 81) | | | | |
| 051 | DIVLTQSPASLAVSLGQRATISCRA | DIVLTQSPAS | WFQQKPG | GVPARFSGSGSGT | GGGTKLEIK (SEQ |
| | SESVDNSGISFMNWFQQKPGQP | LAVSLGQRA | QPPKLLIY | DFSLNIHPMEEDD | ID NO: 206) |
| | PKLLIYAASNQGSGVPARFSGSGS | TISC (SEQ ID | (SEQ ID | TAMYF (SEQ ID | |
| | GTDFSLNIHPMEEDDTAMYFCQ | NO: 204) | NO: 202) | NO: 205) | |
| | QSKEVPWTFGGGTKLEIK (SEQ | | | | |
| | ID NO: 83) | | | | |
| 055 | DIKMTQSPSSLYASLGERVTITCKA | DIKMTQSPS | WFQQKPG | GVPSRFIGSGSGQ | FGGGTKLEIK (SEQ |
| | SQDINRYLTWFQQKPGKSPKTLIF | SLYASLGERV | KSPKTLIF | EYSLTISSLEYEDM | ID NO: 187) |
| | RANRLVTGVPSRFIGSGSGQEYSL | TITC (SEQ ID | (SEQ ID | GIYFC (SEQ ID | |
| | TISSLEYEDMGIYFCLQYDESPYTF | NO: 207) | NO: 208) | NO: 209) | |
| | GGGTKLEIK (SEQ ID NO: 85) | | | | |
| 079 | DIQMTQSPSSLSASLGERVNLTCR | DIQMTQSPS | WLQQKPD | GVPERFSGSRSGS | FGGGTKLEIK (SEQ |
| | ASQEISGYLSWLQQKPDGTIKRLIY | SLSASLGERV | GTIKRLIY | DYSLTISSLESEDFA | ID NO: 187) |
| | VASTLDSGVPERFSGSRSGSDYSL | NLTC (SEQ | (SEQ ID | DYYC | |
| | TISSLESEDFADYYCLQYASYPYTF | ID NO: 210) | NO: 211) | (SEQ ID NO: 212) | |
| | GGGTKLEIK (SEQ ID NO: 87) | | | | |
| 080 | DILLTQSPSSMSVSLGDTVSITCHA | DILLTQSPSS | WLRQKPG | GVPSRFSGSGSGA | FGGGTKLEIK (SEQ |
| | SQDISRNIGWLRQKPGKSFKGLIY | MSVSLGDTV | KSFKGLIY | DYSLTISSLESEDFA | ID NO: 187) |
| | HGTNLEDGVPSRFSGSGSGADYS | SITC (SEQ ID | (SEQ ID | DYYC | |
| | LTISSLESEDFADYYCVQYAQFPYT | NO: 213) | NO: 214) | (SEQ ID NO: 215) | |
| | FGGGTKLEIK (SEQ ID NO: 89) | | | | |

TABLE 5
Exemplary nucleic acid sequences encoding an anti-PD1 portion

| Clone | Heavy Chain | Light Chain |
|---|---|---|
| 001 | GAGGTGAAGCTTCTCGAGTCTGGAGGTGGCCT | AATATTGTGATGACTCAGGCTACACCCTCTGT |
| | GGTGCAGCCTGGAGGATCCCTGAAACTCTCCTG | ACCTGTCACTCCTGGAGAGTCAGTATCCATCT |
| | TGCAGCCTCAGGATTCGATTITAGTAGATACTG | CCTGCAGGTCTAGTAAGAGTCTCCTGCATAG |
| | GATGAGTTGGGTCCGGCAGGCTCCAGGGAAAG | TGATGGCGACACTTACTTGTATTGGTTCCTGC |
| | GGCTAGAATGGATTGGAGAAATTAATCCAGAT | AGAGGCCAGGCCAGTCTCCTCAGCTCCTGAT |
| | AGCAGTACGATAAAATACACGCCATCTCTAAAG | ATATCGGATGTCCAACCTTGTCTCAGGAGTC |
| | GATAAATTCATCATCTCCAGAGACAACGCCAAA | CCAGACAGGTTCAGTGGCAGTGGGTCAGGA |
| | AATACGCTGTGCCTGCAATTGAGCAAAGTGAGA | ACTGCTTTCACACTGAGAATCAGTAGAGTGG |
| | TCTGAGGACTCAGCCCTTTATTACTGTGCAAGA | AGGCTGAGGATGTGGGTGTTTATTACTGTAT |
| | ATGGGCTATAGGCTCTTTGACTCCTGGGGCCAA | GCAACATCTAGAATATCCGTACACGTTCGGA |
| | GGCACCACTCTCACAGTCTCCTCA (SEQ ID NO: | GGGGGGACCAAGCTGGAAATAAAA (SEQ ID |
| | 216) | NO: 217) |
| 002 | GAGGTTCAGCTCCAGCAGTCTGGGACTGTGCTG | CAAATTGTTCTCACCCAGTCTCCAGCAATCAT |
| | GCAAGGCCTGGGGCTTCAGTGAAGATGTCCTG | GTCTTCATCTCTAGGGGAACGGGTCACCATG |
| | CAAGGCTTCTGGCAACACCTTTACCAGCTACTG | ACCTGCACTGCCAGCTCAAGTGTAAGTTCCA |
| | GATGCACTGGGTAAAACAGAGGCCTGGACAGG | GTTACTTGCACTGGTACCAGCAGAAGCCAGG |
| | GTCTGGAATGGATTGGCGCTATTTATCCTGGAT | ATCCTCCCCCAAACTCTGGATTTATAGCACAT |
| | ATAGTGATACTAGGTACAATCAGAAGTTTAAGG | CCAACCTGGCTTCTGGAGTCCCAGCTCGCTTC |
| | GCAAGGCCACACTGACTGCAGTCACATCCACCA | AGTGGCAGTGGGTCTGGGACCTCTTACTCTC |
| | GCACTGCCTACATGGAGCTCAGCAGCCTGACAA | TCACAATCAGCAGCATGGAGGCTGAAGATG |
| | ATGAGGACTCTGCGGTCTATTACTGTACAAAAT | CTGCCACTTATTACTGCCACCAGTATCATCGT |
| | TTATTGCTACGGTAGGGGGGTACTTCGATGTCT | TCCCCACCCATATTCACGTTCGGCTCGGGGA |
| | GGGGCGCAGGGACCACGGTCACCGTCTCCTCA | CAAAGTTGGAAATAAAA (SEQ ID NO: 219) |
| | (SEQ ID NO: 218) | |
| 003 | CAGATCCAGTTGGTGCAGTCTGGACCTGAGCTG | CAAATTGTTCTCACCCAGTCTCCAGCAATCAT |
| | AAGAAGCCTGGAGAGACAGTCAAGATCTCCTG | GTCTGCATCTCTAGGGGAACGGGTCACCATG |
| | CAAGGCTTCTGGTTATACCTTCACAGACTATTCA | ACCTGCACTGTTAGCTCAAGTATAAGTTCCA |
| | ATGCACTGGGTGAAGCAGGCTCCAGGAAAGGG | GTTACTTGCACTGGTACCAGCAGAAGCCAGG |
| | TTTAAAGTGGATGGGCTGGATAAAAATTGAGA | ATCCTCCCCCAAACTCTGGATTTATAGCACAT |
| | CTGGTAACCCAACATATGCAGATGACTTCAAGG | CCAACCTGGCTTCTGGAGTCCCAGCTCGCTTC |
| | GACGGTTTGCCTTCTCTTTGGAAACCTCTGCCAG | AGTGGCAGTGGGTCTGGGACCTCTTACTCTC |
| | CACTGCCTATTTGCAGATCAACAACCTCAAAAAT | TCACAATCAGCAGCATGGAGGCTGAAGATG |
| | GAGGACACGGCTACATATTTCTGTGCGAGAGA | CTGCCACTTATTACTGCCACCAGTATCATCGT |
| | CTACTATGGTTATTATTACTATGCTATGGACTAC | TCCCCGCTCACGTTCGGTGCTGGGACCAAGC |
| | TGGGGTCAAGGAACCTCAGTCACCGTCTCCTCA | TGGAGCTGAAA (SEQ ID NO: 221) |
| | (SEQ ID NO: 220) | |
| 004 | GAAGTGAAGCTGGTGGAGTCTGGGGGAGGTTT | AACATTGTAATGACCCAATCTCCCAAATCCAT |
| | AGTGCAGCCTGGAGGGTCCCTGAAACTCTCCTG | GTCCATGTCAGTAGGAGAGAGGGTCACCTTG |
| | TGCAGCCTCTGGATTCACTTTCAGTAGCTATACC | AGCTGCAAGGCCAGCGAGAATGTGGGTACT |
| | ATGTCTTGGGTTCGCCAGACTCCAGAGAAGAG | AATGTATCCTGGTATCAACAGAAACCAGAGC |
| | GCTGGAGTGGGTCGCATACATTAGTTATGGTG | AGTCTCCTAGACTGCTGATATATGGGGCTTC |
| | GAGGTGACACCTACTATTCAGACACTGTAAAGG | CAACCGGTACACTGGGGTCCCCGATCGCTTC |
| | GCCGATTCACCATCTCCAGAGACAATGCCAAGA | ACAGGCAGTGGATCTGTAACAGATTTCACTC |
| | ACACCCTGTACCTACAAATGAGCAGTCTGAAGT | TGACCATCAGCAGTGTGCAGGCTGAGGACCT |
| | CTGAGGACACGGCCATGTATTACTGTGCAAGAC | TGTAGATTATCACTGTGGACAGAGTTACAGC |
| | AGAAGGTCGATGGTTACTACGTTGGTATGGACT | TCTCCGCTCACGTTCGGTTCTGGGACCAAGCT |
| | ACTGGGGTCAAGGATCCTCAGTCACCGTCTCCT | GGAGCTGAAA (SEQ ID NO: 223) |
| | CA (SEQ ID NO: 222) | |
| 007 | GAGGTGCAGCTGGTGGAGTCTGGGGGAGGCTT | GATATCCAGATGACACAGACTACATCCTCCCT |
| | AGTGCAGCCTGGAGGGTCCCTGAGACTCTCCTG | GTCTGCCTCTCTGGGAGACAGAGTCACCATC |
| | TGCAGCCTCTGGATTCACTTTCAGTCACTATGGC | AGTTGCAGTGCGAGTCAGGGCATTAGTAATT |
| | ATGTCTTGGGTTCGCCAGACTCCAGACAAGAGG | ATTTAAACTGGTATCAGCAGAAACCAGATGG |
| | CTGGAGTTGGTCGCAACCATTGATAATAATGGT | AACTGTGAAACTCCTGATCTATTACACATCAA |
| | GGTAACACCTATTATCCAGACAGTGTGAAGGGC | GTTTACATTCAGGAGTCCCATCAAGGTTCAG |
| | CGATTCACCATCTCCAGAGACAATGCCAAGAAC | TGGCAGTGGGTCTGGGACAGATTATTCTCTC |
| | ACCCTGTACCTGCAAATGAGCAGTCTGAAGTCT | ACCATCAGCAACCTAGAACCTGAAGATATTG |
| | GAGGACACAGCCATGTATTACTGTGCAAGAGA | CCACTTACTATTGTCAACAGTATCATAAGCTT |
| | CGCCTACTATACTTACGACTACTGGTACTTCGAT | CCGTGGACGTTCGGTGGAGGCACCAAGCTG |
| | GTCTGGGGCGCAGGGACCACGGTCACCGTCTC | GAAATCAAA (SEQ ID NO: 225) |
| | CTCA (SEQ ID NO: 224) | |
| 009 | GAAGTGAAGCTGGTGGAGTCTGGGGGAGGTTT | GTCATTGTGCTGACCCAATCTCCAGCTTCTTT |
| | AGTGCAGCCTGGAGGGTCCCTGAAACTCTCCTG | GGCTGTGTCTCTAGGGCAGAGGGCCACCATC |
| | TGCAGCCTCTGGATTCACTTTCAGTAGCTATACC | TCCTGCAGAGCCAGCGAGCGTGTTGATGATT |
| | ATGTCTTGGGTTCGCCAGACTCCAGAGAAGAG | ATGGCATTAGTTTTATGAACTGGTTCCAACA |
| | GCTGGAGTGGGTCGCATACATTAGTTATGGTG | GAAACCAGGACAGCCACCCAAACTCCTCATC |
| | GAGGTGACACGTACTATCCAGACACTGTAAAG | TATGCTGCATCCAACCAAGGATCCGGGGTCC |
| | GGCCGATTCACCATCTCCAGAGACAATGCCAAG | CTGCCAGGTTTAGTGGCAGTGGGTCTGGGAC |
| | AACACCCTGTACCTGCAAATGAGCAGTCTGAAG | AGACTTCAGCCTCAACATCCATCCTATGGAG |
| | TCTGAGGACACGGCCATGTATTACTGTGCCAGA | GAGGATGATACTGCAATGTATTTCTGTCAGC |
| | CAGAAGGTCGATGGTTACTTCGTTGCTATGGAC | AAAATAAGGAGGTTCCGTGGACGTTCGGTG |
| | TATTGGAGTCAAGGAACCTCAGTCACCGTCTCC | GAGGCACCAAGCTGGAAATCAAA (SEQ ID |
| | TCA (SEQ ID NO: 226) | NO: 227) |
| 051 | GAAGTGAAGCTGGTGGAGTCTGGGGGAGGCTT | GACATTGTGCTGACCCAATCTCCAGCTTCTTT |
| | AGTGAAGCCTGGAGGGTCCCTGAAACTCTCCTG | GGCTGTGTCTCTAGGGCAGAGGGCCACCATC |
| | TGCAGCCTCTGGATTCACTTTCAGTAGCTATGG | TCCTGCAGAGCCAGCGAAAGTGTTGATAATT |
| | CATGTCTTGGGTTCGCCAGACTCCGGAGAAGA | CTGGCATTAGTTTTATGAACTGGTTCCAACAG |
| | GGCTGGAGTGGGTCGCAACCATCAGTGGTGGT | AAACCAGGACAGCCACCCAAACTCCTCATCT |
| | GGTAGATACACCTACTATCCAGACAGTGTGAAG | ATGCTGCATCCAACCAAGGATCCGGGGTCCC |
| | GGGCGATTCACCATCTCCAGAGACAATGCCAAG | TGCCAGGTTTAGTGGCAGTGGGTCTGGGAC |
| | AACATCCTGTACCTGCAAATGAGTAGTCTGAGG | AGACTTCAGCCTCAACATCCATCCTATGGAG |
| | TCTGAGGACACGGCCTTGTATTACTGTGCAAGC | GAGGATGATACTGCAATGTATTTCTGTCAGC |
| | CCCTATGATGGTTACTACGGTGCTATGGACTAC | AAAGTAAGGAGGTTCCGTGGACGTTCGGTG |
| | TGGGGTCAAGGAACCTCAGTCACCGTCTCCTCA | GAGGCACCAAGCTGGAAATCAAA (SEQ ID |
| | (SEQ ID NO: 228) | NO: 229) |
| 055 | GAGGTTCAGCTCCAGCAGTCTGGGACTGTGCTG | GACATCAAGATGACCCAGTCTCCATCTTCCTT |
| | GCAAGGCCTGGGGCTTCCGTGAGGATGTCCTG | GTATGCATCTCTAGGAGAGAGAGTCACTATC |
| | CAAGGCTTCTGGCTACAGCTTTTCCACCTACTGG | ACTTGCAAGGCGAGTCAGGACATTAATCGAT |
| | ATGCACTGGGTAAAACAGAGGCCTGGACAGGG | ATTTAACCTGGTTCCAGCAGAAACCAGGGAA |
| | TCTAGAATGGATTGGTGGTATTTATCCTGGAAA | ATCTCCTAAGACCCTGATCTTTCGTGCAAACA |
| | TAGTGACACTAACTACAACCAGAAGTTCAAGGG | GATTGGTAACTGGGGTCCCATCAAGGTTCAT |
| | CAAGGCCAAACTGACTGCAGTCACTTCCGCCAG | TGGCAGTGGATCTGGGCAAGAATATTCTCTC |
| | CACTGCCTACATGGACCTCAGCAGCCTGACAAA | ACCATCAGCAGCCTGGAGTATGAAGACATG |
| | TGAGGACTCTGCGGTCTATTACTGTACAGGCGG | GGAATTTATTTTTGTCTACAGTATGATGAGTC |
| | TTACTTCTTCGATGTCTGGGGCGCAGGGACCAC | TCCGTACACGTTCGGAGGGGGGACCAAGTT |
| | GGTCACCGTCTCCTCA (SEQ ID NO: 230) | GGAAATAAAA (SEQ ID NO: 231) |
| 079 | CAGATCCAGCTGCAGCAGTCTGGACCTGAGCA | GACATCCAGATGACCCAGTCTCCATCCTCCTT |
| | GGTGAAGCCTGGGGCTTCAGTGAAGATATCCT | ATCTGCCTCTCTGGGAGAAAGAGTCAATCTC |
| | GCAAGGCTTCTGGCTACATGTTCATCGACTACT | ACTTGTCGGGCAAGTCAGGAAATTAGTGGTT |
| | ATATGAACTGGGTGAAGCAGAAGCCTGGACAG | ACTTAAGCTGGCTTCAGCAGAAACCAGATGG |
| | GGACTTGAGTGGATTGGATGGATTTATCCTGGA | AACTATTAAACGCCTGATCTACGTCGCATCCA |
| | AGCGGTAGTACTAAGGACAATGAGAATTTCAA | CTTTAGATTCTGGTGTCCCAGAAAGGTTCAG |
| | GGGCAAGGCCACATTGACTGTAGACACATCCTC | TGGCAGTAGGTCTGGGTCAGATTATTCTCTC |
| | CAGCACAGCCTACATGCAGCTCAGTAGCCTGAC | ACCATCAGCAGCCTTGAGTCTGAGGATTTTG |
| | ATCTGAGGACACTGCTGTCTATTTCTGTGCAAG | CAGACTATTACTGTCTACAATATGCTAGTTAT |
| | ATATGGGCCCAGGTTCTTCGATGTCTGGGGCGC | CCGTACACGTTCGGAGGGGGGACCAAGCTG |
| | AGGGACCACGGTCACCGTCTCCTCA (SEQ ID | GAAATAAAA (SEQ ID NO: 233) |
| | NO: 232) | |
| 080 | GAGGTGAAGCTTCTCGAGTCTGGAGGTGGCCT | GACATCCTGTTGACCCAATCTCCATCCTCCAT |
| | GGTGCAGCCTGGAGGATCCCTGAAACTCTCCTG | GTCTGTTTCTCTGGGAGACACAGTCAGCATA |
| | TGAAGTCTCAGGATTCGATTTTAGCAGAGACTG | ACGTGTCATGCAAGTCAGGACATTAGCAGGA |
| | GATGAGTTGGGTCCGGCAGGCTCCAGGGAAAG | ATATAGGGTGGTTGCGGCAGAAACCAGGGA |
| | GGCTAGAGTGGATTGGACAAATTTCTCCAGATA | AATCATTTAAGGGCCTGATCTATCATGGAAC |
| | GCACTTCGATAAACTATAAGCCATCTCTAAAGG | CAATTTGGAAGATGGAGTTCCATCAAGGTTC |
| | ATAAATTCATCATCTCCAGAGACAACGCCAAAA | AGCGGCAGTGGATCTGGAGCAGATTATTCTC |
| | ATACGCTGTACCTGCAATTGAGCGGAGTGAGAT | TCACCATCAGCAGCCTGGAATCTGAAGATTT |
| | CTGAGGACACAGCCCTTTATCACTGTGCAAGAG | TGCAGACTATTACTGTGTGCAGTATGCTCAG |
| | ACACCTCGGGCTACCCCGACTACTGGGGCCAAG | TTTCCGTACACGTTCGGAGGGGGGACCAAAC |
| | GCACCAGTCTCACAGTCTCCTCA (SEQ ID NO: | TGGAAATAAAA (SEQ ID NO: 235) |
| | 234) | |

Amino Acid Sequences of Anti-PD-1 Antibodies with Additional Domain

The antibody comprising a dimer of the heterodimer comprising SEQ ID NO: 2 and SEQ ID NO: 3 is represented by antibody “anti-PD-1-Fc” in embodiments described and depicted in this disclosure.

The antibody comprising a dimer of the heterodimer comprising SEQ ID NO: 11 and SEQ ID NO: 3 is represented by antibody “anti-PD-1-CD22 (505-687)” in embodiments described and depicted in this disclosure.

The antibody comprising a dimer of the heterodimer comprising SEQ ID NO: 12 and SEQ ID NO: 3 is represented by antibody “anti-PD-1-CD22 (593-687)” in embodiments described and depicted in this disclosure.

In some embodiments, the molecular three-dimensional structure of an anti-PD-1-Fc or an anti-PD-1-CD22 antibody can be predicted based on X-ray crystallography, and/or cryo-EM, and/or using structure prediction algorithms (e.g., machine learning algorithms) known in the art, such as AlphaFold or RaptorX. In some embodiments, the structure prediction algorithm is a computational method that is used to predict three-dimensional (3D) antibody structures based on a given nucleic acid or amino acid sequence. In some embodiments, the structure prediction algorithm predicts the 3D coordinates of all heavy atoms for a given antibody using a nucleic acid or amino acid sequence and/or aligned sequences of homologues as inputs. In some embodiments, the structure of an anti-PD-1-Fc or an anti-PD-1-CD22 antibody is predicted using a combination of methods, e.g., using a combination of AlphaFold (or any other structure prediction algorithm known in the art) and X-ray crystallography or cryo-EM. In some embodiments, the structure prediction is improved by combining the use of AlphaFold (or any other structure prediction algorithm known in the art) and X-ray crystallography or cryo-EM. In some embodiments, the structure of an anti-PD-1-Fc or an anti-PD-1-CD22 antibody is predicted by using a computational structure prediction algorithm (e.g., AlphaFold or RaptorX) and the structure prediction of the anti-PD-1-Fc or the anti-PD-1-CD22 antibody is then refined by using X-ray crystallography or cryo-EM. In some embodiments, the anti-PD-1-Fc or the anti-PD-1-CD22 antibody comprises a three-dimensional structure that is similar to the three-dimensional structure of anti-PD-1-Fc that comprises SEQ ID NOs: 2 and 3 or an anti-PD-1-CD22 antibody that comprises SEQ ID NOs: 11 and 3 or SEQ ID NOs: 12 and 3.

In some embodiments, the anti-PD-1-Fc antibody comprises a three-dimensional structure that is similar to the three-dimensional structure of anti-PD-1-Fc that comprises a heavy chain comprising SEQ ID NO: 2, 14, 16, 18, 20, 22, 24, 26, 28, 30, 72, or 74 and a light chain comprising SEQ ID NO: 3, 15, 17, 19, 21, 23, 25, 27, 29, 31, 73, or 75. In some embodiments, the anti-PD-1-CD22 antibody comprises a three-dimensional structure that is similar to the three-dimensional structure of anti-PD-1-CD22 that comprises a heavy chain comprising SEQ ID NO: 11, 12, 32, 33, 34, 35, 36, 37, 38, 39, 40, 41, 42, 43, 44, 45, 46, 47, 48, or 49 or the VH of SEQ ID NOs: 72 or 74 and a CD22 domain and a light chain comprising SEQ ID NO: 3, 15, 17, 19, 21, 23, 25, 27, 29, 31, 73 or 75. In some embodiments, the anti-PD-1-HSA antibody comprises a three-dimensional structure that is similar to the three-dimensional structure of anti-PD-1-HSA that comprises a heavy chain comprising one SEQ ID NOs: 51-60 or the VH of SEQ ID NOs: 72 or 74 and a HSA domain and a light chain comprising SEQ ID NO: 3, 15, 17, 19, 21, 23, 25, 27, 29, 31, 73, or 75. In some embodiments, the anti-PD-1-ABD antibody comprises a three-dimensional structure that is similar to the three-dimensional structure of anti-PD-1-ABD that comprises a heavy chain comprising one SEQ ID NOs: 62-71 or the VH of SEQ ID NOs: 72 or 74 and a ABD domain and a light chain comprising SEQ ID NO: 3, 15, 17, 19, 21, 23, 25, 27, 29, 31, 73, or 75.

In some embodiments, the structure prediction algorithm can be used to model the structure of a first antibody (e.g., an anti-PD-1-Fc, an anti-PD-1-CD22, and anti-PD-1-HSA, or an anti-PD-1-ABD antibody) (e.g., a reference anti-PD-1-CD22, and anti-PD-1-HSA, or an anti-PD-1-ABD antibody comprising one or more of SEQ ID NOs: 2, 11, 12, 14, 16, 18, 20, 22, 24, 26, 28, 30, 32-49, 51-60, and 62-75, or a combination thereof) and compare a predicted structure of second antibody (e.g., an anti-PD-1-Fc, an anti-PD-1-CD22, and anti-PD-1-HSA, or an anti-PD-1-ABD antibody) against the predicted structure of the first antibody such that the second antibody can be categorized in the same class as the first antibody based on its structural similarity to the first antibody. In some embodiments, a metric of structural similarity between two antibodies can be obtained based on the output of a structure prediction algorithm known in the art. In some embodiments, the metric of structural similarity between two antibodies is based on a similarity distance.

In some embodiments, the structure of the anti-PD-1-Fc antibody allows the anti-PD-1-Fc antibody to bind to PD-1. In some embodiments, disclosed herein is a new class of anti-PD-1-Fc antibodies which comprise structural similarity to one another such that the new class of anti-PD-1-Fc antibodies are capable of binding to PD-1. In some embodiments, the anti-PD-1-Fc antibody comprises means for binding PD-1. In some embodiments, means for binding PD-1 comprises an anti-PD-1-Fc antibody that comprises a heavy chain comprising SEQ ID NO: 2, 14, 16, 18, 20, 22, 24, 26, 28, 30, 72, or 74 and a light chain comprising SEQ ID NO: 3, 15, 17, 19, 21, 23, 25, 27, 29, 31, 73, or 75.

In some embodiments, the structure of the anti-PD-1-CD22 antibody allows the anti-anti-PD-1-CD22 antibody to bind to PD-1. In some embodiments, disclosed herein is a new class of anti-PD-1-CD22 antibodies which comprise structural similarity to one another such that the new class of anti-PD-1-CD22 antibodies are capable of binding to PD-1. In some embodiments, the anti-PD-1-CD22 antibody comprises means for binding PD-1. In some embodiments, means for binding PD-1 comprises an anti-PD-1-CD22 antibody that comprises a heavy chain comprising one of SEQ ID NO: 11, 12, and 32-49 or the VH of SEQ ID NOs: 72 or 74 and a CD22 domain, and a light chain comprising SEQ ID NO: 3, 15, 17, 19, 21, 23, 25, 27, 29, 31, 73, or 75.

In some embodiments, the structure of the anti-PD-1-HSA antibody allows the anti-anti-PD-1-HSA antibody to bind to PD-1. In some embodiments, disclosed herein is a new class of anti-PD-1-HSA antibodies which comprise structural similarity to one another such that the new class of anti-PD-1-HSA antibodies are capable of binding to PD-1. In some embodiments, the anti-PD-1-HSA antibody comprises means for binding PD-1. In some embodiments, means for binding PD-1 comprises an anti-PD-1-HSA antibody that comprises a heavy chain comprising one of SEQ ID NOS: 51-60 or the VH of SEQ ID NOs: 72 or 74 and a HSA domain, and a light chain comprising SEQ ID NO: 3, 15, 17, 19, 21, 23, 25, 27, 29, 31, 73, or 75.

In some embodiments, the structure of the anti-PD-1-ABD antibody allows the anti-anti-PD-1-ABD antibody to bind to PD-1. In some embodiments, disclosed herein is a new class of anti-PD-1-ABD antibodies which comprise structural similarity to one another such that the new class of anti-PD-1-ABD antibodies are capable of binding to PD-1. In some embodiments, the anti-PD-1-ABD antibody comprises means for binding PD-1. In some embodiments, means for binding PD-1 comprises an anti-PD-1-ABD antibody that comprises a heavy chain comprising one of SEQ ID NOS: 62-71 or the VH of SEQ ID NOs: 72 or 74 and a

ABD domain, and a light chain comprising SEQ ID NO: 3, 15, 17, 19, 21, 23, 25, 27, 29, 31, 73, or 75.

### Anti-PD-1 Antibodies Without Additional Domain

In some embodiments, the monoclonal anti-PD-1 antibody does not contain an additional domain and is about 140 kD in size. In some embodiments, the amino acid sequence of the anti-PD-1 antibody heavy chain comprises the VH domain of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74. In some embodiments, the amino acid sequence of the anti-PD-1 antibody light chain comprises SEQ ID NO: 3, 15, 17, 19, 21, 23, 25, 27, 29, 31, 73, or 75.

In some embodiments, the amino acid sequence of the anti-PD-1 antibody heavy chain comprises SEQ ID NO: 1 immediately followed by SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74. In some embodiments, the signal peptide is cleaved during post-translational modifications that occur in vitro or in vivo.

In some embodiments, the amino acid sequence of the anti-PD-1 antibody light chain (without the signal peptide) comprises SEQ ID NO: 3, 15, 17, 19, 21, 23, 25, 27, 29, 31, 73, or 75 as provided above for the anti-PD-1 antibody. In some embodiments, the amino acid sequence of the anti-PD-1 antibody light chain comprises SEQ ID NO: 1 immediately followed by SEQ ID NO: 3, 15, 17, 19, 21, 23, 25, 27, 29, 31, 73, or 75. In some embodiments, the signal peptide is cleaved during post-translational modifications that occur in vitro or in vivo.

In some embodiments, the monoclonal antibody comprises a first and third chain each comprises the VH domain of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74 and a second and fourth chains each comprises SEQ ID NO: 3, 15, 17, 19, 21, 23, 25, 27, 29, 31, 73, or 75. In some embodiments, the first and second chains are linked by one or more covalent disulfide bonds and the third and fourth chains are linked by one or more covalent disulfide bonds. In some embodiments, the first and third chains are linked by one or more disulfide bonds. In some embodiments, the monoclonal antibody is about 140 kD.

In some embodiments, the signal peptide of the anti-PD-1 antibody heavy or light chain comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-PD-1 antibody heavy chain comprises an amino acid sequence 80, 81, 82, 83, 84, 85, 86, 87, 88, 89, 90, 91, 92, 93, 94, 95, 96, 97, 98 or 99% identical the VH domain of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74. In some embodiments, the anti-PD-1 antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 3, 15, 17, 19, 21, 23, 25, 27, 29, 31, 73, or 75. In some embodiments, the anti-PD-1 antibody comprises an amino acid sequence comprising the CDRs of the variable heavy chain domain and the CDRs of the variable light chain domain of the anti-PD-1 antibody, wherein the CDR sequences are indicated above (in bold and underline), in FIG. 11, or in Tables 1 and 3 for SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, 74 3, 15, 17, 19, 21, 23, 25, 27, 29, 31, 73, or 75. In some embodiments, the framework regions (FRs) of the variable heavy chain domain and the FRs of the variable light chain domain of the anti-PD-1 antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, 74, 3, 15, 17, 19, 21, 23, 25, 27, 29, 31, 73, or 75, or the FRs in Tables 2 and 4.

In some embodiments, one or more amino acids of an amino acid sequence encoding one or more CDRs of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, 74, 3, 15, 17, 19, 21, 23, 25, 27, 29, 31, 73, or 75 is substituted. In some embodiments, one or more amino acids of an amino acid sequence encoding one or more variable heavy chain CDRs of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, 74, 3, 15, 17, 19, 21, 23, 25, 27, 29, 31, 73, or 75 is substituted. In some embodiments, one or more amino acids of an amino acid sequence encoding one or more variable light chain CDRs of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, 74, 3, 15, 17, 19, 21, 23, 25, 27, 29, 31, 73, or 75 is substituted.

In some embodiments the monoclonal antibody is a scFv-Fc antibody comprising a first and second chain that associate together, each chain comprising a variable heavy chain (VH) domain of SEQ ID NO: 2, 14, 16, 18, 20, 22, 24, 26, 28, 30, 72, or 74, a linker

(SEQ ID NO: 13)
(e.g., GGGGSGGGGSGGGGS),

a variable light chain (VL) domain of SEQ ID NO: 3, 15, 17, 19, 21, 23, 25, 27, 29, 31, 73, or 75, and an Fc domain of SEQ ID NO: 2.

### Non-Limiting Embodiments of the Subject Matter

In certain aspects, the present application provides a monoclonal antibody or a fragment thereof, comprising: a first arm comprising a first variable heavy chain domain and a first variable light chain domain, wherein a portion of the first arm is capable of binding to a portion of a PD-1 protein; and a second arm comprising a second variable heavy chain domain and a second variable light chain domain, wherein a portion of the second arm is capable of binding to a portion of the PD-1 protein, wherein the first and second arms each further comprise a fragment crystallizable (Fc) domain wherein the first and second arms each further comprise at least one additional domain.

In some embodiments, the first and second arms each further comprise a CH1 domain, a hinge domain, and a CL domain.

In some embodiments, the at least one additional domain comprises a second Fc domain. In some embodiments, the first and second arms each further comprise one additional domain comprising a second Fc domain. In some embodiments, the at least one additional domain comprises a CD22 domain, a human serum albumin (HSA), or an albumin binding domain (ABD).

In some embodiments, the first and second arms each further comprise one additional domain comprising a CD22 domain. In some embodiments, the CD22 domain comprises the SEQ ID NO: 8, or a fragment thereof. In some embodiments, the CD22 domain comprises SEQ ID NO: 9. In some embodiments, the CD22 domain comprises SEQ ID NO: 10. In some embodiments, the monoclonal antibody comprises an ABD domain, wherein the ABD domain comprises any one of SEQ ID NOs: 62-71, or a fragment thereof. In some embodiments, the monoclonal antibody comprises a HSA domain, wherein the HSA domain comprises any one of SEQ ID NOs: 51-60, or a fragment thereof.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first PD-1 binding site, and the second variable heavy chain domain and second variable light chain domain form a second PD-1 binding site.

In some embodiments, the first and second PD-1 binding sites are the same.

In some embodiments, the first and third polypeptide chain each further encode a hinge domain, a CH1 domain, the Fc domain, and the at least one additional domain, and the second and fourth polypeptide chain each further encode a CL domain. In some embodiments, the first and third polypeptide chains comprise the same sequence, and the second and fourth polypeptide chains comprise the same sequence.

In some embodiments, the first variable heavy chain domain comprises an amino acid sequence of the variable heavy chain portion of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74, and the first variable light chain domain comprises an amino acid sequence of the variable light chain portion of SEQ ID NO: 3, 15, 17, 19, 21, 23, 25, 27, 29, 31, 73, or 75.

In some embodiments, the first arm and second arm each comprises an amino acid sequence comprising SEQ ID NO: 2 and SEQ ID NO: 3. In some embodiments, the first arm and second arm each comprises an amino acid sequence comprising SEQ ID NO: 11 and SEQ ID NO: 3. In some embodiments, the first arm and second arm each comprises an amino acid sequence comprising SEQ ID NO: 12 and SEQ ID NO: 3.

In some embodiments, the first and third polypeptide chain each comprises an amino acid sequence comprising SEQ ID NO: 2 and the second and fourth polypeptide chain each comprises an amino acid sequence comprising SEQ ID NO: 3. In some embodiments, the first and third polypeptide chain each comprises an amino acid sequence comprising SEQ ID NO: 11 and the second and fourth polypeptide chain each comprises an amino acid sequence comprising SEQ ID NO: 3. In some embodiments, the first and third polypeptide chain each comprises an amino acid sequence comprising SEQ ID NO: 12 and the second and fourth polypeptide chain each comprises an amino acid sequence comprising SEQ ID NO: 3.

In some embodiments, the first and second polypeptide chains are linked by one or more covalent disulfide bonds and the third and fourth polypeptide chains are linked by one or more covalent disulfide bonds.

In some embodiments, the first and third polypeptide chains are linked by one or more covalent disulfide bonds.

In some embodiments, the monoclonal antibody is capable of localizing a PD-1 protein away from an immune synapse.

In some embodiments, the monoclonal antibody is capable of disrupting downstream signaling of a PD-1 mediated response in a T cell.

In some embodiments, the monoclonal antibody is capable of enhancing T cell function.

In some embodiments, the PD-1 protein is located on a T cell, and the monoclonal antibody is capable of preventing the PD-1 protein from binding to a PDL-1 protein or a PDL-2 protein on a tumor cell.

In some embodiments, the monoclonal antibody is capable of inducing a cytokine secretion in a T cell. In some embodiments, the cytokine secretion is a secretion of IL-2.

In some embodiments, the monoclonal antibody is larger than 140 kD in size. In some embodiments, the monoclonal antibody is about 160 kD in size. In some embodiments, the monoclonal antibody is about 200 kD in size. In some embodiments, the monoclonal antibody is about 230 kD in size.

In certain aspects, the present application provides a pharmaceutical composition comprising: a monoclonal antibody as described above and a pharmaceutically acceptable carrier.

In certain aspects, the present application provides a method of preventing or treating cancer in a subject comprising administering to the subject an effective amount of the composition of the composition described above.

In some embodiments, the cancer is selected from colorectal cancer, lung cancer, bladder cancer, breast cancer, cervical cancer, kidney cancer, leukemia, Hodgkin lymphoma, non-Hodgkin lymphoma, prostate cancer, skin cancer (e.g., melanoma), head and neck cancer, endometrial cancer, colon cancer, rectal cancer, liver cancer, thyroids cancer, esophageal cancer, renal cell cancer, and a combination thereof.

In certain aspects, the present application provides a method of preventing or treating a viral infection in a subject comprising administering to the subject an effective amount of the composition described above. In some embodiments, the viral infection is HIV.

In certain aspects, the present application provides a kit for generating a monoclonal antibody or fragment thereof, the kit comprising one or more vectors comprising a polynucleotide sequence encoding any of the monoclonal antibodies described above.

In certain aspects, the present application provides a kit for generating a monoclonal antibody or fragment thereof, the kit comprising: a first vector comprising a polynucleotide sequence encoding the first polypeptide chain of the monoclonal antibody with the first variable heavy chain domain of the first arm encoded by a first polypeptide chain; and a second vector comprising a polynucleotide sequence encoding the second polypeptide chain of the monoclonal antibody with the first variable light chain domain of the first arm encoded by a second polypeptide chain, the first variable heavy chain domain and first variable light chain domain form a first PD-1 binding site.

In some embodiments, the first and second PD-1 binding sites are the same. In some embodiments, the first and the third polypeptide chains each further encode a hinge domain, a CH1 domain, the Fc domain, and the at least one additional domain and the second and fourth polypeptide chain each further encode a CL domain. In some embodiments, the first and third polypeptide chains comprise the same sequence, and the second and fourth polypeptide chains comprise the same sequence. In some embodiments, the first variable heavy chain domain comprises an amino acid sequence of the variable heavy chain portion of SEQ ID NO: 22, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74, wherein the first variable light chain domain comprises an amino acid sequence of the variable light chain portion of SEQ ID NO: 3, 15, 17, 19, 21, 23, 25, 27, 29, 31, 73, or 75.

In some embodiments, the first arm and second arm each comprises an amino acid sequence comprising SEQ ID NO: 2 and SEQ ID NO: 3. In some embodiments, the first arm and second arm each comprises an amino acid sequence comprising SEQ ID NO: 11 and SEQ ID NO: 3. In some embodiments, the first arm and second arm each comprises an amino acid sequence comprising SEQ ID NO: 12 and SEQ ID NO: 3. In some embodiments, the first and third polypeptide chain each comprises an amino acid sequence comprising SEQ ID NO: 2 and the second and fourth polypeptide chain each comprises an amino acid sequence comprising SEQ ID NO: 3. In some embodiments, the first and third polypeptide chain each comprises an amino acid sequence comprising SEQ ID NO: 11 and the second and fourth polypeptide chain each comprises an amino acid sequence comprising SEQ ID NO: 3. In some embodiments, the first and third polypeptide chain each comprises an amino acid sequence comprising SEQ ID NO: 12 and the second and fourth polypeptide chain each comprises an amino acid sequence comprising SEQ ID NO: 3.

In some embodiments, the first vector and the second vector are the same vector. In some embodiments, the first vector and the second vector are two different vectors.

In certain aspects, the present application provides one or more host cells comprising: one or more vectors comprising a polynucleotide sequence encoding any of the monoclonal antibodies described above.

In certain aspects, the present application provides one or more host cells comprising: a first vector comprising a polynucleotide sequence encoding the first polypeptide chain of the monoclonal antibody with the first variable heavy chain domain of the first arm encoded by a first polypeptide chain; and a second vector comprising a polynucleotide sequence encoding the second polypeptide chain of the monoclonal antibody with the first variable light chain domain of the first arm encoded by a second polypeptide chain, the first variable heavy chain domain and first variable light chain domain form a first PD-1 binding site.

In some embodiments, the first and second PD-1 binding sites are the same. In some embodiments, the first and the third polypeptide chains each further encode a hinge domain, a CH1 domain, the Fc domain, and the at least one additional domain and the second and fourth polypeptide chain each further encode a CL domain. In some embodiments, the first and third polypeptide chains comprise the same sequence and the second and fourth polypeptide chains comprise the same sequence. In some embodiments, the first variable heavy chain domain comprises an amino acid sequence of the variable heavy chain portion of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74, wherein the first variable light chain domain comprises an amino acid sequence of the variable light chain portion of SEQ ID NO: 3, 15, 17, 19, 21, 23, 25, 27, 29, 31, 73, or 75.

In some embodiments, the first arm and second arm each comprises an amino acid sequence comprising SEQ ID NO: 2 and SEQ ID NO: 3. In some embodiments, the first arm and second arm each comprises an amino acid sequence comprising SEQ ID NO: 11 and SEQ ID NO: 3. In some embodiments, the first arm and second arm each comprises an amino acid sequence comprising SEQ ID NO: 12 and SEQ ID NO: 3. In some embodiments, the first and third polypeptide chain each comprises an amino acid sequence comprising SEQ ID NO: 2 and the second and fourth polypeptide chain each comprises an amino acid sequence comprising SEQ ID NO: 3. In some embodiments, the first and third polypeptide chain each comprises an amino acid sequence comprising SEQ ID NO: 11 and the second and fourth polypeptide chain each comprises an amino acid sequence comprising SEQ ID NO: 3. In some embodiments, the first and third polypeptide chain each comprises an amino acid sequence comprising SEQ ID NO: 12 and the second and fourth polypeptide chain each comprises an amino acid sequence comprising SEQ ID NO: 3.

In some embodiments, the first vector and the second vector are the same vector. In some embodiments, the first vector and the second vector are two different vectors.

In some embodiments, the one or more host cells are cultured under conditions suitable for an expression of the one or more vectors; and recovering the monoclonal antibody or fragment thereof. In some embodiments, the one or more host cells are cultured under conditions suitable for an expression of the first vector and the second vector; and recovering the monoclonal antibody or fragment thereof.

In certain aspects, the present application provides a composition comprising: one or more vectors comprising a polynucleotide sequence encoding any of the monoclonal antibodies described above.

In certain aspects, the present application provides a composition comprising: a first vector comprising a polynucleotide sequence encoding the first polypeptide chain of the monoclonal antibody with the first variable heavy chain domain of the first arm encoded by a first polypeptide chain; and a second vector comprising a polynucleotide sequence encoding the second polypeptide chain of the monoclonal antibody with the first variable light chain domain of the first arm encoded by a second polypeptide chain, the first variable heavy chain domain and first variable light chain domain form a first PD-1 binding site.

In some embodiments, the first and second PD-1 binding sites are the same. In some embodiments, the first and the third polypeptide chains each further encode a hinge domain, a CH1 domain, the Fc domain, and the at least one additional domain and the second and fourth polypeptide chain each further encode a CL domain. In some embodiments, the first and third polypeptide chains comprise the same sequence and the second and fourth polypeptide chains comprise the same sequence. In some embodiments, the first variable heavy chain domain comprises an amino acid sequence of the variable heavy chain portion of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74, wherein the first variable light chain domain comprises an amino acid sequence of the variable light chain portion of SEQ ID NO: 3, 15, 17, 19, 21, 23, 25, 27, 29, 31, 73, or 75.

In some embodiments, the first arm and second arm each comprises an amino acid sequence comprising SEQ ID NO: 2 and SEQ ID NO: 3. In some embodiments, the first arm and second arm each comprises an amino acid sequence comprising SEQ ID NO: 11 and SEQ ID NO: 3. In some embodiments, the first arm and second arm each comprises an amino acid sequence comprising SEQ ID NO: 12 and SEQ ID NO: 3. In some embodiments, the first and third polypeptide chain each comprises an amino acid sequence comprising SEQ ID NO: 2 and the second and fourth polypeptide chain each comprises an amino acid sequence comprising SEQ ID NO: 3. In some embodiments, the first and third polypeptide chain each comprises an amino acid sequence comprising SEQ ID NO: 11 and the second and fourth polypeptide chain each comprises an amino acid sequence comprising SEQ ID NO: 3. In some embodiments, the first and third polypeptide chain each comprises an amino acid sequence comprising SEQ ID NO: 12 and the second and fourth polypeptide chain each comprises an amino acid sequence comprising SEQ ID NO: 3.

In some embodiments, the first vector and the second vector are the same vector. In some embodiments, the first vector and the second vector are two different vectors.

In certain aspects, the present application provides a means for binding a portion of a PD-1 protein. In some embodiments, the means comprises a monoclonal antibody comprising a domain for increasing the size of the monoclonal antibody over 140 kD. In some embodiments, the monoclonal antibody or a fragment thereof comprises: a first arm comprising a first variable heavy chain domain and a first variable light chain domain, wherein a portion of the first arm is capable of binding to the portion of the PD-1 protein; and a second arm comprising a second variable heavy chain domain and a second variable light chain domain, wherein a portion of the second arm is capable of binding to the portion of the PD-1 protein wherein the first and second arms each further comprise a fragment, crystallizable (Fc) domain wherein the first and second arms each further comprise the domain for increasing the size of the monoclonal antibody over 140 kD.

In some embodiments, the first and second arms each further comprise a CH1 domain, a hinge domain, and a CL domain.

In some embodiments, the domain for increasing the size of the monoclonal antibody over 140 kD comprises a second Fc domain. In some embodiments, the domain for increasing the size of the monoclonal antibody over 140 kD comprises a CD22 domain.

In some embodiments, the first and second arms each further comprise one additional domain comprising a CD22 domain, a human serum albumin (HSA), or an albumin binding domain (ABD). In some embodiments, the CD22 domain comprises the SEQ ID NO: 8, or a fragment thereof. In some embodiments, the CD22 domain comprises SEQ ID NO: 9. In some embodiments, the CD22 domain comprises SEQ ID NO: 10. In some embodiments, the monoclonal antibody comprises an ABD domain, wherein the ABD domain comprises any one of SEQ ID NOs: 62-71, or a fragment thereof. In some embodiments, the monoclonal antibody comprises a HSA domain, wherein the HSA domain comprises any one of SEQ ID NOs: 51-60, or a fragment thereof.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first PD-1 binding site and wherein the second variable heavy chain domain and second variable light chain domain form a second PD-1 binding site. In some embodiments, the first and second PD-1 binding sites are the same.

In some embodiments, the first and third polypeptide chain each further encode a hinge domain, a CH1 domain, the Fc domain, and the at least one additional domain for increasing the size of the monoclonal antibody over 140 kD, and wherein the second and fourth polypeptide chain each further encode a CL domain. In some embodiments, the first and third polypeptide chains comprise the same sequence and the second and fourth polypeptide chains comprise the same sequence.

In some embodiments, the first variable heavy chain domain comprises an amino acid sequence of the variable heavy chain portion of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74 wherein the first variable light chain domain comprises an amino acid sequence of the variable light chain portion of SEQ ID NO: 3, 15, 17, 19, 21, 23, 25, 27, 29, 31, 73, or 75.

In some embodiments, the first arm and second arm each comprises an amino acid sequence comprising SEQ ID NO: 2 and SEQ ID NO: 3. In some embodiments, the first arm and second arm each comprises an amino acid sequence comprising SEQ ID NO: 11 and SEQ ID NO: 3. In some embodiments, the first arm and second arm each comprises an amino acid sequence comprising SEQ ID NO: 12 and SEQ ID NO: 3.

In some embodiments, the monoclonal antibody is capable of localizing a PD-1 protein away from an immune synapse.

In some embodiments, the monoclonal antibody is capable of disrupting downstream signaling of a PD-1 mediated response in a T cell. In some embodiments, the monoclonal antibody is capable of enhancing T cell function.

In some embodiments, the PD-1 protein is located on a T cell, and wherein the monoclonal antibody is capable of preventing the PD-1 protein from binding to a PDL-1 protein or a PDL-2 protein on a tumor cell.

In some embodiments, the monoclonal antibody is capable of inducing a cytokine secretion in a T cell. In some embodiments, the cytokine secretion is a secretion of IL-2.

In some embodiments, the monoclonal antibody is about 200 kD in size.

### Compositions

In some embodiments, a prophylactic or therapeutic composition of this disclosure comprises one or more antibodies (or one or more polynucleotides encoding one or more antibodies) and is administered in a pharmaceutical composition that includes a pharmaceutically acceptable carrier. In some embodiments, the prophylactic or therapeutic composition is comprised of one or more antibodies (or one or more polynucleotides encoding one or more antibodies) comprising SEQ ID NOs: 2 and 3 (e.g., antibody “anti-PD-1-Fc”), SEQ ID NOs: 11 and 3, (e.g., “anti-PD-1-CD22 (505-687)”), or comprising SEQ ID NOs: 12 and 3 (“anti-PD-1-CD22 (593-687)”), or comprising SEQ ID NOs: 14 and 15, or comprising SEQ ID NOs: 16 and 17, or comprising SEQ ID NOs: 18 and 19, or comprising SEQ ID NOs: 20 and 21, or comprising SEQ ID NOs: 22 and 23, or comprising SEQ ID NOs: 24 and 25, or comprising SEQ ID NOs: 26 and 27, or comprising SEQ ID NOs: 28 and 29, or comprising SEQ ID NOs: 30 and 31. In some embodiments, the prophylactic or therapeutic composition is comprised of one or more antibodies (or one or more polynucleotides encoding one or more antibodies) comprising a heavy chain comprising one of SEQ ID NOs: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74 and a light chain comprising SEQ ID NO: 3, 15, 17, 19, 21, 23, 25, 27, 29, 31, 73, or 75. In some embodiments, the pharmaceutical composition is in the form of a spray, aerosol, gel, solution, emulsion, nanoparticle (e.g., lipid nanoparticle), or suspension.

The composition is preferably administered to a subject with a pharmaceutically acceptable carrier. Typically, in some embodiments, an appropriate amount of a pharmaceutically acceptable salt is used in the formulation, which in some embodiments can render the formulation isotonic.

In certain embodiments, the one or more antibodies (or one or more polynucleotides encoding one or more antibodies) are provided as a composition comprising any one of the antibodies described herein (e.g., “anti-PD-1-Fc”, anti-PD-1 antibodies referred to as clones 01, 02. 03, 07, 09, 51, 55, 79, and 80 with an Fc, “anti-PD-1-CD22 (505-687)”, “anti-PD-1-CD22 (593-687)”, anti-PD-1 antibodies referred to as clones 01, 02. 03, 07, 09, 51, 55, 79, and 80 with CD22 antibody, anti-PD-1-HSA, or anti-PD-1-ABD antibodies) and a pharmaceutically acceptable carrier. In certain embodiments, the composition further comprises an adjuvant. In certain embodiments, the antibodies are conjugated with other molecules to increase their effectiveness as is known by those practiced in the art.

In some embodiments, the pharmaceutically acceptable carrier is selected from the group consisting of saline, Ringer's solution, dextrose solution, and a combination thereof. Other suitable pharmaceutically acceptable carriers known in the art are contemplated. Suitable carriers and their formulations are described in Remington's Pharmaceutical Sciences, 2005, Mack Publishing Co. The pH of the solution is preferably from about 5 to about 8, and more preferably from about 7 to about 7.5. The formulation may also comprise a lyophilized powder. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of antibodies being administered.

The phrase pharmaceutically acceptable carrier as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier is acceptable in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term carrier denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency. The composition may also include additional agents such as an isotonicity agent, a preservative, a surfactant, and, a divalent cation, preferably, zinc.

The composition can also include an excipient, or an agent for stabilization of an antibody composition, such as a buffer, a reducing agent, a bulk protein, amino acids (such as e.g., glycine or praline) or a carbohydrate. Typical carbohydrates useful in formulating compositions include but are not limited to sucrose, mannitol, lactose, trehalose, or glucose.

Surfactants may also be used to prevent soluble and insoluble aggregation and/or precipitation of antibodies included in the composition. Suitable surfactants include but are not limited to sorbitan trioleate, soya lecithin, and oleic acid. In certain cases, solution aerosols are preferred using solvents such as ethanol. Thus, formulations including antibodies can also include a surfactant that can reduce or prevent surface-induced aggregation of antibodies by atomization of the solution in forming an aerosol. Various conventional surfactants can be employed, such as polyoxyethylene fatty acid esters and alcohols, and polyoxyethylene sorbitol fatty acid esters. Amounts will generally range between 0.001% and 4% by weight of the formulation. In some embodiments, surfactants used with the present disclosure are polyoxyethylene sorbitan mono-oleate, polysorbate 80, polysorbate 20. Additional agents known in the art can also be included in the composition.

In some embodiments, the pharmaceutical compositions and dosage forms further comprise one or more compounds that reduce the rate by which an active ingredient will decay, or the composition will change in character. So called stabilizers or preservatives may include, but are not limited to, amino acids, antioxidants, pH buffers, or salt buffers. Nonlimiting examples of antioxidants include butylated hydroxy anisole (BHA), ascorbic acid and derivatives thereof, tocopherol and derivatives thereof, butylated hydroxy anisole and cysteine. Nonlimiting examples of preservatives include parabens, such as methyl or propyl p-hydroxybenzoate and benzalkonium chloride. Additional nonlimiting examples of amino acids include glycine or proline.

The present invention also teaches the stabilization (preventing or minimizing thermally or mechanically induced soluble or insoluble aggregation and/or precipitation of an inhibitor protein) of liquid solutions containing antibodies at neutral pH or less than neutral pH by the use of amino acids including proline or glycine, with or without divalent cations resulting in clear or nearly clear solutions that are stable at room temperature or preferred for pharmaceutical administration.

In one embodiment, the composition is a pharmaceutical composition of single unit or multiple unit dosage forms. Pharmaceutical compositions of single unit or multiple unit dosage forms of the invention comprise a prophylactically or therapeutically effective amount of one or more compositions (e.g., a compound of the invention, or other prophylactic or therapeutic agent), typically, one or more vehicles, carriers, or excipients, stabilizing agents, and/or preservatives. Preferably, the vehicles, carriers, excipients, stabilizing agents and preservatives are pharmaceutically acceptable.

In some embodiments, the pharmaceutical compositions and dosage forms comprise anhydrous pharmaceutical compositions and dosage forms. Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Suitable vehicles are well known to those skilled in the art of pharmacy, and non-limiting examples of suitable vehicles include glucose, sucrose, starch, lactose, gelatin, rice, silica gel, glycerol, talc, sodium chloride, dried skim milk, propylene glycol, water, sodium stearate, ethanol, and similar substances well known in the art. Saline solutions and aqueous dextrose and glycerol solutions can also be employed as liquid vehicles. Whether a particular vehicle is suitable for incorporation into a pharmaceutical composition or dosage form depends on a variety of factors well known in the art including, but not limited to, the way in which the dosage form will be administered to a patient and the specific active ingredients in the dosage form. Pharmaceutical vehicles can be sterile liquids, such as water and oils, including those of petroleum, animal, vegetable or synthetic origin, such as peanut oil, soybean oil, mineral oil, sesame oil and the like.

The invention also provides that a pharmaceutical composition can be packaged in a hermetically sealed container such as an ampoule or sachette indicating the quantity. In one embodiment, the pharmaceutical composition can be supplied as a dry sterilized lyophilized powder in a delivery device suitable for administration to the lower airways of a patient. The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for administration may be in the form of powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention (e.g., antibodies) as an active ingredient.

A liquid composition herein can be used as such with a delivery device, or they can be used for the preparation of pharmaceutically acceptable formulations comprising antibodies that are prepared for example by the method of spray drying. The methods of spray freeze-drying proteins for pharmaceutical administration disclosed in Maa et al., Curr. Pharm. Biotechnol., 2001, 1, 283-302, are incorporated herein. In another embodiment, the liquid solutions herein are freeze spray dried and the spray-dried product is collected as a dispersible peptide-containing powder that is therapeutically effective when administered to an individual.

The compounds and pharmaceutical compositions of the present invention can be employed in combination therapies, that is, the compounds and pharmaceutical compositions can be administered concurrently with, prior to, or subsequent to, one or more other desired therapeutics or medical procedures (e.g., antibodies can be used in combination treatment with another treatment such as antivirals or with a vaccine, and/or another treatment). The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another therapeutic or prophylactic).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The current invention provides for dosage forms comprising peptides suitable for treating cancer or other diseases. The dosage forms can be formulated, e.g., as sprays, aerosols, nanoparticles, liposomes, or other forms known to one of skill in the art. See, e.g., Remington's Pharmaceutical Sciences; Remington: The Science and Practice of Pharmacy supra; Pharmaceutical Dosage Forms and Drug Delivery Systems by Howard C., Ansel et al., Lippincott Williams & Wilkins; 7th edition (Oct. 1, 1999).

Generally, a dosage form used in the acute treatment of a disease may contain larger amounts of one or more of the active ingredients it comprises than a dosage form used in the chronic treatment of the same disease. In addition, the prophylactically and therapeutically effective dosage form may vary among different conditions. For example, a therapeutically effective dosage form may contain one or more antibodies that have an appropriate therapeutic action when intending to treat cancer or a viral infection such as HIV. On the other hand, a different effective dosage may contain one or more antibodies that have an appropriate prophylactic action when intending to prevent cancer or an infection caused by a virus (e.g, HIV). These and other ways in which specific dosage forms encompassed by this invention will vary from one another and will be readily apparent to those skilled in the art. See, e.g.,

Remington's Pharmaceutical Sciences, 2005, Mack Publishing Co.; Remington: The Science and Practice of Pharmacy by Gennaro, Lippincott Williams & Wilkins; 20th edition (2003); Pharmaceutical Dosage Forms and Drug Delivery Systems by Howard C. Ansel et al., Lippincott Williams & Wilkins; 7th edition (Oct. 1, 1999); and Encyclopedia of Pharmaceutical Technology, edited by Swarbrick, J. & J. C. Boylan, Marcel Dekker, Inc., New York, 1988, which are incorporated herein by reference in their entirety.

The pH of a pharmaceutical composition or dosage form may also be adjusted to improve delivery and/or stability of one or more active ingredients. Similarly, the polarity of a solvent carrier, its ionic strength, or tonicity can be adjusted to improve delivery. Compounds such as stearates can also be added to pharmaceutical compositions or dosage forms to alter advantageously the hydrophilicity or lipophilicity of one or more active ingredients to improve delivery. In this regard, stearates can also serve as a lipid vehicle for the formulation, as an emulsifying agent or surfactant, and as a delivery enhancing or penetration-enhancing agent. Different salts, hydrates, or solvates of the active ingredients can be used to adjust further the properties of the resulting composition.

Compositions can be formulated with appropriate carriers and adjuvants using techniques to yield compositions suitable for prophylaxis or treatment. The compositions can include an adjuvant, such as, for example but not limited to, alum, poly IC, MF-59, squalene-based adjuvants, or liposomal based adjuvants suitable for prophylaxis or treatment.

In some embodiments, the antibodies described herein are encoded by nucleic acids which are prepared in a mRNA-LNP or a DNA-LNP formulation for administration to a subject. Antibody Production

The antibodies disclosed herein can be produced by any method known in the art. In some embodiments, the antibodies disclosed herein are produced by culturing a cell transfected or transformed with a vector comprising nucleic acid sequences encoding an antibody described herein and isolating the antibody.

In some embodiments, antibodies are synthesized by the hybridoma culture method which results in antibodies that are not contaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques known in the art, including, for example, the hybridoma method (e.g., Kohler and Milstein., Nature, 256:495-97 (1975); Hongo et al, Hybridoma, 14 (3): 253-260 (1995), Harlow et al, Antibodies: A Laboratory Manual, (Cold Spring Harbor Laboratory Press, 2nd ed. 1988); Hammerling et al, in: Monoclonal Antibodies and T-Cell Hybridomas 563-681 (Elsevier, N. Y., 1981)), recombinant DNA methods, phage-display technologies (see, e.g., Clackson et al, Nature, 352:624-628 (1991); Marks et al, J. Mol Biol. 222:581-597 (1992); Sidhu et al, J. Mol Biol. 338 (2): 299-310 (2004); Lee et al, J. Mol Biol. 340 (5): 1073-1093 (2004); Fellouse, Proc. Natl. Acad. ScL USA 101 (34): 12467-12472 (2004); and Lee et al, J. Immunol. Methods 284 (1-2): 119-132 (2004), and technologies for producing human or humanlike antibodies in animals that have parts or all of the human immunoglobulin loci or genes encoding human immunoglobulin sequences (see, e.g., Lonberg et al, Nature 368:856-859 (1994); Morrison, Nature 368:812-813 (1994); Fishwild et al, Nature Biotechnol 14:845-851 (1996); Neuberger, Nature Biotechnol. 14:826 (1996); and Lonberg and Huszar, Intern. Rev. Immunol. 13:65-93 (1995).

In some embodiments, expression of an antibody comprises expression vector(s) containing a polynucleotide that encodes an anti-PD-1-Fc, an anti-PD-1-CD22, and anti-PD-1-HSA, or an anti-PD-1-ABD antibody. Methods that are well known to those skilled in the art can be used to construct expression vectors comprising antibody coding sequences and appropriate transcriptional and translational control signals. These methods include, for example, in vitro recombinant DNA techniques, synthetic techniques, and in vivo genetic recombination. Particular embodiments provide replicable vectors comprising a nucleotide sequence encoding an anti-PD-1-Fc or an anti-PD-1-CD22 antibody disclosed herein operably linked to a promoter. In preferred embodiments, such vectors may include a nucleotide sequence encoding the heavy chain of an antibody molecule (or fragment thereof), a nucleotide sequence encoding the light chain of an antibody (or fragment thereof), or both the heavy and light chain.

The polynucleotide encoding the antibody may be modified, for example, by substituting the coding sequence for human heavy- and light-chain constant domains in place of the homologous murine sequences (U.S. Pat. No. 4,816,567; Morrison, et al, Proc. Natl Acad. ScL USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen. The monoclonal antibodies described herein may by monovalent, the preparation of which is well known in the art. For example, one method involves recombinant expression of immunoglobulin light chain and a modified heavy chain. The heavy chain is truncated generally at any point in the Fc domain so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art. Chimeric or hybrid antibodies also may be prepared in vitro using known methods in synthetic protein chemistry, including those involving crosslinking agents.

Various expression systems for producing antibodies are known in the art, and include, prokaryotic (e.g., bacteria), plant, insect, yeast, and mammalian expression systems. Suitable cell lines, can be transformed, transduced, or transfected with nucleic acids containing coding sequences for antibodies or portions of antibodies disclosed herein in order to produce the antibody of interest. Expression vectors containing such nucleic acid sequences, which can be linked to at least one regulatory sequence in a manner that allows expression of the nucleotide sequence in a host cell, can be introduced via methods known in the art. Practitioners in the art understand that designing an expression vector can depend on factors, such as the choice of host cell to be transfected and/or the type and/or amount of desired protein to be expressed. Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication. For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (e.g., antibodies and fragments thereof) is stably integrated into the genome of eukaryotic cells (for example mammalian cells), resulting in the stable expression of transfected genes. A gene that encodes a selectable marker (for example, resistance to antibiotics or drugs) can be introduced into host cells along with the gene of interest in order to identify and select clones that stably express a gene encoding a protein of interest. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired antibody molecule.

In some embodiments, the antibodies disclosed herein are encoded in a vector for expression in a cell line. In some embodiments, a vector comprises a polynucleotide sequence that encodes an anti-PD-1-Fc, anti-PD-1-CD22, anti-PD-1-HSA, or anti-PD-1-ABD antibody and the vector is transfected into one or more cell lines for expression. In some embodiments, one or more vectors comprise polynucleotide sequences encoding a light chain and a heavy chain of the antibody. For example, in some embodiments, a first vector may comprise a polynucleotide sequence encoding a light chain, a second vector may comprise a polynucleotide sequence encoding a heavy chain, of anti-PD-1-Fc, anti-PD-1-CD22, anti-PD-1-HSA, or anti-PD-1-ABD antibody. In some embodiments, both vectors are transfected into one or more cell lines for expression. A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of protein products may be important for the function of the antibody. Different host cell strains have characteristic and specific mechanisms for the post-translational processing and modification of proteins and gene products. As such, appropriate host systems or cell lines can be chosen to ensure the correct modification and processing of the foreign antibody expressed. Thus, eukaryotic host cells possessing the cellular machinery for proper processing of the primary transcript, glycosylation, and phosphorylation of the gene product may be used.

Various culturing parameters can be used with respect to the host cell being cultured. Appropriate culture conditions for mammalian cells are well known in the art (Cleveland W L, et al., J Immunol Methods, 1983, 56 (2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available media can be utilized.

Antibodies disclosed herein can be purified from any human or non-human cell which expresses the antibody, including those which have been transfected with expression constructs that express the antibody or fragments thereof. For antibody recovery, isolation and/or purification, the cell culture medium or cell lysate is centrifuged to remove particulate cells and cell debris. The desired antibody molecule is isolated or purified away from contaminating soluble proteins and polypeptides by suitable purification techniques. Non-limiting purification methods for proteins/antibodies include: size exclusion chromatography; affinity chromatography; ion exchange chromatography; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

### Methods of Treatment

In one embodiment, the subject matter disclosed herein relates to a preventive medical treatment started after following diagnosis of a disease (e.g., cancer) in order to prevent the disease from worsening or curing the disease. In one embodiment, the subject matter disclosed herein relates to prophylaxis of subjects who are believed to be at risk for moderate or severe disease associated with cancer or have previously been diagnosed with another disease, such as cancer. In one embodiment, the subjects can be administered the pharmaceutical composition described herein. The invention contemplates using any of the antibodies produced by the systems and methods described herein. In one embodiment, the compositions described herein can be administered subcutaneously via syringe or any other suitable method know in the art.

The compound(s) or combination of compounds disclosed herein, or pharmaceutical compositions may be administered to a cell, mammal, or human by any suitable means. Non-limiting examples of methods of administration include, among others, (a) administration though oral pathways, which includes administration in capsule, tablet, granule, spray, syrup, or other such forms; (b) administration through non-oral pathways such as intraocular, intranasal, intraauricular, rectal, vaginal, intraurethral, transmucosal, buccal, or transdermal, which includes administration as an aqueous suspension, an oily preparation or the like or as a drip, spray, suppository, salve, ointment or the like; (c) administration via injection, including subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; (e) administration topically; as deemed appropriate by those of skill in the art for bringing the compound or combination of compounds disclosed herein into contact with living tissue; (f) administration via inhalation, including through aerosolized, nebulized, and powdered formulations; (g) administration through implantation; and administration via electroporation.

In some embodiments, one or more antibodies disclosed herein are prepared in a cocktail of DNA-encoding antibodies or mRNA-encoding antibodies and delivered by electroporation to a subject for in vivo expression of the encoded antibodies.

As will be readily apparent to one skilled in the art, the effective in vivo dose to be administered and the particular mode of administration will vary depending upon the age, weight and species treated, and the specific use for which the compound or combination of compounds disclosed herein are employed. The determination of effective dose levels, that is the dose levels necessary to achieve the desired result, can be accomplished by one skilled in the art using routine pharmacological methods. Typically, human clinical applications of products are commenced at lower dose levels, with dose level being increased until the desired effect is achieved. Alternatively, acceptable in vitro studies can be used to establish useful doses and routes of administration of the compositions identified by the present methods using established pharmacological methods. Effective animal doses from in vivo studies can be converted to appropriate human doses using conversion methods known in the art (e.g., see Nair A B, Jacob S. A simple practice guide for dose conversion between animals and human. Journal of basic and clinical pharmacy. 2016 March; 7(2):27.)

### Methods of Prevention

In some embodiments, the compositions prepared using methods of the invention can be used as a vaccine to promote an immune response against future disease (e.g., cancer) or infection (e.g., HIV). In some embodiments, the antibodies are neutralizing antibodies.

In some embodiments, the antibodies (or polynucleotides encoding antibodies) prepared using methods of the invention can be combined with additional pharmaceutical components.

### Dosage

A prophylactically effective or therapeutically effective amount is typically dependent on the weight of the subject being treated, the subject's physical condition, the extensiveness of the condition to be treated, and the age of the subject being treated. In general, an anti-PD-1-Fc, an anti-PD-1-CD22, an anti-PD-1-HSA, or an anti-PD-1-ABD antibody, or polynucleotides encoding one or more antibodies, disclosed herein may be administered in an amount in the range of about 10 ng/kg body weight to about 100 mg/kg body weight per dose. In some embodiments, antibodies may be administered in an amount in the range of about 50 μg/kg body weight to about 5 mg/kg body weight per dose. In some embodiments, antibodies may be administered in an amount in the range of about 100 μg/kg body weight to about 10 mg/kg body weight per dose. In some embodiments, antibodies may be administered in an amount in the range of about 100 μg/kg body weight to about 20 mg/kg body weight per dose. In some embodiments, antibodies may be administered in an amount in the range of about 0.5 mg/kg body weight to about 20 mg/kg body weight per dose. In some embodiments, antibodies may be administered in an amount in the range of about 0.5 mg/kg body weight to about 10 mg/kg body weight per dose. In some embodiments, antibodies may be administered in an amount in the range of about 1 mg/kg body weight to about 5 mg/kg body weight per dose. In some embodiments, antibodies may be administered in an amount in the range of about 0.1 mg/kg body weight to about 0.5 mg/kg body weight per dose. In some embodiments, antibodies may be administered in a dose of at least about 100 μg/kg body weight, at least about 250 μg/kg body weight, at least about 500 μg/kg body weight, at least about 750 μg/kg body weight, at least about 3 mg/kg body weight, at least about 5 mg/kg body weight, or at least about 10 mg/kg body weight.

In some methods, the dosage is adjusted to achieve a plasma antibody concentration of about 1-1000 μg/mL or about 25-300 μg/mL. In some embodiments, the dosage is adjusted to achieve a plasma antibody concentration of about 0.001 μg/mL to about 10 μg/mL. In some embodiments, the dosage is adjusted to achieve a plasma antibody concentration of about 1 μg/mL to about 10 μg/mL. In some embodiments, the dosage is adjusted to achieve a plasma antibody concentration of about 0.01 μg/mL to about 1 μg/mL. In some embodiments, the dosage is adjusted to achieve a plasma antibody concentration of about 0.01 μg/mL to about 0.1 μg/mL.

### EXAMPLES

### Example 1-Anti-PD-1-Fc and Anti-PD-1-CD22 Antibodies

Targeting immune checkpoint receptors on T cells is a common cancer treatment strategy. Frequently, this is accomplished through monoclonal antibodies targeting the ligand binding sites of inhibitory co-receptors. Blocking the immune checkpoint PD-1 binding to its ligands PD-L1 and PD-L2 prevents downstream signaling and enhances specific T functions. Since 2013 the FDA has approved nine monoclonal antibodies to inhibit PD-1 and PD-L1 signaling. This therapeutic approach significantly improved the care of patients with cancers. However, many patients are unresponsive, and some develop immune-related adverse events (irAEs). One reason for that is the lack of consideration for the localization of PD-1 on the surface of the immune cells in the context of the immune synapse.

The immune synapse is the interface between T cells and tumor cells. This interface, or microenvironment, includes the checkpoint receptors and other immune receptors and ligands needed for T cells to function. The immune synapse is organized into three compartments; every protein has a specific location. For example, the T cell receptor is localized to the center of the synapse, PD-1 and CD28 are located in the peripheral synapse, and LFA-1 and CD45 are found in the distal compartment of the synapse. This organization is critical for the function of the synapse, where specific clusters of proteins are formed.

Previous study has shown that monoclonal antibodies, due to their size (~140 kd), are too large to enter the immune synapse and, therefore, are excluded from it. Using scanning confocal imaging, we demonstrated that nivolumab and pembrolizumab (FDA-approved anti-PD-1 antibodies) were excluded from the immune synapse. But more interesting was that these antibodies, at least in some of the cells, removed their target, PD-1, away from the synapse. Removing PD-1 from the synapse prevented its inhibitory functions in several in vitro assays. Complementary to that, shorter versions of the same antibodies, such as Fab′ and Fab″, while able to bind to PD-1 with the same affinity as the full-length antibodies, were less potent in inhibiting PD-1 functions, suggesting that some of the mechanisms of the full-length antibodies were due to its size, in changing PD-1 location, an effect that the shorter version of the antibodies was not able to achieve.

Based on these findings, removing PD-1 from the immune synapse could serve as an alternative, efficient, and safer approach to treating cancer patients. To achieve that we designed improved versions of the anti-PD-1 antibody: anti-PD-1-Fc and anti-PD-1-CD22.

The rationale behind this approach is that disruption of PD-1 interactions through altered localization would serve as a strategy to affect T cell function. The PD-1 pathway provides a clear example of how changing a protein's localization on the cell surface can impact T cell function. Another advantage of removing PD-1 from the synapse is its ability to prevent PD-1 ligand-independent downstream tonic signaling.

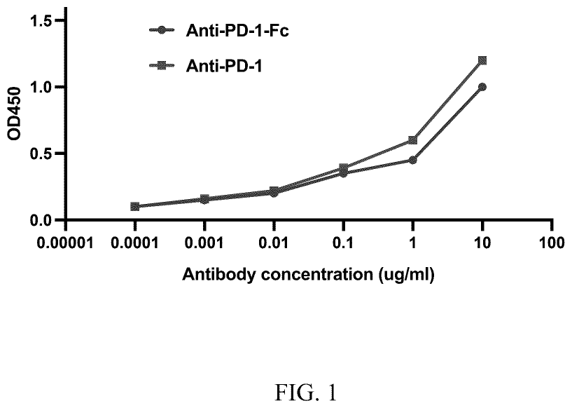

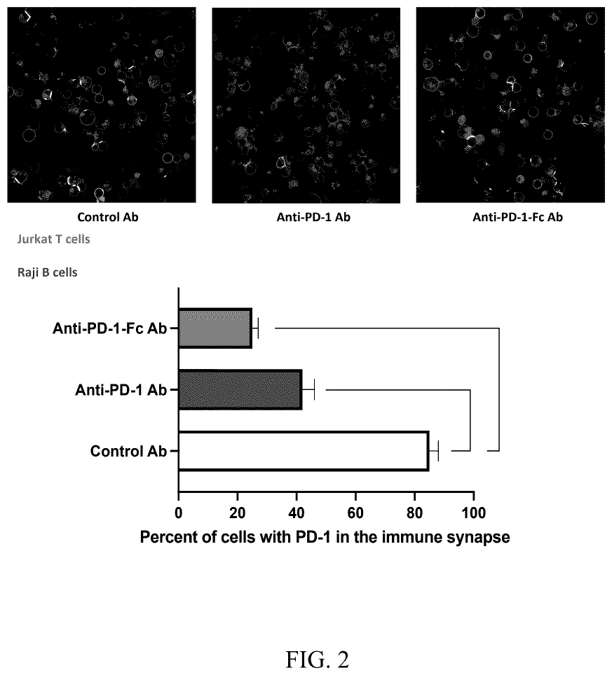

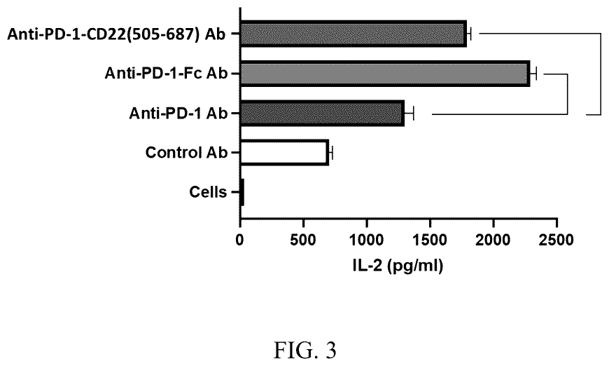

For anti-PD-1-Fc, another Fc domain was added to the Fc tail of pembrolizumab. FIG. 1 shows that anti-PD-1 antibody and anti-PD-1-FC antibody bind to PD-1. In the case of anti-PD-1-CD22, a CD22 domain was added to the Fc tail of pembrolizumab. Both antibodies are 200 kD. Both antibodies are excluded from the synapse and treating T cells that express PD-1 in the context of multicellular assay resulted in complete removal of PD-1 from the synapse (FIG. 2). In comparison to full length pembrolizumab, anti-PD-1-Fc had a complete and superior inhibition of PD-1 signaling and functions, leading to more cytokine secretion and improved primary T cell killing of tumor cells in vitro (FIG. 3).

To further support this concept, described herein shows a correlation between the degree of PD-1 exclusion from the synapse, the size of the antibody, and its lack of ability to prevent cell proliferation and cytokine secretion. More importantly, in a Jurkat/Raji coculture assay, described herein shows that the engineered antibodies can activate T cells better than the generic anti-PD-1 antibody. Ongoing experiments are designed to test these antibodies in vivo using syngeneic tumor model, in comparison to anti-PD-1 monoclonal antibodies.

Therefore, use of the 200 kD anti-PD-1-Fc has several advantaged over the canonical anti-PD-1 monoclonal antibodies: It prevents PD-1 interaction with its ligand, it removes PD-1 away from the synapse and prevents it from interacting with its downstream effectors, further neutralizing its activity. This approach offers an innovative, more potent, and safer technology to treat cancer patients resistant to current immunotherapies.

### Example 2—Anti-PD-1 Antibodies

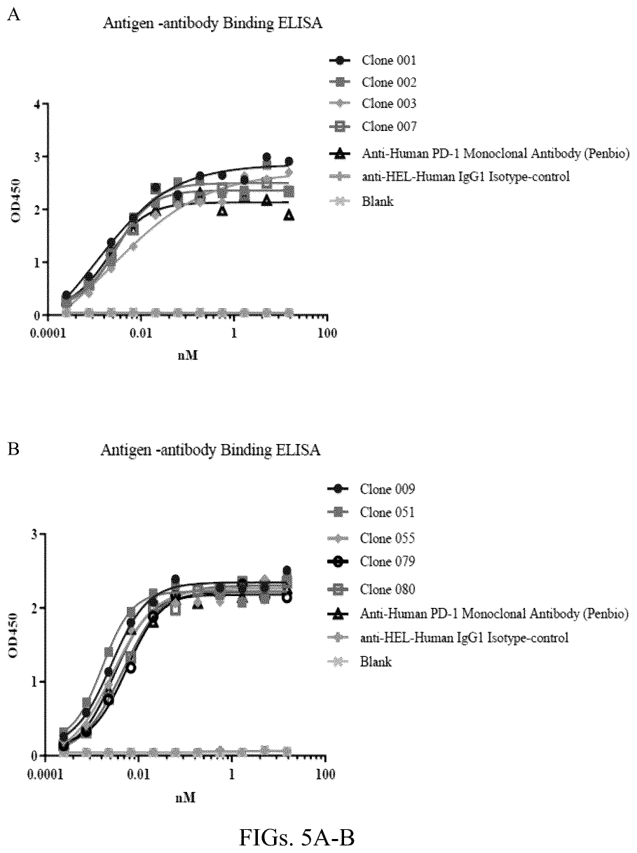

As shown in FIG. 5A-B, binding curves to human-PD-1-His-coated plates was quantified by ELISA. FIG. 5A shows binding curves for anti-PD1 antibody clones 01, 02, 03, and 07, anti-human PD-1 antibody Penbio (pembrolizumab), anti-HEL-human IgG1 isotype control, and blank. FIG. 5B shows binding curves for anti-PD1 antibody clones 09, 51, 55, 79, and 80, anti-human PD-1 antibody Penbio (pembrolizumab), anti-HEL-human IgG1 isotype control, and blank.

Table 1 shows EC50 values for anti-PD1 antibody clones 01, 02, 03, and 07, anti-human PD-1 antibody Penbio (pembrolizumab), anti-HEL-human IgG1 isotype control, and blank. Table 2 shows EC50 values for anti-PD1 antibody clones 09, 51, 55, 79, and 80, anti-human PD-1 antibody Penbio (pembrolizumab), anti-HEL-human IgG1 isotype control, and blank. EC50 values were calculated with GraphPad Prism (v10.2.1). These results show that the anti-PD1 antibody clones are capable of binding to PD-1, in some cases with lower or similar EC50 values when compared to the commercially available pembrolizumab.

TABLE 1

| Antibody | EC₅₀(nM) |
|---|---|
| Clone 01 | 0.001054 |
| Clone 02 | 0.003222 |
| Clone 03 | 0.002326 |
| Clone 07 | 0.002939 |
| Anti-Human PD-1 | 0.002 |
| Monoclonal Antibody | |
| (Penbio) | |
| anti-HEL-Human IgGl | No Binding |
| Isotype-control | |

TABLE 2

| Antibody | EC₅₀(nM) |
|---|---|
| Clone 09 | 0.002673 |
| Clone 51 | 0.00176 |
| Clone 55 | 0.003264 |
| Clone 79 | 0.006451 |
| Clone 80 | 0.004642 |
| Anti-Human PD-1 | 0.003025 |
| Monoclonal Antibody | |
| (Penbio) | |
| anti-HEL-Human IgGl | No Binding |
| Isotype-control | |

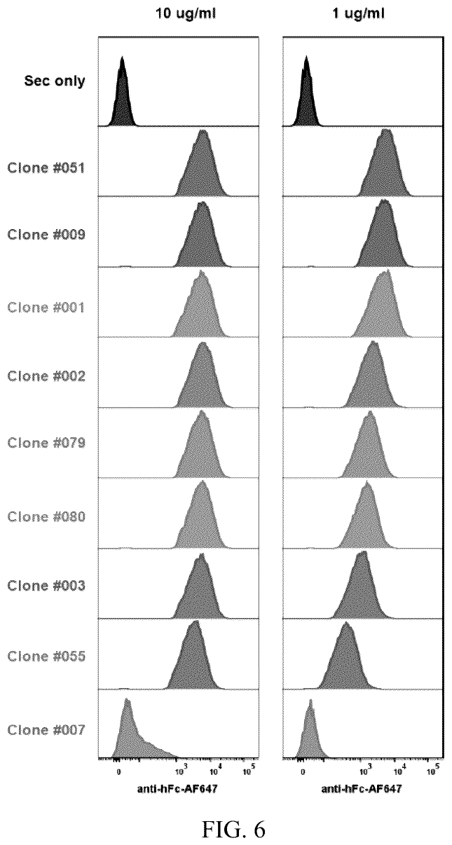

FIG. 6 shows binding of anti-PD-1 clones 01, 02, 03, 07, 09, 51, 55, 79, and 80 to cell-expressed PD-1 by flow cytometry. Jurkat T cells stably overexpressing human-PD-1 were incubated with 10 μg/ml (FIG. 6 left) or 1 μg/ml (FIG. 6 right) of each anti-PD-1 clone on ice for 30 minutes, washed twice, and then incubated for 30 minutes on ice with a fluorescent anti-human-Fc secondary antibody. Following two additional washes, cells were analyzed on a BD LSRFortessa™ flow cytometer. These results show that the anti-PD-1 clones bind to cell-expressed PD-1.

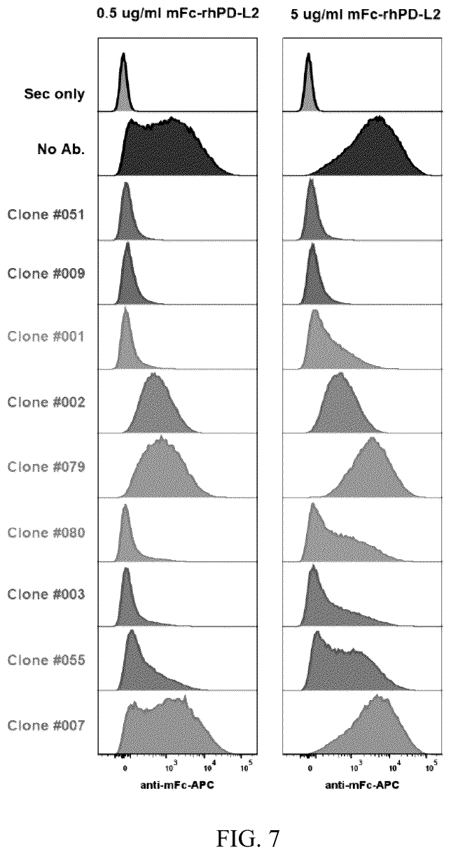

FIG. 7 shows anti-PD-1 clones 01, 02, 03, 07, 09, 51, 55, 79, and 80 blocking the binding of rhPD-L2 to cell-expressed PD-1. Jurkat T cells stably overexpressing human-PD-1 were incubated with 10 μg/ml of each anti-PD-1 clone on ice for 30 minutes, washed twice, and then incubated for 1 hour on ice with 0.5 g/ml (FIG. 7 left) or 5 μg/ml (FIG. 7 right) of recombinant human PD-L2 (with mouse-Fc). Following two additional washes, the cells were incubated for 30 minutes on ice with a fluorescent anti-mouse-Fc secondary antibody, washed twice, and analyzed on a BD LSRFortessa™ flow cytometer. These results show that the anti-PD-1 clones block the binding of rhPD-L2 to cell-expressed PD-1.

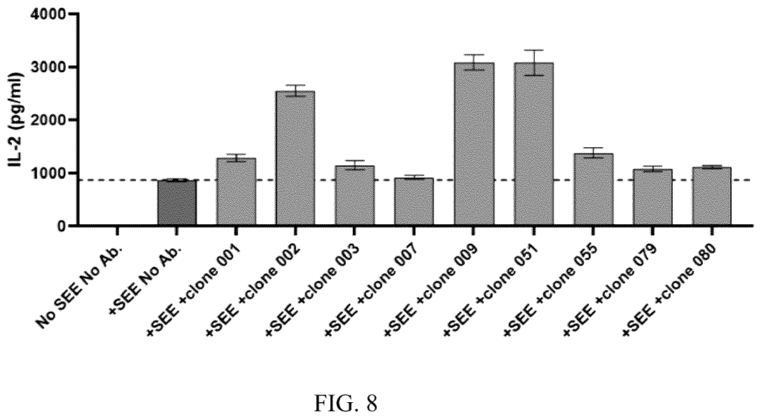

FIG. 8 shows IL-2 concentrations following addition of anti-PD-1 clones 01, 02, 03, 07, 09, 51, 55, 79, or 80 and SEE (Staphylococcal Enterotoxin E) in Jurkat-Raji co-culture compared to no SEE and no antibody control and SEE and no antibody control. Jurkat T cells stably overexpressing human-PD-1 and Raji B cells stably overexpressing human-PD-L1 were co-cultured for 24 hours in the presence of 10 μg/ml of each anti-PD-1 clone and 100 μg/ml SEE (Staphylococcal Enterotoxin E). IL-2 concentrations in the supernatants were determined by

ELISA. These results show that anti-PD-1 clones can increase IL-2 concentrations in the supernatants compared to no antibody controls.

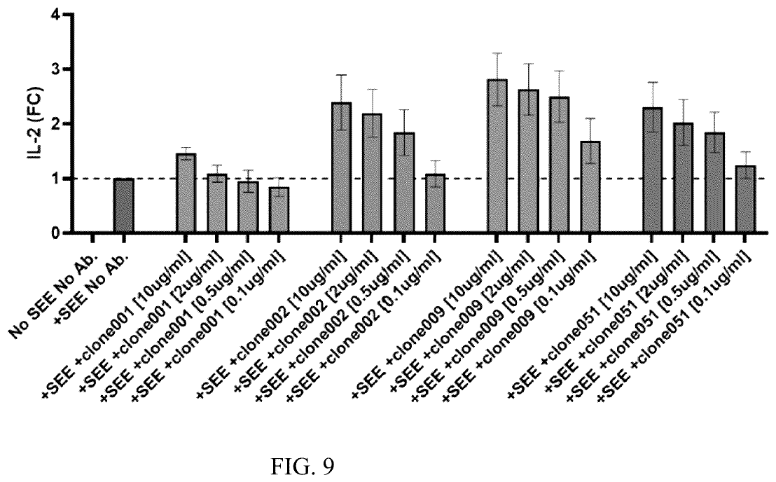

FIG. 9 shows IL-2 concentrations determined by ELISA following addition of anti-PD-1 clones 01, 02, 03, 07, 09, 51, 55, 79, or 80 at different concentrations (10, 2, 0.5 or 0.1 μg/ml) and SEE (Staphylococcal Enterotoxin E) in Jurkat-Raji co-culture compared to no SEE and no antibody control and SEE and no antibody control. Jurkat T cells stably overexpressing human-PD-1 and Raji B cells stably overexpressing human-PD-L1 were co-cultured for 24 hours in the presence of different concentrations (10, 2, 0.5 or 0.1 μg/ml) of each anti-PD-1 clone and 100 μg/ml SEE (Staphylococcal Enterotoxin E). These results show that anti-PD-1 clones can increase IL-2 concentrations in a dose dependent manner in the supernatants compared to no antibody controls.

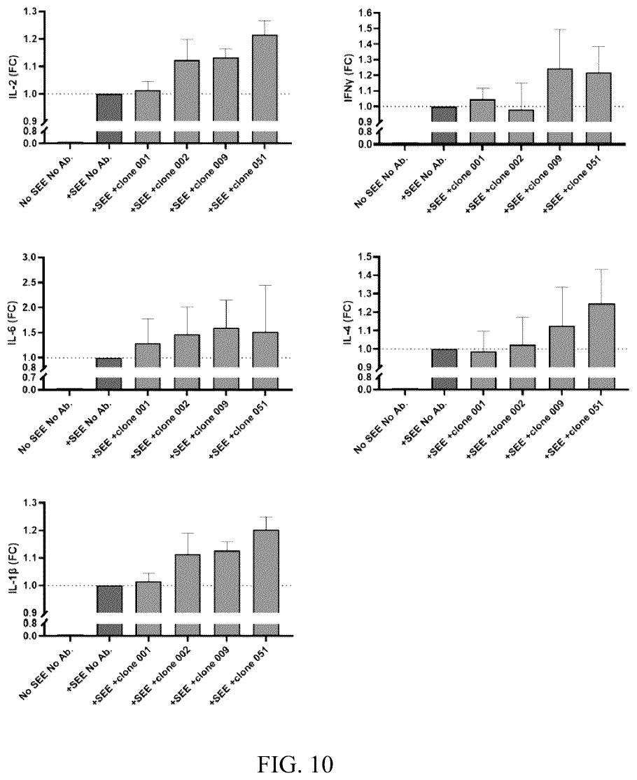

FIG. 10 shows concentrations of IL-2, IFNγ, IL-6, IL-4 and IL-1β determined by ELISA in PBMCs in the presence of anti-PD-1 clones 01, 02, 03, 07, 09, 51, 55, 79, or 80 and SEE (Staphylococcal Enterotoxin E). PBMCs from 3 healthy donors were incubated for 24 hours in the presence of 10 μg/ml of each anti-PD-1 clone and 100 μg/ml SEE (Staphylococcal Enterotoxin E). The concentrations of IL-2, IFNγ, IL-6, IL-4 and IL-1β in the supernatants were determined by ELISA. These results show that anti-PD-1 clones can increase IL-2, IFNγ, IL-6, IL-4 and IL-1β concentrations in the supernatants compared to no antibody controls.

### Example 3—Anti-PD-1-ABD and Anti-PD-1-HSA Antibodies

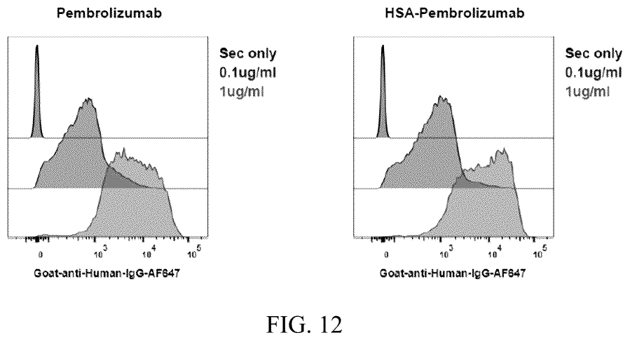

HEK-293T cells were transiently transfected with a human-PD-1-CFP expression vector. After 48 hours the cells were harvested and incubated with 0.1 μg/ml or 1 μg/ml of Pembrolizumab (left) or HSA-Pembrolizumab (right) on ice for 30 minutes, washed twice, and then incubated for 30 minutes on ice with a fluorescent anti-human-Fc secondary antibody. Following two additional washes, cells were analyzed on a BD LSRFortessa flow cytometer. FIG. 12 shows binding of Pembrolizumab (left) and HSA-Pembrolizumab (right) to cell-expressed PD-1 determined by flow cytometry. Histograms show CFP+ cells. These data show that HSA-Pembrolizumab binds to cell-expressed PD-1 was well as Pembrolizumab.

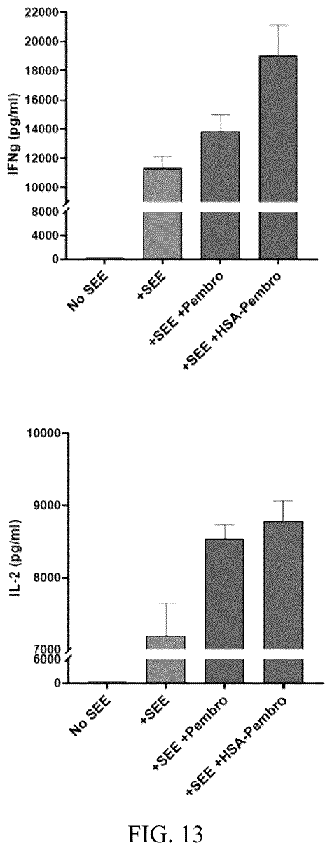

FIG. 13 shows IFNg (top) and IL-2 (bottom) concentrations determined by ELISA following addition of SEE (Staphylococcal Enterotoxin E), Pembrolizumab with SEE, HSA-Pembrolizumab with SEE, or no antibody and no SEE. For INFg concentrations, PBMCs were incubated for 24 hours in the presence of 10 μg/ml of Pembrolizumab or HSA-Pembrolizumab and 100 μg/ml SEE (Staphylococcal Enterotoxin E). For IL-2 concentrations, PBMCs were incubated for 24 hours in the presence of 1 μg/ml of Pembrolizumab or HSA-Pembrolizumab and 100 μg/ml SEE (Staphylococcal Enterotoxin E). The concentrations of IFNγ in the supernatants were determined by ELISA. These data show that HSA-Pembrolizumab produces higher level of IFNg and IL-2 concentrations than Pembrolizumab without the HSA domain.

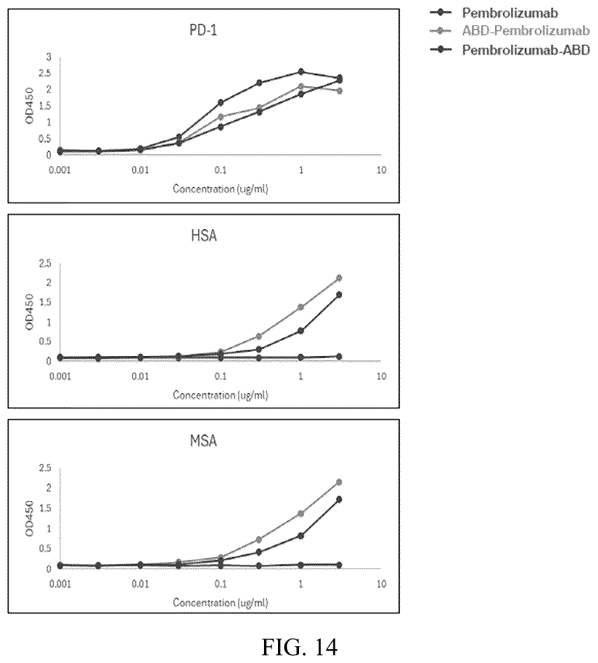

FIG. 14 shows binding determined by ELISA of Pembrolizumab, ABD-Pembrolizumab, and Pembrolizumab-ABD to PD-1 (top), human serum albumin (HSA) (middle), and mouse serum albumin (MSA) (bottom). ABD-Pembrolizumab is a fusion Ab with the ABD peptide at the N-terminus of the heavy chain of pembrolizumab, and Pembrolizumab-ABD is a similar fusion AB with the ABD peptide at the C-terminus of the heavy chain of pembrolizumab. Binding to plates coated with human-PD-1, human serum albumin (HSA), or mouse serum albumin (MSA). These data show that ABD-Pembrolizumab and Pembrolizumab-ABD result in stronger binding to HSA and MSA (but similar binding to PD-1) compared to Pembrolizumab without the ABD.

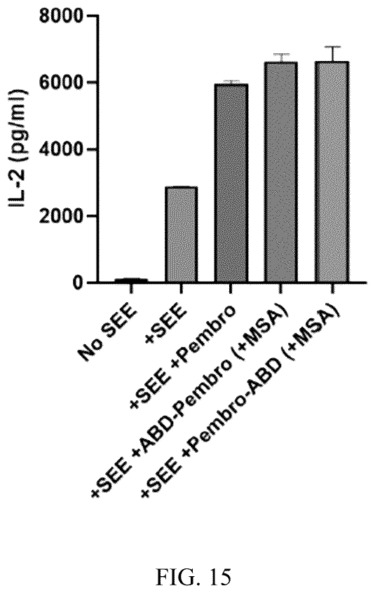

FIG. 15 shows IL-2 concentrations determined by ELISA following addition of SEE (Staphylococcal Enterotoxin E), Pembrolizumab with SEE, ABD-Pembrolizumab with SEE and MSA, Pembrolizumab-ABD with SEE and MSA, or no antibody and no SEE. PBMCs were incubated for 24 hours in the presence of 10 μg/ml of Pembrolizumab, ABD-Pembrolizumab, or Pembrolizumab-ABD, 50 μg/ml mouse serum albumin (MSA), and 100 μg/ml SEE (Staphylococcal Enterotoxin E). The concentrations of IL-2 in the supernatants were determined by ELISA. These data show that ABD-Pembrolizumab and Pembrolizumab-ABD tended to increase IL-2 concentrations compared to Pembrolizumab without the ABD.

### Example 4—In Vivo Treatment with Anti-PD1-AD

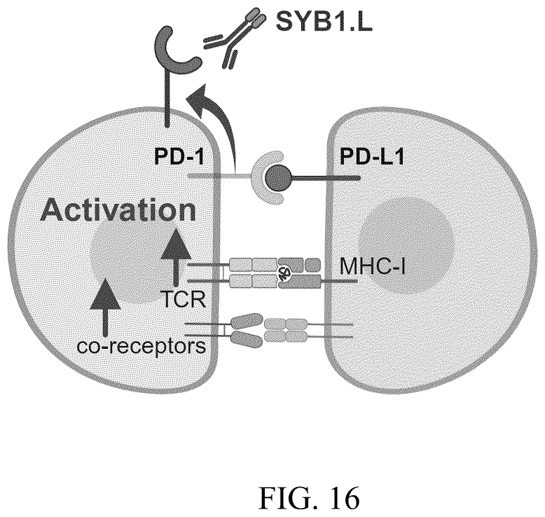

FIG. 16 shows a schematic of how anti-PD1-antibody (SYB1.L) affects PD-1 immune synapse. SYB1.L works via a novel mechanism of action and is a more potent anti-PD-1 antibody than Pembrolizumab

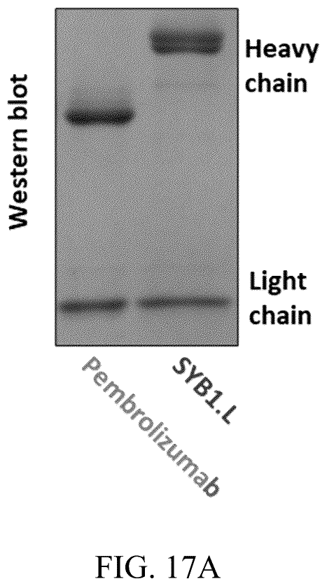

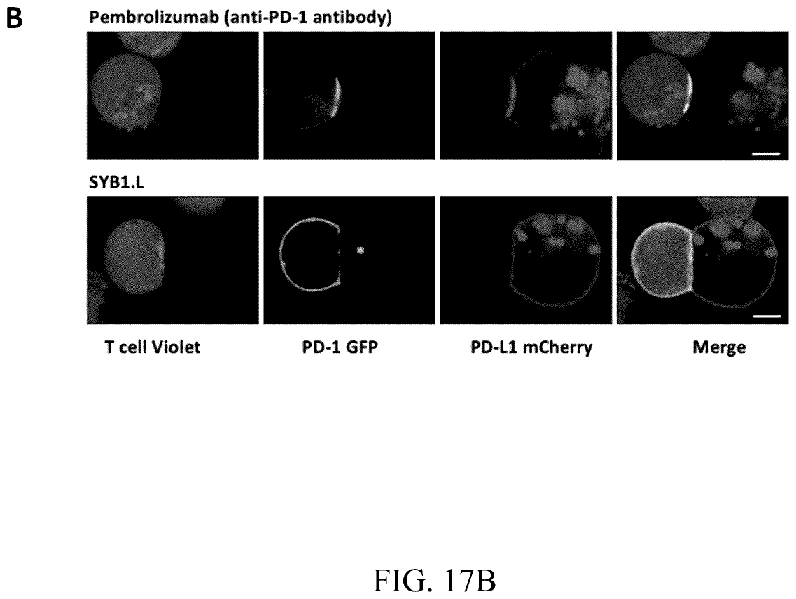

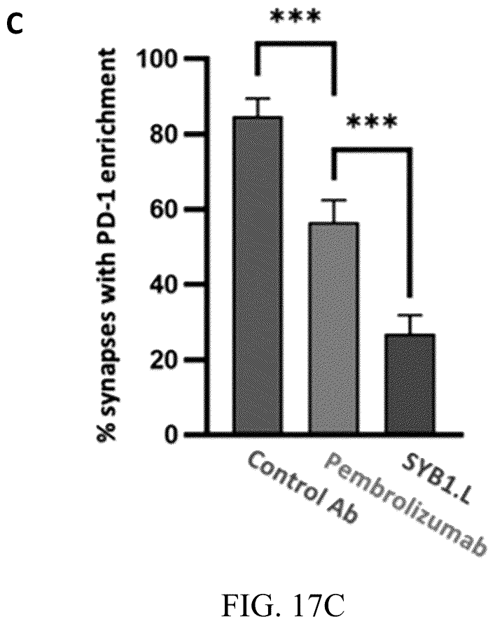

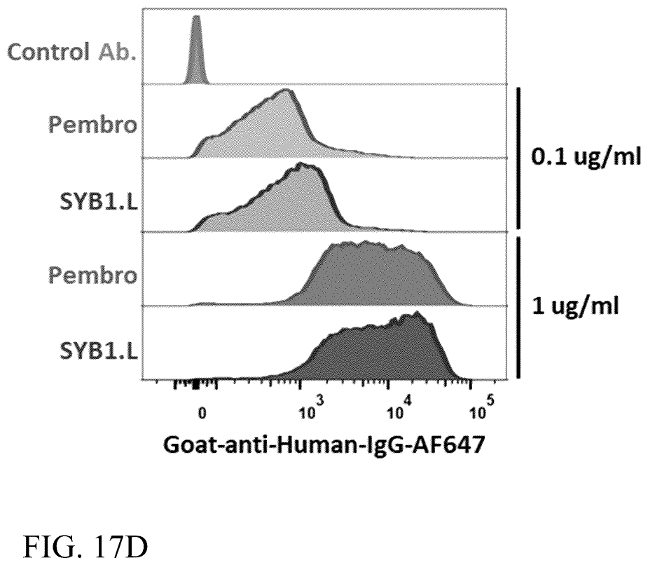

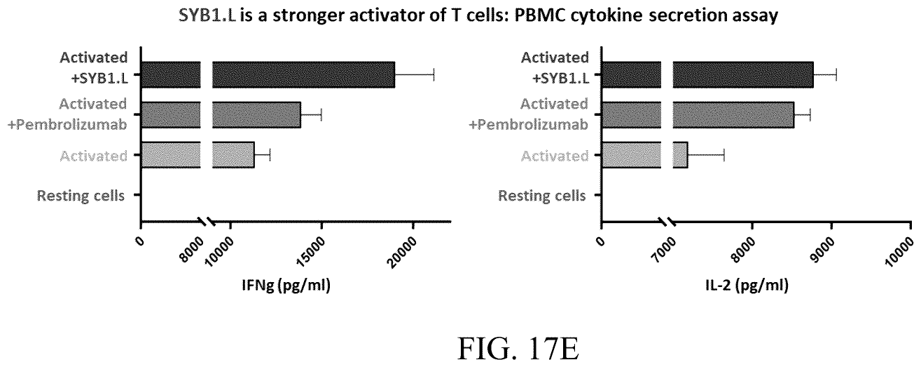

SYB1.L has a longer heavy chain than Pembrolizumab (FIG. 17A). SYB1.L removes the PD-1 receptor away from the immune synapse (FIG. 17B). Treatment with SYB1.L shows significantly decreases the % of synapses with PD-1 enrichment compared to control or Pembrolizumab treatment (FIG. 17C). SYB1.L binds to PD-1 on the surface of cells better than Pembrolizumab (FIG. 17D). SYB1.L is a stronger activator of T cells compared to Pembrolizumab (FIG. 17E).

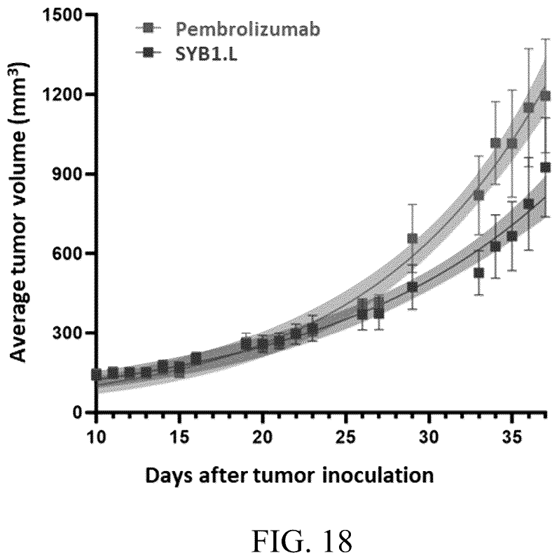

FIG. 18 shows that treatment with SYB1.L in vivo decreases H1299 lung tumor volume compared to treatment with Pembrolizumab.

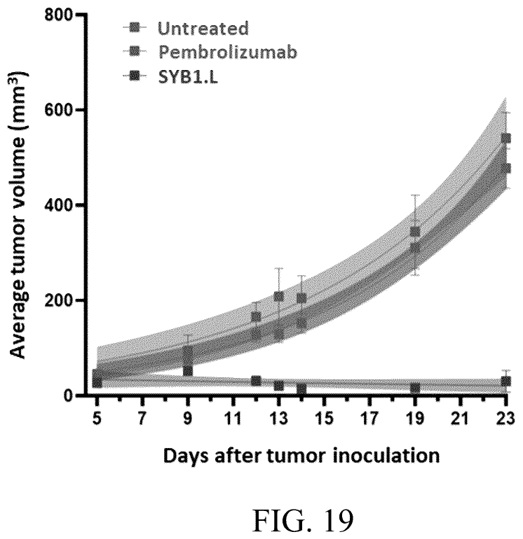

FIG. 19 shows that treatment with SYB1.L in vivo decreases E0771 breast tumor volume compared to treatment with Pembrolizumab. Notably, EO771 tumors are know to be resistant to conventional PD-1 inhibition (Pembrolizumab).

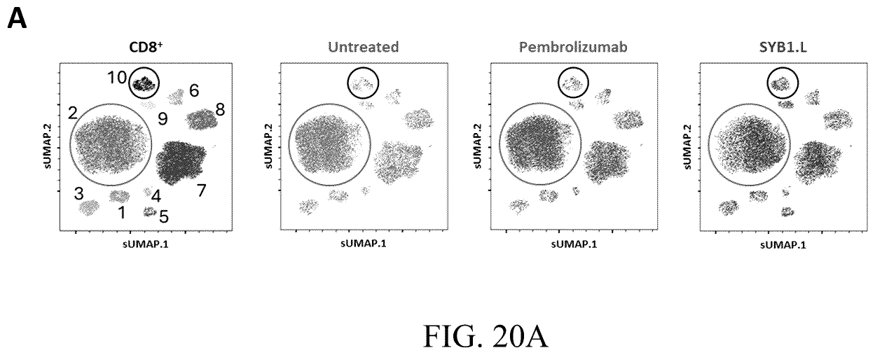

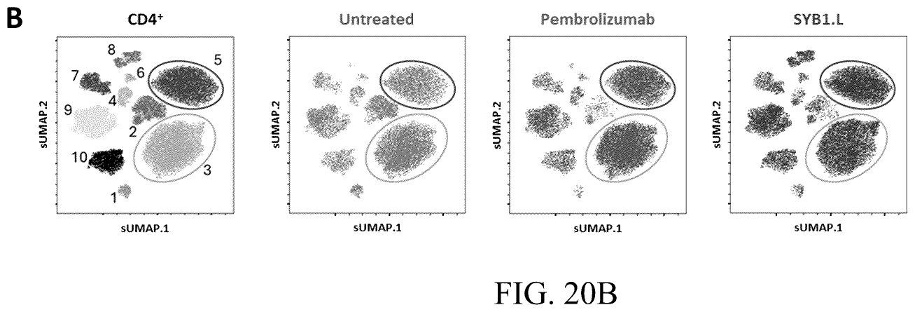

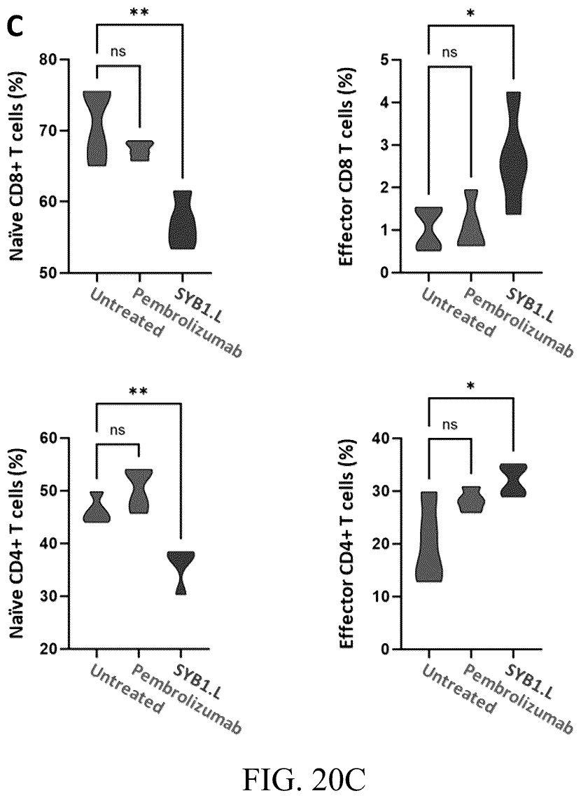

SYB1.L changes the distribution of T cells in human PD-1 B6 mice carrying MC38 tumors (FIG. 20A-B). Treatment with SYB1.L shows decreased naïve CD8+ T cells, increased effector CD8+ T cells, decreased naïve CD4+ T cells, and increased effector CD4+ T cells compared to treatment with Pembrolizumab or control treated mice (FIG. 20C).

### Example 5—In Vivo Efficacy of PD-1HSA Antibodies

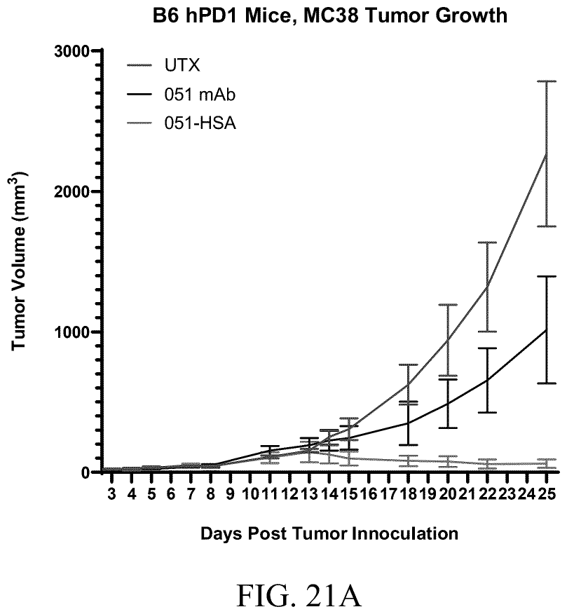

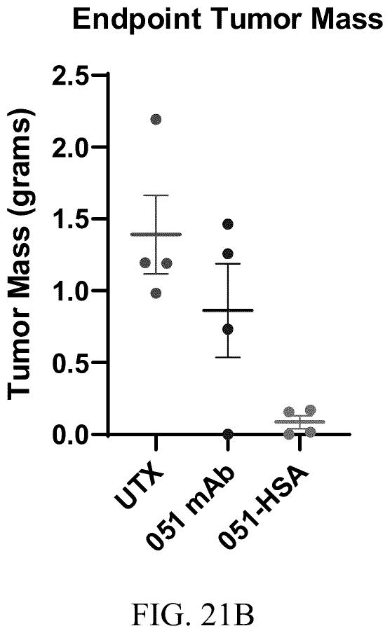

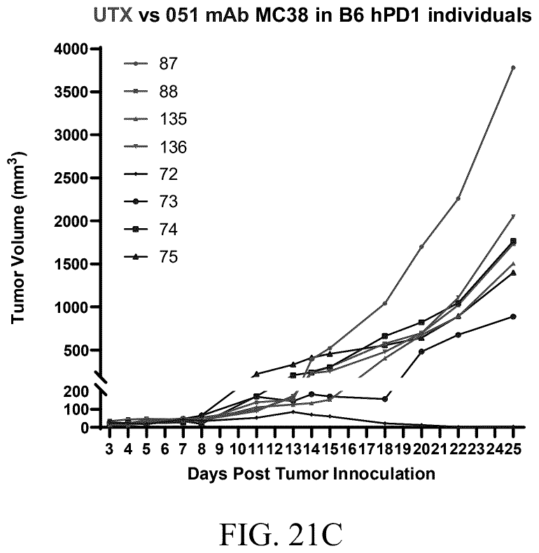

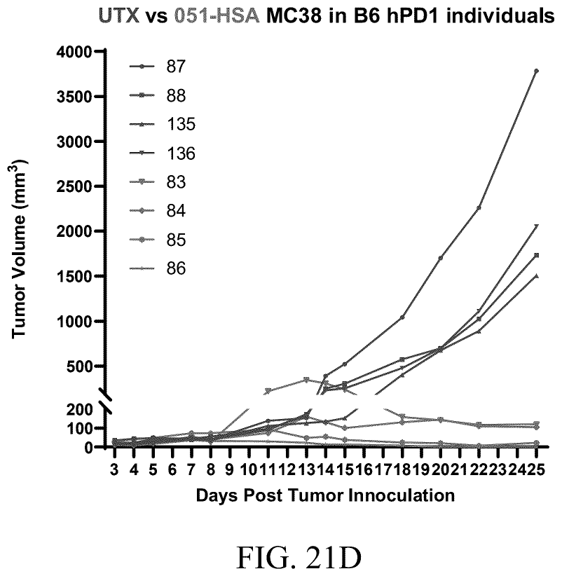

Clone 051-HSA antibodies reduces tumor volume in human PD-1 B6 mice carrying MC38 tumors (FIGS. 21A-D). Mice were treated with 051-HSA and compared to 051 mAb as well as untreated. Each mice received 4 doses of treatment total. The dosage amounts of 051-HSA and 051 mAb are matched by molar concentration (051-HSA 385 μg/dose; 051 mAb 200 μg/dose).

What is claimed is:

1. A monoclonal antibody or a fragment thereof, comprising:
a first arm comprising a first variable heavy chain domain and a first variable light chain domain, wherein a portion of the first arm is capable of binding to a portion of a PD-1 protein; and
a second arm comprising a second variable heavy chain domain and a second variable light chain domain, wherein a portion of the second arm is capable of binding to a portion of the PD-1 protein,
wherein the first arm and the second arm each further comprise a first fragment crystallizable (Fc) domain,
wherein the first arm and the second arm each further comprise at least one additional domain.

2. The monoclonal antibody of claim 1, wherein the first arm and the second arm each further comprise a CH1 domain, a hinge domain, and a CL domain.

3. The monoclonal antibody of any of claims 1-2, wherein the at least one additional domain of the first arm and the second arm comprises a second Fc domain.

4. The monoclonal antibody of claim 3, wherein the at least one additional domain of the first arm and the second arm comprises an albumin binding domain (ABD).

5. The monoclonal antibody of any of claims 1-2, wherein the at least one additional domain of the first arm and the second arm comprises a CD22 domain.

6. The monoclonal antibody of claim 5, wherein the at least one additional domain of the first arm and the second arm comprises a human serum albumin (HSA) domain.

7. The monoclonal antibody of claim 5, wherein the CD22 domain comprises SEQ ID NO: 8, SEQ ID NO: 9, or SEQ ID NO: 10, or a fragment thereof.

8. The monoclonal antibody of claim 4, wherein the ABD domain comprises any one of SEQ ID NOs: 62-71, or a fragment thereof.

9. The monoclonal antibody of claim 6, wherein the HSA domain comprises any one of SEQ ID NOs: 51-60, or a fragment thereof.

10. The monoclonal antibody of any of claims 1-7, wherein:
the first variable heavy chain domain of the first arm is encoded by a first polypeptide chain,
the first variable light chain domain of the first arm is encoded by a second polypeptide chain,
the second variable heavy chain domain of the second arm is encoded by a third polypeptide chain,
the second variable light chain domain of the second arm is encoded by a fourth polypeptide chain,
the first variable heavy chain domain and the first variable light chain domain form a first PD-1 binding site, and
the second variable heavy chain domain and the second variable light chain domain form a second PD-1 binding site.

11. The monoclonal antibody of claim 10, wherein the first PD-1 binding site and the second PD-1 binding site are the same.

12. The monoclonal antibody of claim 10, wherein the first polypeptide chain and the third polypeptide chain each further encode a hinge domain, a CH1 domain, the Fc domain, and the at least one additional domain, and wherein the second polypeptide chain and the fourth polypeptide chain each further encode a CL domain.

13. The monoclonal antibody of claim 12, wherein the first polypeptide chain and the third polypeptide chain comprise the same sequence, and wherein the second polypeptide chain and the fourth polypeptide chain comprise the same sequence.

14. The monoclonal antibody of any of claims 1-13, wherein the first variable heavy chain domain comprises an amino acid sequence of the variable heavy chain portion of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74 wherein the first variable light chain domain comprises an amino acid sequence of the variable light chain portion of SEQ ID NO: 3, 15, 17, 19, 21, 23, 25, 27, 29, 31, 73, or 75.

15. The monoclonal antibody of any of claims 1-13, wherein the first arm and the second arm each comprise an amino acid sequence comprising SEQ ID NO: 2 and SEQ ID NO: 3.

16. The monoclonal antibody of any of claims 1-13, wherein the first arm and second arm each comprise an amino acid sequence comprising SEQ ID NO: 11 and SEQ ID NO: 3.

17. The monoclonal antibody of any of claims 1-13, wherein the first arm and the second arm each comprise an amino acid sequence comprising SEQ ID NO: 12 and SEQ ID NO: 3.

18. The monoclonal antibody of claim 10, wherein the first polypeptide chain and the third polypeptide chain each comprise an amino acid sequence comprising SEQ ID NO: 2 and the second polypeptide chain and the fourth polypeptide chain each comprises an amino acid sequence comprising SEQ ID NO: 3.

19. The monoclonal antibody of claim 10, wherein the first polypeptide chain and the third polypeptide chain each comprise an amino acid sequence comprising SEQ ID NO: 11 and the second polypeptide chain and the fourth polypeptide chain each comprise an amino acid sequence comprising SEQ ID NO: 3.

20. The monoclonal antibody of claim 10, wherein the first polypeptide chain and the third polypeptide chain each comprises an amino acid sequence comprising SEQ ID NO: 12 and the second polypeptide chain and the fourth polypeptide chain each comprises an amino acid sequence comprising SEQ ID NO: 3.

21. The monoclonal antibody of claim 10, wherein the first polypeptide chain and the third polypeptide chain each comprises an amino acid sequence comprising SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74 and the second polypeptide chain and the fourth polypeptide chain each comprises an amino acid sequence comprising SEQ ID NO: 3, 15, 17, 19, 21, 23, 25, 27, 29, 31, 73, or 75.

22. The monoclonal antibody of any of claims 10-21, wherein the first polypeptide chain and the second polypeptide chain are linked by one or more covalent disulfide bonds and the third polypeptide chain and the fourth polypeptide chain are linked by one or more covalent disulfide bonds.

23. The monoclonal antibody of any of claims 10-22, wherein the first polypeptide chain and the third polypeptide chain are linked by one or more covalent disulfide bonds.

24. The monoclonal antibody of any of claims 1-23, wherein the monoclonal antibody is capable of localizing the PD-1 protein away from an immune synapse.

25. The monoclonal antibody of claim 24, wherein the monoclonal antibody is capable of disrupting a downstream signaling of a PD-1 mediated response in a T cell.

26. The monoclonal antibody of claim 25, wherein the monoclonal antibody is capable of enhancing a T cell function.

27. The monoclonal antibody of claim 26, wherein the PD-1 protein is located on a T cell, and wherein the monoclonal antibody is capable of preventing the PD-1 protein from binding to a PDL-1 protein or a PDL-2 protein on a tumor cell.

28. The monoclonal antibody of claim 27, wherein the monoclonal antibody is capable of inducing a cytokine secretion in a T cell.

29. The monoclonal antibody of claim 28, wherein the cytokine secretion is a secretion of IL-2.

30. The monoclonal antibody of claim 29, wherein the monoclonal antibody is larger than 140 kD in size.

31. The monoclonal antibody of claim 29, wherein the monoclonal antibody is about 160 kD to about 230 kD in size.

32. A pharmaceutical composition comprising: the monoclonal antibody of any of claims 1-31; and a pharmaceutically acceptable carrier.

33. A method of preventing or treating cancer in a subject comprising administering to the subject an effective amount of the composition of claim 32.

34. The method of claim 33, wherein the cancer is selected from colorectal cancer, lung cancer, bladder cancer, breast cancer, cervical cancer, kidney cancer, leukemia, Hodgkin lymphoma, non-Hodgkin lymphoma, prostate cancer, skin cancer (e.g., melanoma), head and neck cancer, endometrial cancer, colon cancer, rectal cancer, liver cancer, thyroids cancer, esophageal cancer, renal cell cancer, and a combination thereof.

35. A method of preventing or treating a viral infection in a subject comprising administering to the subject an effective amount of the composition of claim 32.

36. The method of claim 35, wherein the viral infection is HIV.

37. A kit for generating a monoclonal antibody or fragment thereof, the kit comprising one or more vectors comprising a polynucleotide sequence encoding any of the monoclonal antibodies of any of claims 1-31.

38. A kit for generating a monoclonal antibody or fragment thereof, the kit comprising:
a first vector comprising a polynucleotide sequence encoding the first polypeptide chain of the monoclonal antibody of any of claims 10-21; and
a second vector comprising a polynucleotide sequence encoding the second polypeptide chain of the monoclonal antibody of any of claims 10-21.

39. The kit of claim 38, wherein the first vector and the second vector are the same vector.

40. The kit of claim 38, wherein the first vector and the second vector are two different vectors.

41. One or more host cells comprising: one or more vectors comprising a polynucleotide sequence encoding any of the monoclonal antibodies of any of claims 1-31.

42. One or more host cells comprising:
a first vector comprising a polynucleotide sequence encoding the first polypeptide chain of the monoclonal antibody of any of claims 10-21; and
a second vector comprising a polynucleotide sequence encoding the second polypeptide chain of the monoclonal antibody of any of claims 10-21.

43. The one or more host cells of claim 42, wherein the first vector and the second vector are the same vector.

44. The one or more host cells of claim 42, wherein the first vector and the second vector are two different vectors.

45. A method of making a monoclonal antibody or fragment thereof comprising:
culturing the one or more host cells of claim 42 under conditions suitable for an expression of the one or more vectors; and
recovering the monoclonal antibody or fragment thereof.

46. A method of making a monoclonal antibody or fragment thereof comprising:
culturing the one or more host cells of any of claims 42-44 under conditions suitable for an expression of the first vector and the second vector; and
recovering the monoclonal antibody or fragment thereof.

47. A composition comprising: one or more vectors comprising a polynucleotide sequence encoding any of the monoclonal antibodies of any of claims 1-31.

48. A composition comprising:
a first vector comprising a polynucleotide sequence encoding the first polypeptide chain of the monoclonal antibody of any of claims 10-21; and
a second vector comprising a polynucleotide sequence encoding the second polypeptide chain of the monoclonal antibody of any of claims 10-21.

49. The composition of claim 48, wherein the first vector and the second vector are the same vector.

50. The composition of claim 48, wherein the first vector and the second vector are two different vectors.

51. A means for binding a portion of a PD-1 protein.

52. The means of claim 51, wherein the means comprises a monoclonal antibody comprising a domain for increasing a size of the monoclonal antibody over 140 kD.

53. The means of claim 52, wherein the monoclonal antibody or a fragment thereof comprises:
a first arm comprising a first variable heavy chain domain and a first variable light chain domain, wherein a portion of the first arm is capable of binding to the portion of the PD-1 protein; and
a second arm comprising a second variable heavy chain domain and a second variable light chain domain, wherein a portion of the second arm is capable of binding to the portion of the PD-1 protein,
wherein the first arm and the second arm each further comprise a fragment, crystallizable (Fc) domain,
wherein the first and second arms each further comprise the domain for increasing the size of the monoclonal antibody over 140 kD.

54. The means of claim 53, wherein the first arm and the second arm each further comprise a CH1 domain, a hinge domain, and a CL domain.

55. The means of any of claims 53-54, wherein the domain for increasing the size of the monoclonal antibody over 140 kD comprises a second Fc domain.

56. The means of claim 53, wherein the domain for increasing the size of the monoclonal antibody over 140 kD comprises a CD22 domain, an albumin binding domain (ABD), or a human serum albumin (HAS) domain.

57. The means of claim 56, wherein the CD22 domain comprises SEQ ID NO: 8, SEQ ID NO: 9, or SEQ ID NO: 10, or a fragment thereof.

58. The means of claim 56, wherein the ABD domain comprises any one of SEQ ID NOs: 62-71, or a fragment thereof.

59. The means of claim 56, wherein the HSA domain comprises any one of SEQ ID NOs: 51-60, or a fragment thereof.

60. The means of any of claims 53-59, wherein:
the first variable heavy chain domain of the first arm is encoded by a first polypeptide chain,
the first variable light chain domain of the first arm is encoded by a second polypeptide chain,
the second variable heavy chain domain of the second arm is encoded by a third polypeptide chain,
the second variable light chain domain of the second arm is encoded by a fourth polypeptide chain,
the first variable heavy chain domain and the first variable light chain domain form a first PD-1 binding site, and
the second variable heavy chain domain and the second variable light chain domain form a second PD-1 binding site.

61. The means of claim 60, wherein the first PD-1 binding site and the second PD-1 binding site are the same.

62. The means of claim 61, wherein the first polypeptide chain and the third polypeptide chain each further encode a hinge domain, a CH1 domain, the Fc domain, and the at least one additional domain for increasing the size of the monoclonal antibody over 140 kD, and wherein the second polypeptide chain and the fourth polypeptide chain each further encode a CL domain.

63. The means of claim 62, wherein the first polypeptide chain and the third polypeptide chain comprise the same sequence, and the second polypeptide chain and the fourth polypeptide chain comprise the same sequence.

64. The means of any of claims 53-63, wherein the first variable heavy chain domain comprises an amino acid sequence of the variable heavy chain portion of SEQ ID NO: 2, 11, 12, 14, 16, 18, 20, 22, 24, 26, 28, 30, 32, 33, 34, 35, 36, 37, 38, 39, 40, 41, 42, 43, 44, 45, 46, 47, 48, 49, 51, 52, 53, 54, 55, 56, 57, 58, 59, 60, 62, 63, 64, 65, 66, 67, 68, 69, 70, 71, 72, or 74 and wherein the first variable light chain domain comprises an amino acid sequence of the variable light chain portion of SEQ ID NO: 3, 15, 17, 19, 21, 23, 25, 27, 29, 31, 73, or 75.

65. The means of and of claims 53-64, wherein the first arm and the second arm each comprise an amino acid sequence comprising SEQ ID NO: 2 and SEQ ID NO: 3.

66. The means of any of claims 53-64, wherein the first arm and the second arm each comprise an amino acid sequence comprising SEQ ID NO: 11 and SEQ ID NO: 3.

67. The means of any of claims 53-64, wherein the first arm and the second arm each comprise an amino acid sequence comprising SEQ ID NO: 12 and SEQ ID NO: 3.

68. The means of any of claims 51-67, wherein the monoclonal antibody is capable of localizing a PD-1 protein away from an immune synapse.

69. The means of claim 68, wherein the monoclonal antibody is capable of disrupting a downstream signaling of a PD-1 mediated response in a T cell.

70. The means of claim 69, wherein the monoclonal antibody is capable of enhancing T cell function.

71. The means of claim 70, wherein the PD-1 protein is located on a T cell, and wherein the monoclonal antibody is capable of preventing the PD-1 protein from binding to a PDL-1 protein or a PDL-2 protein on a tumor cell.

72. The means of claim 71, wherein the monoclonal antibody is capable of inducing a cytokine secretion in a T cell.

73. The means of claim 72, wherein the cytokine secretion is a secretion of IL-2.

74. The means of claim 73, wherein the monoclonal antibody is about 160 kD to about 220 kD in size.
